SEQUENCE LISTING (9 sequences)

SEQ ID NO: 1 - protein, 394 aa, Artificial Sequence
  Synthetic polypeptide
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 2 - protein, 381 aa, Artificial Sequence
  Synthetic polypeptide
HMGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 3 - protein, 5 aa, Artificial Sequence
  Synthetic polypeptide
IWGTL

SEQ ID NO: 4 - protein, 5 aa, Artificial Sequence
  Synthetic polypeptide
IWGEL

SEQ ID NO: 5 - protein, 414 aa, Artificial Sequence
  Synthetic polypeptide
MGSSHHHHHHSGMSGLNDIFEAQKIEWHESSGENLYFQGMSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 6 - protein, 392 aa, Artificial Sequence
  Synthetic polypeptide
AHMGLNDIFEAQKIEWHESKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 7 - protein, 392 aa, Artificial Sequence
  Synthetic polypeptide
AHMGLNDIFEAQKIEWHESKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKLIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 8 - protein, 371 aa, Artificial Sequence
  Synthetic polypeptide
AHMGLNDIFEAQKIEWHEGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF

SEQ ID NO: 9 - protein, 71 aa, Artificial Sequence
  Synthetic polypeptide
MASNNTASIAQARKLVEQLKMEANIDRIKVSKAAADLMAYCEAHAKEDPLLTPVPASENPFREKKFFCAIL

(19) United States
(12) Patent Application Publication
(10) Pub. No.: US 2024/0336657 A1
(43) Pub. Date: Oct. 10, 2024

(54) G-ALPHA-S PEPTIDE INHIBITORS AND USES THEREOF

(71) Applicants: The Regents of the University of California, Oakland, CA (US); The University of Tokyo, Tokyo (JP)

(72) Inventors: Kevan M. Shokat, San Francisco, CA (US); Qi Hu, Hangzhou (CN); Shizhong Dai, San Francisco, CA (US); Hiroaki Suga, Tokyo (JP); Rong Gao, Shanghai (CN); Hayden Peacock, Tokyo (JP)

(21) Appl. No.: 18/287,637

(22) PCT Filed: Apr. 26, 2022
(86) PCT No.: PCT/US2022/026346

§ 371 Date: Oct. 19, 2023

Related U.S. Application Data
(60) Provisional application No. 63/179,958, filed on Apr. 26, 2021.

(51) Int. Cl. C07K 7/56; A61K 38/00; C07K 7/64

(52) U.S. Cl. CPC C07K 7/56; C07K 7/64; A61K 38/00

(57) ABSTRACT

Described herein, inter alia, are Gas peptide inhibitors and uses thereof.

### CROSS-REFERENCES TO RELATED APPLICATIONS

This application claims the benefit of U.S. Provisional Application No. 63/179,958, filed Apr. 26, 2021, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. R01 CA244550 awarded by The National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-708001WO_Sequence_Listing_ST25.TXT, created Apr. 19, 2022, 22,109 bytes, machine format IBM-PC, MS Windows operating system, is hereby incorporated by reference.

### BACKGROUND

The GNAS gene encodes the Gas stimulatory subunit of heterotrimeric G proteins, which mediate G-protein-coupled receptor (GPCR) signaling, a central mechanism by which cells sense and respond to extracellular stimuli. Multiple human cancer types exhibit recurrent gain-of-function mutations in the pathway, most frequently targeting GNAS. The most lethal tumor type where GNAS is frequently mutated is the intraductal papillary mucinous neoplasm (IPMN), a precursor of invasive pancreatic cancer. Disclosed herein, inter alia, are solutions to these and other problems in the art.

### BRIEF SUMMARY

In an aspect is provided a compound having the formula:

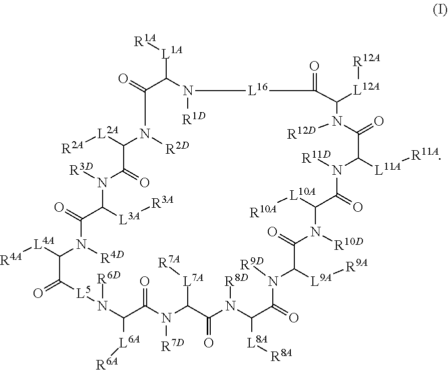

L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene, or substituted or unsubstituted heteroalkylene.

L⁵ is

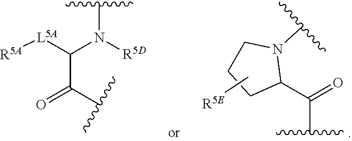

R^{1A} is substituted or unsubstituted aryl.

R^{2A} and R^{5A} are independently hydrogen, —OH, —NH₂, substituted or unsubstituted alkyl, substituted or unsubstituted cycloalkyl, substituted or unsubstituted aryl, or substituted or unsubstituted heteroaryl.

R^{3A}, R^{4A}, and R^{11A} are independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

R^{6A} is —NH₂, —CONH₂, substituted or unsubstituted alkyl, substituted or unsubstituted heteroalkyl, or substituted or unsubstituted aryl.

R^{7A}, R^{8A}, and R^{12A} are independently hydrogen, substituted or unsubstituted alkyl, substituted or unsubstituted cycloalkyl, substituted or unsubstituted aryl, or substituted or unsubstituted heteroaryl.

R^{9A} is substituted or unsubstituted cycloalkyl, substituted or unsubstituted heterocycloalkyl, substituted or unsubstituted aryl, or substituted or unsubstituted heteroaryl.

R^{10A} is hydrogen or substituted or unsubstituted alkyl.

R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₈ alkyl.

R^{5E} is hydrogen, —OH, —NH₂, substituted or unsubstituted alkyl, or substituted or unsubstituted heteroalkyl.

L¹⁶ is a covalent linker.

In an aspect is provided a compound having the formula:

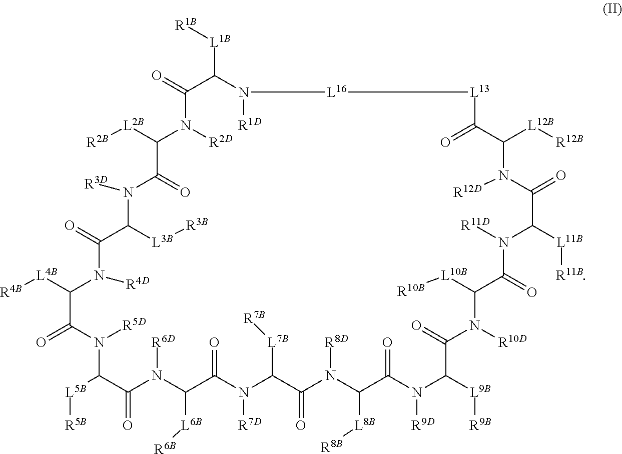

R^{1D}, R^{2D}, R^{3D}, R^{4D}, R^{5D}, R^{6D}, R^{7D}, R^{8D}, R^{9D}, R^{10D}, R^{11D}, R^{12D}, and L¹⁶ are as described herein, including in embodiments.

L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene.

L¹³ is a bond,

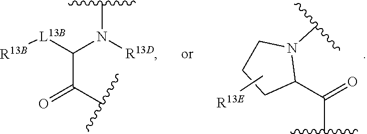

R^{1B} is substituted or unsubstituted aryl.

R^{2B}, R^{4B}, R^{5B}, R^{8B}, R^{9B}, and R^{13B} are independently hydrogen, —OH, —NH₂, —C(O)OH, —C(O)NH₂, —NO₂, —SO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{3B} is hydrogen, —OH, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl.

R^{6B}, R^{7B}, R^{10B}, R^{1B}, and R^{12B} are independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{13D} is independently hydrogen or unsubstituted C₁-C₄ alkyl.

R^{13E} is hydrogen, —OH, —NH₂, substituted or unsubstituted alkyl, substituted or unsubstituted heteroalkyl.

In an aspect is provided a pharmaceutical composition including a compound described herein, or a pharmaceutically acceptable salt thereof, and a pharmaceutically acceptable excipient.

In an aspect is provided a method of treating a cancer in a patient in need of such treatment, the method including administering a therapeutically effective amount of a compound described herein, or a pharmaceutically acceptable salt thereof, to the patient.

In an aspect is provided a method of treating a bone condition in a patient in need of such treatment, the method including administering a therapeutically effective amount of a compound described herein, or a pharmaceutically acceptable salt thereof, to the patient.

In an aspect is provided a method of treating McCune-Albright syndrome in a patient in need of such treatment, the method including administering a therapeutically effective amount of a compound described herein, or a pharmaceutically acceptable salt thereof, to the patient.

In an aspect is provided a method of treating cholera in a patient in need of such treatment, the method including administering a therapeutically effective amount of a compound described herein, or a pharmaceutically acceptable salt thereof, to the patient.

In an aspect is provided a method of treating a G protein-associated disease in a patient in need of such treatment, the method including administering a therapeutically effective amount of a compound described herein, or a pharmaceutically acceptable salt thereof, to the patient.

In an aspect is provided a method of modulating (e.g., reducing) the activity of a human Gαs protein, the method including contacting the human Gαs protein with an effective amount of a compound described herein, or a pharmaceutically acceptable salt thereof.

### BRIEF DESCRIPTION OF THE DRAWINGS

FIGS. 1A-1B. WT Gαs targeting cyclic peptides do not inhibit the R201C mutant in the assay tested.

FIG. 2. Summary of the R201C focused selection. Two families of sequences are observed in the enriched library: (1) IWGTL (SEQ ID NO: 3) motif (present in GN13) is conserved; and (2) IWGEL (SEQ ID NO: 4) motif is present. One negatively-charged residue is maintained in all new sequences, but its position has changed from GN13.

FIG. 3. Preliminary test of R201C-GDP specific peptides.

FIG. 4. GR6 potently inhibits Gαs in the presence of Gβγ.

FIG. 5. A close-up view of the GN13-WT Gαs/GNP binding pocket. Gαs S275 residue shown.

FIGS. 6A-6B. GR6 inhibits R201H/C and WT Gαs in the presence of Gβγ. The inhibition is blocked by S275L mutation in the cyclic peptide binding site in the assay tested.

FIGS. 7A-7B. GR6 has increased binding to R201C Gαs/GDP.

FIGS. 8A-8B. GR6 structure-activity relationship studies.

FIGS. 9A-9B. GR6 structure-activity relationship studies tested with a lower concentration of Gαs.

FIGS. 10A-10B. FIG. 10A: Structures of selected GN13 derivatives. FIG. 10B: Normalized fluorescence vs. concentration of GN13 peptide derivatives. Assay conditions: 8 hours of drug incubation at 37° C. without FBS.

FIGS. 11A-11C. Crystal Structure of GppNHp-bound Gαs in complex with GN13. FIG. 11A: Left: Overall structure of the GN13/GppNHp-bound Gαs complex. GN13 binds in between the switch II region and the α3 helix. GN13 and GppNHp are shown as sticks. Right: Structural details of the GN13-Gαs interaction. Hydrogen bonds are represented by 5 dashed lines. FIG. 11B: Close-up view of two hydrophobic pockets that accommodate the tryptophan and isoleucine side chains of GN13. Residues that form those pockets are depicted as stick models and labeled. FIG. 11C: Structural basis for nucleotide-state-selective binding of GN13 to Gαs. In GDP-bound Gαs, switch II is partially disordered, which disrupts polar contacts with GN13 and creates extensive steric hindrance. In particular, R232 of switch II (shown in space filling) is in a restrictive position relative to 18 of GN13.

FIGS. 12A-12B. GDP-state selective cyclic peptides inhibit Gαs steady-state GTPase activity.

FIGS. 13A-13B. GD20 decreased the GDP-GTP exchange rate to inhibit Gαs activation.

FIG. 14. Crystal structure of the Gαs/GDP/GD20 complex.

FIG. 15. GD20 stabilizes the switch II of Gαs in an inactive conformation and blocks the interaction between Gαs and Gβγ.

FIGS. 16A-16G. RaPID selection of state-selective Gαs binding cyclic peptides.

FIG. 16A: The molecular switch Gαs adopts distinct conformations, governed by its guanine nucleotide binding state. Switch regions are highlighted with circle. FIG. 16B: A selection strategy to achieve state-selectivity of Gαs binders. FIG. 16C: Schematic representation of RaPID selection (e.g., Gαs active state binder selection, positive selection=Gαs/GppNHp (light grey), negative selection=Gαs/GDP (dark grey)). The mRNA library was ligated with puromycin, translated, and reverse-transcribed to yield our peptide-mRNA-cDNA complex library, which was subjected to sequential negative and positive selections. Negative selection was not included in the first round of selection. DNA sequences of cyclic peptide binders were identified by PCR. FIGS. 16D-16E: Sequence alignment of top 20 cyclic peptides from the last round of positive selections. The sulfur bridge cyclizes peptides between D-tyrosine and cysteine. The 18th peptide (asterisk) from the active state binder selection was not selected because it has the same sequence as the 1st peptide except a Gly to Ala mutation in the linker region. FIGS. 16F-16G: Comparison selection was performed by analyzing peptide-mRNA-cDNA complex binding to GDP-bound or GppNHp-bound Gαs-immobilized beads from the last round of selections, respectively. A high ratio means a better selectivity between the positive/negative selection. Cyclic peptides with high selectivity are marked with triangles in FIG. 16D or FIG. 16E and were selected for solid phase synthesis.

FIGS. 17A-17F. Gαs active state inhibitor GN13 inhibits Gαs-mediated adenylyl cyclase activation. FIG. 17A: Schematic representation of active state binders inhibiting Gαs mediated adenylyl cyclase activation. FIG. 17B: Activation of adenylyl cyclase by Gαs was inhibited by active state binders in a dose-dependent manner. GppNHp-bound Gαs was mixed with various concentrations of cyclic peptides, adenylyl cyclase (VC1/IIC2), and forskolin. After adding ATP, the reaction was carried out at 30° C. for 10 min. Production of cAMP was evaluated by the LANCE Ultra cAMP kit. The data represent the mean±SE of three independent replicates. FIG. 17C: Active state binders inhibited the protein-protein interaction between biotinylated Gαs WT and His-tagged adenylyl cyclase in a dose-dependent manner. The data represent the mean±SD of three independent replicates. FIG. 17D: Chemical structure of the resynthesized cyclic peptide GN13. Cyclization linkage is highlighted (box). FIG. 17E: Schematic representation of GN13 inhibiting GPCR-stimulated Gαs activity in cell membrane. FIG. 17F: Cell membranes were prepared from HEK293 cells and were preincubated with GTP/GDP mixture (500/50 μM) and various concentrations of GN13 for 2 hours, and then stimulated with 40 μM of β2AR agonist isoproterenol. After adding ATP, the reaction was carried out at 30° C. for 30 min. Production of cAMP was evaluated by the LANCE Ultra cAMP kit. The data represent the mean±SD of three independent replicates.

FIGS. 18A-18F. Crystal Structure of GppNHp-bound Gαs in complex with GN13. FIG. 18A: Overall structure of the GN13/GppNHp-bound Gαs complex. GN13 binds in between the switch II region and the α3 helix. GN13 and GppNHp are shown as sticks. Inset: Structural details of the GN13-Gαs interaction. Ion pair and hydrogen bonds are represented by dashed lines. FIG. 18B: Close-up view of two hydrophobic pockets that accommodate the tryptophan and isoleucine side chains of GN13. Residues that form those pockets are depicted as stick models and labeled. FIG. 18C: Alignment of Gαs/GN13 complex structure with the structure of GTPγS-bound Gαs (PDB: 1AZT). Root mean square deviation (RMSD)=0.479 Å. FIG. 18D: Left panel: Gαs/GN13 complex structure was aligned with the structure of GTPγS-bound Gαs/adenylyl cyclase complex (PDB: 1AZS). GN13 blocks H989/F991 of adenylyl cyclase from binding to the same pocket in Gαs. Middle panel: Close-up view of the interaction between GN13 and the Gαs α3 helix. Right panel: Close-up view of the interaction between adenylyl cyclase and the Gαs α3 helix (PDB: 1AZS). S275 is shown as sticks. FIG. 18E: WT Gαs and the S275L mutant have comparable biochemical activities in the adenylyl cyclase activation assay in the presence of Gβ1/γ2 (circles). 6.25 μM of GN13 inhibits adenylyl cyclase activation by Gαs WT (squares, left) but not by Gαs S275L (squares, right). The data represent the mean±SD of three independent measurements. FIG. 18F: The Gαs S275L mutation confers resistance to GN13 inhibition in HEK293 cell membranes. GNAS KO HEK 293 cells were transiently transfected with Gαs WT (circles) or S275L mutant (squares) constructs and followed by cell membrane preparation. Cell membranes were treated with various concentrations of GN13 for 2 hours, and then stimulated with 40 μM of β2AR agonist isoproterenol. After adding ATP, the reaction was carried out at 30° C. for 30 min. Production of cAMP was evaluated by the LANCE Ultra cAMP kit. The data represent the mean±SD of three independent replicates.

FIGS. 19A-19E. Gαs inactive state inhibitor GD20 inhibits Gαs steady-state GTPase activity by preventing GDP dissociation. FIG. 19A: Schematic representation of inactive state binders inhibiting Gαs steady-state GTPase activity. FIG. 19B: Gαs steady-state GTPase activity was inhibited by inactive state binders in a dose-dependent manner. The data represent one measurement. FIG. 19C: Chemical structure of the resynthesized cyclic peptide GD20. Cyclization linkage was highlighted (box). FIG. 19D: GD20 slows down the rates of GDP dissociation from Gαs. Gαs preloaded with [³H]GDP was assayed in a buffer containing 1 mM MgCl₂, 0.5 mM GDP, and the indicated concentration of GD20. The data represent the mean±SD of three independent replicates. FIG. 19E: The rates of GTPγS binding to Gαs in the presence (squares) or absence (circles) of 10 μM GD20 were determined by mixing GDP-bound Gαs with a mixture of [³⁵S] GTPγS and GTPγS in a buffer containing 1 mM MgCl₂. The data represent the mean±SD of three independent replicates.

FIGS. 20A-20G. Crystal Structure of GDP-bound Gαs in complex with GD20. FIG. 20A: Overall structure of the GD20/GDP-bound Gαs complex. GD20 binds in between the switch II region and the α3 helix. GD20 and GDP are shown as sticks. Inset: Structural details of the GD20-Gαs interaction. Ion pair and hydrogen bonds are represented by dashed lines. FIG. 20B: Close-up view of a hydrophobic pocket in Gαs that accommodates the Phe5 and Trp8 side chains of GD20. GD20 is shown as cartoon, and Gαs is shown as surface. Residues that form the hydrophobic pocket are depicted as stick models and labeled. FIG. 20C: Alignment of Gαs/GD20 complex structure with the structure of GTPγS-bound Gαs (PDB: 1AZT). FIG. 20D: Alignment of Gαs/GD20 complex structure with the structure of GDP-bound Gαs in the crystal structure of Gαs/Gβ1/γ2 heterotrimer (PDB: 6EG8). Gβγ was hidden for clarity. Inset: Close-up view of Gαs nucleotide binding pocket in the Gαs/GD20 complex structure. GD20 is shown as surface. Gαs is shown as cartoon. GDP is shown as sticks. Residues that stabilize GDP binding are depicted as stick models and labeled. Hydrogen bonds are represented by dashed lines. FIG. 20E: Structural details of Gαs and Gβγ binding interface (PDB: 6EG8). Gαs is shown as surface, and Gβγ are shown as cartoon. FIG. 20F: The Gβγ binding interface of Gαs is significantly rearranged when GD20 binds to Gαs. FIG. 20G: GD20, but not the GD20-F5A mutant, inhibited the protein-protein interaction between biotinylated Gαs WT and His-tagged Gβγ(C68S) in a dose-dependent manner. The data represent the mean±SD of three independent replicates.

FIGS. 21A-21F. A cell permeable cyclic peptide GD20-F10L inhibits Gαs Gβγ reassociation in HEK293 cells. FIG. 21A: Schematic representation of PPI inhibitors inhibiting Gαs Gβγ reassociation in HEK 239 cells. Gαs/Gβγ trimer is first dissociated by GPCR activation. PPI inhibitors captures the monomeric Gαs/GDP after Gαs/GTP hydrolysis, which prevents Gαs Gβγ reassociation. FIG. 21B: CAPA cell permeability assay results for ct-GD20 (circles) and ct-GD20-F10L (squares). Each point is the median ct-TAMRA fluorescence of 10,000 cells. The data were normalized using cells that were only treated with ct-TAMRA as 100% signal and cells that were not treated with any ct-compound as 0% signal. The data represent the mean±SD of three independent replicates. FIG. 21C: Schematic representation of GD20-F10L inhibiting the protein-protein interaction between GαsShort_Rluc and Gβ1/GFP2_γ2 in a BRET2 assay. FIG. 21D: HEK293 cells transfected with β2AR, Gαs-RLuc8, Gβ1, and Gγ2-GFP2 were pretreated with 25 μM GD20-F10L, GD20-F10L/F5A or DMSO for 16 hours. Gαs/Gβγ dissociation was measured by BRET2 signal reduction after 10 nM isoproterenol application. BRET2 signal was normalized to cells that were not treated with isoproterenol. The data represent the mean±SD of three independent replicates. Two-tailed unpaired t-tests were performed and P<0.05 was considered significant. *p<0.05, **p<0.005, ns >0.05. FIG. 21E: HEK293 cells transfected with β2AR, Gαs-RLuc8, Gβ1, and Gγ2-GFP2 were pretreated with various concentrations of GD20-F10L for 16 hours. Gαs/Gβγ dissociation was measured by BRET2 signal reduction after 10 nM isoproterenol application. BRET2 signal was normalized to cells that were not treated with isoproterenol. The data moving from left to right in the graph correspond to the legend moving from top to bottom. The data represent the mean±SD of three independent replicates. FIG. 21F: HEK293 cells transfected with M2R, Gαi1-RLuc8, Gβ1, and Gγ2-GFP2 were pretreated with 25 μM GD20-F10L or DMSO for 16 hours. Gαi1/Gβγ dissociation was measured by BRET2 signal reduction after 100 nM acetylcholine application. BRET2 signal was normalized to cells that were not treated with acetylcholine.

The data moving from left to right in the graph correspond to the legend moving from top to bottom. The data represent the mean±SD of three independent replicates. Two-tailed unpaired t-tests were performed and P<0.05 was considered significant. *p<0.05, **p<0.005, ns >0.05.

FIGS. 22A-22K. GD20 specifically inhibits Gαs through binding to a crystallographically defined pocket, related to FIGS. 20A-20G. FIGS. 22A-22B: GD20 adopts a highly ordered three-dimensional structure through intramolecular and intermolecular hydrogen bonding network. GD20 is shown as cyan sticks (FIG. 22A) or cartoon (FIG. 22B). Four water molecules with well-defined electron density are shown as red spheres. Hydrogen bonds are represented by dashed lines. FIGS. 22C-22D: Electron density map of GD20. GD20 is shown as sticks. Four water molecules with well-defined electron density are shown as spheres. The 2mFo-DFc electron density map of the structure is contoured at 1.0 σ. FIG. 22E: Electron density map of GDP. GDP and the side chain of R201 are shown as sticks. The Mg²⁺ and two water molecules coordinated with the Mg²⁺ are shown as spheres. The 2mFo-DFc electron density map of the structure is contoured at 1.0 σ. FIGS. 22F-22H: Binding kinetics of GD20 and GD20-F5A to Gα proteins were quantified using bio-layer Interferometry. The assay was performed in duplicate, and the data represent one of the two replicates. Biotinylated Gα proteins were immobilized to give a relative intensity of 2.5 nm on streptavidin biosensors. Association (t=0-120 s) and dissociation (t=120-240 s) cycles of compounds were started by dipping sensors into cyclic peptide dilutions and control buffer. FIG. 22F: GD20 binding to GDP-bound Gαs. FIG. 22G: 333.3 nM of GD20 binding to different Gα proteins. FIG. 22H: 333.3 nM of GD20 or GD20-F5A binding to GDP-bound Gαs. FIG. 22I: Structural basis for G protein class-specific binding of GD20 to Gαs. Gαs interacts with GD20 though three major specificity-determining sites. However, Gαi misses those critical GD20-binding residues. FIG. 22J: Schematic representation of inactive state binders inhibiting the protein-protein interaction between biotinylated Gαs WT and His-tagged Gβγ(C68S). FIG. 22K: GD20 inhibited the protein-protein interaction between biotinylated Gαs WT and His-tagged Gβγ(C68S) in a dose-dependent manner. GD20 was 100-fold more selective for Gαs than Gαi. The data represent the mean±SD of three independent replicates.

FIGS. 23A-23J. A cell permeable GD20 analog F10L specifically inhibits Gαs/Gβγ interaction through a Gαs-specific manner, related to FIGS. 21A-21F. FIGS. 23A-23D: Structure of derivatized cyclic peptides. ct-GD20 (FIG. 23A), GD20-F10L (FIG. 23B), ct-GD20-F10L (FIG. 23C), ct-GN13-E3Q (FIG. 23D). Mutations are highlighted in the dashed line box. The ct tag is highlighted in the solid line box. Cell penetration of ct-GN13-E3Q was also measured using the CAPA assay. FIGS. 23E-23F: Binding kinetics of GD20-F10L to different Gα proteins were quantified using bio-layer Interferometry. The assay was performed in duplicate, and the data represent one of the two replicates. Biotinylated Gα proteins were immobilized to give a relative intensity of 2.5 nm on streptavidin biosensors. Association (t=0-120 s) and dissociation (t=120-240 s) cycles of compounds were started by dipping sensors into cyclic peptide dilutions and control buffer. FIG. 23E: GD20-F10L binding to GDP-bound Gαs. FIG. 23F: 333.3 nM of GD20-F10L binding to different Gα proteins. FIGS. 23G-23H: GD20-F10L inhibited the protein-protein interaction between biotinylated Gαs WT and His-tagged Gβγ(C68S) in a dose-dependent manner (FIG. 23G). GD20-F10L was 100-fold more selective for Gαs than Gαi (FIG. 23H). The data represent the mean±SD of three independent replicates. FIG. 23I: Schematic representation of the chloroalkane penetration assay (CAPA). HeLa cells stably express GFP-tagged HaloTag on the mitochondrial outer membrane. If the pre-dosed chloroalkane-tagged molecule (ct-molecule) penetrates the cell membrane, it will covalently label HaloTag and block subsequent HaloTag labeling with ct-TAMRA. Intracellular ct-TAMRA fluorescence intensity is inversely related to the amount of cytosolic ct-molecule. FIG. 23J: The GD20/Gαs complex structure provides structural basis for the Rluc8 insertion. Rluc8 is inserted between αB and αC helices.

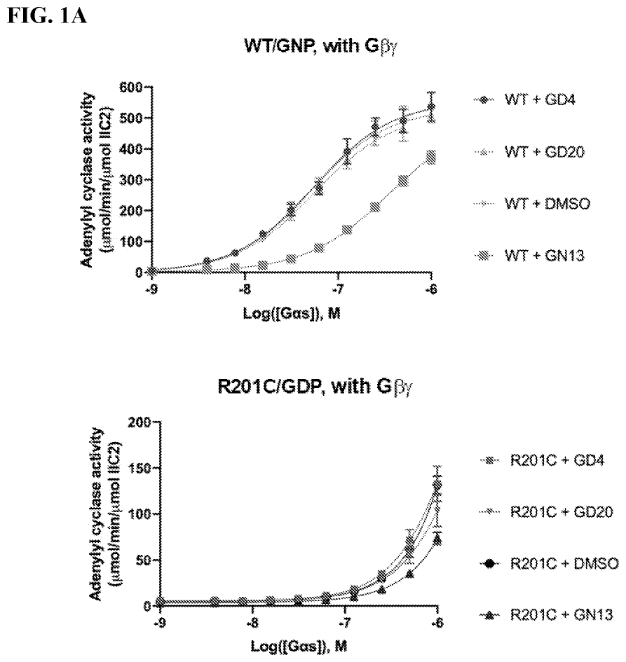

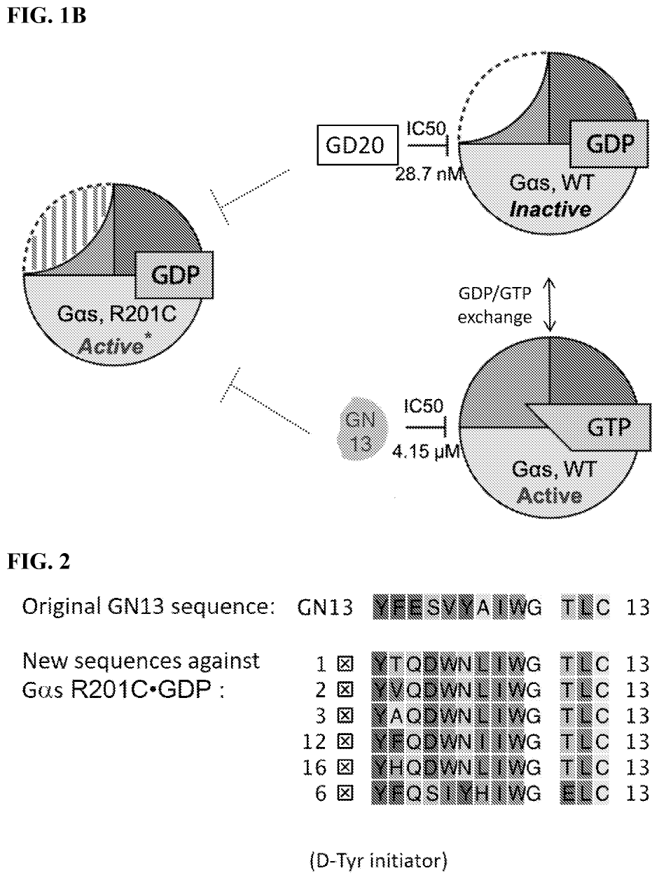

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C1-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

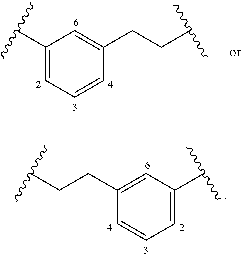 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR′″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₅ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₅ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₅ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1} L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.1}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{L100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

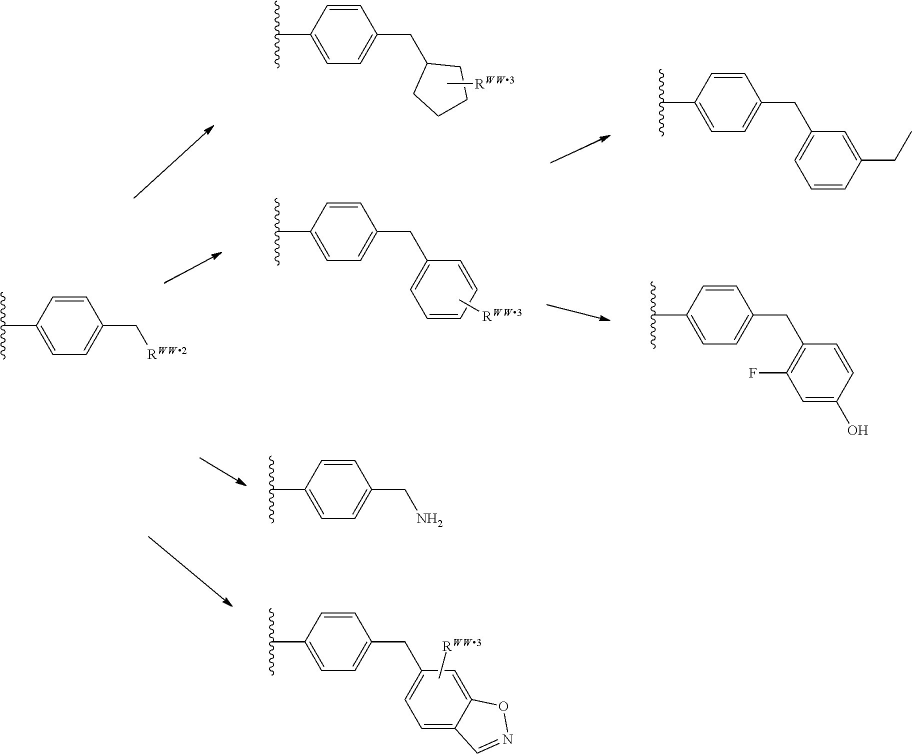

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂—CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}, —CHX^{WW.3}₂, —CH₂X^{WW.3}, —OCX^{WW.3}, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100B.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}, R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHXLWW.2₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a cancer.

The term “bone condition” as used herein refers to a disease, disorder or condition caused by abnormal bone tissues (e.g., osteoblast, osteoclast, osteocyte, and hematopoietic). In embodiments, the bone condition is caused by, but not limited to, cancerous or noncancerous tissues, infection, osteoporosis, tumor, blood cells, and fibrous tissues, which is developed in various sites of bones of a subject such as thighbone, skull, ribs, pelvis, humerus, shinbone, trunk, sternum, wrist bones, tarsals, spine, shoulder blade, collar bone, radius, ulna, metacarpals, phalanges, kneecap, fibula, metatarsals and phalanges. In certain embodiments, the bone condition may be caused by cancerous bone tissues or noncancerous bone tissues. In certain embodiments, the bone condition may be related to abnormal fibrous tissue development/occurrence in place of normal bone.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

“G protein associated cancer” (also referred to herein as “G-protein related cancer”) refers to a cancer caused by aberrant activity or signaling of G protein or one or more of its subunits (e.g., alpha (α)-, beta (β)-, or gamma (γ) subunits; Gαs, Gβs, or Gγs). In certain embodiments, a “cancer associated with aberrant Gαs activity” (also referred to herein as “Gαs related cancer”) is a cancer caused by aberrant Gαs activity or signaling (e.g., a mutant Gαs). In certain embodiments, a “cancer associated with aberrant Gβs activity” (also referred to herein as “Gβs related cancer”) is a cancer caused by aberrant Gβs activity or signaling (e.g., a mutant Gβs). In certain embodiments, a “cancer associated with aberrant Gγs activity” (also referred to herein as “Gγs related cancer”) is a cancer caused by aberrant Gγs activity or signaling (e.g., a mutant Gγs). In certain embodiments, some cancers that are associated with aberrant activity of one or more of G protein or its subunits (Gαs, Gβs, or Gγs), mutant G protein, or mutants subunits (Gαs, Gβs, or Gγs) are well known in the art and determining such cancers are within the skill of a person of skill in the art. In certain embodiments, some cancers may be sensitive to Gαs inhibition. In certain embodiments, the cancer that may be sensitive to Gαs inhibition may include a solid cancer or a tumor. In certain embodiments, the cancer that may be sensitive to Gαs inhibition may include a pancreatic cancer, a brain tumor, a pituitary tumor, or a bone tumor. In certain embodiments, the Gαs related cancers may include a pancreatic cancer, a brain tumor, a pituitary tumor, or a bone tumor.

“G protein-associated disease” (also referred to herein as “G protein-related disease”) refers to a cancer caused by aberrant activity or signaling of G protein or one or more of its subunits (e.g., alpha (α)-, beta (β)-, or gamma (γ) subunits; Gαs, Gβs, or Gγs). In certain embodiments, a “disease associated with aberrant Gαs activity” (also referred to herein as “Gαs related disease”) is a cancer caused by aberrant Gαs activity or signaling (e.g., a mutant Gαs). In certain embodiments, a “disease associated with aberrant Gβs activity” (also referred to herein as “Gβs related disease”) is a disease caused by aberrant Gβs activity or signaling (e.g., a mutant Gβs). In certain embodiments, a “disease associated with aberrant Gγs activity” (also referred to herein as “Gγs related disease”) is a disease caused by aberrant Gγs activity or signaling (e.g., a mutant Gγs). In certain embodiments, some diseases that are associated with aberrant activity of one or more of G protein or its subunits (Gαs, Gβs, or Gγs), mutant G protein, or mutants subunits (Gαs, Gβs, or Gγs) are well known in the art and determining such diseases are within the skill of a person of skill in the art. In certain embodiments, some diseases may be sensitive to Gαs inhibition.

The term “guanine nucleotide-binding protein” or “G protein” refers to one or more of the family of proteins that are bound to GTP (“on” state) or GDP (“off” state”) so the proteins can regulate their activity involved in signaling pathway of a cell. In certain embodiments, G protein includes subunits, alpha (α)-, beta (β)-, and gamma (γ) subunits (Gαs, Gβs, or Gγs). In particular, the term human “Gαs” as used herein refers to a G-protein-alpha-subunit having nucleotide sequences as set forth or corresponding to Entrez 2778, UniProt Q59FM5, UniProt P63092 (e.g., UniProt P6309-1 and UniProt P63092-2), RefSeq (protein) NP_000507.1, RefSeq (protein) NP_001070956.1, RefSeq (protein) NP_001070957.1, RefSeq (protein) NP_001070958.1, RefSeq (protein) NP_001296769.1, RefSeq (protein) NP_536350.2, or RefSeq (protein) NP_536351.1. In embodiments, the GNAS gene has the nucleic acid sequence set forth in RefSeq (mRNA) NM_000516.5, RefSeq (mRNA) NM_001077488.3, RefSeq (mRNA) NM_001077489.3, RefSeq (mRNA) NM_001077490.2, RefSeq (mRNA) NM_001309840.1, RefSeq (mRNA) NM_080425.3, or RefSeq (mRNA) NM_080426.3. In embodiments, the amino acid sequence or nucleic acid sequence is the sequence known at the time of filing of the present application.

The term “Gαs” includes both the wild-type form of the nucleotide sequences or proteins as well as any mutants thereof. In certain embodiments, the human Gαs refers to the protein including (e.g., consisting of) the amino acid sequence corresponding to UniProt P63092-1 (SEQ ID NO: 1). In embodiments, the human Gαs includes the sequence below with one or more mutations (e.g., R201C and C237S at the underlined position at SEQ ID NO: 1):

| | (SEQ ID NO: 1) |
|---|---|
| 1 | MGCLGNSKTE DQRNEEKAQR EANKKIEKQL QKDKQVYRAT HRLLLLGAGE SGKSTIVKQM |
| 61 | RILHVNGFNG EGGEEDPQAA RSNSDGEKAT KVQDIKNNLK EAIETIVAAM SNLVPPVELA |
| 121 | NPENQFRVDY ILSVMNVPDF DFPPEFYEHA KALWEDEGVR ACYERSNEYQ LIDCAQYFLD |
| 181 | KIDVIKQADY VPSDQDLLRC RVLTSGIFET KFQVDKVNFH MFDVGGQRDE RRKWIQCFND |
| 241 | VTAIIFVVAS SSYNMVIRED NQTNRLQEAL NLFKSIWNNR WLRTISVILF LNKQDLLAEK |
| 301 | VLAGKSKIED YFPEFARYTT PEDATPEPGE DPRVTRAKYF IRDEFLRIST ASGDGRHYCY |
| 361 | PHFTCAVDTE NIRRVFNDCR DIIQRMHLRQ YELL |

In embodiments, the human Gαs has the sequence of residues 7-380 of the short isoform of human Gαs corresponding to UniProt P63092-2 (SEQ ID NO: 2). In embodiments, the human Gαs includes the sequence below with one or more mutations (e.g., at R187 and/or C223 at the underlined position at SEQ ID NO: 2):

(SEQ ID NO: 2)
HMGCLGNSKTEDQRNEEKAQREANKKIEKQLQKDKQVYRATHRLLLLGAG
ESGKSTIVKQMRILHVNGFNGDSEKATKVQDIKNNLKEAIETIVAAMSNL
VPPVELANPENQFRVDYILSVMNVPDFDFPPEFYEHAKALWEDEGVRACY
ERSNEYQLIDCAQYFLDKIDVIKQADYVPSDQDLLRCRVLTSGIFETKFQ
VDKVNFHMFDVGGQRDERRKWIQCFNDVTAIIFVVASSSYNMVIREDNQT
NRLQEALNLFKSIWNNRWLRTISVILFLNKQDLLAEKVLAGKSKIEDYFP
EFARYTTPEDATPEPGEDPRVTRAKYFIRDEFLRISTASGDGRHYCYPHF
TCAVDTENIRRVFNDCRDIIQRMHLRQYELL

An amino acid residue in Gαs “corresponds” to a given residue when it occupies the same essential structural position within the protein as the given residue. For example, a selected residue in a selected protein corresponds to R201 of Gαs protein when the selected residue occupies the same essential spatial or other structural relationship as R201 of Gαs protein. In some embodiments, where a selected protein is aligned for maximum homology with the Gαs protein, the position in the aligned selected protein aligning with R201 is said to correspond to R201. Further, a selected residue in a selected protein corresponds to C237 of Gαs protein when the selected residue occupies the same essential spatial or other structural relationship as C237 of Gαs protein. In some embodiments, where a selected protein is aligned for maximum homology with the Gαs protein, the position in the aligned selected protein aligning with C237 is said to correspond to C237. Instead of a primary sequence alignment, a three dimensional structural alignment can also be used, e.g., where the structure of the selected protein is aligned for maximum correspondence with the Gαs protein and the overall structures compared. In this case, an amino acid that occupies the same essential position as R201 in the structural model is said to correspond to the R201 residue, and an amino acid that occupies the same essential position as C237 in the structural model is said to correspond to the C237 residue. For example, R201 of SEQ ID NO: 1 corresponds to R187 of SEQ ID NO: 2, and C237 of SEQ ID NO: 1 corresponds to C223 of SEQ ID NO: 2.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸¹Gd, ¹⁶¹Tb, ¹⁶⁶Dy, ¹⁶⁶H, ¹⁶⁹Er, ¹⁷⁵Lu, ¹⁷⁷Lu ¹⁸⁶Re, ¹⁸⁸Re, ¹⁸⁹Re, ¹⁹⁴I, ¹⁹⁸Au, ¹⁹⁹Au, ^{211A}t, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ^{111A}g, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I ¹⁹⁸Au, ¹⁹⁹Au, ^{211A}t, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., X^{L518}, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or r1L.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol.™ (i.e., paclitaxel), Taxotere.™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DAIE), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “amino acid side chain” refers to the side chain of an amino acid. For example, if an amino acid has the formula

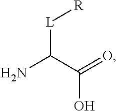

then -L-R is the amino acid side chain. As an example, D-tyrosine has the formula

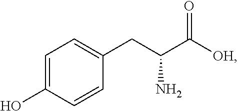 [SMILES: N[C@H](Cc1ccc(O)cc1)C(=O)O]

and the D-tyrosine side chain is

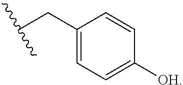 [SMILES: *Cc1ccc(O)cc1]

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to C237 of Gαs protein when the selected residue occupies the same essential spatial or other structural relationship as C237 of Gαs protein. In some embodiments, where a selected protein is aligned for maximum homology with the Gαs protein, the position in the aligned selected protein aligning with C237 is said to correspond to C237. Instead of a primary sequence alignment, a three dimensional structural alignment can also be used, e.g., where the structure of the selected protein is aligned for maximum correspondence with the Gαs protein and the overall structures compared. In this case, an amino acid that occupies the same essential position as C237 in the structural model is said to correspond to the C237 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

### II. Compounds

In an aspect is provided a compound having the formula:

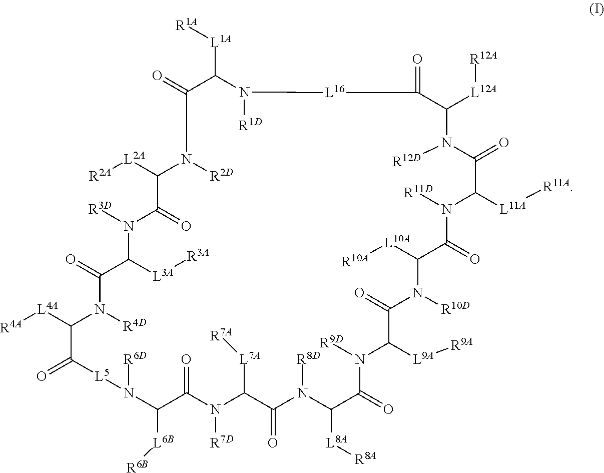

L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

L⁵ is

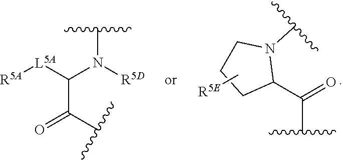

R^{1A} is substituted or unsubstituted aryl (e.g., C₆-C10 or phenyl).

R^{2A} and R^{5A} are independently hydrogen, —OH, —NH₂, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3A}, R^{4A}, and R^{11A} are independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{6A} is —NH₂, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted aryl (e.g., C₆-C₁₀ or phenyl).

R^{7A}, R^{8A}, and R^{12A} are independently hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{9A} is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{10A} is hydrogen or substituted or unsubstituted alkyl (e.g., C₁-C₈, C₁-C₆, C₁-C₄, or C₁-C₂).

R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₈ alkyl.

R^{5E} is hydrogen, —OH, —NH₂, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

L¹⁶ is a covalent linker.

In embodiments, the compound has the formula:

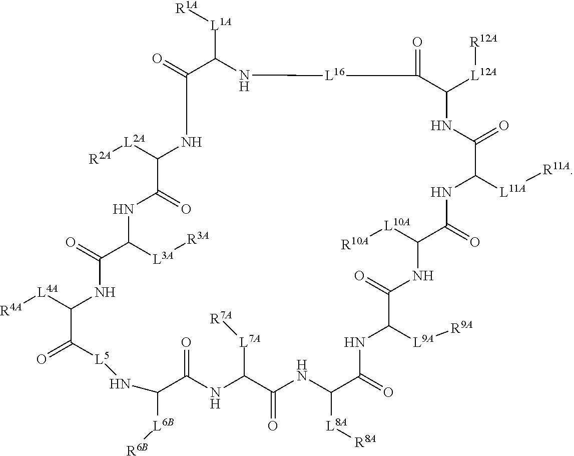

L^{1A}, L^{2A}, L^{3A}, L^{4A}, L⁵, L^{6A}, L^{7A}, L^{8A}, L^{9A}, L^{10A}, L^{11A}, L^{12A}, L¹⁶, R^{1A}, R^{2A}, R^{3A}, R^{4A}, R^{6A}, R^{7A}, R^{8A}, R^{9A}, R^{10A}, R^{11A}, and R^{12A} are as described herein, including in embodiments.

In embodiments, the compound has the formula:

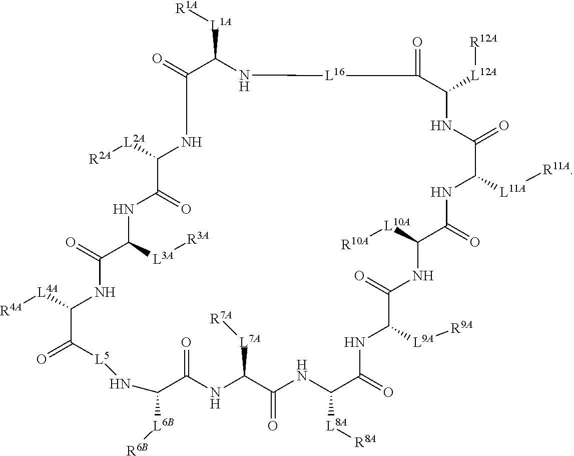

L^{1A}, L^{2A}, L^{3A}, L^{4A}, L⁵, L^{6A}, L^{7A}, L^{8A}, L^{9A}, L^{10A}, L^{11A}, L^{12A}, L¹⁶, R^{1A}, R^{2A}, R^{3A}, R^{4A}, R^{6A}, R^{7A}, R^{8A}, R^{9A}, R^{10A}, R^{11A}, and R^{12A} are as described herein, including in embodiments.

In embodiments, the compound has the formula:

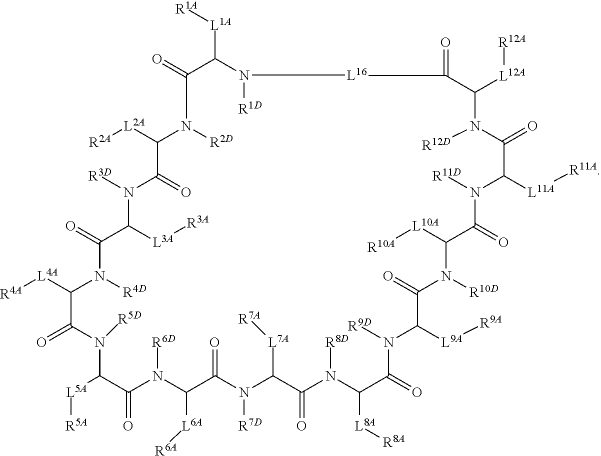

L^{1A}, L^{2A}, L^{3A}, L^{4A}, L^{5A}, L^{6A}, L^{7A}, L^{8A}, L^{9A}, L^{10A}, L^{11A}, L^{12A}, L¹⁶, R^{1A}, R^{2A}, R^{3A}, R^{4A}, R^{5A}, R^{6A}, R^{7A}, R^{8A}, R^{9A}, R^{10A}, R^{11A}, R^{12A}, R^{1D}, R^{2D}, R^{3D}, R^{4D}, R^{5D}, R^{6D}, R^{7D}, R^{8D}, R^{9D}, R^{10D}, R^{11D}, and R^{12D} are as described herein, including in embodiments.

In embodiments, the compound has the formula:

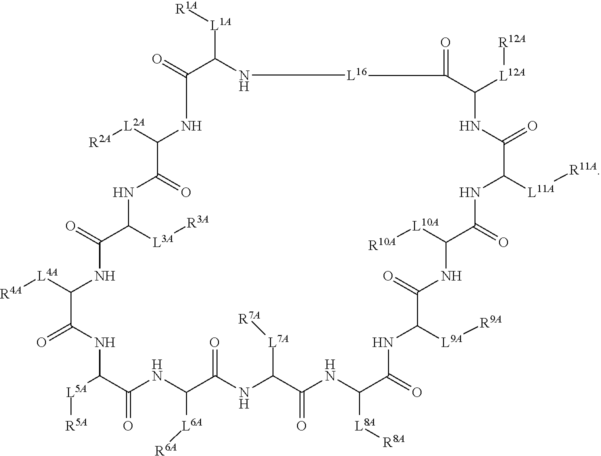

L^{1A}, L^{2A}, L^{3A}, L^{4A}, L^{5A}, L^{6A}, L^{7A}, L^{8A}, L^{9A}, L^{10A}, L^{11A}, L^{12A}, L¹⁶, R^{1A}, R^{2A}, R^{3A}, R^{4A}, R^{5A}, R^{6A}, R^{7A}, R^{8A}, R^{9A}, R^{10A}, R^{11A}, and R^{12A} are as described herein, including in embodiments.

In embodiments, the compound has the formula:

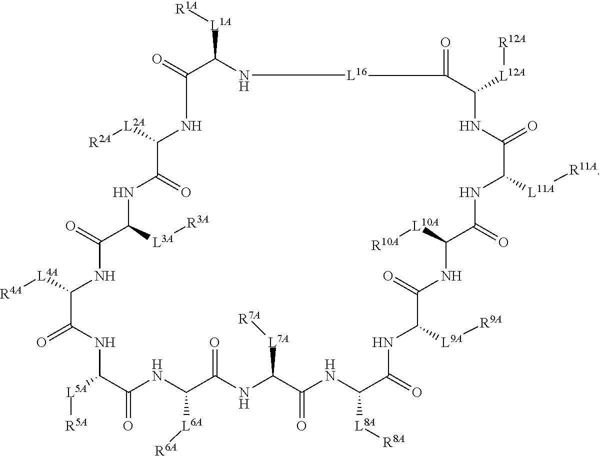

L^{1A}, L^{2A}, L^{3A}, L^{4A}, L^{5A}, L^{6A}, L^{7A}, L^{8A}, L^{9A}, L^{10A}, L^{11A}, L^{12A}, L¹⁶, R^{1A}, R^{2A}, R^{3A}, R^{4A}, R^{5A}, R^{6A}, R^{7A}, R^{8A}, R^{9A}, R^{10A}, R^{11A}, and R^{12A} are as described herein, including in embodiments.

In embodiments, the compound includes at least one negatively charged amino acid side chain. In embodiments, at least one of R^{3A}, R^{4A}, and R^{11A} is independently —COGH.

In embodiments, -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A}-R^{7A}-L^{8A}-R^{8A}-L^{9A}-R^{9A}-L^{10A}-R^{10A}-L^{11A}-R^{11A}, or -L^{12A}-R^{12A} are independently a natural amino acid side chain or an unnatural amino acid side chain. In embodiments, -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A}-R^{7A}-L^{8A}-R^{8A}-L^{9A}-R^{9A}-L^{10A}-R^{10A}-L^{11A}-R^{11A}, or -L^{12A}-R^{12A} are independently a natural amino acid side chain. In embodiments, -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A}-R^{7A}-L^{8A}-R^{8A}-L^{9A}-R^{9A}-L^{10A}-R^{10A}-L^{11A}-R^{11A}, or -L^{12A}-R^{12A} are independently an unnatural amino acid side chain.

In embodiments, a substituted L^{1A} (e.g., substituted alkylene and/or substituted heteroalkylene) is substituted with at least one substituent group, size-limited substituent group, or lower substituent group; wherein if the substituted L^{1A} is substituted with a plurality of groups selected from substituent groups, size-limited substituent groups, and lower substituent groups; each substituent group, size-limited substituent group, and/or lower substituent group may optionally be different. In embodiments, when L^{1A} is substituted, it is substituted with at least one substituent group. In embodiments, when L^{1A} is substituted, it is substituted with at least one size-limited substituent group. In embodiments, when L^{1A} is substituted, it is substituted with at least one lower substituent group.

In embodiments, a substituted L^{2A} (e.g., substituted alkylene and/or substituted heteroalkylene) is substituted with at least one substituent group, size-limited substituent group, or lower substituent group; wherein if the substituted L^{2A} is substituted with a plurality of groups selected from substituent groups, size-limited substituent groups, and lower substituent groups; each substituent group, size-limited substituent group, and/or lower substituent group may optionally be different. In embodiments, when L^{2A} is substituted, it is substituted with at least one substituent group. In embodiments, when L^{2A} is substituted, it is substituted with at least one size-limited substituent group. In embodiments, when L^{2A} is substituted, it is substituted with at least one lower substituent group.

In embodiments, a substituted L^{3A} (e.g., substituted alkylene and/or substituted heteroalkylene) is substituted with at least one substituent group, size-limited substituent group, or lower substituent group; wherein if the substituted L^{3A} is substituted with a plurality of groups selected from substituent groups, size-limited substituent groups, and lower substituent groups; each substituent group, size-limited substituent group, and/or lower substituent group may optionally be different. In embodiments, when L^{3A} is substituted, it is substituted with at least one substituent group. In embodiments, when L^{3A} is substituted, it is substituted with at least one size-limited substituent group. In embodiments, when L^{3A} is substituted, it is substituted with at least one lower substituent group.

In embodiments, a substituted L^{4A} (e.g., substituted alkylene and/or substituted heteroalkylene) is substituted with at least one substituent group, size-limited substituent group, or lower substituent group; wherein if the substituted L^{4A} is substituted with a plurality of groups selected from substituent groups, size-limited substituent groups, and lower substituent groups; each substituent group, size-limited substituent group, and/or lower substituent group may optionally be different. In embodiments, when L^{4A} is substituted, it is substituted with at least one substituent group. In embodiments, when L^{4A} is substituted, it is substituted with at least one size-limited substituent group. In embodiments, when L^{4A} is substituted, it is substituted with at least one lower substituent group.

In embodiments, a substituted L^{5A} (e.g., substituted alkylene and/or substituted heteroalkylene) is substituted with at least one substituent group, size-limited substituent group, or lower substituent group; wherein if the substituted L^{5A} is substituted with a plurality of groups selected from substituent groups, size-limited substituent groups, and lower substituent groups; each substituent group, size-limited substituent group, and/or lower substituent group may optionally be different. In embodiments, when L^{5A} is substituted, it is substituted with at least one substituent group. In embodiments, when L^{5A} is substituted, it is substituted with at least one size-limited substituent group. In embodiments, when L^{5A} is substituted, it is substituted with at least one lower substituent group.

In embodiments, a substituted L^{6A} (e.g., substituted alkylene and/or substituted heteroalkylene) is substituted with at least one substituent group, size-limited substituent group, or lower substituent group; wherein if the substituted L^{6A} is substituted with a plurality of groups selected from substituent groups, size-limited substituent groups, and lower substituent groups; each substituent group, size-limited substituent group, and/or lower substituent group may optionally be different. In embodiments, when L^{6A} is substituted, it is substituted with at least one substituent group. In embodiments, when L^{6A} is substituted, it is substituted with at least one size-limited substituent group. In embodiments, when L^{6A} is substituted, it is substituted with at least one lower substituent group.

In embodiments, a substituted L^{7A} (e.g., substituted alkylene and/or substituted heteroalkylene) is substituted with at least one substituent group, size-limited substituent group, or lower substituent group; wherein if the substituted L^{7A} is substituted with a plurality of groups selected from substituent groups, size-limited substituent groups, and lower substituent groups; each substituent group, size-limited substituent group, and/or lower substituent group may optionally be different. In embodiments, when L^{7A} is substituted, it is substituted with at least one substituent group. In embodiments, when L^{7A} is substituted, it is substituted with at least one size-limited substituent group. In embodiments, when L^{7A}, is substituted, it is substituted with at least one lower substituent group.

In embodiments, a substituted L^{8A} (e.g., substituted alkylene and/or substituted heteroalkylene) is substituted with at least one substituent group, size-limited substituent group, or lower substituent group; wherein if the substituted L^{8A} is substituted with a plurality of groups selected from substituent groups, size-limited substituent groups, and lower substituent groups; each substituent group, size-limited substituent group, and/or lower substituent group may optionally be different. In embodiments, when L^{8A} is substituted, it is substituted with at least one substituent group. In embodiments, when L^{8A} is substituted, it is substituted with at least one size-limited substituent group. In embodiments, when L^{8A} is substituted, it is substituted with at least one lower substituent group.

In embodiments, a substituted L^{9A} (e.g., substituted alkylene and/or substituted heteroalkylene) is substituted with at least one substituent group, size-limited substituent group, or lower substituent group; wherein if the substituted L^{9A} is substituted with a plurality of groups selected from substituent groups, size-limited substituent groups, and lower substituent groups; each substituent group, size-limited substituent group, and/or lower substituent group may optionally be different. In embodiments, when L^{9A} is substituted, it is substituted with at least one substituent group. In embodiments, when L^{9A} is substituted, it is substituted with at least one size-limited substituent group. In embodiments, when L^{9A} is substituted, it is substituted with at least one lower substituent group.

In embodiments, a substituted L^{10A} (e.g., substituted alkylene and/or substituted heteroalkylene) is substituted with at least one substituent group, size-limited substituent group, or lower substituent group; wherein if the substituted L^{10A} is substituted with a plurality of groups selected from substituent groups, size-limited substituent groups, and lower substituent groups; each substituent group, size-limited substituent group, and/or lower substituent group may optionally be different. In embodiments, when L^{10A} is substituted, it is substituted with at least one substituent group. In embodiments, when L^{10A} is substituted, it is substituted with at least one size-limited substituent group. In embodiments, when L^{10A} is substituted, it is substituted with at least one lower substituent group.

In embodiments, a substituted L^{11A} (e.g., substituted alkylene and/or substituted heteroalkylene) is substituted with at least one substituent group, size-limited substituent group, or lower substituent group; wherein if the substituted L^{11A} is substituted with a plurality of groups selected from substituent groups, size-limited substituent groups, and lower substituent groups; each substituent group, size-limited substituent group, and/or lower substituent group may optionally be different. In embodiments, when L^{11A} is substituted, it is substituted with at least one substituent group. In embodiments, when L^{11A} is substituted, it is substituted with at least one size-limited substituent group. In embodiments, when L^{11A} is substituted, it is substituted with at least one lower substituent group.

In embodiments, a substituted L^{12A} (e.g., substituted alkylene and/or substituted heteroalkylene) is substituted with at least one substituent group, size-limited substituent group, or lower substituent group; wherein if the substituted L^{12A} is substituted with a plurality of groups selected from substituent groups, size-limited substituent groups, and lower substituent groups; each substituent group, size-limited substituent group, and/or lower substituent group may optionally be different. In embodiments, when L^{12A} is substituted, it is substituted with at least one substituent group. In embodiments, when L^{12A} is substituted, it is substituted with at least one size-limited substituent group. In embodiments, when L^{12A} is substituted, it is substituted with at least one lower substituent group.

In embodiments, a substituted R^{11A} (e.g., substituted aryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, a substituted R^{2A} (e.g., substituted alkyl, substituted cycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{3A} (e.g., substituted alkyl and/or substituted heteroalkyl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{4A} (e.g., substituted alkyl and/or substituted heteroalkyl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{5A} (e.g., substituted alkyl, substituted cycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{6A} (e.g., substituted alkyl, substituted heteroalkyl, and/or substituted aryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{7A} (e.g., substituted alkyl, substituted cycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{8A} (e.g., substituted alkyl, substituted cycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, a substituted R^{9A} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{9A} is substituted with a plurality of groups selected from substituent groups, size-limited substituent groups, and lower substituent groups; each substituent group, size-limited substituent group, and/or lower substituent group may optionally be different. In embodiments, when R^{9A} is substituted, it is substituted with at least one substituent group. In embodiments, when R^{9A} is substituted, it is substituted with at least one size-limited substituent group. In embodiments, when R^{9A} is substituted, it is substituted with at least one lower substituent group.

In embodiments, a substituted R^{10A} (e.g., substituted alkyl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted R^{11A} (e.g., substituted alkyl and/or substituted heteroalkyl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, a substituted R^{12A} (e.g., substituted alkyl, substituted cycloalkyl, substituted aryl, and/or substituted heteroaryl) is substituted with at least one substituent group, size-limited substituent group, or lower substituent group; wherein if the substituted R^{12A} is substituted with a plurality of groups selected from substituent groups, size-limited substituent groups, and lower substituent groups; each substituent group, size-limited substituent group, and/or lower substituent group may optionally be different. In embodiments, when R^{12A} is substituted, it is substituted with at least one substituent group. In embodiments, when R^{12A} is substituted, it is substituted with at least one size-limited substituent group. In embodiments, when R^{12A} is substituted, it is substituted with at least one lower substituent group.

In embodiments, a substituted R^{5E} (e.g., substituted alkyl and/or substituted heteroalkyl) is substituted with at least one substituent group, size-limited substituent group, or lower substituent group; wherein if the substituted R^{5E} is substituted with a plurality of groups selected from substituent groups, size-limited substituent groups, and lower substituent groups; each substituent group, size-limited substituent group, and/or lower substituent group may optionally be different. In embodiments, when R^{5E} is substituted, it is substituted with at least one substituent group. In embodiments, when R^{5E} is substituted, it is substituted with at least one size-limited substituent group. In embodiments, when R^{5E} is substituted, it is substituted with at least one lower substituent group.

In embodiments,

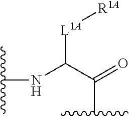

is a divalent form of an unnatural amino acid. In embodiments, L^{1A} is a bond or unsubstituted C₁-C₄ alkylene. In embodiments, R^{1A} is a substituted aryl. In embodiments,

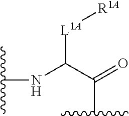

is a divalent form of a natural amino acid.

In embodiments,

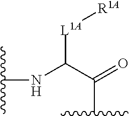

is a divalent form of tyrosine. In embodiments, -L^{1A}-R^{1A} is

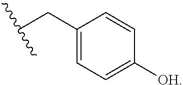 [SMILES: *Cc1ccc(O)cc1]

In embodiments,

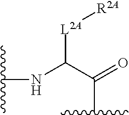

is a divalent form of an unnatural amino acid. In embodiments, L^{2A} is a bond or unsubstituted C₁-C₄ alkylene. In embodiments, R^{2A} is —OH, —NH₂, substituted or unsubstituted alkyl, substituted or unsubstituted aryl, or substituted or unsubstituted heteroaryl. In embodiments,

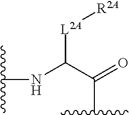

is a divalent form of a natural amino acid. In embodiments,

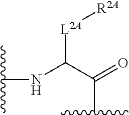

is a divalent form of phenylalanine, a divalent form of histidine, a divalent form of alanine, a divalent form of valine a divalent form of threonine, or a divalent form of tyrosine. In embodiments,

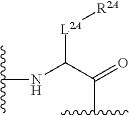

is a divalent form of phenylalanine. In embodiments,

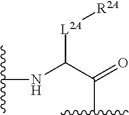

is a divalent form of histidine. In embodiments,

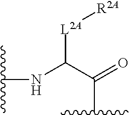

is a divalent form of alanine. In embodiments,

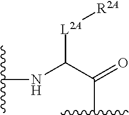

is a divalent form of valine. In embodiments,

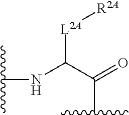

is a divalent form of threonine. In embodiments,

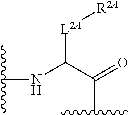

is a divalent form of tyrosine. In embodiments, -L^{2A}-R^{2A} is

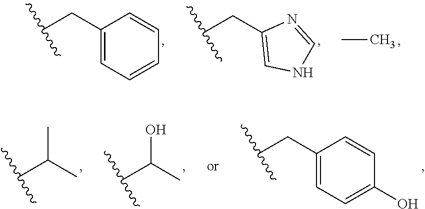

In embodiments, L^{2A}-R^{2A} is

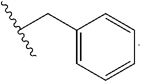 [SMILES: *Cc1ccccc1]

In embodiments, -L^{2A}-R^{2A} is

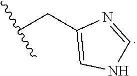 [SMILES: *Cc1c[nH]cn1]

In embodiments, -L^{2A}-R^{2A} is —CH₃. In embodiments, -L^{2A}-R^{2A} is

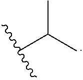 [SMILES: *C(C)C]

In embodiments, -L^{2A}-R^{2A} is

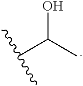 [SMILES: *C(C)O]

In embodiments, -L^{2A}-R^{2A} is

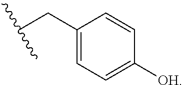 [SMILES: *Cc1ccc(O)cc1]

In embodiments,

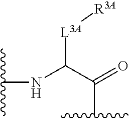

is a divalent form of an unnatural amino acid. In embodiments, L^{3A} is a bond or unsubstituted C₁-C₄ alkylene. In embodiments, R^{3A} is —OH, —NH₂, —COOH, —CONH₂, substituted or unsubstituted alkyl, or substituted or unsubstituted heteroalkyl. In embodiments,

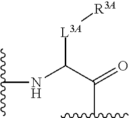

is a divalent form of a natural amino acid. In embodiments,

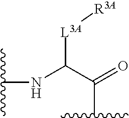

is a divalent form of glutamine or a divalent form of glutamic acid. In embodiments,

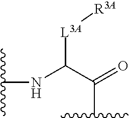

is a divalent form of glutamine. In embodiments,

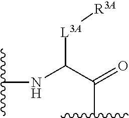

is a divalent form of glutamic acid. In embodiments, -L^{3A}-R^{3A} is

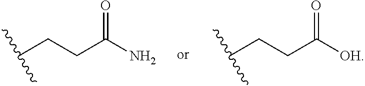 [SMILES: *CCC(N)=O ; "or" ; *CCC(=O)O]

In embodiments, -L^{3A}-R^{3A} is

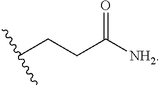 [SMILES: *CCC(N)=O]

In embodiments, -L^{3A}-R^{3A} is

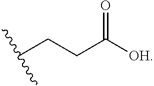 [SMILES: *CCC(=O)O]

In embodiments,

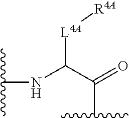

is a divalent form of an unnatural amino acid. In embodiments, L^{4A} is a bond or unsubstituted C₁-C₄ alkylene. In embodiments, R^{4A} is —OH, —COOH, or substituted or unsubstituted alkyl. In embodiments,

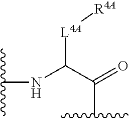

is a divalent form of a natural amino acid. In embodiments,

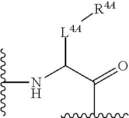

is a divalent form of serine or a divalent form of aspartic acid. In embodiments,

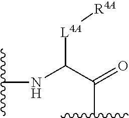

is a divalent form of serine. In embodiments,

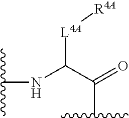

is a divalent form of aspartic acid. In embodiments, -L^{4A}-R^{4A} is

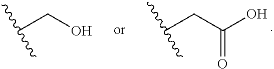

In embodiments, -L^{4A}-R^{4A} is

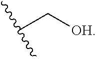 [SMILES: *CO]

In embodiments, -L^{4A}-R^{4A} is

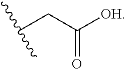 [SMILES: *CC(=O)O]

In embodiments,

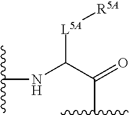

is a divalent form of an unnatural amino acid. In embodiments, L^{5A} is a bond or unsubstituted C₁-C₄ alkylene. In embodiments, R^{5A} is a hydrogen, or unsubstituted alkyl, or unsubstituted heteroaryl. In embodiments,

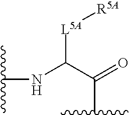

is a divalent form of a natural amino acid. In embodiments,

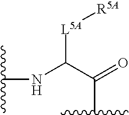

is a divalent form of isoleucine, a divalent form of tryptophan, or a divalent form of valine. In embodiments,

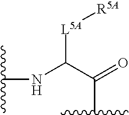

is a divalent form of isoleucine. In embodiments,

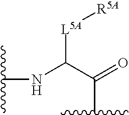

is a divalent form of tryptophan. In embodiments,

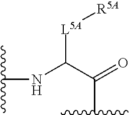

is a divalent form of valine. In embodiments, -L^{5A}-R^{5A} is

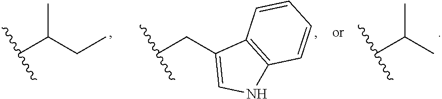

In embodiments, -L^{5A}-R^{5A} is

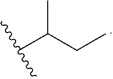 [SMILES: *C(C)CC]

In embodiments, -L^{5A}-R^{5A} is

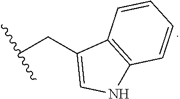 [SMILES: *Cc1c[nH]c2ccccc12]

In embodiments, -L^{5A}-R^{5A} is

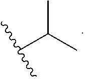 [SMILES: *C(C)C]

In embodiments,

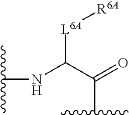

is a divalent form of an unnatural amino acid. In embodiments, L^{6A} is a bond or unsubstituted C₁-C₆ alkylene. In embodiments, R^{6A} is —NH₂, —CONH₂, substituted or unsubstituted alkyl, or substituted or unsubstituted heteroaryl. In embodiments,

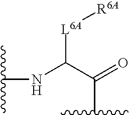

is a divalent form of a natural amino acid. In embodiments,

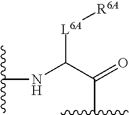

a divalent form of tyrosine or a divalent form of asparagine. In embodiments,

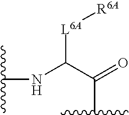

is a divalent form of tyrosine. In embodiments,

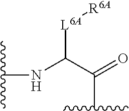

is a divalent form of asparagine. In embodiments,

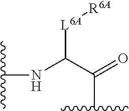

is a divalent form of arginine. In embodiments, -L^{6A}-R^{6A} is

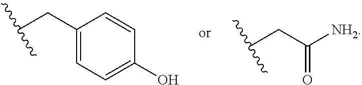 [SMILES: *Cc1ccc(O)cc1 ; *CC(N)=O]

In embodiments, -L^{6A}-R^{6A} is

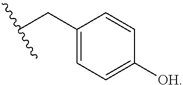 [SMILES: *Cc1ccc(O)cc1]

In embodiments, -L^{6A}-R^{6A} is

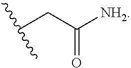 [SMILES: *CC(N)=O]

In embodiments,

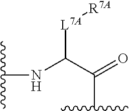

is a divalent form of an unnatural amino acid. In embodiments, L^{7A} is a bond or unsubstituted C₁-C₄ alkylene. In embodiments, R^{7A} is hydrogen, unsubstituted alkyl, or unsubstituted heteroaryl. In embodiments,

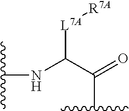

is a divalent form of a natural amino acid. In embodiments,

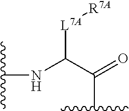

is a divalent form of histidine, a divalent form of leucine, a divalent form of isoleucine, or a divalent form of alanine. In embodiments,

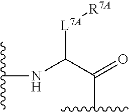

is a divalent form of histidine. In embodiments,

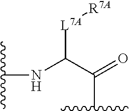

is a divalent form of leucine. In embodiments,

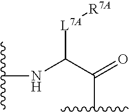

is a divalent form of isoleucine. In embodiments,

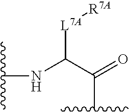

is a divalent form of alanine. In embodiments, -L^{7A}-R^{7A} is

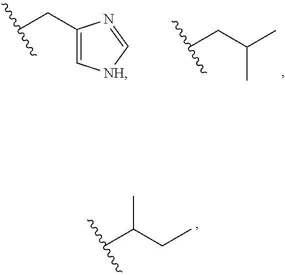

or —CH₃. In embodiments, -L^{7A}-R^{7A} is

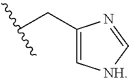 [SMILES: *Cc1c[nH]cn1]

In embodiments, -L^{7A}-R^{7A} is

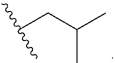 [SMILES: *CC(C)C]

In embodiments, -L^{7A}-R^{7A} is

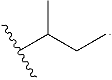 [SMILES: *C(C)CC]

In embodiments, -L^{7A}-R^{7A} is —CH₃.

In embodiments,

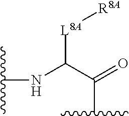

is a divalent form of an unnatural amino acid. In embodiments, L^{8A} is a bond or unsubstituted C₁-C₄ alkylene. In embodiments, R^{8A} is a hydrogen or unsubstituted alkyl. In embodiments,

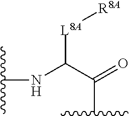

is a divalent form of a natural amino acid. In embodiments,

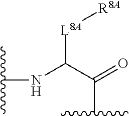

is a divalent form of isoleucine. In embodiments, -L^{8A}-R^{8A}, is

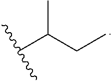 [SMILES: *C(C)CC]

In embodiments,

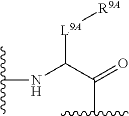

is a divalent form of an unnatural amino acid. In embodiments, L^{9A} is a bond or unsubstituted C₁-C₆ alkylene. In embodiments, R^{9A} is an unsubstituted heteroaryl. In embodiments,

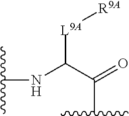

is a divalent form of a natural amino acid. In embodiments,

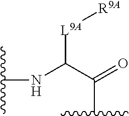

is a divalent form of tryptophan. In embodiments, -L^{9A}-R^{9A} is

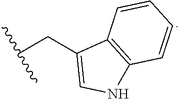 [SMILES: *Cc1c[nH]c2ccccc12]

In embodiments,

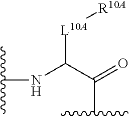

is a divalent form of an unnatural amino acid. In embodiments, L^{10A} is a bond or unsubstituted C₁-C₄ alkylene. In embodiments, R^{10A} is a hydrogen or unsubstituted alkyl. In embodiments,

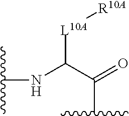

is a divalent form of a natural amino acid. In embodiments,

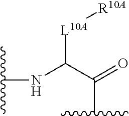

is a divalent form of glycine. In embodiments, -L^{10A}-R^{10A} is —H.

In embodiments,

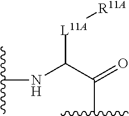

is a divalent form of an unnatural amino acid. In embodiments, L^{11A} is a bond or unsubstituted C₁-C₄ alkylene. In embodiments, R^{11A} is —OH, —COGH, —CONH₂, or substituted alkyl. In embodiments,

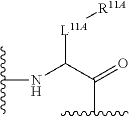

is a divalent form of a natural amino acid. In embodiments,

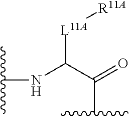

is a divalent form of glutamic acid, a divalent form of threonine, or a divalent form of glutamine. In embodiments,

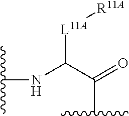

is a divalent form of glutamic acid. In embodiments,

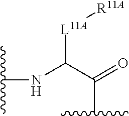

is a divalent form of threonine. In embodiments,

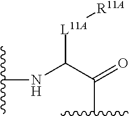

is a divalent form of glutamine. In embodiments, -L^{11A}-R^{11A} is

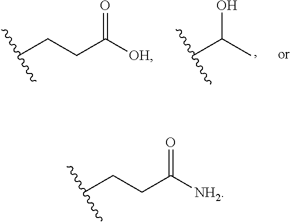

In embodiments, -L^{11A}-R^{11A} is

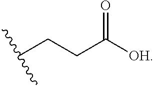 [SMILES: *CCC(=O)O]

In embodiments, -L^{11A}-R^{11A} is

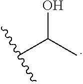 [SMILES: *C(C)O]

In embodiments, -L^{11A}-R^{11A} is

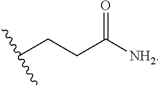 [SMILES: *CCC(N)=O]

In embodiments,

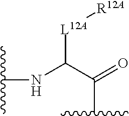

is a divalent form of an unnatural amino acid. In embodiments, L^{12A} is a bond or unsubstituted C₁-C₆ alkylene. In embodiments, R^{12A} is a hydrogen or unsubstituted alkyl. In embodiments,

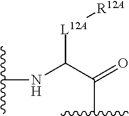

is a divalent form of a natural amino acid. In embodiments,

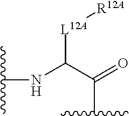

is a divalent form of leucine. In embodiments, -L^{12A}-R^{12A} is

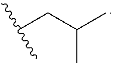 [SMILES: *CC(C)C]

In embodiments, the compound has the formula:

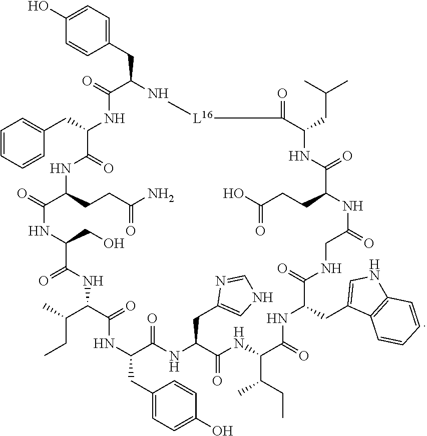

L¹⁶ is as described herein, including in embodiments.

In embodiments, the compound has the formula:

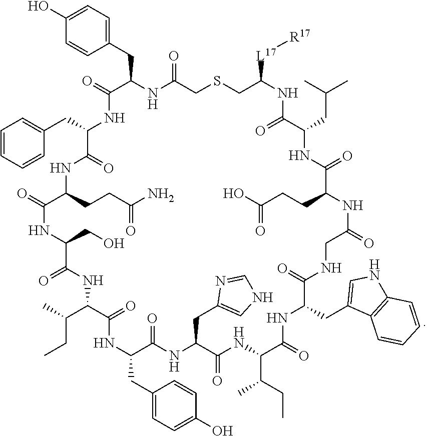

L¹⁷ and R¹⁷ are as described herein, including in embodiments.

In embodiments, the compound has the formula:

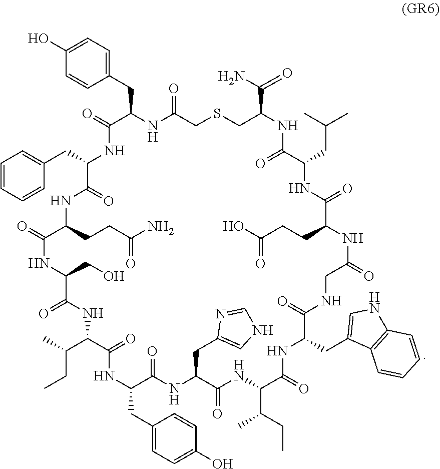 [SMILES: CC[C@H](C)[C@@H]1NC(=O)[C@H](Cc2c[nH]cn2)NC(=O)[C@H](Cc2ccc(O)cc2)NC(=O)[C@H]([C@@H](C)CC)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(N)=O)NC(=O)[C@H](CC(C)C)NC(=O)[C@H](CCC(=O)O)NC(=O)CNC(=O)[C@H](Cc2c[nH]c3ccccc23)NC1=O]

In embodiments, the compound has the formula:

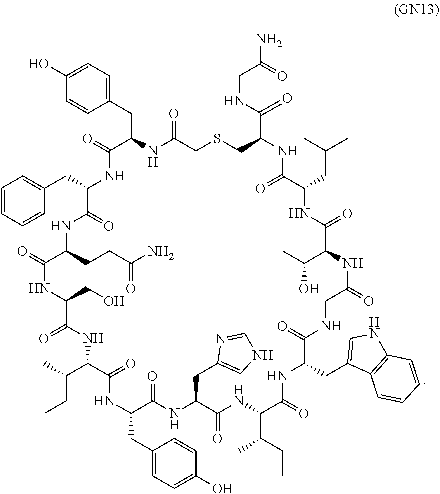 [SMILES: CC[C@H](C)[C@@H]1NC(=O)[C@H](Cc2c[nH]cn2)NC(=O)[C@H](Cc2ccc(O)cc2)NC(=O)[C@H]([C@@H](C)CC)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(=O)NCC(N)=O)NC(=O)[C@H](CC(C)C)NC(=O)[C@H]([C@@H](C)O)NC(=O)CNC(=O)[C@H](Cc2c[nH]c3ccccc23)NC1=O]

In embodiments, the compound does not have the formula:

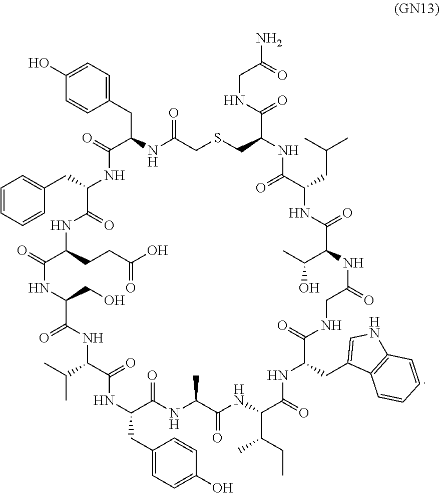 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(=O)O)NC(=O)[C@H](Cc2ccccc2)NC(=O)[C@@H](Cc2ccc(O)cc2)NC(=O)CSC[C@@H](C(=O)NCC(N)=O)NC(=O)[C@H](CC(C)C)NC(=O)[C@H]([C@@H](C)O)NC(=O)CNC(=O)[C@H](Cc2c[nH]c3ccccc23)NC1=O]

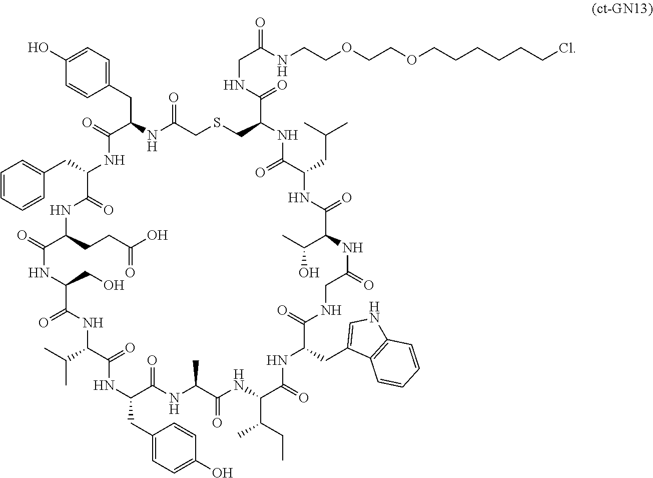 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(=O)O)NC(=O)[C@H](Cc2ccccc2)NC(=O)[C@@H](Cc2ccc(O)cc2)NC(=O)CSC[C@@H](C(=O)NCC(=O)NCCOCCOCCCCCCCl)NC(=O)[C@H](CC(C)C)NC(=O)[C@H]([C@@H](C)O)NC(=O)CNC(=O)[C@H](Cc2c[nH]c3ccccc23)NC1=O]

In embodiments, the compound has the formula:

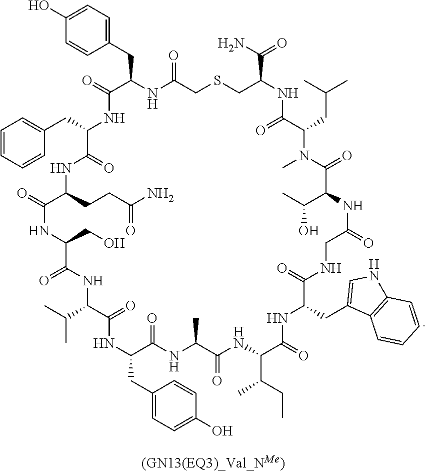 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(N)=O)NC(=O)[C@H](CC(C)C)N(C)C(=O)[C@H]([C@@H](C)O)NC(=O)CNC(=O)[C@H](Cc2c[nH]c3ccccc23)NC1=O]

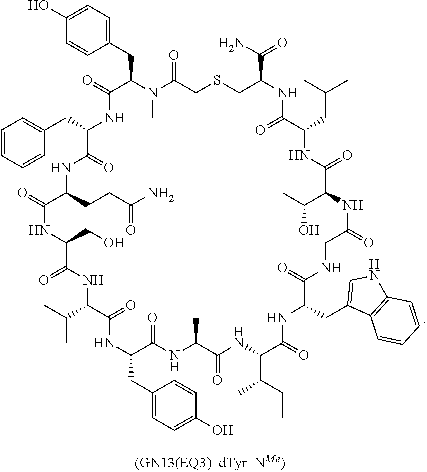 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(N)=O)NC(=O)[C@H](Cc2ccccc2)NC(=O)[C@@H](Cc2ccc(O)cc2)N(C)C(=O)CSC[C@@H](C(N)=O)NC(=O)[C@H](CC(C)C)NC(=O)[C@H]([C@@H](C)O)NC(=O)CNC(=O)[C@H](Cc2c[nH]c3ccccc23)NC1=O]

In embodiments, the compound has the formula:

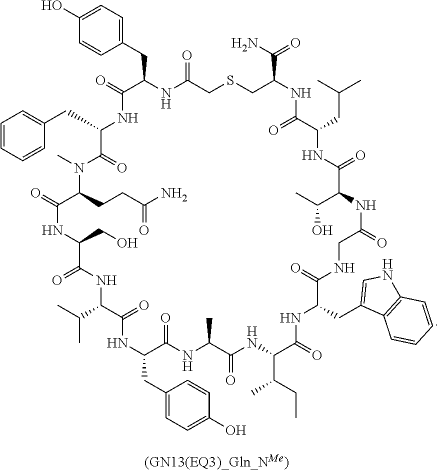 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(N)=O)N(C)C(=O)[C@H](Cc2ccccc2)NC(=O)[C@@H](Cc2ccc(O)cc2)NC(=O)CSC[C@@H](C(N)=O)NC(=O)[C@H](CC(C)C)NC(=O)[C@H]([C@@H](C)O)NC(=O)CNC(=O)[C@H](Cc2c[nH]c3ccccc23)NC1=O]

In embodiments, the compound has the formula:

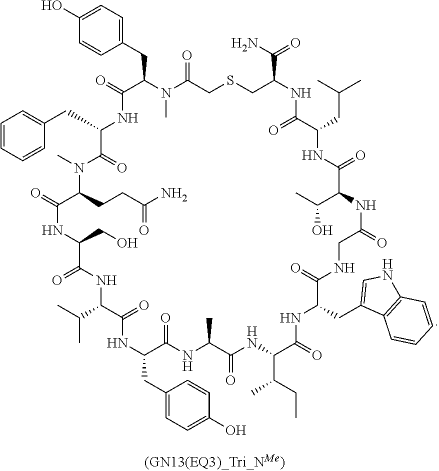 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(N)=O)N(C)C(=O)[C@H](Cc2ccccc2)NC(=O)[C@@H](Cc2ccc(O)cc2)N(C)C(=O)CSC[C@@H](C(N)=O)NC(=O)[C@H](CC(C)C)NC(=O)[C@H]([C@@H](C)O)NC(=O)CNC(=O)[C@H](Cc2c[nH]c3ccccc23)NC1=O]

In embodiments, the compound has the formula:

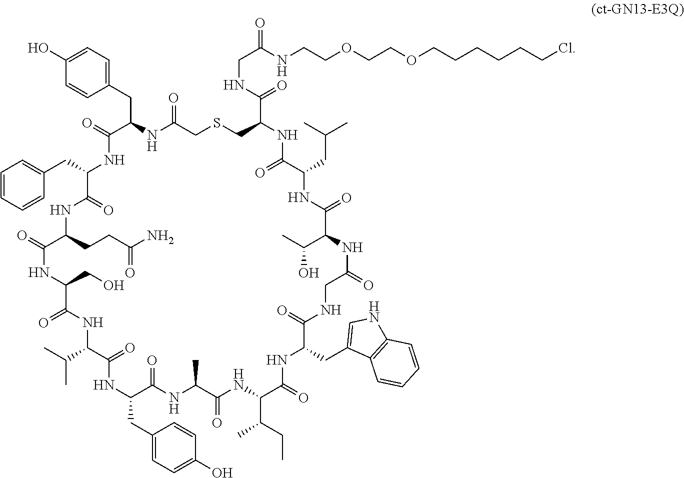 [SMILES: CC[C@H](C)[C@@H]1NC(=O)[C@H](C)NC(=O)[C@H](Cc2ccc(O)cc2)NC(=O)[C@H](C(C)C)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(=O)NCC(=O)NCCOCCOCCCCCCCl)NC(=O)[C@H](CC(C)C)NC(=O)[C@H]([C@@H](C)O)NC(=O)CNC(=O)[C@H](Cc2c[nH]c3ccccc23)NC1=O]

In embodiments, the compound of formula (I) is a peptide of FIG. 2. In embodiments, the compound of formula (I) is peptide GN13, 1, 2, 3, 12, 16, or 6 of FIG. 2.

For peptide GN13 of FIG. 2, -L^{1A}-R^{1A} is a tyrosine side chain; -L^{2A}-R^{2A} is a phenylalanine side chain; -L^{3A}-R^{3A} is a glutamic acid side chain; -L^{4A}-R^{4A} is a serine side chain; -L^{5A}-R^{5A} is a valine side chain; -L^{6A}-R^{6A} is a tyrosine side chain; -L^{7A}-R^{7A} is an alanine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide 1 of FIG. 2, -L^{1A}-R^{1A} is a tyrosine side chain; -L^{2A}-R^{2A} is a threonine side chain; -L^{3A}-R^{3A} is a glutamine side chain; -L^{4A}-R^{4A} is an aspartic acid side chain; -L^{5A}-R^{5A} is a tryptophan side chain; -L^{6A}-R^{6A} is an asparagine side chain; -L^{7A}-R^{7A} is a leucine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide 2 of FIG. 2, -L^{1A}-R^{1A} is a tyrosine side chain; -L^{2A}-R^{2A} is a valine side chain; -L^{3A}-R^{3A} is a glutamine side chain; -L^{4A}-R^{4A} is an aspartic acid side chain; -L^{5A}-R^{5A} is a tryptophan side chain; -L^{6A}-R^{6A} is an asparagine side chain; -L^{7A}-R^{7A} is a leucine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide 3 of FIG. 2, -L^{1A}-R^{1A} is a tyrosine side chain; -L^{2A}-R^{2A} is an alanine side chain; -L^{3A}-R^{3A} is a glutamine side chain; -L^{4A}-R^{4A} is an aspartic acid side chain; -L^{5A}-R^{5A} is a tryptophan side chain; -L^{6A}-R^{6A} is an asparagine side chain; -L^{7A}-R^{7A} is a leucine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide 12 of FIG. 2, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is an aspartic acid side chain; -L^{5A}-R^{5A} is a tryptophan side chain; -L^{6A}-R^{6A} is an asparagine side chain; -L^{7A}-R^{7A} is an isoleucine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide 16 of FIG. 2, -L^{1A}-R^{1A} is a tyrosine side chain; -L^{2A}-R^{2A} is a histidine side chain; -L^{3A}-R^{3A} is a glutamine side chain; -L^{4A}-R^{4A} is an aspartic acid side chain; -L^{5A}-R^{5A} is a tryptophan side chain; -L^{6A}-R^{6A} is an asparagine side chain; -L^{7A}-R^{7A} is a leucine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide 6 of FIG. 2, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

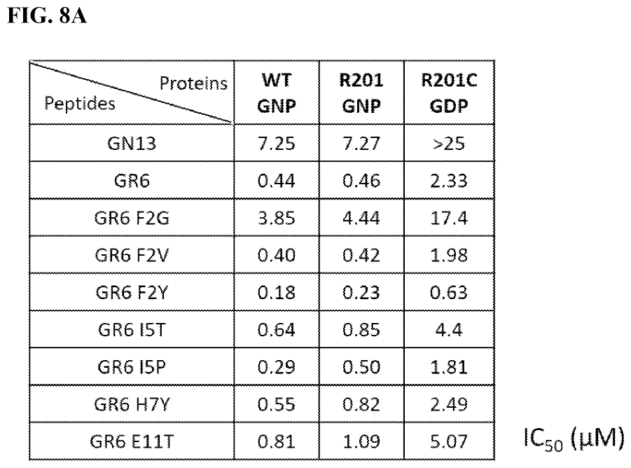

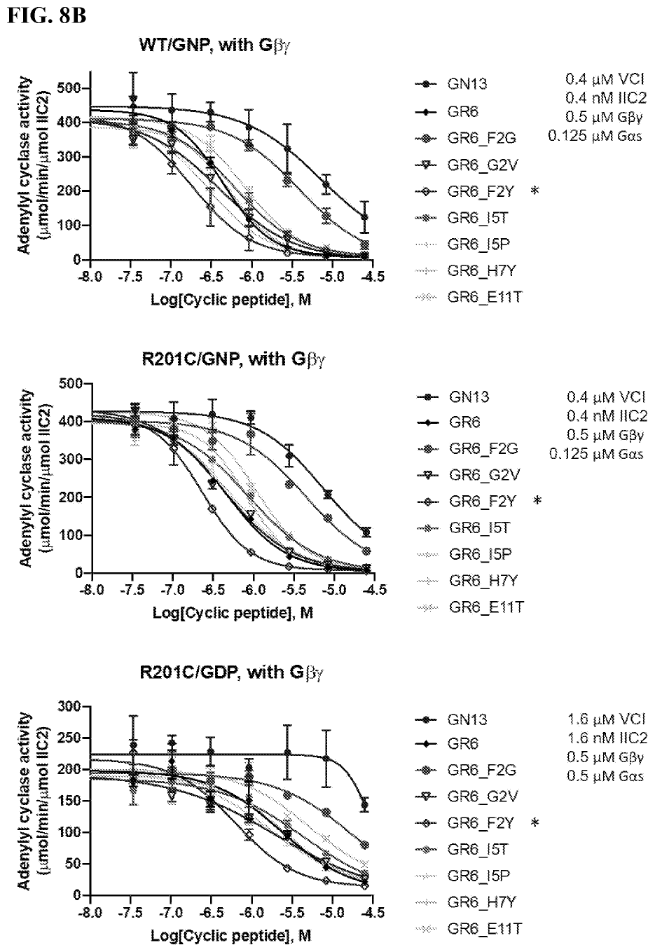

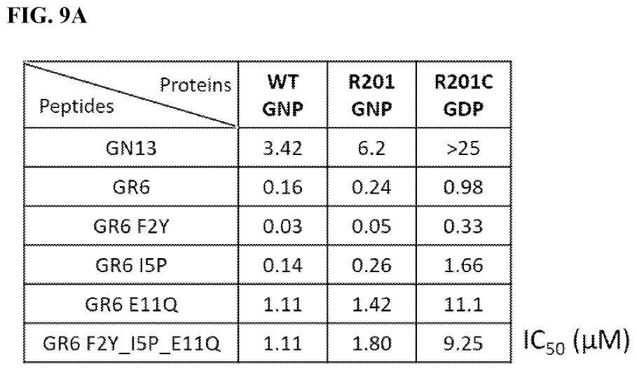

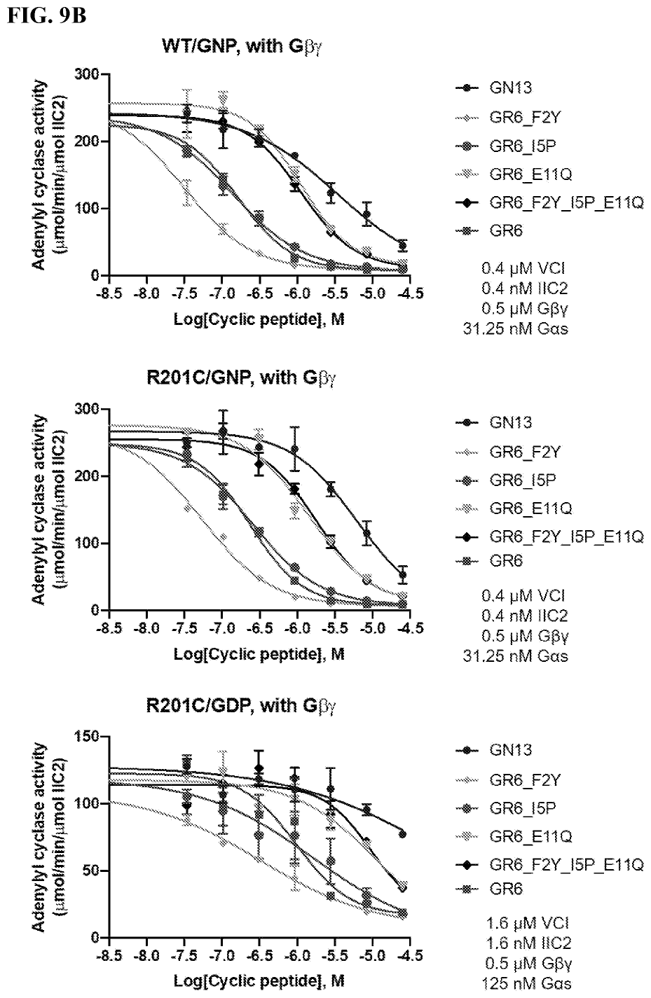

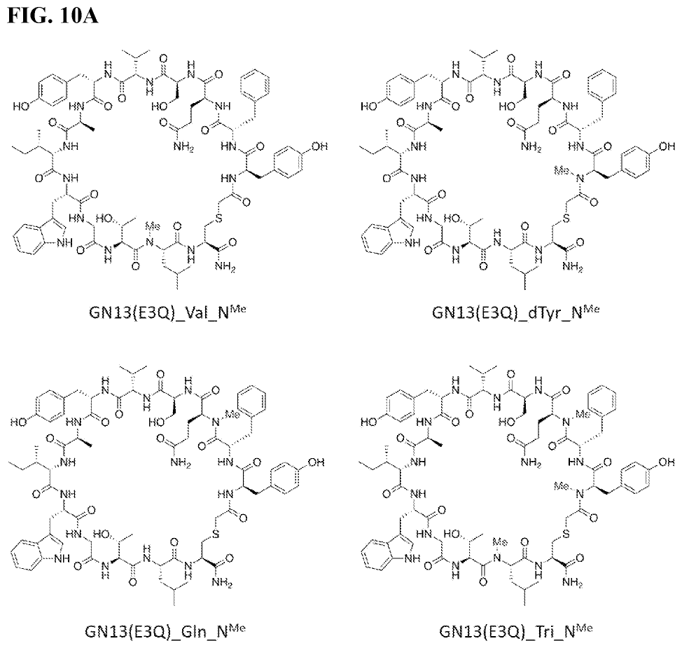

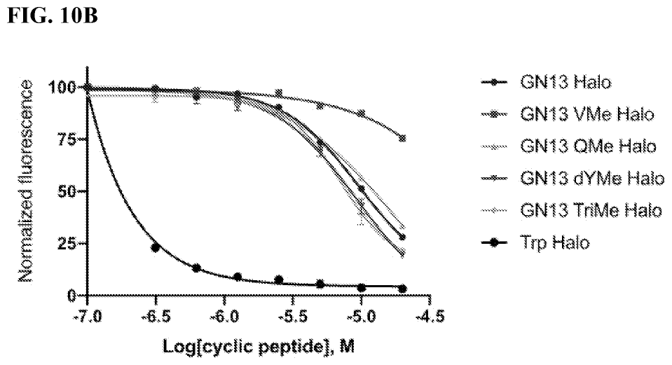

In embodiments, the compound of formula (I) is a peptide of FIG. 8A. In embodiments, the compound of formula (I) is peptide GR6 F2G, GR6 F2V, GR6 F2Y, GR6 I5T, GR6 ISP, GR6 H7Y, or GR6 E11T of FIG. 8A. In embodiments, the compound of formula (I) is a peptide of FIG. 9A. In embodiments, the compound of formula (I) is peptide GR6 F2Y, GR6 ISP, GR6 E11Q, or GR6 F2Y_I5P_E 11Q of FIG. 9A.

For peptide GR6 F2G of FIG. 8A, -L^{1A}-R^{1A} is a tyrosine side chain; -L^{2A}-R^{2A} is a glycine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

For peptide GR6 F2V of FIG. 8A, -L^{1A}-R^{1A} is a tyrosine side chain; -L^{2A}-R^{2A} is a valine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

For peptide GR6 F2Y of FIG. 8A or FIG. 9A, -L^{1A}-R^{1A} is a tyrosine side chain; -L^{2A}-R^{2A} is a tyrosine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

For peptide GR6 I5T of FIG. 8A, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is a serine side chain; -L^{5A}-R^{5A} is a threonine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

For peptide GR6 I5P of FIG. 8A or FIG. 9A, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is a serine side chain; L⁵ is

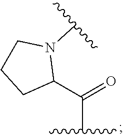 [SMILES: *C(=O)C1CCCN1*]

-L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

For peptide GR6 H7Y of FIG. 8A, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a tyrosine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamic acid side chain; and -L^{12A}-R^{12A} is a leucine side chain.

For peptide GR6 E11T of FIG. 8A, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a threonine side chain; and -L^{12A}-R^{12A} is a leucine side chain.

For peptide GR6 E11Q of FIG. 9A, -L^{1A}-R^{1A} is a tyrosine side chain; -L^{2A}-R^{2A} is a phenylalanine side chain; -L^{3A}-R^{3A} is a glutamine side chain; -L^{4A}-R^{4A} is a serine side chain; -L^{5A}-R^{5A} is an isoleucine side chain; -L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamine side chain; and -L^{12A}-R^{12A} is a leucine side chain.

For peptide GR6 F2Y_I5P_E11Q of FIG. 9A, -L^{1A}-R^{1A} is a tyrosine side chain; -L^{2A}-R^{2A} is a tyrosine side chain; -L^{3A}-R^{3A} is a glutamine side chain; -L^{4A}-R^{4A} is a serine side chain; L⁵ is

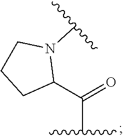 [SMILES: *C(=O)C1CCCN1*]

-L^{6A}-R^{6A} is a tyrosine side chain; -L^{7A}-R^{7A} is a histidine side chain; -L^{8A}-R^{8A} is an isoleucine side chain; -L^{9A}-R^{9A} is a tryptophan side chain; -L^{10A}-R^{10A} is a glycine side chain; -L^{11A}-R^{11A} is a glutamine side chain; and -L^{12A}-R^{12A} is a leucine side chain.

In embodiments, the compound binds a human Gαs protein-GTP complex more strongly than the compound binds a human Gαs protein-GDP complex under identical conditions. In embodiments, the compound binds a human Gαs protein-GTP complex at least 2-fold stronger than the compound binds a human Gαs protein-GDP complex under identical conditions. In embodiments, the compound binds a human Gαs protein-GTP complex at least 5-fold stronger than the compound binds a human Gαs protein-GDP complex under identical conditions. In embodiments, the compound binds a human Gαs protein-GTP complex at least 10-fold stronger than the compound binds a human Gαs protein-GDP complex under identical conditions. In embodiments, the compound binds a human Gαs protein-GTP complex at least 20-fold stronger than the compound binds a human Gαs protein-GDP complex under identical conditions. In embodiments, the compound binds a human Gαs protein-GTP complex at least 40-fold stronger than the compound binds a human Gαs protein-GDP complex under identical conditions. In embodiments, the compound binds a human Gαs protein-GTP complex at least 60-fold stronger than the compound binds a human Gαs protein-GDP complex under identical conditions. In embodiments, the compound binds a human Gαs protein-GTP complex at least 80-fold stronger than the compound binds a human Gαs protein-GDP complex under identical conditions. In embodiments, the compound binds a human Gαs protein-GTP complex at least 100-fold stronger than the compound binds a human Gαs protein-GDP complex under identical conditions. In embodiments, the compound binds a human Gαs protein-GTP complex at least 500-fold stronger than the compound binds a human Gαs protein-GDP complex under identical conditions.

In an aspect is provided a compound having the formula:

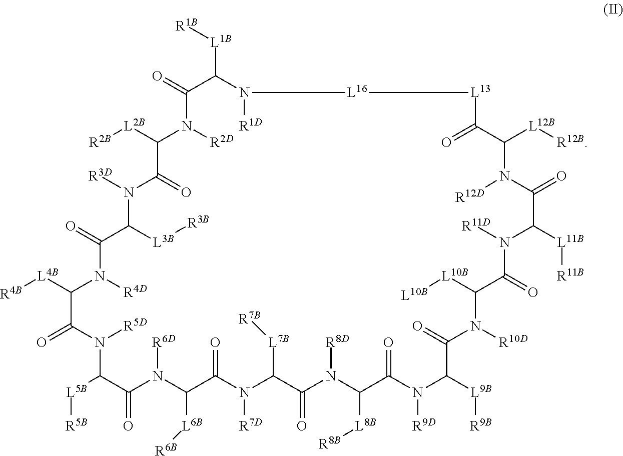

R^{1D}, R^{2D}, R^{3D}, R^{4D}, R^{5D}, R^{6D}, R^{7D}, R^{8D}, R^{9D}, R^{10D}, R^{11D}, R^{12D}, and L¹⁶ are as described herein, including in embodiments.

L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

L¹³ is a bond,

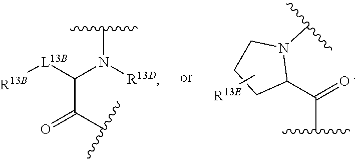

R^{1B} is substituted or unsubstituted aryl (e.g., C₆-C₁₀ or phenyl).

R^{2B}, R^{4B}, R^{5B}, R^{8B}, R^{9B}, and R^{13B} are independently hydrogen, —OH, —NH₂, —C(O)OH, —C(O)NH₂, —NO₂, —SO₃H, —OSO₃H, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3B} is hydrogen, —OH, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{6B}, R^{7B}, R^{10B}, R^{1B}, and R^{12B} are independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{13D} is independently hydrogen or unsubstituted C₁-C₄ alkyl.

R^{13E} is hydrogen, —OH, —NH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered).

In embodiments, the compound has the formula:

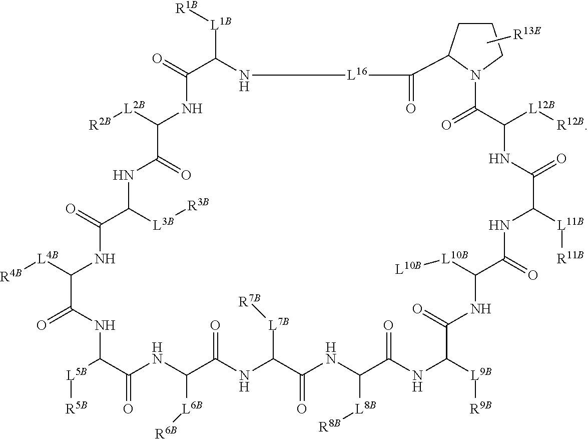

L^{1B}, L^{2B}, L^{3B}, L^{4B}, L^{5B}, L^{6B}, L^{7B}, L^{8B}, L^{9B}, L^{10B}, L^{11B}, L^{12B}, L¹⁶, R^{1B}, R^{2B}, R^{3B}, R^{4B}, R^{5B}, R^{6B}, R^{7B}, R^{8B}, R^{9B}, R^{10B}, R^{11B}, R^{12B} and R^{13E} are as described herein, including in embodiments.

In embodiments, the compound has the formula:

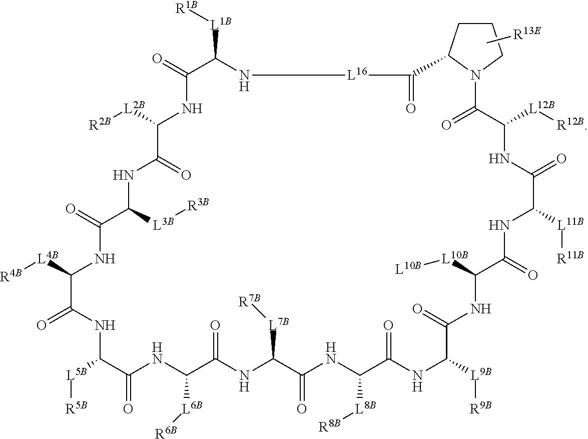

L^{1B}, L^{2B}, L^{3B}, L^{4B}, L^{5B}, L^{6B}, L^{7B}, L^{8B}, L^{9B}, L^{10B}, L^{11B}, L^{12B}, L¹⁶, R^{1B}, R^{2B}, R^{3B}, R^{4B}, R^{5B}, R^{6B}, R^{7B}, R^{8B}, R^{9B}, R^{10B}, R^{11B}, R^{12B} and R^{13E} are as described herein, including in embodiments.

In embodiments, -L^{1B}-R^{1B}, -L^{2B}-R^{2B}, -L^{3B}-R^{3B}, -L^{4B}-R^{4B}, -L^{5B}-R^{5B}, -L^{6B}-R^{6B}, -L^{7B}-R^{7B}, -L^{8B}-R^{8B}-L^{9B}-R^{9B}-L^{10B}-R^{10B}-L^{11B}-R^{11B}-L^{12B}-R^{12B}, or -L^{13B}-R^{13B} are independently a natural amino acid side chain or an unnatural amino acid side chain. In embodiments, -L^{1B}-R^{1B}, -L^{2B}-R^{2B}, -L^{3B}-R^{3B}, -L^{4B}-R^{4B}, -L^{5B}-R^{5B}, -L^{6B}-R^{6B}, -L^{7B}-R^{7B}, -L^{8B}-R^{8B}, -L^{9B}-R^{9B}, -L^{10B}-R^{10B}-L^{11B}-R^{11B}-L^{12B}-R^{12B}, or -L^{13B}-R^{13B} are independently a natural amino acid side chain. In embodiments, -L^{1B}-R^{1B}, -L^{2B}-R^{2B}, -L^{3B}-R^{3B}, -L^{4B}-R^{4B},-L^{5B}-R^{5B}-L^{6B}-R^{6B}-L^{7B}-R^{7B}, -L^{8B}-R^{8B}-L^{9B}-R^{9B}-L^{10B}-R^{10B}-L^{11B}-R^{11B}-L^{12B}-R^{12B}, or -L^{13B}-R^{13B} are independently an unnatural amino acid side chain.

In embodiments, a substituted L^{1B} (e.g., substituted alkylene and/or substituted heteroalkylene) is substituted with at least one substituent group, size-limited substituent group, or lower substituent group; wherein if the substituted L^{1B} is substituted with a plurality of groups selected from substituent groups, size-limited substituent groups, and lower substituent groups; each substituent group, size-limited substituent group, and/or lower substituent group may optionally be different. In embodiments, when L^{1B} is substituted, it is substituted with at least one substituent group. In embodiments, when L^{1B} is substituted, it is substituted with at least one size-limited substituent group. In embodiments, when L^{1B} is substituted, it is substituted with at least one lower substituent group.

In embodiments, a substituted L^{2B} (e.g., substituted alkylene and/or substituted heteroalkylene) is substituted with at least one substituent group, size-limited substituent group, or lower substituent group; wherein if the substituted L^{2B} is substituted with a plurality of groups selected from substituent groups, size-limited substituent groups, and lower substituent groups; each substituent group, size-limited substituent group, and/or lower substituent group may optionally be different. In embodiments, when L^{2B} is substituted, it is substituted with at least one substituent group. In embodiments, when L^{2B} is substituted, it is substituted with at least one size-limited substituent group. In embodiments, when L^{2B} is substituted, it is substituted with at least one lower substituent group.

In embodiments, a substituted L^{3B} (e.g., substituted alkylene and/or substituted heteroalkylene) is substituted with at least one substituent group, size-limited substituent group, or lower substituent group; wherein if the substituted L^{3B} is substituted with a plurality of groups selected from substituent groups, size-limited substituent groups, and lower substituent groups; each substituent group, size-limited substituent group, and/or lower substituent group may optionally be different. In embodiments, when L^{3B} is substituted, it is substituted with at least one substituent group. In embodiments, when L^{3B} is substituted, it is substituted with at least one size-limited substituent group. In embodiments, when L^{3B} is substituted, it is substituted with at least one lower substituent group.

In embodiments, a substituted L^{4B} (e.g., substituted alkylene and/or substituted heteroalkylene) is substituted with at least one substituent group, size-limited substituent group, or lower substituent group; wherein if the substituted L^{4B} is substituted with a plurality of groups selected from substituent groups, size-limited substituent groups, and lower substituent groups; each substituent group, size-limited substituent group, and/or lower substituent group may optionally be different. In embodiments, when L^{4B} is substituted, it is substituted with at least one substituent group. In embodiments, when L^{4B} is substituted, it is substituted with at least one size-limited substituent group. In embodiments, when L^{4B} is substituted, it is substituted with at least one lower substituent group.

In embodiments, a substituted L^{5B} (e.g., substituted alkylene and/or substituted heteroalkylene) is substituted with at least one substituent group, size-limited substituent group, or lower substituent group; wherein if the substituted L^{5B} is substituted with a plurality of groups selected from substituent groups, size-limited substituent groups, and lower substituent groups; each substituent group, size-limited substituent group, and/or lower substituent group may optionally be different. In embodiments, when L^{5B} is substituted, it is substituted with at least one substituent group. In embodiments, when L^{5B} is substituted, it is substituted with at least one size-limited substituent group. In embodiments, when L^{5B} is substituted, it is substituted with at least one lower substituent group.

In embodiments, a substituted L^{6B} (e.g., substituted alkylene and/or substituted heteroalkylene) is substituted with at least one substituent group, size-limited substituent group, or lower substituent group; wherein if the substituted L^{6B} is substituted with a plurality of groups selected from substituent groups, size-limited substituent groups, and lower substituent groups; each substituent group, size-limited substituent group, and/or lower substituent group may optionally be different. In embodiments, when L^{6B} is substituted, it is substituted with at least one substituent group. In embodiments, when L^{6B} is substituted, it is substituted with at least one size-limited substituent group. In embodiments, when L^{6B} is substituted, it is substituted with at least one lower substituent group.

In embodiments, a substituted L^{7B}, (e.g., substituted alkylene and/or substituted heteroalkylene) is substituted with at least one substituent group, size-limited substituent group, or lower substituent group; wherein if the substituted L^{7B}, is substituted with a plurality of groups selected from substituent groups, size-limited substituent groups, and lower substituent groups; each substituent group, size-limited substituent group, and/or lower substituent group may optionally be different. In embodiments, when L^{7B}, is substituted, it is substituted with at least one substituent group. In embodiments, when L^{7B}, is substituted, it is substituted with at least one size-limited substituent group. In embodiments, when L^{7B}, is substituted, it is substituted with at least one lower substituent group.

In embodiments, a substituted L^{8B} (e.g., substituted alkylene and/or substituted heteroalkylene) is substituted with at least one substituent group, size-limited substituent group, or lower substituent group; wherein if the substituted L^{8B} is substituted with a plurality of groups selected from substituent groups, size-limited substituent groups, and lower substituent groups; each substituent group, size-limited substituent group, and/or lower substituent group may optionally be different. In embodiments, when L^{8B} is substituted, it is substituted with at least one substituent group. In embodiments, when L^{8B} is substituted, it is substituted with at least one size-limited substituent group. In embodiments, when L^{8B} is substituted, it is substituted with at least one lower substituent group.

In embodiments, a substituted L^{9B} (e.g., substituted alkylene and/or substituted heteroalkylene) is substituted with at least one substituent group, size-limited substituent group, or lower substituent group; wherein if the substituted L^{9B} is substituted with a plurality of groups selected from substituent groups, size-limited substituent groups, and lower substituent groups; each substituent group, size-limited substituent group, and/or lower substituent group may optionally be different. In embodiments, when L^{9B} is substituted, it is substituted with at least one substituent group. In embodiments, when L^{9B} is substituted, it is substituted with at least one size-limited substituent group. In embodiments, when L^{9B} is substituted, it is substituted with at least one lower substituent group.

In embodiments, a substituted L^{10B} (e.g., substituted alkylene and/or substituted heteroalkylene) is substituted with at least one substituent group, size-limited substituent group, or lower substituent group; wherein if the substituted L^{10B} is substituted with a plurality of groups selected from substituent groups, size-limited substituent groups, and lower substituent groups; each substituent group, size-limited substituent group, and/or lower substituent group may optionally be different. In embodiments, when L^{10B} is substituted, it is substituted with at least one substituent group. In embodiments, when L^{10B} is substituted, it is substituted with at least one size-limited substituent group. In embodiments, when L^{10B} is substituted, it is substituted with at least one lower substituent group.

In embodiments, a substituted L^{11B} (e.g., substituted alkylene and/or substituted heteroalkylene) is substituted with at least one substituent group, size-limited substituent group, or lower substituent group; wherein if the substituted L^{11B} is substituted with a plurality of groups selected from substituent groups, size-limited substituent groups, and lower substituent groups; each substituent group, size-limited substituent group, and/or lower substituent group may optionally be different. In embodiments, when L^{11B} is substituted, it is substituted with at least one substituent group. In embodiments, when L^{11B} is substituted, it is substituted with at least one size-limited substituent group. In embodiments, when L^{11B} is substituted, it is substituted with at least one lower substituent group.

In embodiments, a substituted L^{12B}(e.g., substituted alkylene and/or substituted heteroalkylene) is substituted with at least one substituent group, size-limited substituent group, or lower substituent group; wherein if the substituted L^{12B} is substituted with a plurality of groups selected from substituent groups, size-limited substituent groups, and lower substituent groups; each substituent group, size-limited substituent group, and/or lower substituent group may optionally be different. In embodiments, when L^{12B} is substituted, it is substituted with at least one substituent group. In embodiments, when L^{12B} is substituted, it is substituted with at least one size-limited substituent group. In embodiments, when L^{12B} is substituted, it is substituted with at least one lower substituent group.

In embodiments, a substituted L^{13B} (e.g., substituted alkylene and/or substituted heteroalkylene) is substituted with at least one substituent group, size-limited substituent group, or lower substituent group; wherein if the substituted L^{13B} is substituted with a plurality of groups selected from substituent groups, size-limited substituent groups, and lower substituent groups; each substituent group, size-limited substituent group, and/or lower substituent group may optionally be different. In embodiments, when L^{13B} is substituted, it is substituted with at least one substituent group. In embodiments, when L^{13B} is substituted, it is substituted with at least one size-limited substituent group. In embodiments, when L^{13B} is substituted, it is substituted with at least one lower substituent group.

In embodiments, a substituted R^{1B} (e.g., substituted aryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted R^{3B} (e.g., substituted alkyl and/or substituted heteroalkyl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B}, is substituted, it is substituted with at least one lower substituent group.

In embodiments, a substituted R^{8B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8B} is substituted with a plurality of groups selected from substituent groups, size-limited substituent groups, and lower substituent groups; each substituent group, size-limited substituent group, and/or lower substituent group may optionally be different. In embodiments, when R^{8B} is substituted, it is substituted with at least one substituent group. In embodiments, when R^{8B} is substituted, it is substituted with at least one size-limited substituent group. In embodiments, when R^{8B} is substituted, it is substituted with at least one lower substituent group.

In embodiments, a substituted R^{9B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{9B} is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R^{9B} is substituted, it is substituted with at least one size-limited substituent group. In embodiments, when R^{9B} is substituted, it is substituted with at least one lower substituent group.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted R^{11B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted R^{12B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12B} is substituted with a plurality of groups selected from substituent groups, size-limited substituent groups, and lower substituent groups; each substituent group, size-limited substituent group, and/or lower substituent group may optionally be different. In embodiments, when R^{12B} is substituted, it is substituted with at least one substituent group. In embodiments, when R^{12B} is substituted, it is substituted with at least one size-limited substituent group. In embodiments, when R^{12B} is substituted, it is substituted with at least one lower substituent group.

In embodiments, a substituted R^{13B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13B} is substituted with a plurality of groups selected from substituent groups, size-limited substituent groups, and lower substituent groups; each substituent group, size-limited substituent group, and/or lower substituent group may optionally be different. In embodiments, when R^{13B} is substituted, it is substituted with at least one substituent group. In embodiments, when R^{13B} is substituted, it is substituted with at least one size-limited substituent group. In embodiments, when R^{13B} is substituted, it is substituted with at least one lower substituent group.

In embodiments, a substituted R^{13E} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13E} is substituted with a plurality of groups selected from substituent groups, size-limited substituent groups, and lower substituent groups; each substituent group, size-limited substituent group, and/or lower substituent group may optionally be different. In embodiments, when R^{13E} is substituted, it is substituted with at least one substituent group. In embodiments, when R^{13E} is substituted, it is substituted with at least one size-limited substituent group. In embodiments, when R^{13E} is substituted, it is substituted with at least one lower substituent group.

In embodiments,

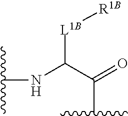

is a divalent form of an unnatural amino acid. In embodiments, L^{1B} is a bond or unsubstituted C₁-C₄ alkylene. In embodiments, R^{1B} is a substituted aryl. In embodiments,

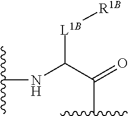

is a divalent form of a natural amino acid. In embodiments,

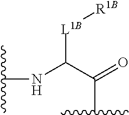

is a divalent form of tyrosine. In embodiments, -L^{1B}-R^{1B} is

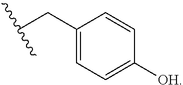 [SMILES: *Cc1ccc(O)cc1]

In embodiments,

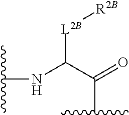

is a divalent form of an unnatural amino acid. In embodiments, L^{2B} is a bond or unsubstituted C₁-C₆ alkylene. In embodiments, R^{2B} is hydrogen, —OH, —NH₂, —C(O)OH, —C(O)NH₂, substituted or unsubstituted alkyl, or substituted or unsubstituted heteroaryl. In embodiments,

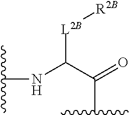

is a divalent form of a natural amino acid. In embodiments,

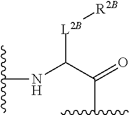

is a divalent form of lysine, a divalent form of leucine, a divalent form of serine, a divalent form of asparagine, a divalent form of glutamine, a divalent form of histidine, a divalent form of aspartic acid, or a divalent form of glycine. In embodiments,

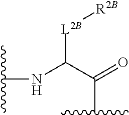

is a divalent form of lysine. In embodiments,

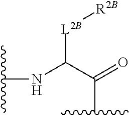

is a divalent form of leucine. In embodiments,

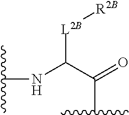

is a divalent form of serine. In embodiments,

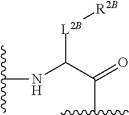

is a divalent form of asparagine. In embodiments,

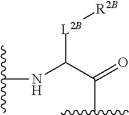

is a divalent form of glutamine. In embodiments,

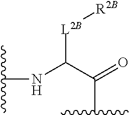

is a divalent form of histidine. In embodiments,

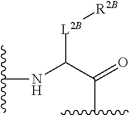

is a divalent form of aspartic acid. In embodiments,

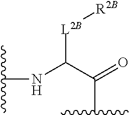

is a divalent form of glycine. In embodiments, -L^{2B}R^{2B} is

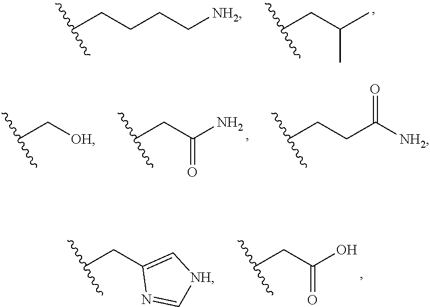

or —H. In embodiments, -L^{2B}-R^{2B} is

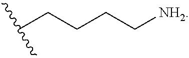 [SMILES: *CCCCN]

In embodiments, -L^{2B}-R^{2B} is

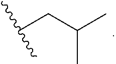 [SMILES: *CC(C)C]

In embodiments, -L^{2B}-R^{2B} is

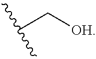 [SMILES: *CO]

In embodiments, -L^{2B}-R^{2B} is

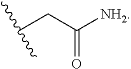 [SMILES: *CC(N)=O]

In embodiments, -L^{2B}-R^{2B} is

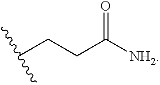 [SMILES: *CCC(N)=O]

In embodiments, -L^{2B}-R^{2B} is

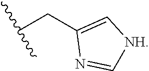 [SMILES: *Cc1c[nH]cn1]

In embodiments, -L^{2B}-R^{2B} is

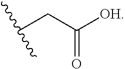 [SMILES: *CC(=O)O]

In embodiments, -L^{2B}-R^{2B} is —H.

In embodiments

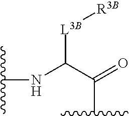

is a divalent form of an unnatural amino acid. In embodiments, L^{3B} is a bond or unsubstituted C₁-C₆ alkylene. In embodiments, R^{3B} is hydrogen, —NH₂, —C(O)NH₂, —NHC(NH)NH₂, or substituted or unsubstituted alkyl. In embodiments

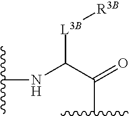

is a divalent form of a natural amino acid. In embodiments,

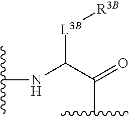

is a divalent form of leucine, a divalent form of valine, a divalent form of isoleucine, a divalent form of lysine, a divalent form of glycine, a divalent form of glutamine, or a divalent form of arginine. In embodiments,

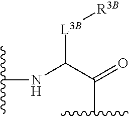

is a divalent form of leucine. In embodiments,

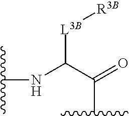

is a divalent form of valine. In embodiments,

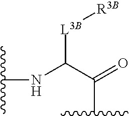

is a divalent form of isoleucine. In embodiments,

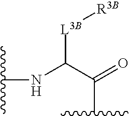

is a divalent form of lysine. In embodiments,

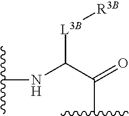

is a divalent form of glycine. In embodiments,

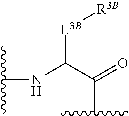

is a divalent form of glutamine. In embodiments,

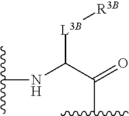

is a divalent form of arginine. In embodiments, -L^{3B}-R^{3B} is

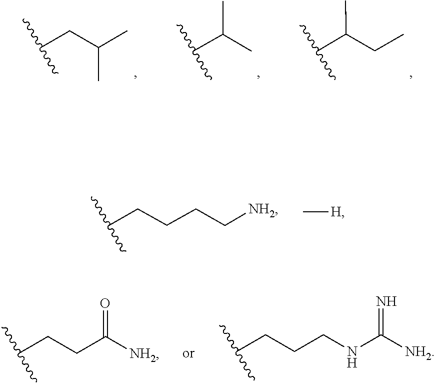

In embodiments, -L^{3B}-R^{3B} is

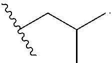 [SMILES: *CC(C)C]

In embodiments, -L^{3B}-R^{3B} is

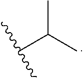 [SMILES: *C(C)C]

In embodiments, -L^{3B}-R^{3B} is

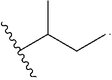 [SMILES: *C(C)CC]

In embodiments, -L^{3B}-R^{3B} is

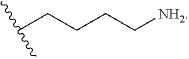 [SMILES: *CCCCN]

In embodiments, -L^{3B}-R^{3B} is —H. In embodiments, -L^{3B}-R^{3B} is

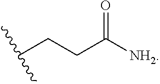 [SMILES: *CCC(N)=O]

In embodiments, -L^{3B}-R^{3B} is

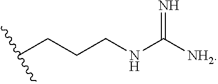 [SMILES: *CCCNC(=N)N]

In embodiments,

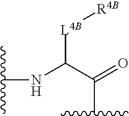

is a divalent form of an unnatural amino acid. In embodiments, L^{4B} is a bond or unsubstituted C₁-C₆ alkylene. In embodiments, R^{4B} is —OH, —NH₂, substituted or unsubstituted alkyl, substituted or unsubstituted aryl, or substituted or unsubstituted heteroaryl. In embodiments,

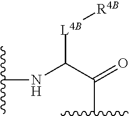

is a divalent form of a natural amino acid. In embodiments,

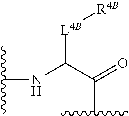

is a divalent form of threonine, a divalent form of lysine, a divalent form of leucine, a divalent form of phenylalanine, a divalent form of histidine, or a divalent form of isoleucine. In embodiments,

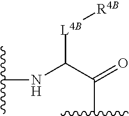

is a divalent form of threonine. In embodiments,

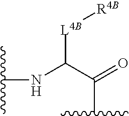

is a divalent form of lysine. In embodiments,

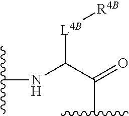

is a divalent form of leucine. In embodiments,

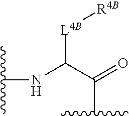

is a divalent form of phenylalanine. In embodiments,

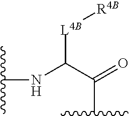

is a divalent form of histidine. In embodiments,

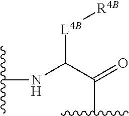

is a divalent form of isoleucine. In embodiments, -L^{4B}-R^{4B} is

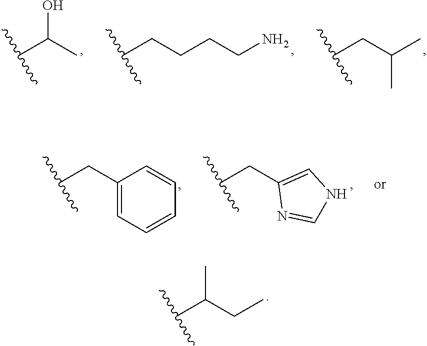

In embodiments, -L^{4B}-R^{4B} is

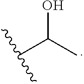 [SMILES: *C(C)O]

In embodiments, -L^{4B}-R^{4B} is

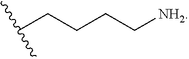 [SMILES: *CCCCN]

In embodiments, -L^{4B}-R^{4B} is

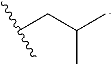 [SMILES: *CC(C)C]

In embodiments, -L^{4B}-R^{4B} is

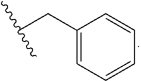 [SMILES: *Cc1ccccc1]

In embodiments, -L^{4B}-R^{4B} is

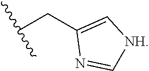 [SMILES: *Cc1c[nH]cn1]

In embodiments, -L^{4B}-R^{4B} is

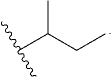 [SMILES: *C(C)CC]

In embodiments,

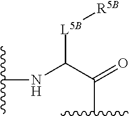

is a divalent form of an unnatural amino acid. In embodiments, L^{5B} is a bond or unsubstituted C₁-C₄ alkylene. In embodiments, R^{5B} is —OH, substituted or unsubstituted alkyl, substituted or unsubstituted aryl, or substituted or unsubstituted heteroaryl. In embodiments,

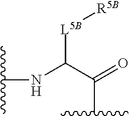

is a divalent form of a natural amino acid. In embodiments,

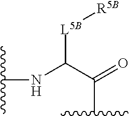

is a divalent form of valine, a divalent form of isoleucine, a divalent form of tryptophan, a divalent form of leucine, a divalent form of threonine, or a divalent form of phenylalanine. In embodiments,

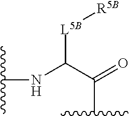

is a divalent form of valine. In embodiments,

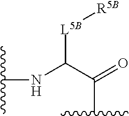

is a divalent form of isoleucine. In embodiments,

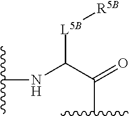

is a divalent form of tryptophan. In embodiments,

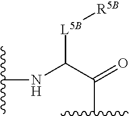

is a divalent form of leucine. In embodiments,

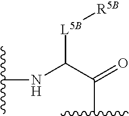

is a divalent form of threonine. In embodiments,

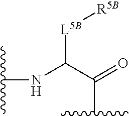

is a divalent form of phenylalanine. In embodiments, -L^{5B}-R^{5B} is

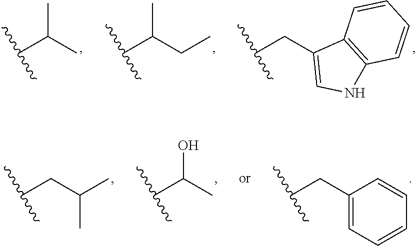

In embodiments, -L^{5B}-R^{5B} is

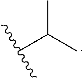 [SMILES: *C(C)C]

In embodiments, -L^{5B}-R^{5B} is

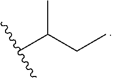 [SMILES: *C(C)CC]

In embodiments, -L^{5B}-R^{5B} is

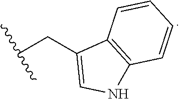 [SMILES: *Cc1c[nH]c2ccccc12]

In embodiments, -L^{5B}-R^{5B} is

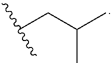 [SMILES: *CC(C)C]

In embodiments, -L^{5B}-R^{5B} is

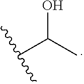 [SMILES: *C(C)O]

In embodiments, L^{5B}-R^{5B} is

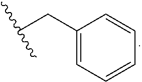 [SMILES: *Cc1ccccc1]

In embodiments,

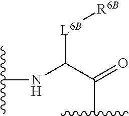

is a divalent form of an unnatural amino acid. In embodiments, L^{6B} is a bond or unsubstituted C₁-C₄ alkylene. In embodiments, R^{6B} is —OH, —C(O)NH₂, —NHC(NH)NH₂, substituted or unsubstituted alkyl, substituted or unsubstituted aryl, or substituted or unsubstituted heteroaryl. In embodiments,

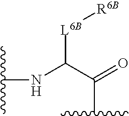

is a divalent form of a natural amino acid. In embodiments,

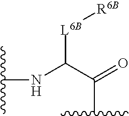

is a divalent form of tyrosine, a divalent form of leucine, a divalent form of threonine, a divalent form of valine, a divalent form of arginine, a divalent form of isoleucine, a divalent form of asparagine, or a divalent form of tryptophan. In embodiments,

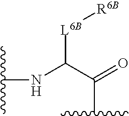

is a divalent form of tyrosine. In embodiments,

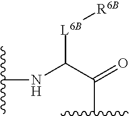

is a divalent form of leucine. In embodiments,

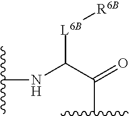

is a divalent form of threonine. In embodiments,

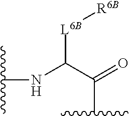

is a divalent form of valine. In embodiments,

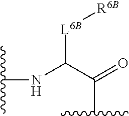

is a divalent form of arginine. In embodiments,

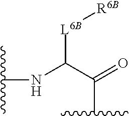

is a divalent form of isoleucine. In embodiments,

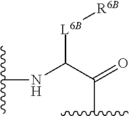

is a divalent form of asparagine. In embodiments,

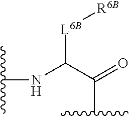

is a divalent form of tryptophan. In embodiments, -L^{6B}-R^{6B} is

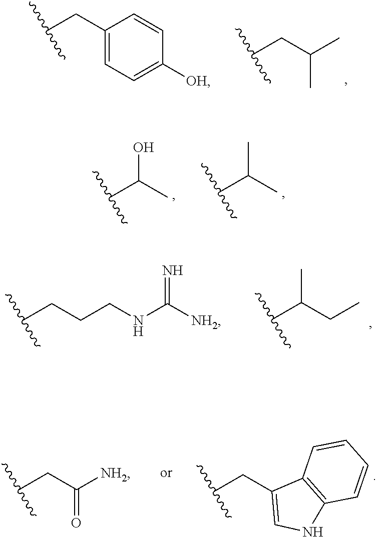

In embodiments, -L^{6B}-R^{6B} is

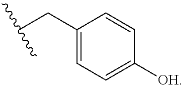 [SMILES: *Cc1ccc(O)cc1]

In embodiments, -L^{6B}-R^{6B} is

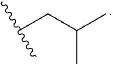 [SMILES: *CC(C)C]

In embodiments, -L^{6B}-R^{6B} is

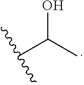 [SMILES: *C(C)O]

In embodiments, L^{6B}R^{6B} is

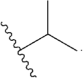 [SMILES: *C(C)C]

In embodiments, -L^{6B}-R^{6B} is

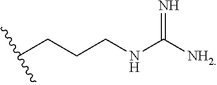 [SMILES: *CCCNC(=N)N]

In embodiments, -L^{6B}-R^{6B} is

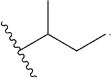 [SMILES: *C(C)CC]

In embodiments, -L^{6B}-R^{6B} is

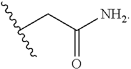 [SMILES: *CC(N)=O]

In embodiments, -L^{6B}-R^{6B} is

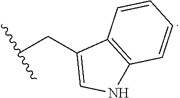 [SMILES: *Cc1c[nH]c2ccccc12]

In embodiments,

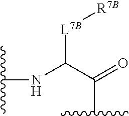

is a divalent form of an unnatural amino acid. In embodiments, L^{7B} is a bond or unsubstituted C₁-C₄ alkylene. In embodiments, R^{7B} is —OH, —C(O)OH, —C(O)NH₂, —NHC(NH)NH₂, or substituted or unsubstituted alkyl. In embodiments,

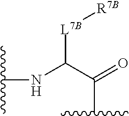

is a divalent form of a natural amino acid. In embodiments,

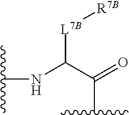

is a divalent form of glutamic acid, a divalent form of threonine, a divalent form of isoleucine, a divalent form of leucine, a divalent form of arginine, a divalent form of glutamine, or a divalent form of aspartic acid. In embodiments,

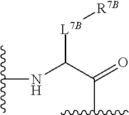

is a divalent form of glutamic acid. In embodiments,

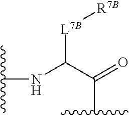

is a divalent form of threonine. In embodiments,

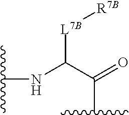

is a divalent form of isoleucine. In embodiments,

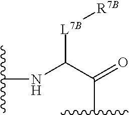

is a divalent form of leucine. In embodiments,

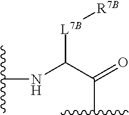

is a divalent form of arginine. In embodiments,

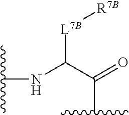

is a divalent form of glutamine. In embodiments,

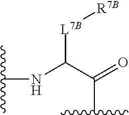

is a divalent form of aspartic acid. In embodiments, -L^{7B}-R^{7B} is

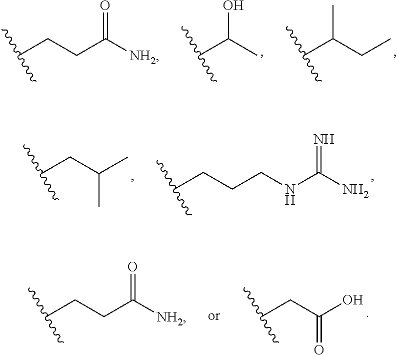

In embodiments, -L^{7B}-R^{7B} is

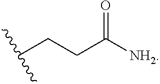 [SMILES: *CCC(N)=O]

In embodiments, -L^{7B}-R^{7B} is

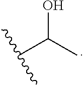 [SMILES: *C(C)O]

In embodiments, -L^{7B}-R^{7B}, is

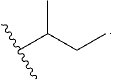 [SMILES: *C(C)CC]

In embodiments, -L^{7B}-R^{7B}, is

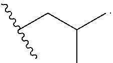 [SMILES: *CC(C)C]

In embodiments, -L^{7B}-R^{7B} is

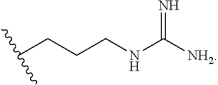 [SMILES: *CCCNC(=N)N]

In embodiments, -L^{7B}-R^{7B}, is

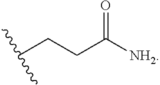 [SMILES: *CCC(N)=O]

In embodiments, -L^{7B}-R^{7B} is

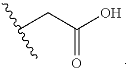 [SMILES: *CC(=O)O]

In embodiments,

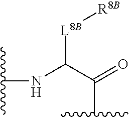

is a divalent form of an unnatural amino acid. In embodiments, L^{8B} is a bond or unsubstituted C₁-C₄ alkylene. In embodiments, R^{8B} is —C(O)OH, —C(O)NH₂, substituted or unsubstituted alkyl, substituted or unsubstituted aryl, or substituted or unsubstituted heteroaryl. In embodiments,

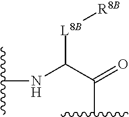

is a divalent form of a natural amino acid. In embodiments,

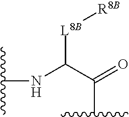

is a divalent form of phenylalanine, a divalent form of valine, a divalent form or tyrosine, a divalent form of asparagine, a divalent form of leucine, a divalent form of glutamic acid, or a divalent form of tryptophan. In embodiments,

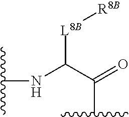

is a divalent form of phenylalanine. In embodiments,

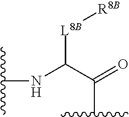

is a divalent form of valine. In embodiments,

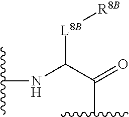

is a divalent form or tyrosine. In embodiments,

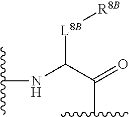

is a divalent form of asparagine. In embodiments,

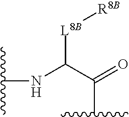

is a divalent form of leucine. In embodiments,

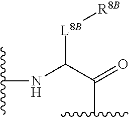

is a divalent form of glutamic acid. In embodiments,

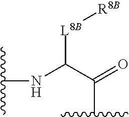

is a divalent form of tryptophan. In embodiments, -L^{8B}-R^{8B} is

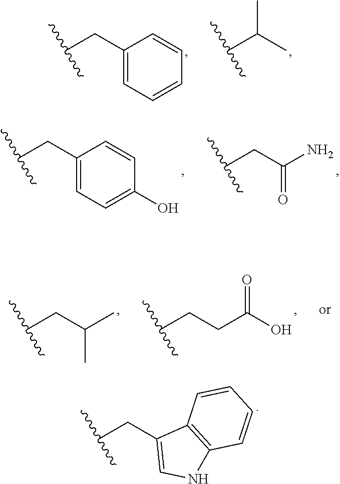

In embodiments, -L^{8B}-R^{8B} is

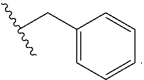 [SMILES: *Cc1ccccc1]

In embodiments, -L^{8B}-R^{8B} is

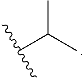 [SMILES: *C(C)C]

In embodiments, -L^{8B}-R^{8B} is

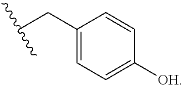 [SMILES: *Cc1ccc(O)cc1]

In embodiments, -L^{8B}-R^{8B} is

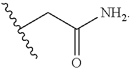 [SMILES: *CC(N)=O]

In embodiments, -L^{8B}-R^{8B} is

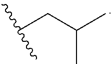 [SMILES: *CC(C)C]

In embodiments, -L^{8B}-R^{8B} is

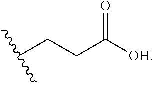 [SMILES: *CCC(=O)O]

In embodiments, -L^{8B}-R^{8B} is

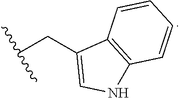 [SMILES: *Cc1c[nH]c2ccccc12]

In embodiments,

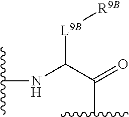

is a divalent form of an unnatural amino acid. In embodiments, L^{9B} is a bond or unsubstituted C₁-C₄ alkylene. In embodiments, R^{9B} is —C(O)OH, —C(O)NH₂, substituted or unsubstituted alkyl, substituted or unsubstituted aryl, or substituted or unsubstituted heteroaryl. In embodiments,

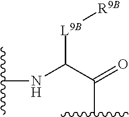

is a divalent form of a natural amino acid. In embodiments,

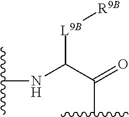

is a divalent form of leucine, a divalent form of valine, a divalent form of glutamic acid, a divalent form of asparagine, a divalent form of phenylalanine, a divalent form of isoleucine, a divalent form of tryptophan, a divalent form of alanine, or a divalent form of histidine. In embodiments,

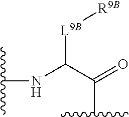

divalent form of isoleucine. In embodiments, -L^{9B}R^{9B} is

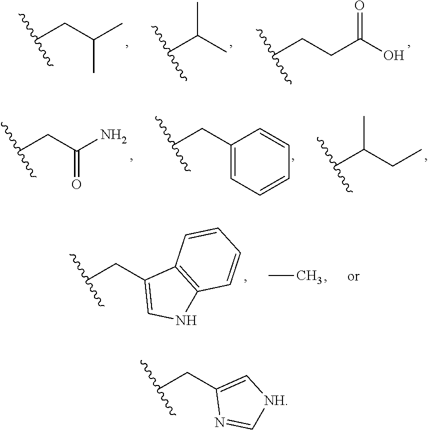

In embodiments, -L^{9B}-R^{9B} is

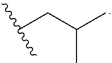 [SMILES: *CC(C)C]

In embodiments, -L^{9B}-R^{9B} is

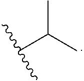 [SMILES: *C(C)C]

In embodiments, -L^{9B}-R^{9B} is

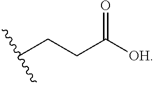 [SMILES: *CCC(=O)O]

In embodiments, -L^{9B}-R^{9B} is

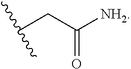 [SMILES: *CC(N)=O]

In embodiments, -L^{9B}-R^{9B} is

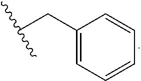 [SMILES: *Cc1ccccc1]

In embodiments, -L^{9B}R^{9B} is

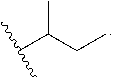 [SMILES: *C(C)CC]

In embodiments, -L^{9B}-R^{9B} is

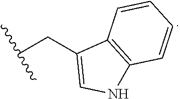 [SMILES: *Cc1c[nH]c2ccccc12]

In embodiments, -L^{9B}-R^{9B} is —CH₃. In embodiments, -L^{9B}-R^{9B} is

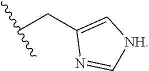 [SMILES: *Cc1c[nH]cn1]

In embodiments

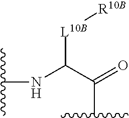

is a divalent form of an unnatural amino acid. In embodiments, L^{10B} is a bond or unsubstituted C₁-C₄ alkylene. In embodiments, R^{10B} is —OH, —C(O)OH, —C(O)NH₂, —NHC(NH)NH₂, substituted or unsubstituted alkyl, substituted or unsubstituted aryl, or substituted or unsubstituted heteroaryl. In embodiments,

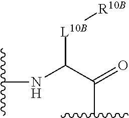

is a divalent form of a natural amino acid. In embodiments,

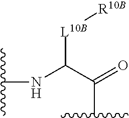

is a divalent form of leucine, a divalent form of phenylalanine, a divalent form of alanine, a divalent form of aspartic acid, a divalent form of arginine, a divalent form of serine, a divalent form of glutamic acid, a divalent form of isoleucine, a divalent form of glutamine, or a divalent form of tryptophan. In embodiments,

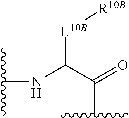

is a divalent form of leucine. In embodiments,

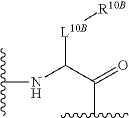

is a divalent form of phenylalanine. In embodiments,

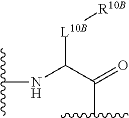

is a divalent form of alanine. In embodiments,

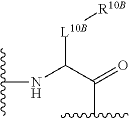

is a divalent form of aspartic acid. In embodiments,

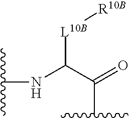

is a divalent form of arginine. In embodiments,

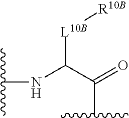

is a divalent form of serine. In embodiments,

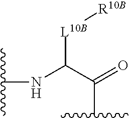

is a divalent form of glutamic acid. In embodiments,

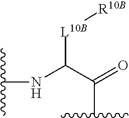

is a divalent form of isoleucine. In embodiments,

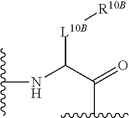

is a divalent form of glutamine. In embodiments,

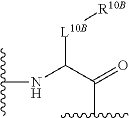

is a divalent form of tryptophan. In embodiments, -L^{10B}-R^{10B} is

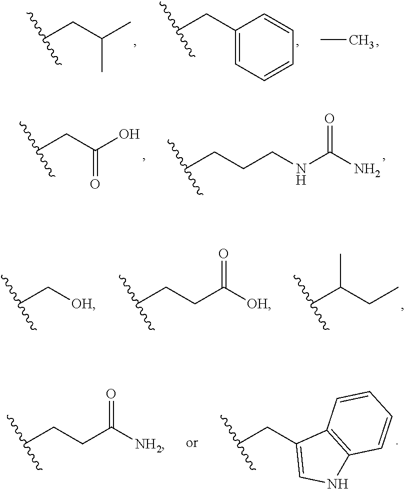

In embodiments, -L^{10B}-R^{10B} is

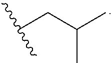 [SMILES: *CC(C)C]

In embodiments, -L^{10B}-R^{10B} is

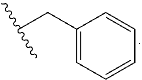 [SMILES: *Cc1ccccc1]

In embodiments, -L^{10B}-R^{10B} is —CH₃. In embodiments, -L^{10B}-R^{10B} is

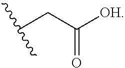 [SMILES: *CC(=O)O]

In embodiments, -L^{10B}-R^{10B} is

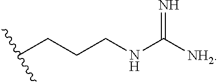 [SMILES: *CCCNC(=N)N]

In embodiments, -L^{10B}-R^{10B} is

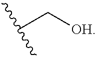 [SMILES: *CO]

In embodiments, -L^{10B}-R^{10B} is

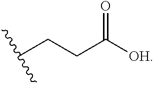 [SMILES: *CCC(=O)O]

In embodiments, -L^{10B}-R^{10B} is

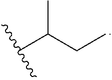 [SMILES: *C(C)CC]

In embodiments, -L^{10B}-R^{10B} is

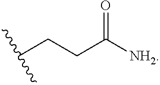 [SMILES: *CCC(N)=O]

In embodiments, -L^{10B}-R^{10B} is

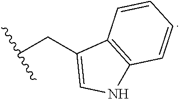 [SMILES: *Cc1c[nH]c2ccccc12]

In embodiments,

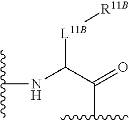

is a divalent form of an unnatural amino acid. In embodiments, L^{11B} is a bond or unsubstituted C₁-C₄ alkylene. In embodiments, R^{1B} is hydrogen, —OH, —C(O)OH, —C(O)NH₂, substituted or unsubstituted alkyl, substituted or unsubstituted aryl, or substituted or unsubstituted heteroaryl. In embodiments,

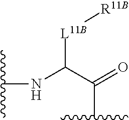

is a divalent form of a natural amino acid. In embodiments,

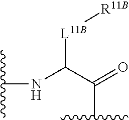

is a divalent form of glutamic acid, a divalent form of alanine, a divalent form of aspartic acid, a divalent form of glycine, a divalent form of asparagine, a divalent form of histidine, a divalent form of phenylalanine, a divalent form of leucine, a divalent form of serine, or a divalent form of isoleucine. In embodiments,

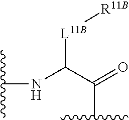

is a divalent form of glutamic acid. In embodiments,

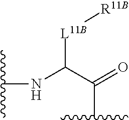

is a divalent form of alanine. In embodiments,

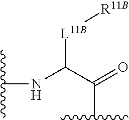

is a divalent form of aspartic acid. In embodiments,

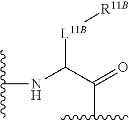

is a divalent form of glycine. In embodiments,

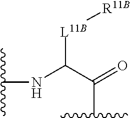

is a divalent form of asparagine. In embodiments,

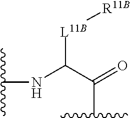

is a divalent form of histidine. In embodiments,

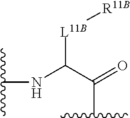

is a divalent form of phenylalanine. In embodiments,

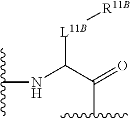

is a divalent form of leucine. In embodiments,

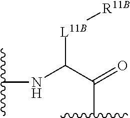

is a divalent form of serine. In embodiments,

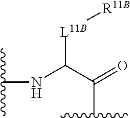

is a divalent form of isoleucine. In embodiments, -L^{11B}-R^{11B} is

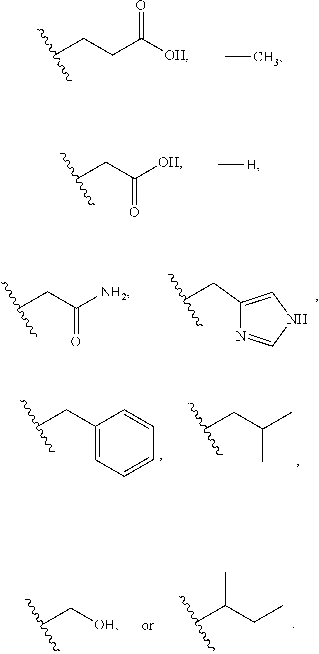

In embodiments, -L^{11B}-R^{11B} is

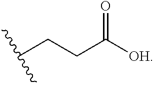 [SMILES: *CCC(=O)O]

In embodiments, -L^{11B}-R^{11B} is —CH₃. In embodiments, -L^{11B}-R^{11B} is

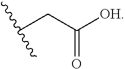 [SMILES: *CC(=O)O]

In embodiments, -L^{11B}-R^{11B} is —H. In embodiments, -L^{11B}-R^{11B} is

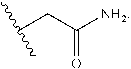 [SMILES: *CC(N)=O]

In embodiments, -L^{11B}-R^{11B} is

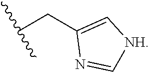 [SMILES: *Cc1c[nH]cn1]

In embodiments, -L^{11B}-R^{11B} is

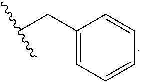 [SMILES: *Cc1ccccc1]

In embodiments, -L^{11B}-R^{11B} is

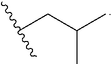 [SMILES: *CC(C)C]

In embodiments, -L^{11B}-R^{11B} is

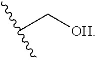 [SMILES: *CO]

In embodiments, -L^{11B}-R^{11B} is

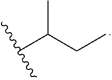 [SMILES: *C(C)CC]

In embodiments,

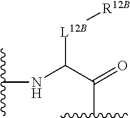

is a divalent form of an unnatural amino acid. In embodiments, L^{12B} is a bond or unsubstituted C₁-C₆ alkylene. In embodiments, R^{12B} is —OH, —NH₂, —C(O)NH₂, —NHC(NH)NH₂, substituted or unsubstituted alkyl, substituted or unsubstituted aryl, or substituted or unsubstituted heteroaryl. In embodiments,

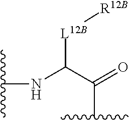

is a divalent form of a natural amino acid. In embodiments,

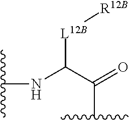

is a divalent form of arginine, a divalent form of tyrosine, a divalent form of isoleucine, a divalent form of threonine, a divalent form of lysine, a divalent form of leucine, a divalent form of histidine, a divalent form of asparagine, a divalent form of serine, or a divalent form of valine. In embodiments,

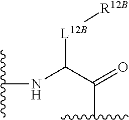

is a divalent form of arginine. In embodiments,

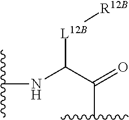

is a divalent form of tyrosine. In embodiments,

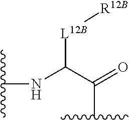

is a divalent form of isoleucine. In embodiments,

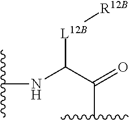

is a divalent form of threonine. In embodiments,

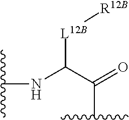

is a divalent form of lysine. In embodiments,

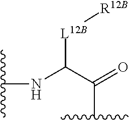

is a divalent form of leucine. In embodiments,

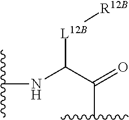

is a divalent form of histidine. In embodiments,

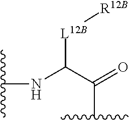

is a divalent form of asparagine. In embodiments,

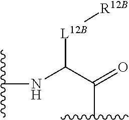

is a divalent form of serine. In embodiments,

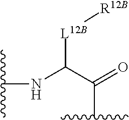

is a divalent form of valine. In embodiments, -L^{12B}-R^{12B} is

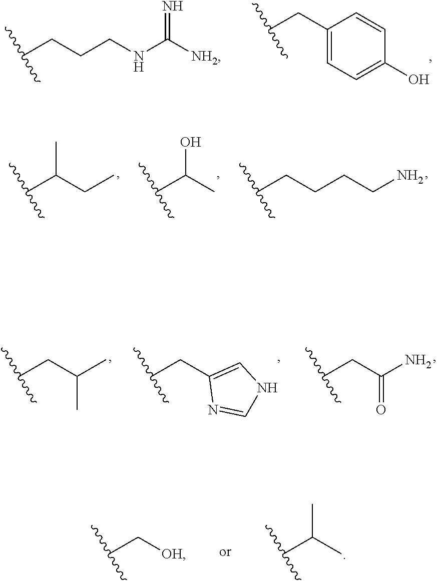

In embodiments, -L^{12B}-R^{12B} is

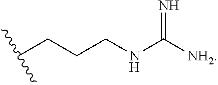 [SMILES: *CCCNC(=N)N]

In embodiments, -L^{12B}-R^{2B} is

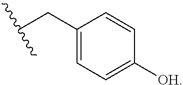 [SMILES: *Cc1ccc(O)cc1]

In embodiments, -L^{12B}-R^{12B} is

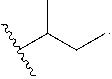 [SMILES: *C(C)CC]

In embodiments, -L^{2B}-R^{2B} is

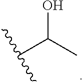 [SMILES: *C(C)O]

In embodiments, -L^{12B}-R^{12B} is

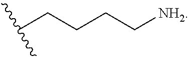 [SMILES: *CCCCN]

In embodiments, -L^{12B}-R^{12B} is

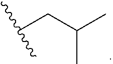 [SMILES: *CC(C)C]

In embodiments, -L^{2B}-R^{12B} is

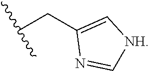 [SMILES: *Cc1c[nH]cn1]

In embodiments, -L^{2B}-R^{2B} is

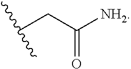 [SMILES: *CC(N)=O]

In embodiments, -L^{12B}-R^{12B} is

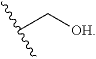 [SMILES: *CO]

In embodiments, -L^{12B}-R^{12B} is

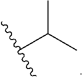 [SMILES: *C(C)C]

In embodiments,

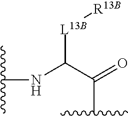

is a divalent form of an unnatural amino acid. In embodiments, L^{13B} is a bond or unsubstituted C₁-C₆ alkylene. In embodiments, R^{13B} is —NH₂, —C(O)OH, —C(O)NH₂, substituted or unsubstituted alkyl, or substituted or unsubstituted aryl. In embodiments,

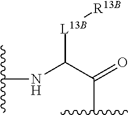

is a divalent form of a natural amino acid. In embodiments,

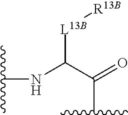

is a divalent form of lysine, a divalent form of aspartic acid, a divalent form of tyrosine, a divalent form of leucine, a divalent form of alanine, or a divalent form of asparagine. In embodiments,

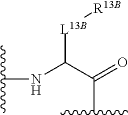

is a divalent form of lysine. In embodiments,

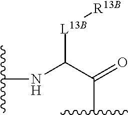

is a divalent form of aspartic acid. In embodiments,

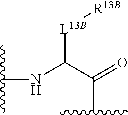

is a divalent form of tyrosine. In embodiments,

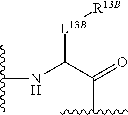

is a divalent form of leucine. In embodiments,

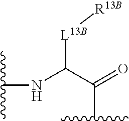

is a divalent form of alanine. In embodiments,

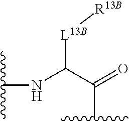

is a divalent form of asparagine. In embodiments, -L^{13B}-R^{13B} is

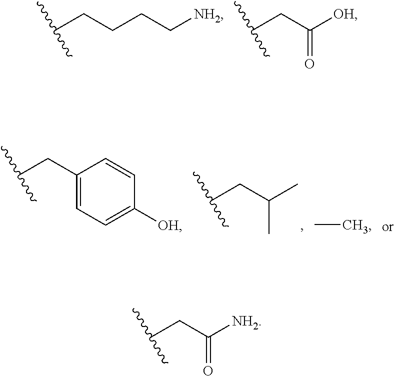

In embodiments, -L^{13B}-R^{13B} is

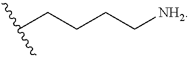 [SMILES: *CCCCN]

In embodiments, -L^{13B}-R^{13B} is

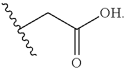 [SMILES: *CC(=O)O]

In embodiments, -L^{13B}-R^{13B} is

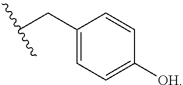 [SMILES: *Cc1ccc(O)cc1]

In embodiments, -L^{13B}-R^{13B} is

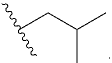 [SMILES: *CC(C)C]

In embodiments, -L^{13B}-R^{13B} is —CH₃. In embodiments, -L^{13B}-R^{13B} is

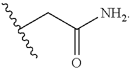 [SMILES: *CC(N)=O]

In embodiments, the compound has the formula:

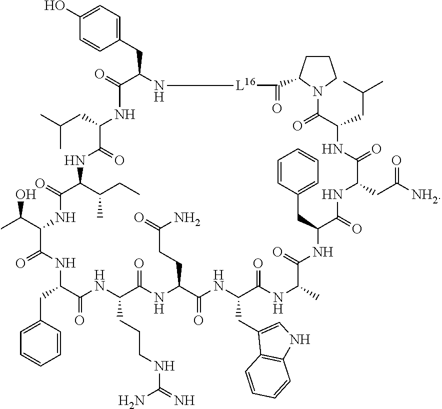

L¹⁶ is as described herein, including in embodiments.

In embodiments, the compound has the formula:

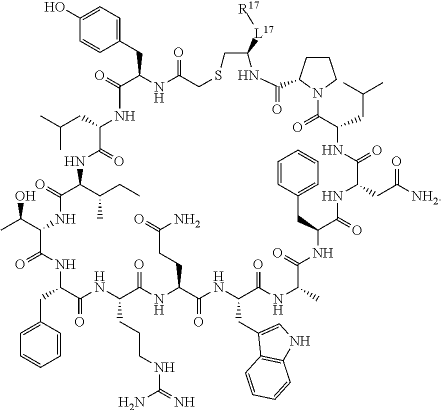

L¹⁷ and R¹⁷ are as described herein, including in embodiments.

In embodiments, the compound has the formula:

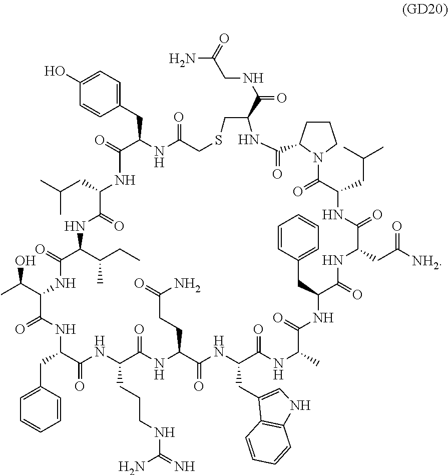 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](Cc2ccccc2)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

In embodiments, the compound has the formula:

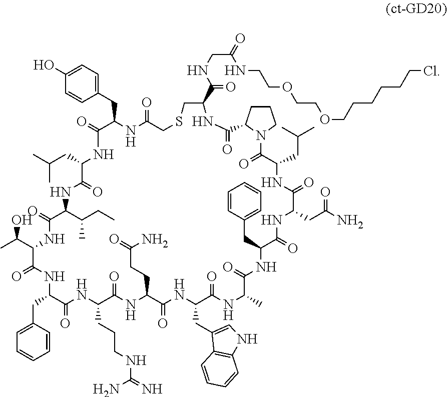 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(=O)NCCOCCOCCCCCCCl)NC(=O)[C@@H]2CCCN2C(=O)[C@H](CC(C)C)NC(=O)[C@H](CC(N)=O)NC(=O)[C@H](Cc2ccccc2)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

In embodiments, the compound has the formula:

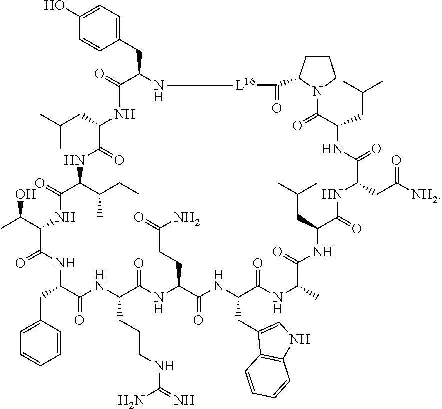

L¹⁶ is as described herein, including in embodiments.

In embodiments, the compound has the formula:

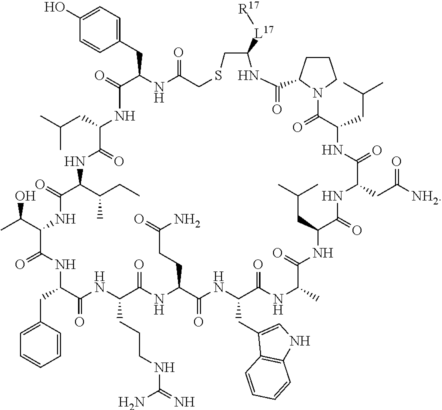

L¹⁷ and R¹⁷ are as described herein, including in embodiments.

In embodiments, the compound has the formula:

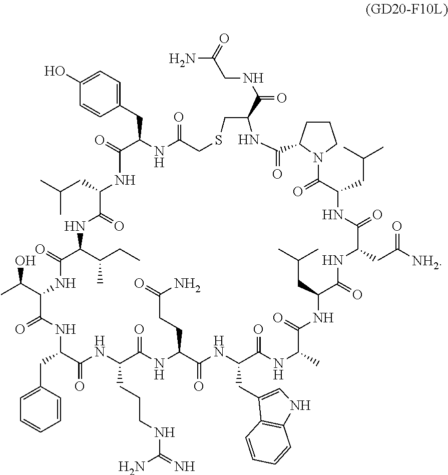 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](CC(C)C)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

In embodiments, the compound has the formula:

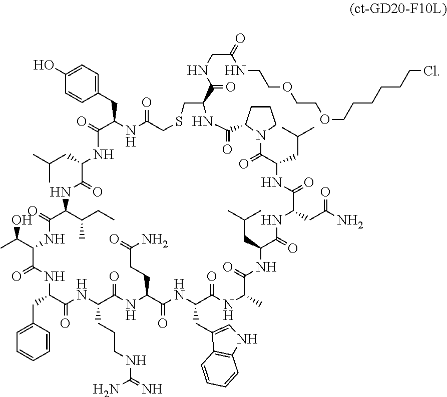 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(=O)NCCOCCOCCCCCCCl)NC(=O)[C@@H]2CCCN2C(=O)[C@H](CC(C)C)NC(=O)[C@H](CC(N)=O)NC(=O)[C@H](CC(C)C)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

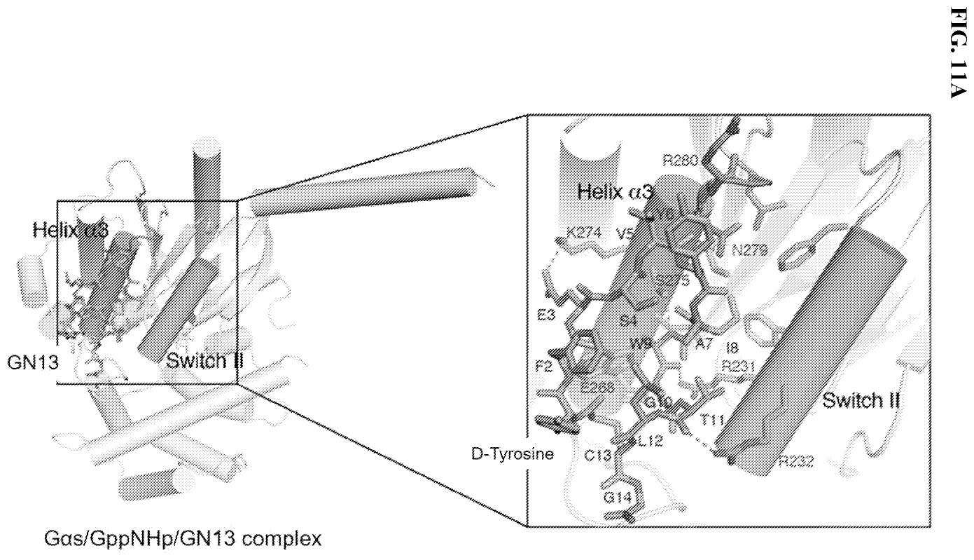

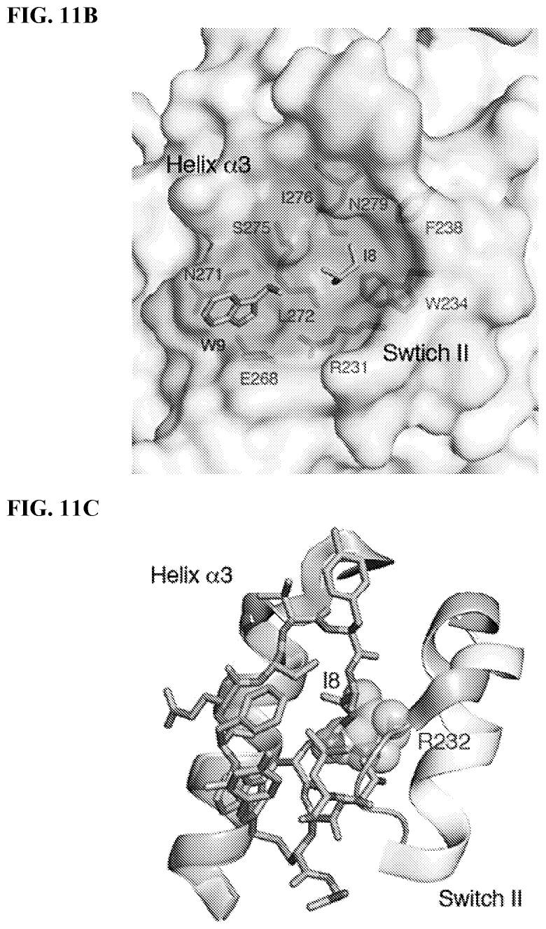

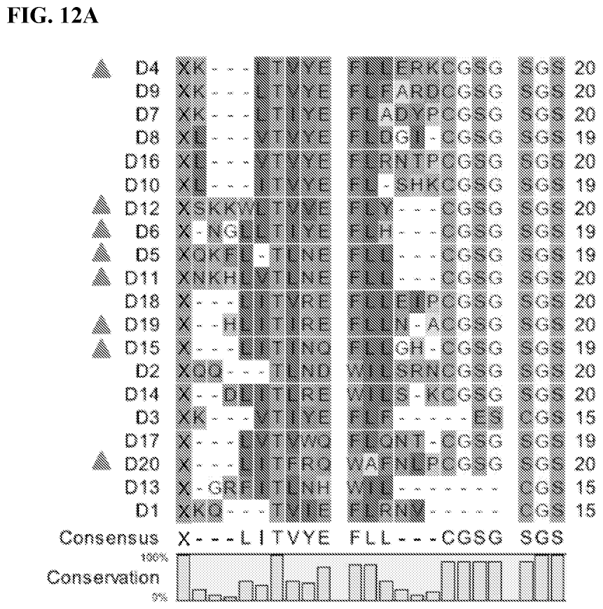

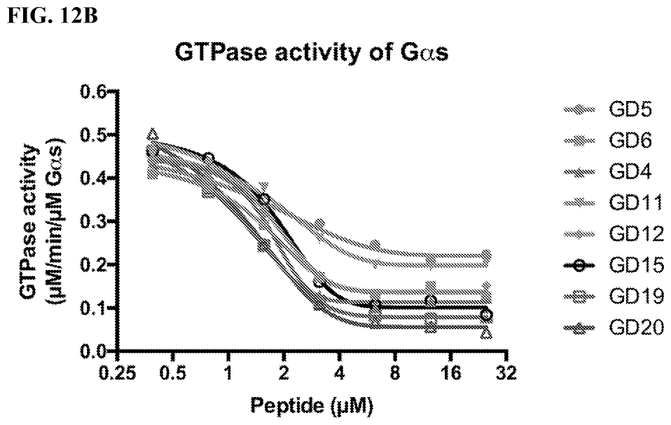

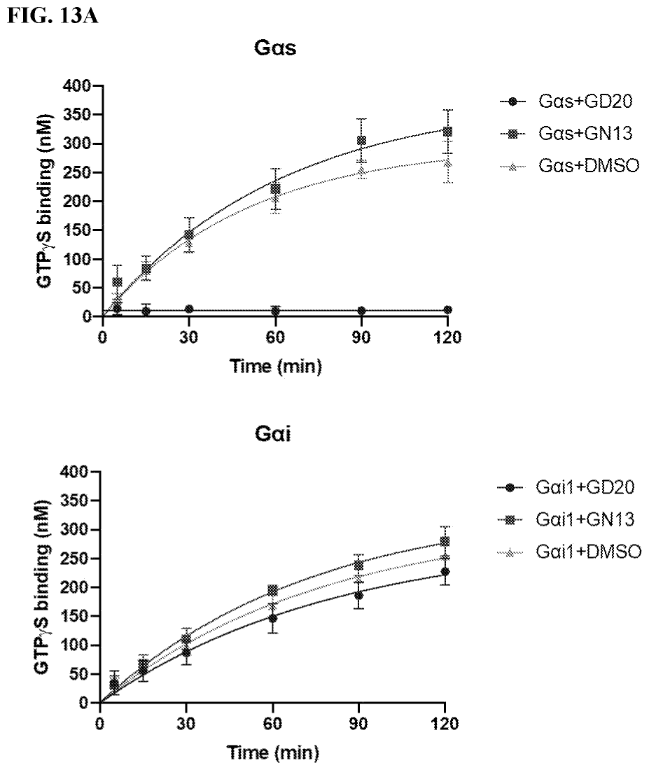

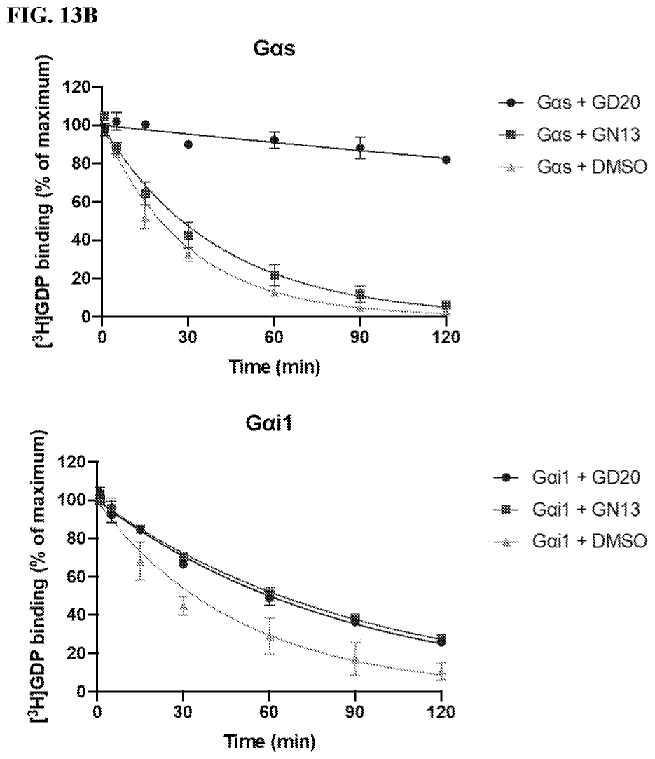

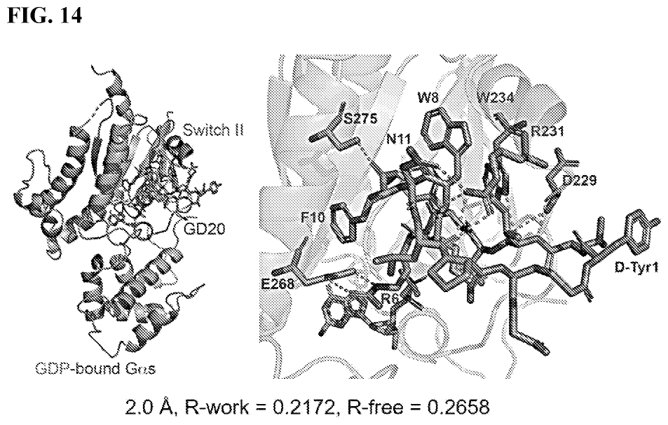

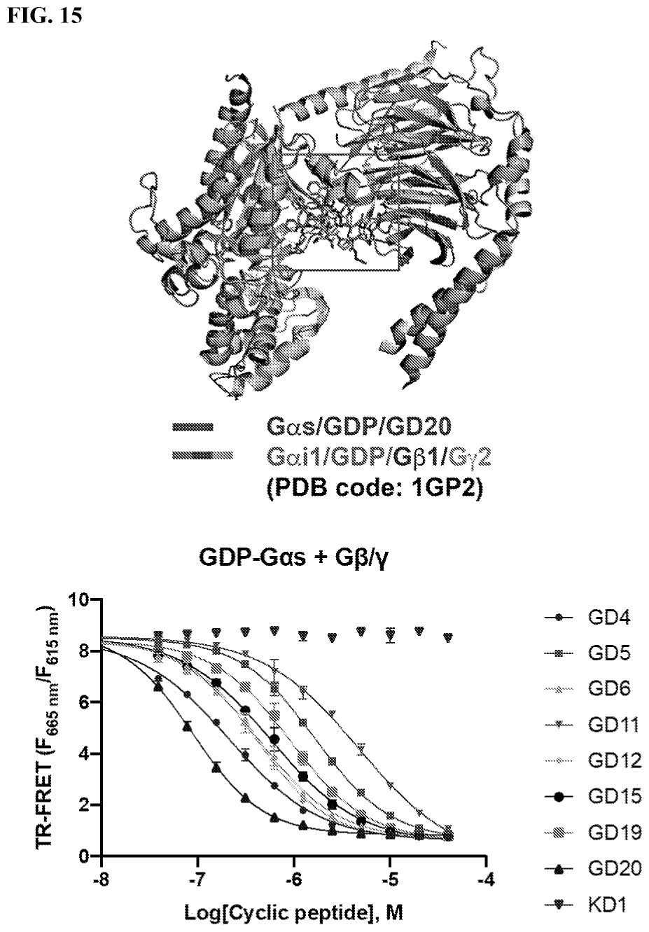

In embodiments, the compound of formula (II) is a peptide of FIG. 12A. In embodiments, the compound of formula (II) is peptide D4, D9, D7, D8, D16, D10, D12, D6, D5, D11, D18, D19, D15, D2, D14, D3, D17, D20, D13, or DI of FIG. 12A.

For peptide D4 of FIG. 12A, -L^{1B}-R^{1B} is a D-tyrosine side chain; -L^{2B}-R^{2B} is a lysine side chain; -L^{3B}-R^{3B} is a leucine side chain; -L^{4B}-R^{4B} is a threonine side chain; -L^{5B}-R^{5B} is a valine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is a leucine side chain; -L^{11B}-R^{11B} is a glutamic acid side chain; -L^{12B}-R^{12B} is an arginine side chain; and -L^{13B}-R^{13B} is a lysine side chain.

For peptide D9 of FIG. 12A, -L^{1B}-R^{1B} is a D-tyrosine side chain; -L^{2B}-R^{2B} is a lysine side chain; -L^{3B}-R^{3B} is a leucine side chain; -L^{4B}-R^{4B} is a threonine side chain; -L^{5B}-R^{5B} is a valine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is a phenylalanine side chain; -L^{11B}-R^{11B} is an alanine side chain; -L^{12B}-R^{12B} is an arginine side chain; and -L^{13B}-R^{13B} is an aspartic acid side chain.

For peptide D7 of FIG. 12A, -L^{1B}-R^{1B} is a D-tyrosine side chain; -L^{2B}-R^{2B} is a lysine side chain; -L^{3B}-R^{3B} is a leucine side chain; -L^{4B}-R^{4B} is a threonine side chain; -L^{5B}-R^{5B} is an isoleucine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is an alanine side chain; -L^{11B}-R^{11B} is an aspartic acid side chain; -L^{12B}-R^{12B} is a tyrosine side chain; and L¹³ is

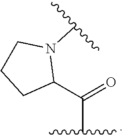 [SMILES: *C(=O)C1CCCN1*]

For peptide D8 of FIG. 12A, -L^{1B}-R^{1B} is a D-tyrosine side chain; -L^{2B}-R^{2B} is a leucine side chain; -L^{3B}-R^{3B} is a valine side chain; -L^{4B}-R^{4B} is a threonine side chain; -L^{5B}-R^{5B} is a valine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is an aspartic acid side chain; -L^{11B}-R^{11B} is a glycine side chain; -L^{12B}-R^{12B} is an isoleucine side chain; and L¹³ is a bond.

For peptide D16 of FIG. 12A, -L^{1B}-R^{1B} is a D-tyrosine side chain; -L^{2B}-R^{2B} is a leucine side chain; -L^{3B}-R^{3B} is a valine side chain; -L^{4B}-R^{4B} is a threonine side chain; -L^{5B}-R^{5B} is a valine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is an arginine side chain; -L^{11B}-R^{11B} is an asparagine side chain; -L^{12B}-R^{12B} is a threonine side chain; and L¹³ is

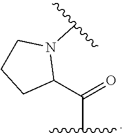 [SMILES: *C(=O)C1CCCN1*]

For peptide D10 of FIG. 12A, -L^{1B}-R^{1B} is a D-tyrosine side chain; -L^{2B}-R^{2B} is a leucine side chain; -L^{3B}-R^{3B} is an isoleucine side chain; -L^{4B}-R^{4B} is a threonine side chain; -L^{5B}-R^{5B} is a valine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is a serine side chain; -L^{11B}-R^{11B} is a histidine side chain; -L^{12B}-R^{12B} is a lysine side chain; and L¹³ is a bond.

For peptide D12 of FIG. 12A, -L^{1B}-R^{1B} is a D-tyrosine side chain; -L^{2B}-R^{2B} is a serine side chain; -L^{3B}-R^{3B} is a lysine side chain; -L^{4B}-R^{4B} is a lysine side chain; -L^{5B}-R^{5B} is a tryptophan side chain; -L^{6B}-R^{6B} is a leucine side chain; -L^{7B}-R^{7B} is a threonine acid side chain; -L^{8B}-R^{8B} is a valine side chain; -L^{9B}-R^{9B} is a valine side chain; -L^{10B}-R^{10B} is a glutamic acid side chain; -L^{11B}-R^{11B} is a phenylalanine side chain; -L^{12B}-R^{12B} is a leucine side chain; and -L^{13B}-R^{13B} is a tyrosine side chain.

For peptide D6 of FIG. 12A, -L^{1B}-R^{1B} is a D-tyrosine side chain; -L^{2B}-R^{2B} is an asparagine side chain; -L^{3B}-R^{3B} is a glycine side chain; -L^{4B}-R^{4B} is a leucine side chain; -L^{5B}-R^{5B} is a leucine side chain; -L^{6B}-R^{6B} is a threonine side chain; -L^{7B}-R^{7B} is an isoleucine side chain; -L^{8B}-R^{8B} is a tyrosine side chain; -L^{9B}-R^{9B} is a glutamic acid side chain; -L^{10B}-R^{10B} is a phenylalanine side chain; -L^{11B}-R^{11B} is a leucine side chain; -L^{12B}-R^{12B} is a histidine side chain; and L¹³ is a bond.

For peptide D5 of FIG. 12A, -L^{1B}-R^{1B} is a D-tyrosine side chain; -L^{2B}-R^{2B} is a glutamine side chain; -L^{3B}-R^{3B} is a lysine side chain; -L^{4B}-R^{4B} is a phenylalanine side chain; -L^{5B}-R^{5B} is a leucine side chain; -L^{6B}-R^{6B} is a threonine side chain; -L^{7B}-R^{7B} is a leucine side chain; -L^{8B}-R^{8B} is an asparagine side chain; -L^{9B}-R^{9B} is a glutamic acid side chain; -L^{10B}-R^{10B} is a phenylalanine side chain; -L^{11B}-R^{11B} is a leucine side chain; -L^{12B}-R^{12B} is a leucine side chain; and L¹³ is a bond.

For peptide D11 of FIG. 12A, -L^{1B}-R^{1B} is a D-tyrosine side chain; -L^{2B}-R^{2B} is an asparagine side chain; -L^{3B}-R^{3B} is a lysine side chain; -L^{4B}-R^{4B} is a histidine side chain; -L^{5B}-R^{5B} is a leucine side chain; -L^{6B}-R^{6B} is a valine side chain; -L^{7B}-R^{7B} is a threonine side chain; -L^{8B}-R^{8B} is a leucine side chain; -L^{9B}-R^{9B} is an asparagine side chain; -L^{10B}-R^{10B} is a glutamic acid side chain; -L^{11B}-R^{11B} is a phenylalanine side chain; -L^{12B}-R^{12B} is a leucine side chain; and -L^{13B}-R^{13B} is a leucine side chain.

For peptide D18 of FIG. 12A, -L^{1B}-R^{1B} is a D-tyrosine side chain; -L^{2B}-R^{2B} is a leucine side chain; -L^{3B}-R^{3B} is an isoleucine side chain; -L^{4B}-R^{4B} is a threonine side chain; -L^{5B}-R^{5B} is a valine side chain; -L^{6B}-R^{6B} is an arginine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is a leucine side chain; -L^{11B}-R^{11B} is a glutamic acid side chain; -L^{12B}-R^{12B} is an isoleucine side chain; and L¹³ is

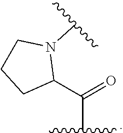 [SMILES: *C(=O)C1CCCN1*]

For peptide D19 of FIG. 12A, -L^{1B}-R^{1B} is a D-tyrosine side chain; -L^{2B}-R^{2B} is a histidine side chain; -L^{3B}-R^{3B} is a leucine side chain; -L^{4B}-R^{4B} is an isoleucine side chain; -L^{5B}-R^{5B} is a threonine side chain; -L^{6B}-R^{6B} is an isoleucine side chain; -L^{7B}R^{1B} is an arginine side chain; -L^{8B}-R^{8B} is a glutamic acid side chain; -L^{9B}-R^{9B} is a phenylalanine side chain; -L^{10B}-R^{10B} is a leucine side chain; -L^{11B}-R^{11B} is a leucine side chain; -L^{12B}-R^{12B} is an asparagine side chain; and -L^{13B}-R^{13B} is an alanine side chain.

For peptide D15 of FIG. 12A, -L^{1B}-R^{1B} is a D-tyrosine side chain; -L^{2B}-R^{2B} is a leucine side chain; -L^{3B}-R^{3B} is an isoleucine side chain; -L^{4B}-R^{4B} is a threonine side chain; -L^{5B}-R^{5B} is an isoleucine side chain; -L^{6B}-R^{6B} is an asparagine side chain; -L^{7B}-R^{7B} is a glutamine side chain; -L^{8B}-R^{8B} is a phenylalanine side chain; -L^{9B}-R^{9B} is a leucine side chain; -L^{10B}-R^{10B} is a leucine side chain; -L^{11B}-R^{11B} is a glycine side chain; -L^{12B}-R^{12B} is a histidine side chain; and L¹³ is a bond.

For peptide D2 of FIG. 12A, -L^{1B}-R^{1B} is a D-tyrosine side chain; -L^{2B}-R^{2B} is a glutamine side chain; -L^{3B}-R^{3B} is a glutamine side chain; -L^{4B}-R^{4B} is a threonine side chain; -L^{5B}-R^{5B} is a leucine side chain; -L^{6B}-R^{6B} is an asparagine side chain; -L^{7B}-R^{7B} is an aspartic acid side chain; -L^{8B}-R^{8B} is a tryptophan side chain; -L^{9B}-R^{9B} is an isoleucine side chain; -L^{10B}-R^{10B} is a leucine side chain; -L^{11B}-R^{11B} is a serine side chain; -L^{12B}-R^{12B} is an arginine side chain; and -L^{13B}-R^{13B} is an asparagine side chain.

For peptide D14 of FIG. 12A, -L^{1B}-R^{1B} is a D-tyrosine side chain; -L^{2B}-R^{2B} is an aspartic acid side chain; -L^{3B}-R^{3B} is a leucine side chain; -L^{4B}-R^{4B} is an isoleucine side chain; -L^{5B}-R^{5B} is a threonine side chain; -L^{6B}-R^{6B} is a leucine side chain; -L^{7B}-R^{7B} is an arginine side chain; -L^{8B}-R^{8B} is a glutamic acid side chain; -L^{9B}-R^{9B} is a tryptophan side chain; -L^{10B}-R^{10B} is an isoleucine side chain; -L^{11B}-R^{11B} is a leucine side chain; -L^{12B}-R^{12B} is a serine side chain; and -L^{13B}-R^{13B} is a lysine side chain.

For peptide D3 of FIG. 12A, -L^{1B}-R^{1B} is a D-tyrosine side chain; -L^{2B}-R^{2B} is a lysine side chain; -L^{3B}-R^{3B} is a valine side chain; -L^{4B}-R^{4B} is a threonine side chain; -L^{5B}-R^{5B} is an isoleucine side chain; -L^{6B}-R^{6B} is a tyrosine side chain; -L^{7B}-R^{7B} is a glutamic acid side chain; -L^{8B}-R^{8B} is a phenylalanine side chain; -L^{9B}-R^{9B} is a leucine side chain; -L^{10B}R^{10B} is a phenylalanine side chain; -L^{11B}-R^{11B} is a glutamic acid side chain; -L^{12B}-R^{12B} chain; and L¹³ is a bond.

For peptide D17 of FIG. 12A, -L^{1B}-R^{1B} is a D-tyrosine side chain; -L^{2B}-R^{2B} is a leucine side chain; -L^{3B}-R^{3B} is a valine side chain; -L^{4B}-R^{4B} is a threonine side chain; -L^{5B}-R^{5B} is a valine side chain; -L^{6B}-R^{6B} is a tryptophan side chain; -L^{7B}-R^{7B} is a glutamine side chain; -L^{8B}-R^{8B} is a phenylalanine side chain; -L^{9B}-R^{9B} is a leucine side chain; -L^{10B}-R^{10B} is a glutamine side chain; -L^{11B}-R^{11B} is an asparagine side chain; -L^{12B}-R^{12B} is a threonine side chain; and L¹³ is a bond.

For peptide D20 of FIG. 12A, -L^{1B}-R^{1B} is a D-tyrosine side chain; -L^{2B}-R^{2B} is a leucine side chain; -L^{3B}-R^{3B} is an isoleucine side chain; -L^{4B}-R^{4B} is a threonine side chain; -L^{5B}-R^{5B} is a phenylalanine side chain; -L^{6B}-R^{6B} is an arginine side chain; -L^{7B}-R^{7B} is a glutamine side chain; -L^{8B}-R^{8B} is a tryptophan side chain; -L^{9B}-R^{9B} is an alanine side chain; -L^{10B}-R^{10B} is a phenylalanine side chain; -L^{11B}-R^{11B} is an asparagine side chain; -L^{12B}-R^{12B} is a leucine side chain; and L¹³ is

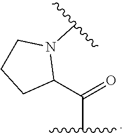 [SMILES: *C(=O)C1CCCN1*]

For peptide D13 of FIG. 12A, -L^{1B}-R^{1B} is a D-tyrosine side chain; -L^{2B}-R^{2B} is a glycine side chain; -L^{3B}-R^{3B} is an arginine side chain; -L^{4B}-R^{4B} is a phenylalanine side chain; -L^{5B}-R^{5B} is an isoleucine side chain; -L^{6B}-R^{6B} is a threonine side chain; -L^{7B}R^{1B} is a leucine side chain; -L^{8B}-R^{8B} is an asparagine side chain; -L^{9B}-R^{9B} is a histidine side chain; -L^{10B}-R^{10B} is a tryptophan side chain; -L^{11B}-R^{11B} is an isoleucine side chain; -L^{12B}-R^{12B} is a leucine side chain; and L¹³ is a bond.

For peptide D1 of FIG. 12A, -L^{1B}-R^{1B} is a D-tyrosine side chain; -L^{2B}-R^{2B} is a lysine side chain; -L^{3B}-R^{3B} is a glutamine side chain; -L^{4B}-R^{4B} is a threonine side chain; -L^{5B}-R^{5B} is a valine side chain; -L^{6B}-R^{6B} is an isoleucine side chain; -L^{7B}-R^{7B} is a glutamic acid side chain; -L^{8B}-R^{8B} is a phenylalanine side chain; -L^{9B}-R^{9B} is a leucine side chain; -L^{10B}-R^{10B} is an arginine side chain; -L^{11B}-R^{11B} is an asparagine side chain; -L^{12B}-R^{12B} is a valine side chain; and L¹³ is a bond.

In embodiments, the compound binds a human Gαs protein-GDP complex more strongly than the compound binds a human Gαs protein-GTP complex under identical conditions. In embodiments, the compound binds a Gαs protein-GDP complex at least 2-fold stronger than the compound binds a human Gαs protein-GTP complex under identical conditions. In embodiments, the compound binds a human Gαs protein-GDP complex at least 5-fold stronger than the compound binds a human Gαs protein-GTP complex under identical conditions. In embodiments, the compound binds a human Gαs protein-GDP complex at least 10-fold stronger than the compound binds a human Gαs protein-GTP complex under identical conditions. In embodiments, the compound binds a human Gαs protein-GDP complex at least 20-fold stronger than the compound binds a human Gαs protein-GTP complex under identical conditions. In embodiments, the compound binds a human Gαs protein-GDP complex at least 40-fold stronger than said compound binds a human Gαs protein-GTP complex under identical conditions. In embodiments, the compound binds a human Gαs protein-GDP complex at least 60-fold stronger than the compound binds a human Gαs protein-GTP complex under identical conditions. In embodiments, the compound binds a human Gαs protein-GDP complex at least 80-fold stronger than the compound binds a human Gαs protein-GTP complex under identical conditions. In embodiments, the compound binds a human Gαs protein-GDP complex at least 100-fold stronger than said compound binds a human Gαs protein-GTP complex under identical conditions. In embodiments, the compound binds a human Gαs protein-GDP complex at least 500-fold stronger than the compound binds a human Gαs protein-GTP complex under identical conditions.

In embodiments, a divalent form of an unnatural amino acid is a divalent form of an unnatural phenylalanine derivative. In embodiments, the divalent form of an unnatural

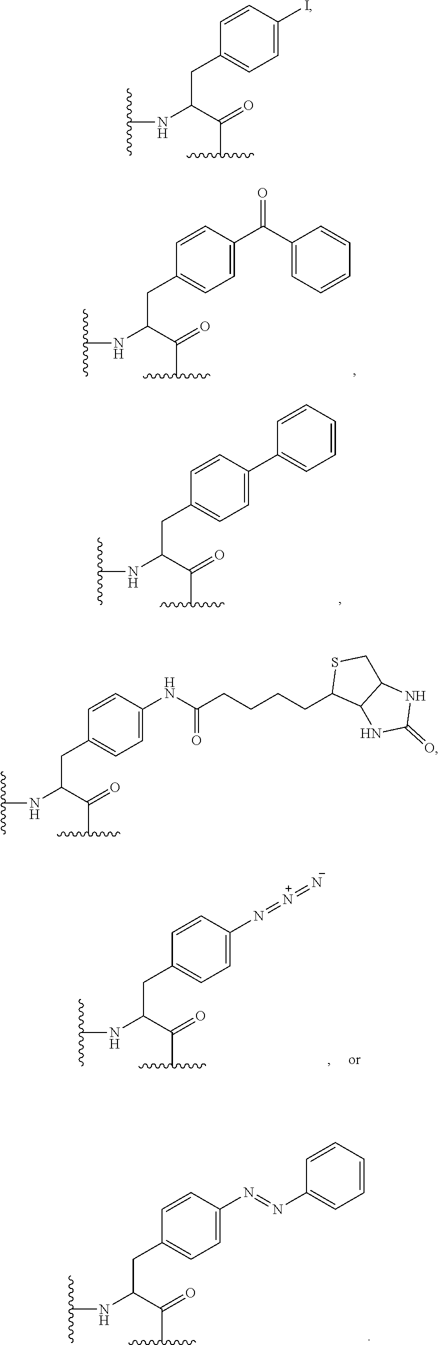 [SMILES: *NC(Cc1ccc(I)cc1)C(*)=O ; *NC(Cc1ccc(C(=O)c2ccccc2)cc1)C(*)=O ; *NC(Cc1ccc(-c2ccccc2)cc1)C(*)=O ; *NC(Cc1ccc(NC(=O)CCCCC2SCC3NC(=O)NC32)cc1)C(*)=O ; *NC(Cc1ccc(N=[N+]=[N-])cc1)C(*)=O ; *NC(Cc1ccc(/N=N/c2ccccc2)cc1)C(*)=O]

In embodiments, RD is hydrogen. In embodiments, R^{1D} is unsubstituted methyl. In embodiments, R^{1D} is unsubstituted ethyl. In embodiments, R^{1D} is unsubstituted propyl. In embodiments, R^{1D} is unsubstituted butyl. In embodiments, R^{2D} is hydrogen. In embodiments, R^{2D} is unsubstituted methyl. In embodiments, R^{2D} is unsubstituted ethyl. In embodiments, R^{2D} is unsubstituted propyl. In embodiments, R^{2D} is unsubstituted butyl. In embodiments, R^{3D} is hydrogen. In embodiments, R^{3D} is unsubstituted methyl. In embodiments, R^{3D} is unsubstituted ethyl. In embodiments, R^{3D} is unsubstituted propyl. In embodiments, R^{3D} is unsubstituted butyl. In embodiments, R^{4D} is hydrogen. In embodiments, R^{4D} is unsubstituted methyl. In embodiments, R^{4D} is unsubstituted ethyl. In embodiments, R^{4D} is unsubstituted propyl. In embodiments, R^{4D} is unsubstituted butyl. In embodiments, R^{5D} is hydrogen. In embodiments, R^{5D} is unsubstituted methyl. In embodiments, R^{5D} is unsubstituted ethyl. In embodiments, R^{5D} is unsubstituted propyl. In embodiments, R^{5D} is unsubstituted butyl. In embodiments, R^{6D} is hydrogen. In embodiments, R^{6D} is unsubstituted methyl. In embodiments, R^{6D} is unsubstituted ethyl. In embodiments, R^{6D} is unsubstituted propyl. In embodiments, R^{6D} is unsubstituted butyl. In embodiments, R^{7D} is hydrogen. In embodiments, R^{7D} is unsubstituted methyl. In embodiments, R^{7D} is unsubstituted ethyl. In embodiments, R^{7D} is unsubstituted propyl. In embodiments, R^{7D} is unsubstituted butyl. In embodiments, R^{8D} is hydrogen. In embodiments, R^{8D} is unsubstituted methyl. In embodiments, R^{8D} is unsubstituted ethyl. In embodiments, R^{8D} is unsubstituted propyl. In embodiments, R^{8D} is unsubstituted butyl. In embodiments, R^{9D} is hydrogen. In embodiments, R^{9D} is unsubstituted methyl. In embodiments, R^{9D} is unsubstituted ethyl. In embodiments, R^{9D} is unsubstituted propyl. In embodiments, R^{9D} is unsubstituted butyl. In embodiments, R^{10D} is hydrogen. In embodiments, R^{10D} is unsubstituted methyl. In embodiments, R^{10D} is unsubstituted ethyl. In embodiments, R^{10D} is unsubstituted propyl. In embodiments, R^{10D} is unsubstituted butyl. In embodiments, R^{11D} is hydrogen. In embodiments, R^{11D} is unsubstituted methyl. In embodiments, R^{11D} is unsubstituted ethyl. In embodiments, R^{11D} is unsubstituted propyl. In embodiments, R^{11D} is unsubstituted butyl. In embodiments, R^{12D} is hydrogen. In embodiments, R^{12D} is unsubstituted methyl. In embodiments, R^{12D} is unsubstituted ethyl. In embodiments, R^{12D} is unsubstituted propyl. In embodiments, R^{12D} is unsubstituted butyl. In embodiments, R^{13D} is hydrogen. In embodiments, R^{13D} is unsubstituted methyl. In embodiments, R^{13D} is unsubstituted ethyl. In embodiments, R^{13D} is unsubstituted propyl. In embodiments, R^{13D} is unsubstituted butyl.

In embodiments, L¹⁶ is a bioconjugate linker. In embodiments, L¹⁶ is a substituted or unsubstituted divalent amino acid. In embodiments, L¹⁶ is a substituted or unsubstituted divalent δ-amino acid.

In embodiments, a substituted L¹⁶ (e.g., substituted divalent amino acid and/or substituted divalent δ-amino acid) is substituted with at least one substituent group, size-limited substituent group, or lower substituent group; wherein if the substituted L¹⁶ is substituted with a plurality of groups selected from substituent groups, size-limited substituent groups, and lower substituent groups; each substituent group, size-limited substituent group, and/or lower substituent group may optionally be different. In embodiments, when L¹⁶ is substituted, it is substituted with at least one substituent group. In embodiments, when L¹⁶ is substituted, it is substituted with at least one size-limited substituent group. In embodiments, when L¹⁶ is substituted, it is substituted with at least one lower substituent group.

In embodiments, L¹⁶ is -L^{16A}-L^{16B}-L^{16C}-L^{16D}-L^{16E}-L^{16F}-.

L^{16A}, L^{16B}, L^{16C}, L^{16D}, L^{16E}, and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L^{16A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16A} is substituted with a plurality of groups selected from substituent groups, size-limited substituent groups, and lower substituent groups; each substituent group, size-limited substituent group, and/or lower substituent group may optionally be different. In embodiments, when L^{16A} is substituted, it is substituted with at least one substituent group. In embodiments, when L^{16A} is substituted, it is substituted with at least one size-limited substituent group. In embodiments, when L^{16A} is substituted, it is substituted with at least one lower substituent group.

In embodiments, a substituted L^{16B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16B} is substituted with a plurality of groups selected from substituent groups, size-limited substituent groups, and lower substituent groups; each substituent group, size-limited substituent group, and/or lower substituent group may optionally be different. In embodiments, when L^{16B} is substituted, it is substituted with at least one substituent group. In embodiments, when L^{16B} is substituted, it is substituted with at least one size-limited substituent group. In embodiments, when L^{16B} is substituted, it is substituted with at least one lower substituent group.

In embodiments, a substituted L^{16C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16C} is substituted with a plurality of groups selected from substituent groups, size-limited substituent groups, and lower substituent groups; each substituent group, size-limited substituent group, and/or lower substituent group may optionally be different. In embodiments, when L^{16C} is substituted, it is substituted with at least one substituent group. In embodiments, when L^{16C} is substituted, it is substituted with at least one size-limited substituent group. In embodiments, when L^{16C} is substituted, it is substituted with at least one lower substituent group.

In embodiments, a substituted L^{16D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16D} is substituted with a plurality of groups selected from substituent groups, size-limited substituent groups, and lower substituent groups; each substituent group, size-limited substituent group, and/or lower substituent group may optionally be different. In embodiments, when L^{16D} is substituted, it is substituted with at least one substituent group. In embodiments, when L^{16D} is substituted, it is substituted with at least one size-limited substituent group. In embodiments, when L^{16D} is substituted, it is substituted with at least one lower substituent group.

In embodiments, a substituted L^{16E} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16E} is substituted with a plurality of groups selected from substituent groups, size-limited substituent groups, and lower substituent groups; each substituent group, size-limited substituent group, and/or lower substituent group may optionally be different. In embodiments, when L^{16E} is substituted, it is substituted with at least one substituent group. In embodiments, when L^{16E} is substituted, it is substituted with at least one size-limited substituent group. In embodiments, when L^{16E} is substituted, it is substituted with at least one lower substituent group.

In embodiments, a substituted L^{16F} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16F} is substituted with a plurality of groups selected from substituent groups, size-limited substituent groups, and lower substituent groups; each substituent group, size-limited substituent group, and/or lower substituent group may optionally be different. In embodiments, when L^{16F} is substituted, it is substituted with at least one substituent group. In embodiments, when L^{16F} is substituted, it is substituted with at least one size-limited substituent group. In embodiments, when L^{16F} is substituted, it is substituted with at least one lower substituent group.

In embodiments, L^{16A} is bond, —SS—, substituted or unsubstituted alkylene, substituted or unsubstituted heteroalkylene, or substituted or unsubstituted heteroarylene. In embodiments, L^{16A} is bond. In embodiments, L^{16A} is —SS—. In embodiments, L^{16A} is substituted or unsubstituted C₁-C₄ alkylene. In embodiments, L^{16A} is substituted or unsubstituted 2 to 6 membered heteroalkylene. In embodiments, L^{16A} is substituted or unsubstituted 3 to 6 membered heteroarylene. In embodiments, L^{16A} is unsubstituted triazolylene. In embodiments, L^{16A} is

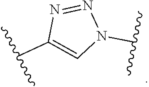 [SMILES: *c1cn(*)nn1]

In embodiments, L^{16B} is bond, —SS—, substituted or unsubstituted alkylene, substituted or unsubstituted heteroalkylene, or substituted or unsubstituted heteroarylene. In embodiments, L^{16B} is bond. In embodiments, L^{16B} is —SS—. In embodiments, L^{16B} is substituted or unsubstituted C₁-C₄ alkylene. In embodiments, L^{16B} is substituted or unsubstituted 2 to 6 membered heteroalkylene. In embodiments, L^{16B} is substituted or unsubstituted 3 to 6 membered heteroarylene. In embodiments, L^{16B} is unsubstituted triazolylene. In embodiments, L^{16B} is

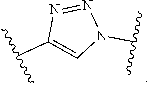 [SMILES: *c1cn(*)nn1]

In embodiments, L^{16C} is bond, —SS—, substituted or unsubstituted alkylene, substituted or unsubstituted heteroalkylene, or substituted or unsubstituted heteroarylene. In embodiments, L^{16C} is bond. In embodiments, L^{16C} is —SS—. In embodiments, L^{16C} is substituted or unsubstituted C₁-C₄ alkylene. In embodiments, L^{16C} is substituted or unsubstituted 2 to 6 membered heteroalkylene. In embodiments, L^{16C} is substituted or unsubstituted 3 to 6 membered heteroarylene. In embodiments, L^{16C} is unsubstituted triazolylene. In embodiments, L^{16C} is

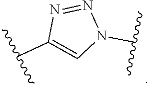 [SMILES: *c1cn(*)nn1]

In embodiments, L^{16D} is bond, —SS—, substituted or unsubstituted alkylene, substituted or unsubstituted heteroalkylene, or substituted or unsubstituted heteroarylene. In embodiments, L^{16D} is bond. In embodiments, L^{16D} is —SS—. In embodiments, L^{16D} is substituted or unsubstituted C₁-C₄ alkylene. In embodiments, L^{16D} is substituted or unsubstituted 2 to 6 membered heteroalkylene. In embodiments, L^{16D} is substituted or unsubstituted 3 to 6 membered heteroarylene. In embodiments, L^{16D} is unsubstituted triazolylene. In embodiments, L^{16D} is

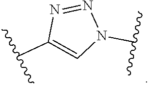 [SMILES: *c1cn(*)nn1]

In embodiments, L^{16E} is bond, —SS—, substituted or unsubstituted alkylene, substituted or unsubstituted heteroalkylene, or substituted or unsubstituted heteroarylene. In embodiments, L^{16E} is bond. In embodiments, L^{16E} is —SS—. In embodiments, L^{16E} is substituted or unsubstituted C₁-C₄ alkylene. In embodiments, L^{16E} is substituted or unsubstituted 2 to 6 membered heteroalkylene. In embodiments, L^{16E} is substituted or unsubstituted 3 to 6 membered heteroarylene. In embodiments, L^{16E} is unsubstituted triazolylene. In embodiments, L^{16E} is

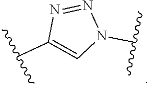 [SMILES: *c1cn(*)nn1]

In embodiments, L^{16F} is bond, —SS—, substituted or unsubstituted alkylene, substituted or unsubstituted heteroalkylene, or substituted or unsubstituted heteroarylene. In embodiments, L^{16F} is bond. In embodiments, L^{16F} is —SS—. In embodiments, L^{16F} is substituted or unsubstituted C₁-C₄ alkylene. In embodiments, L^{16F} is substituted or unsubstituted 2 to 6 membered heteroalkylene. In embodiments, L^{16F} is substituted or unsubstituted 3 to 6 membered heteroarylene. In embodiments, L^{16F} is unsubstituted triazolylene. In embodiments, L^{16F} is

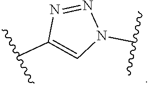 [SMILES: *c1cn(*)nn1]

In embodiments, -L^{16B}-L^{16C}-L^{16D}- is —SS—

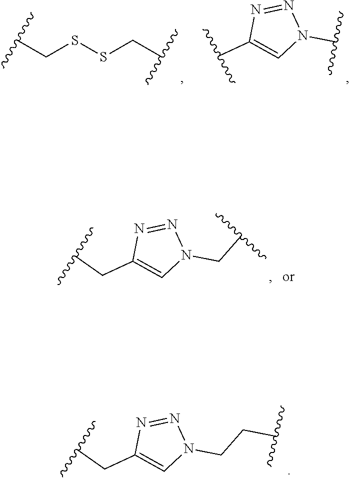 [SMILES: *CSSC* ; *c1cn(*)nn1 ; *Cc1cn(C*)nn1 ; *CCn1cc(C*)nn1]

In embodiments, L¹⁶ is —NH-L^{16B}-L^{16C}-L^{16D}-L^{16E}-C(O)—.

In embodiments, L^{16B} is

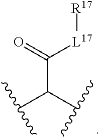

L¹⁷ is -L^{17A}-L^{17B}-L^{17C}-L^{17D}-L^{17E}-L^{17F}-.

L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), a monovalent nucleic acid, a monovalent protein, a detectable moiety, or a drug moiety.

In embodiments, L¹⁶ is a bond,

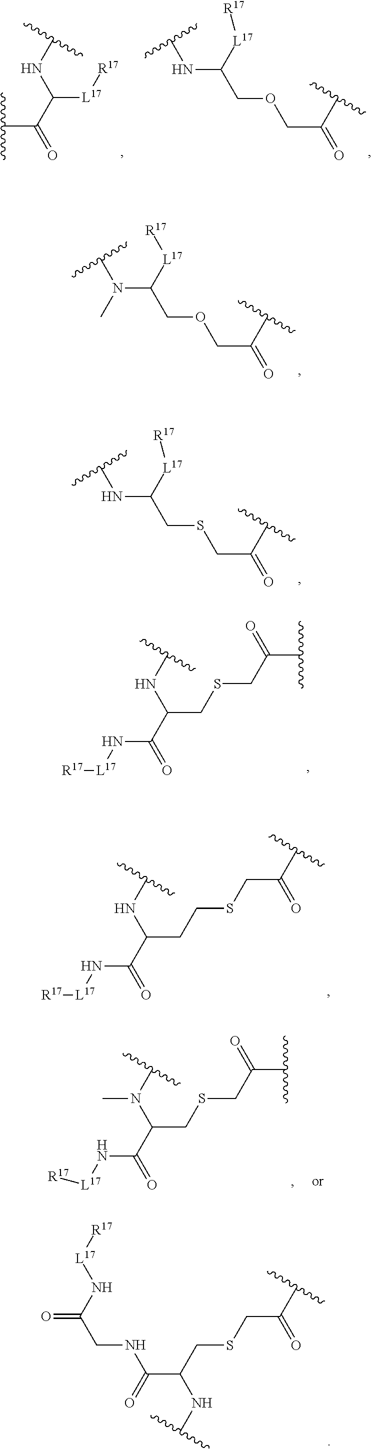

L¹⁷ and R¹⁷ are as described herein, including in embodiments. In embodiments, L¹⁶ is a bond. In embodiments, L¹⁶ is

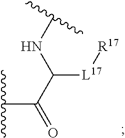

L¹⁷ and R¹⁷ are as described herein, including in embodiments. In embodiments, L¹⁶ is

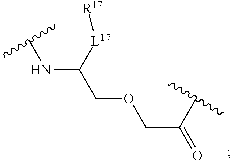

L¹⁷ and R¹⁷ are as described herein, including in embodiments. In embodiments, L¹⁶ is

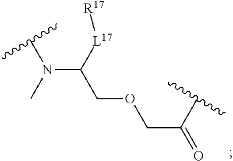

L¹⁷ and R¹⁷ are as described herein, including in embodiments. In embodiments, L¹⁶ is

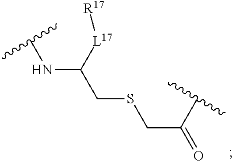

L¹⁷ and R¹⁷ are as described herein, including in embodiments. In embodiments, L¹⁶ is

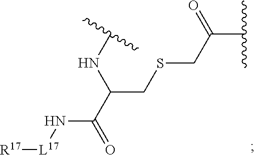

L¹⁷ and R¹⁷ are as described herein, including in embodiments. In embodiments, L¹⁶ is

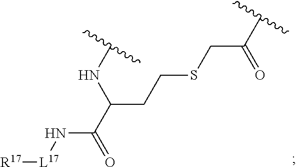

L¹⁷ and R¹⁷ are as described herein, including in embodiments. In embodiments, L¹⁶ is

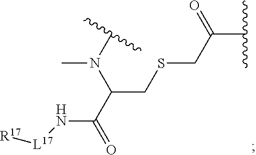

L¹⁷ and R¹⁷ are as described herein, including in embodiments.

In embodiments, L¹⁶ is

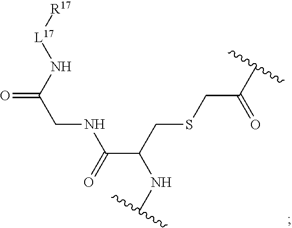

L¹⁷ and R¹⁷ are as described herein, including in embodiments.

In embodiments, a substituted L^{17A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17A} is substituted with a plurality of groups selected from substituent groups, size-limited substituent groups, and lower substituent groups; each substituent group, size-limited substituent group, and/or lower substituent group may optionally be different. In embodiments, when L^{17A} is substituted, it is substituted with at least one substituent group. In embodiments, when L^{17A} is substituted, it is substituted with at least one size-limited substituent group. In embodiments, when L^{17A} is substituted, it is substituted with at least one lower substituent group.

In embodiments, a substituted L^{7B}, (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{7B}, is substituted with a plurality of groups selected from substituent groups, size-limited substituent groups, and lower substituent groups; each substituent group, size-limited substituent group, and/or lower substituent group may optionally be different. In embodiments, when L^{17B} is substituted, it is substituted with at least one substituent group. In embodiments, when L^{17B} is substituted, it is substituted with at least one size-limited substituent group. In embodiments, when L^{17B} is substituted, it is substituted with at least one lower substituent group.

In embodiments, a substituted L^{17C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17C} is substituted with a plurality of groups selected from substituent groups, size-limited substituent groups, and lower substituent groups; each substituent group, size-limited substituent group, and/or lower substituent group may optionally be different. In embodiments, when L^{17C} is substituted, it is substituted with at least one substituent group. In embodiments, when L^{17C} is substituted, it is substituted with at least one size-limited substituent group. In embodiments, when L^{17C} is substituted, it is substituted with at least one lower substituent group.

In embodiments, a substituted L^{7D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{7D} is substituted with a plurality of groups selected from substituent groups, size-limited substituent groups, and lower substituent groups; each substituent group, size-limited substituent group, and/or lower substituent group may optionally be different. In embodiments, when L^{17D} is substituted, it is substituted with at least one substituent group. In embodiments, when L^{17D} is substituted, it is substituted with at least one size-limited substituent group. In embodiments, when L^{17D} is substituted, it is substituted with at least one lower substituent group.

In embodiments, a substituted L¹⁷ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{7E} is substituted with a plurality of groups selected from substituent groups, size-limited substituent groups, and lower substituent groups; each substituent group, size-limited substituent group, and/or lower substituent group may optionally be different. In embodiments, when L^{17E} is substituted, it is substituted with at least one substituent group. In embodiments, when L^{17E} is substituted, it is substituted with at least one size-limited substituent group. In embodiments, when L^{17E} is substituted, it is substituted with at least one lower substituent group.

In embodiments, a substituted L^{17F} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17F} is substituted with a plurality of groups selected from substituent groups, size-limited substituent groups, and lower substituent groups; each substituent group, size-limited substituent group, and/or lower substituent group may optionally be different. In embodiments, when L^{17F} is substituted, it is substituted with at least one substituent group. In embodiments, when L^{17F} is substituted, it is substituted with at least one size-limited substituent group. In embodiments, when L^{17F} is substituted, it is substituted with at least one lower substituent group.

In embodiments, L^{17A} is a bond, unsubstituted alkylene, or unsubstituted heteroalkylene. In embodiments, L^{17A} is a bond, unsubstituted C₁-C₆ alkylene, or unsubstituted 2 to 6 membered heteroalkylene. In embodiments, L^{17A} is a bond. In embodiments, L^{17A} is unsubstituted C₁-C₆ alkylene. In embodiments, L^{17A} is unsubstituted 2 to 6 membered heteroalkylene. In embodiments, L^{17A} is

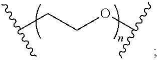

n is independently an integer from 1 to 100. In embodiments, n is independently an integer from 1 to 5.

In embodiments, L^{17A} is a bond, unsubstituted alkylene, or substituted or unsubstituted heteroalkylene. In embodiments, L^{17A} is a bond, unsubstituted C₁-C₆ alkylene, or unsubstituted 2 to 6 membered heteroalkylene. In embodiments, L^{17A} is a bond. In embodiments, L^{17A} is unsubstituted C₁-C₆ alkylene. In embodiments, L^{17A} is substituted 2 to 6 membered heteroalkylene. In embodiments, L^{17A} is

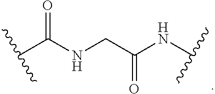 [SMILES: *NC(=O)CNC(*)=O]

In embodiments, L^{17A} is

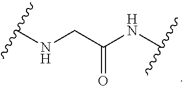 [SMILES: *NCC(=O)N*]

In embodiments, L^{17A} is unsubstituted 2 to 6 membered heteroalkylene. In embodiments, L^{17A} is

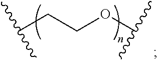

n is independently an integer from 1 to 100. In embodiments, n is independently an integer from 1 to 5.

In embodiments, L^{17B}, is a bond, —NHC(O)—, —C(O)NH—, substituted or unsubstituted alkylene, or substituted or unsubstituted heteroalkylene. In embodiments, L^{17B} is a bond, —NHC(O)—, —C(O)NH—, substituted or unsubstituted C₁-C₆ alkylene, or substituted or unsubstituted 2 to 6 membered heteroalkylene. In embodiments, L^{17B} is a bond. In embodiments, L^{17B} is —NHC(O)—. In embodiments, L^{17B} is —C(O)NH—. In embodiments, L^{17B} is substituted or unsubstituted C₁-C₆ alkylene. In embodiments, L^{17B} is substituted or unsubstituted 2 to 6 membered heteroalkylene. In embodiments, L^{17B} is

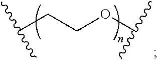

n is independently an integer from 1 to 100. In embodiments, n is independently an integer from 1 to 10. In embodiments, n is independently an integer from 1 to 5.

In embodiments, L^{17C} is a bond, unsubstituted alkylene, or unsubstituted heteroalkylene. In embodiments, L^{17C} is a bond, unsubstituted C₁-C₆ alkylene, or unsubstituted 2 to 6 membered heteroalkylene. In embodiments, L^{17C} is a bond. In embodiments, L^{17C} is unsubstituted C₁-C₆ alkylene. In embodiments, L^{17C} is unsubstituted methylene. In embodiments, L^{17C} is unsubstituted ethylene. In embodiments, L^{17C} is unsubstituted propylene. In embodiments, L^{17C} is unsubstituted n-propylene. In embodiments, L^{17C} is unsubstituted butylene. In embodiments, L^{17C} is unsubstituted n-butylene. In embodiments, L^{17C} is unsubstituted pentylene. In embodiments, L^{17C} is unsubstituted n-pentylene. In embodiments, L^{17C} is unsubstituted hexylene. In embodiments, L^{17C} is unsubstituted n-hexylene. In embodiments, L^{17C} is unsubstituted 2 to 6 membered heteroalkylene. In embodiments, L^{17C} is

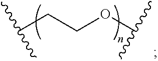

n is independently an integer from 1 to 100. In embodiments, n is independently an integer from 1 to 10. In embodiments, n is independently an integer from 1 to 5.

In embodiments, L^{17D} is a bond, —O—, unsubstituted alkylene, or unsubstituted heteroalkylene. In embodiments, L^{17D} is a bond, —O—, unsubstituted C₁-C₈ alkylene, or unsubstituted 2 to 6 membered heteroalkylene. In embodiments, L^{17D} is a bond. In embodiments, L^{17D} is —O—. In embodiments, L^{17D} is unsubstituted C₁-C₈ alkylene. In embodiments, L^{17D} is unsubstituted methylene. In embodiments, L^{17D} is unsubstituted ethylene. In embodiments, L^{17D} is unsubstituted propylene. In embodiments, L^{17D} is unsubstituted n-propylene. In embodiments, L^{17D} is unsubstituted butylene. In embodiments, L^{17D} is unsubstituted n-butylene. In embodiments, L^{17D} is unsubstituted pentylene. In embodiments, L^{17D} is unsubstituted n-pentylene. In embodiments, L^{17D} is unsubstituted hexylene. In embodiments, L^{17D} is unsubstituted n-hexylene. In embodiments, L^{17D} is unsubstituted heptylene. In embodiments, L^{17D} is unsubstituted n-heptylene. In embodiments, L^{17D} is unsubstituted octylene. In embodiments, L^{17D} is unsubstituted octylene. In embodiments, L^{17D} is unsubstituted 2 to 6 membered heteroalkylene.

In embodiments, L^{17E} is a bond, unsubstituted alkylene, or unsubstituted heteroalkylene. In embodiments, L^{17E} is a bond, unsubstituted C₁-C₈ alkylene, or unsubstituted 2 to 6 membered heteroalkylene. In embodiments, L^{17E} is a bond. In embodiments, L^{17E} is unsubstituted C₁-C₈ alkylene. In embodiments, L^{17E} is unsubstituted methylene. In embodiments, L^{17E} is unsubstituted ethylene. In embodiments, L^{17E} is unsubstituted propylene. In embodiments, L^{17E} is unsubstituted n-propylene. In embodiments, L^{17E} is unsubstituted butylene. In embodiments, L^{17E} is unsubstituted butylene. In embodiments, L^{17E} is unsubstituted pentylene. In embodiments, L^{17E} is unsubstituted n-pentylene. In embodiments, L^{17E} is unsubstituted hexylene. In embodiments, L^{17E} is unsubstituted n-hexylene. In embodiments, L^{17E} is unsubstituted heptylene. In embodiments, L^{17E} is unsubstituted n-heptylene. In embodiments, L^{17E} is unsubstituted octylene. In embodiments, L^{17E} is unsubstituted n-octylene. In embodiments, L^{7E} is unsubstituted 2 to 6 membered heteroalkylene.

In embodiments, L^{17F} is a bond, unsubstituted alkylene, or unsubstituted heteroalkylene. In embodiments, L^{17F} is a bond, unsubstituted C₁-C₈ alkylene, or unsubstituted 2 to 6 membered heteroalkylene. In embodiments, L^{17F} is a bond. In embodiments, L^{17F} is unsubstituted C₁-C₈ alkylene. In embodiments, L^{17F} is unsubstituted methylene. In embodiments, L^{17F} is unsubstituted ethylene. In embodiments, L^{17F} is unsubstituted propylene. In embodiments, L^{17F} is unsubstituted n-propylene. In embodiments, L^{17F} is unsubstituted butylene. In embodiments, L^{17F} is unsubstituted butylene. In embodiments, L^{17F} is unsubstituted pentylene. In embodiments, L^{17F} is unsubstituted n-pentylene. In embodiments, L^{17F} is unsubstituted hexylene. In embodiments, L^{17F} is unsubstituted n-hexylene. In embodiments, L^{17F} is unsubstituted heptylene. In embodiments, L^{17F} is unsubstituted n-heptylene. In embodiments, L^{17F} is unsubstituted octylene. In embodiments, L^{17F} is unsubstituted n-octylene. In embodiments, L^{17F} is unsubstituted 2 to 6 membered heteroalkylene.

In embodiments, n is independently 1. In embodiments, n is independently 2. In embodiments, n is independently 3. In embodiments, n is independently 4. In embodiments, n is independently 5. In embodiments, n is independently 6. In embodiments, n is independently 7. In embodiments, n is independently 8. In embodiments, n is independently 9. In embodiments, n is independently 10.

In embodiments, L¹⁷ is a divalent form of puromycin. In embodiments, L¹⁷ is -L^{17A}-(divalent form of puromycin)-L^{17E}-L^{17F}-; L^{17A}, L^{17E}, and L^{17F} are as described herein, including in embodiments. In embodiments, L¹⁷ is

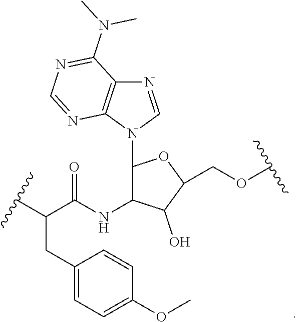 [SMILES: *OCC1OC(n2cnc3c(N(C)C)ncnc32)C(NC(=O)C(*)Cc2ccc(OC)cc2)C1O]

In embodiments, L¹⁷ is

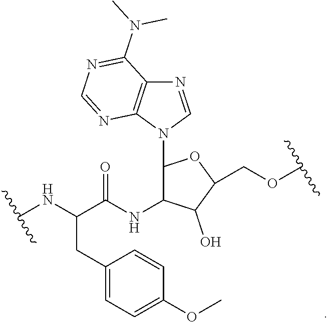 [SMILES: *NC(Cc1ccc(OC)cc1)C(=O)NC1C(O)C(CO*)OC1n1cnc2c(N(C)C)ncnc21]

In embodiments, L¹⁷ is

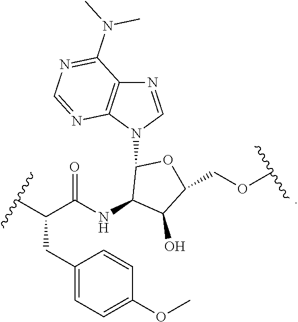 [SMILES: *OC[C@H]1O[C@@H](n2cnc3c(N(C)C)ncnc32)[C@H](NC(=O)[C@@H](*)Cc2ccc(OC)cc2)[C@@H]1O]

In embodiments, L¹⁷ is

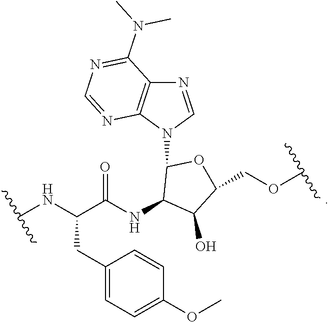 [SMILES: *N[C@@H](Cc1ccc(OC)cc1)C(=O)N[C@@H]1[C@H](O)[C@@H](CO*)O[C@H]1n1cnc2c(N(C)C)ncnc21]

In embodiments, -L^{17B}-L^{17C}-L^{17D}- is a divalent form of puromycin. In embodiments, -L^{17B}-L^{17C}-L^{17D}- is

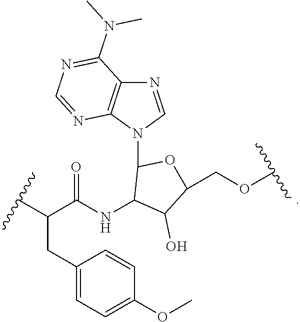 [SMILES: *OCC1OC(n2cnc3c(N(C)C)ncnc32)C(NC(=O)C(*)Cc2ccc(OC)cc2)C1O]

In embodiments, -L^{17B}-L^{17C}-L^{17D}- is

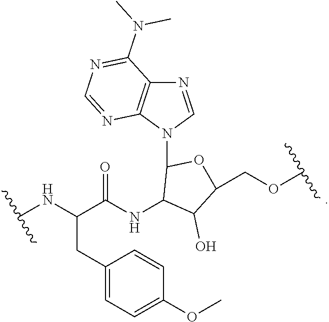 [SMILES: *NC(Cc1ccc(OC)cc1)C(=O)NC1C(O)C(CO*)OC1n1cnc2c(N(C)C)ncnc21]

In embodiments, -L^{17B}-L^{17C}-L^{17D}- is

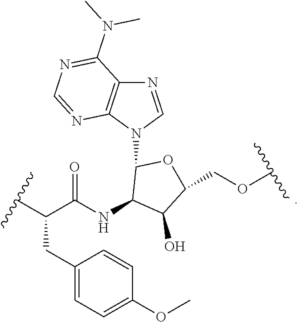 [SMILES: *OC[C@H]1O[C@@H](n2cnc3c(N(C)C)ncnc32)[C@H](NC(=O)[C@@H](*)Cc2ccc(OC)cc2)[C@@H]1O]

In embodiments, -L^{17B}-L^{17C}-L^{17D}- is

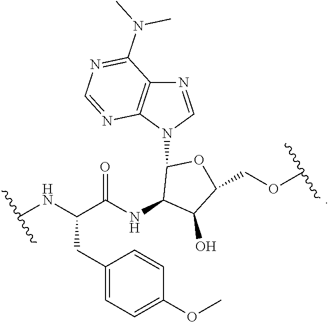 [SMILES: *N[C@@H](Cc1ccc(OC)cc1)C(=O)N[C@@H]1[C@H](O)[C@@H](CO*)O[C@H]1n1cnc2c(N(C)C)ncnc21]

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), a monovalent nucleic acid, a monovalent protein, or a detectable moiety.

In embodiments, R¹⁷ is hydrogen. In embodiments, R¹⁷ is —F. In embodiments, R¹⁷ is —Cl. In embodiments, R¹⁷ is —Br. In embodiments, R¹⁷ is —I. In embodiments, R¹⁷ is —CCl₃. In embodiments, R¹⁷ is —CBr₃. In embodiments, R¹⁷ is —CF₃. In embodiments, R¹⁷ is —Cl₃. In embodiments, R¹⁷ is —CHCl₂. In embodiments, R¹⁷ is —CHBr₂. In embodiments, R¹⁷ is —CHF₂. In embodiments, R¹⁷ is —CHI₂. In embodiments, R¹⁷ is —CH₂C₁. In embodiments, R¹⁷ is —CH₂Br. In embodiments, R¹⁷ is —CH₂F. In embodiments, R¹⁷ is —CH₂I. In embodiments, R¹⁷ is —OH. In embodiments, R¹⁷ is —NH₂. In embodiments, R¹⁷ is substituted or unsubstituted alkyl. In embodiments, R¹⁷ is substituted or unsubstituted C₁-C₆ alkyl. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted n-propyl. In embodiments, R¹⁷ is unsubstituted isopropyl. In embodiments, R¹⁷ is unsubstituted butyl. In embodiments, R¹⁷ is unsubstituted n-butyl. In embodiments, R¹⁷ is unsubstituted isobutyl. In embodiments, R¹⁷ is unsubstituted tert-butyl. In embodiments, R¹⁷ is substituted or unsubstituted heteroalkyl. In embodiments, R¹⁷ is substituted or unsubstituted 2 to 6 membered heteroalkyl. In embodiments, R¹⁷ is substituted or unsubstituted cycloalkyl. In embodiments, R¹⁷ is substituted or unsubstituted C₃-C₅ cycloalkyl. In embodiments, R¹⁷ is substituted or unsubstituted heterocycloalkyl. In embodiments, R¹⁷ is substituted or unsubstituted 3 to 8 membered heterocycloalkyl. In embodiments, R¹⁷ is substituted or unsubstituted aryl. In embodiments, R¹⁷ is substituted or unsubstituted C₆-C₁₀ aryl. In embodiments, R¹⁷ is substituted or unsubstituted heteroaryl. In embodiments, R¹⁷ is substituted or unsubstituted 5 to 10 membered heteroaryl. In embodiments, R¹⁷ is a monovalent nucleic acid. In embodiments, R¹⁷ is a monovalent protein. In embodiments, R¹⁷ is a detectable moiety. In embodiments, R¹⁷ is a drug moiety. In embodiments, R¹⁷ is a monovalent form of thalidomide.

In embodiments, -L¹⁷-R¹⁷ is

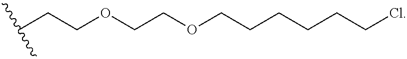 [SMILES: *CCOCCOCCCCCCCl]

In embodiments, -L¹⁷-R¹⁷ is

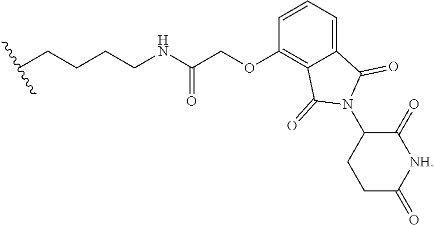 [SMILES: *CCCCNC(=O)COc1cccc2c1C(=O)N(C1CCC(=O)NC1=O)C2=O]

In embodiments, L¹⁶ is —SS—,

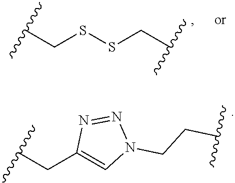 [SMILES: *CSSC* ; *CCn1cc(C*)nn1]

In embodiments, L¹⁶ is a bond,

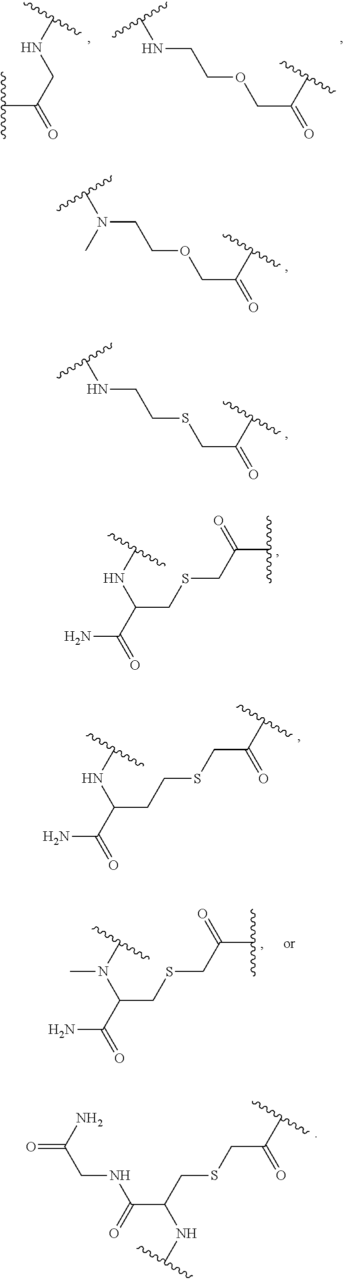

In embodiments, L¹⁶ is a bond. In embodiments, L¹⁶ is

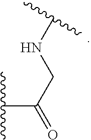 [SMILES: *NCC(*)=O]

In embodiments, L¹⁶ is

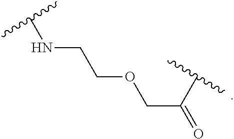 [SMILES: *NCCOCC(*)=O]

In embodiments, L¹⁶ is

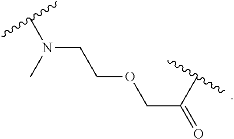 [SMILES: *C(=O)COCCN(*)C]

In embodiments, L¹⁶ is

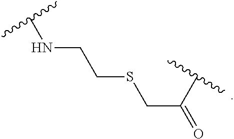 [SMILES: *NCCSCC(*)=O]

In embodiments, L¹⁶ is

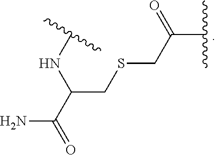 [SMILES: *NC(CSCC(*)=O)C(N)=O]

In embodiments, L¹⁶ is

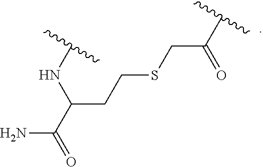 [SMILES: *NC(CCSCC(*)=O)C(N)=O]

In embodiments, L¹⁶ is

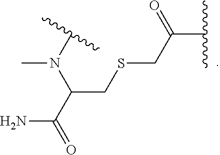 [SMILES: *C(=O)CSCC(C(N)=O)N(*)C]

In embodiments, L¹⁶ is

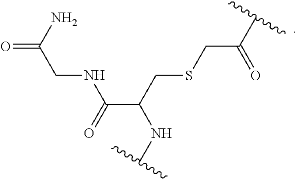 [SMILES: *NC(CSCC(*)=O)C(=O)NCC(N)=O]

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{11A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5E} is substituted, R^{5E} is substituted with one or more first substituent groups denoted by R^{5E.1} as explained in the definitions section above in the description of “first substituent group(s)”. In embodiments, when an R^{5E.1} substituent group is substituted, the R^{5E.1} substituent group is substituted with one or more second substituent groups denoted by R^{5E.2} as explained in the definitions section above in the description of “first substituent group(s)”. In embodiments, when an R^{5E.2} substituent group is substituted, the R^{5E.2} substituent group is substituted with one or more third substituent groups denoted by R^{5E}3 as explained in the definitions section above in the description of “first substituent group(s)”. In the above embodiments, R^{5E}, R^{5E.1}, R^{5E.2}, and R^{5E}3 have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5E}, R^{5E.1}, R^{5E.2}, and R^{5E.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{6B.1}, R^{6B.2}, and R^{6B.3}, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7A}, R^{7A.1}, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7B}, R^{7B.1}, R^{7B.2}, and R^{7B.3}, respectively.

In embodiments, when R^{8A} is substituted, R^{8A} is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A}, R^{8A.1}, R^{8A.2}, and R^{8A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8A}, R^{8A.1}, R^{8A.2}, and R^{8A.3}, respectively.

In embodiments, when R^{8B} is substituted, R^{8B} is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B}, R^{8B.1}, R^{8B.2}, and R^{8B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8B}, R^{8B.1}, R^{8B.2}, and R^{8B.3}, respectively.

In embodiments, when R^{9A} is substituted, R^{9A} is substituted with one or more first substituent groups denoted by R^{9A.1} as explained in the definitions section above in the description of “first substituent group(s)”. In embodiments, when an R^{9A.1} substituent group is substituted, the R^{9A.1} substituent group is substituted with one or more second substituent groups denoted by R^{9A.2} as explained in the definitions section above in the description of “first substituent group(s)”. In embodiments, when an R^{9A.2} substituent group is substituted, the R^{9A.2} substituent group is substituted with one or more third substituent groups denoted by R^{9A.3} as explained in the definitions section above in the description of “first substituent group(s)”. In the above embodiments, R^{9A}, R^{9A.1}, R^{9A.2}, and R^{9A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9A}, R^{9A.1}, R^{9A.2}, and R^{9A.3}, respectively.

In embodiments, when R^{9B} is substituted, R^{9B} is substituted with one or more first substituent groups denoted by R^{9B.1} as explained in the definitions section above in the description of “first substituent group(s)”. In embodiments, when an R^{9B.1} substituent group is substituted, the R^{9B.1} substituent group is substituted with one or more second substituent groups denoted by R^{9B.2} as explained in the definitions section above in the description of “first substituent group(s)”. In embodiments, when an R^{9B.2} substituent group is substituted, the R^{9B.2} substituent group is substituted with one or more third substituent groups denoted by R^{9B.3} as explained in the definitions section above in the description of “first substituent group(s)”. In the above embodiments, R^{9B}, R^{9B.1}, R^{9B.2}, and R^{9B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9B}, R^{9B.1}, R^{9B.2}, and R^{9B.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A.1}, R^{10A.2} and R^{10A.3} respectively.

In embodiments, when R^{10B} is substituted, R^{10B} is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2} and R^{10B.3} respectively.

In embodiments, when R^{11A} is substituted, R^{11A} is substituted with one or more first substituent groups denoted by R^{11A.1} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A}, R^{11A.1}, R^{11A.2}, and R^{11A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11A}, R^{11A.1}R^{11A.2} and R^{11A.3}, respectively.

In embodiments, when R^{11B} is substituted, R^{11B} is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{11B}, R^{1B.1}, R^{11B.2}, and R^{11B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11B}, R^{11B.1}, R^{11B.2} and R^{11B.3} respectively.

In embodiments, when R^{12A} is substituted, R^{12A} is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A.3} as explained in the definitions section above in the description of “first substituent group(s)”. In the above embodiments, R^{12A}, R^{12A.1}, R^{12A.2}, and R^{12A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12A}, R^{12A.1}, R^{12A.2} and R^{12A.3} respectively.

In embodiments, when R^{12B} is substituted, R^{12B} is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B}, R^{12B.1}, R^{12B.2}, and R^{12B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12B}, R^{12B.1}, R^{12B.2} and R^{12B.3} respectively.

In embodiments, when R^{13B} is substituted, R^{13B} is substituted with one or more first substituent groups denoted by R^{13B.1} as explained in the definitions section above in the description of “first substituent group(s)”. In embodiments, when an R^{13B.1} substituent group is substituted, the R^{13B.1} substituent group is substituted with one or more second substituent groups denoted by R^{13B.2} as explained in the definitions section above in the description of “first substituent group(s)”. In embodiments, when an R^{13B.2} substituent group is substituted, the R^{13B.2} substituent group is substituted with one or more third substituent groups denoted by R^{13B.3} as explained in the definitions section above in the description of “first substituent group(s)”. In the above embodiments, R^{13B}, R^{13B.1}, R^{13B.2}, and R^{13B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13B}, R^{13B.1}, R^{13B.2} and R^{13B.3} respectively.

In embodiments, when R^{13E} is substituted, R^{13E} is substituted with one or more first substituent groups denoted by R^{13E.1} as explained in the definitions section above in the description of “first substituent group(s)”. In embodiments, when an R^{13E.1} substituent group is substituted, the R^{13E.1} substituent group is substituted with one or more second substituent groups denoted by R^{13E.2} as explained in the definitions section above in the description of “first substituent group(s)”. In embodiments, when an R^{13E.2} substituent group is substituted, the R^{13E.2} substituent group is substituted with one or more third substituent groups denoted by R^{13E.3} as explained in the definitions section above in the description of “first substituent group(s)”. In the above embodiments, R^{13E}, R^{13E.1}, R^{13E.2}, and R^{13E.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13E}, R^{13E.1}, R^{13E.2} and R^{13E.3} respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R¹?1, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW2}, and R^{WW.3} correspond to R¹⁷, R^{17.1}, R^{17.2}, and R^{17.3}, respectively.

In embodiments, when L^{1A} is substituted, L^{1A} is substituted with one or more first substituent groups denoted by R^{L1A.1} as explained in the definitions section above in the description of “first substituent group(s)”. In embodiments, when an R^{L1A.1} substituent group is substituted, the R^{L1A.1} substituent group is substituted with one or more second substituent groups denoted by R^{L1A.2} as explained in the definitions section above in the description of “first substituent group(s)”. In embodiments, when an R^{L1A.2} substituent group is substituted, the R^{L1A.2} substituent group is substituted with one or more third substituent groups denoted by R^{L1A.3} as explained in the definitions section above in the description of “first substituent group(s)”. In the above embodiments, L^{1A}, R^{L1A.1}, R^{L1A.2}, and R^{L1A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1A}, R^{L1A.1}, R^{L1A.2}, and R^{L1A.3}, respectively.

In embodiments, when L^{1B} is substituted, L^{1B} is substituted with one or more first substituent groups denoted by R^{L1B.1} as explained in the definitions section above in the description of “first substituent group(s)”. In embodiments, when an R^{L1B.1} substituent group is substituted, the R^{L1B.1} substituent group is substituted with one or more second substituent groups denoted by R^{L1B.2} as explained in the definitions section above in the description of “first substituent group(s)”. In embodiments, when an R^{L1B.2} substituent group is substituted, the R^{L1B.2} substituent group is substituted with one or more third substituent groups denoted by R^{L1B.3} as explained in the definitions section above in the description of “first substituent group(s)”. In the above embodiments, L^{1B}, R^{LB.1}, R^{L1B.2}, and R^{L1B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1B}, R^{L1B.1}, R^{L1B.2}, and R^{L1B.3}, respectively.

In embodiments, when L^{2A} is substituted, L^{2A} is substituted with one or more first substituent groups denoted by R^{L2A.1} as explained in the definitions section above in the description of “first substituent group(s)”. In embodiments, when an R^{L2A.1} substituent group is substituted, the R^{L2A.1} substituent group is substituted with one or more second substituent groups denoted by R^{L2A.2} as explained in the definitions section above in the description of “first substituent group(s)”. In embodiments, when an R^{L2A.2} substituent group is substituted, the R^{L2A.2} substituent group is substituted with one or more third substituent groups denoted by R^{L2A.3} as explained in the definitions section above in the description of “first substituent group(s)”. In the above embodiments, L^{2A}, R^{L2A.1}, R^{L2A.2}, and R^{L2A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2A}, R^{L2A.1}, R^{L2A.2}, and R^{L2A.3}, respectively.

In embodiments, when L^{2B} is substituted, L^{2B} is substituted with one or more first substituent groups denoted by R^{L2B.1} as explained in the definitions section above in the description of “first substituent group(s)”. In embodiments, when an R^{L2B.1} substituent group is substituted, the R^{L2B.1} substituent group is substituted with one or more second substituent groups denoted by R^{L2B.2} as explained in the definitions section above in the description of “first substituent group(s)”. In embodiments, when an R^{L2B.2} substituent group is substituted, the R^{L2B.2} substituent group is substituted with one or more third substituent groups denoted by R^{L2B.3} as explained in the definitions section above in the description of “first substituent group(s)”. In the above embodiments, L^{2B}, R^{L2B.1}, R^{L2B.2}, and R^{L2B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2B}, R^{L2B.1}, R^{L2B.2}, and R^{L2B.3}, respectively.

In embodiments, when L^{3A} is substituted, L^{3A} is substituted with one or more first substituent groups denoted by R^{L3A.1} as explained in the definitions section above in the description of “first substituent group(s)”. In embodiments, when an R^{L3A.1} substituent group is substituted, the R^{L3A.1} substituent group is substituted with one or more second substituent groups denoted by R^{L3A.2} as explained in the definitions section above in the description of “first substituent group(s)”. In embodiments, when an R^{L3A.2} substituent group is substituted, the R^{L3A.2} substituent group is substituted with one or more third substituent groups denoted by R^{L3A.3} as explained in the definitions section above in the description of “first substituent group(s)”. In the above embodiments, L^{3A}, R^{L3A.1}, R^{L3A.2}, and R^{L3A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3A}, R^{L3A.1}, R^{L3A.2}, and R^{L3A.3}, respectively.

In embodiments, when L^{3B} is substituted, L^{3B} is substituted with one or more first substituent groups denoted by R^{L3B.1} as explained in the definitions section above in the description of “first substituent group(s)”. In embodiments, when an R^{L3B.1} substituent group is substituted, the R^{L3B.1} substituent group is substituted with one or more second substituent groups denoted by R^{L3B.2} as explained in the definitions section above in the description of “first substituent group(s)”. In embodiments, when an R^{L3B.2} substituent group is substituted, the R^{L3B.2} substituent group is substituted with one or more third substituent groups denoted by R^{L3B.3} as explained in the definitions section above in the description of “first substituent group(s)”. In the above embodiments, L^{3B}, R^{L3B.1}, R^{L3B.2}, and R^{L3B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3B}, R^{L3B.1}, R^{L3B.2}, and R^{L3B.3}, respectively.

In embodiments, when L^{4A} is substituted, L^{4A} is substituted with one or more first substituent groups denoted by R^{L4A.1} as explained in the definitions section above in the description of “first substituent group(s)”. In embodiments, when an R^{L4A.1} substituent group is substituted, the R^{L4A.1} substituent group is substituted with one or more second substituent groups denoted by R^{L4A.2} as explained in the definitions section above in the description of “first substituent group(s)”. In embodiments, when an R^{L4A.2} substituent group is substituted, the R^{L4A.2} substituent group is substituted with one or more third substituent groups denoted by R^{L4A.3} as explained in the definitions section above in the description of “first substituent group(s)”. In the above embodiments, L^{4A}, R^{L4A.1}, R^{L4A.2}, and R^{L4A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{4A}, R^{L4A.1}, R^{L4A.2}, and R^{L4A.3}, respectively.

In embodiments, when L^{4B} is substituted, L^{4B} is substituted with one or more first substituent groups denoted by R^{L4B.1} as explained in the definitions section above in the description of “first substituent group(s)”. In embodiments, when an R^{L4B.1} substituent group is substituted, the R^{L4B.1} substituent group is substituted with one or more second substituent groups denoted by R^{L4B.2} as explained in the definitions section above in the description of “first substituent group(s)”. In embodiments, when an R^{L4B.2} substituent group is substituted, the R^{L4B.2} substituent group is substituted with one or more third substituent groups denoted by R^{L4B.3} as explained in the definitions section above in the description of “first substituent group(s)”. In the above embodiments, L^{4B}, R^{L4B.1}, R^{L4B.2}, and R^{L4B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{4B}, R^{L4B.1}, R^{L4B.2}, and R^{L4B.3}, respectively.

In embodiments, when L^{5A} is substituted, L^{5A} is substituted with one or more first substituent groups denoted by R^{L5A.1} as explained in the definitions section above in the description of “first substituent group(s)”. In embodiments, when an R^{L5A.1} substituent group is substituted, the R^{L5A.1} substituent group is substituted with one or more second substituent groups denoted by R^{L5A.2} as explained in the definitions section above in the description of “first substituent group(s)”. In embodiments, when an R^{L5A.2} substituent group is substituted, the R^{L5A.2} substituent group is substituted with one or more third substituent groups denoted by R^{L5A.3} as explained in the definitions section above in the description of “first substituent group(s)”. In the above embodiments, L^{5A}, R^{L5A.1}, R^{L5A.2}, and R^{L5A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{5A}, R^{L5A.1}, R^{L5A.2}, and R^{L5A.3}, respectively.

In embodiments, when L^{5B} is substituted, L^{5B} is substituted with one or more first substituent groups denoted by R^{L5B.1} as explained in the definitions section above in the description of “first substituent group(s)”. In embodiments, when an R^{L5B.1} substituent group is substituted, the R^{L5B.1} substituent group is substituted with one or more second substituent groups denoted by R^{L5B.2} as explained in the definitions section above in the description of “first substituent group(s)”. In embodiments, when an R^{L5B.2} substituent group is substituted, the R^{L5B.2} substituent group is substituted with one or more third substituent groups denoted by R^{L5B.3} as explained in the definitions section above in the description of “first substituent group(s)”. In the above embodiments, L^{5B}, R^{L5B.1}, R^{L5B.2}, and R^{L5B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{5B}, R^{L5B.1}, R^{L5B.2}, and R^{L5B.3}, respectively.

In embodiments, when L^{6A} is substituted, L^{6A} is substituted with one or more first substituent groups denoted by R^{L6A.1} as explained in the definitions section above in the description of “first substituent group(s)”. In embodiments, when an R^{L6A.1} substituent group is substituted, the R^{L6A.1} substituent group is substituted with one or more second substituent groups denoted by R^{L6A.2} as explained in the definitions section above in the description of “first substituent group(s)”. In embodiments, when an R^{L6A.2} substituent group is substituted, the R^{L6A.2} substituent group is substituted with one or more third substituent groups denoted by R^{L6A.3} as explained in the definitions section above in the description of “first substituent group(s)”. In the above embodiments, L^{6A}, R^{L6A.1}, R^{L6A.2}, and R^{L6A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{6A}, R^{L6A.1}, R^{L6A.2}, and R^{L6A.3}, respectively.

In embodiments, when L^{6B} is substituted, L^{6B} is substituted with one or more first substituent groups denoted by R^{L6B.1} as explained in the definitions section above in the description of “first substituent group(s)”. In embodiments, when an R^{L6B.1} substituent group is substituted, the R^{L6B.1} substituent group is substituted with one or more second substituent groups denoted by R^{L6B.2} as explained in the definitions section above in the description of “first substituent group(s)”. In embodiments, when an R^{L6B.2} substituent group is substituted, the R^{L6B.2} substituent group is substituted with one or more third substituent groups denoted by R^{L6B.3} as explained in the definitions section above in the description of “first substituent group(s)”. In the above embodiments, L^{6B}, R^{L6B.1}, R^{L6B.2}, and R^{L6B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{6B}, R^{L6B.1}, R^{L6B.2}, and R^{L6B.3}, respectively.

In embodiments, when L^{7A} is substituted, L^{7A} is substituted with one or more first substituent groups denoted by R^{L7A.1} as explained in the definitions section above in the description of “first substituent group(s)”. In embodiments, when an R^{L7A.1} substituent group is substituted, the R^{L7A.1} substituent group is substituted with one or more second substituent groups denoted by R^{L7A.2} as explained in the definitions section above in the description of “first substituent group(s)”. In embodiments, when an R^{L7A.2} substituent group is substituted, the R^{L7A.2} substituent group is substituted with one or more third substituent groups denoted by R^{L7A.3} as explained in the definitions section above in the description of “first substituent group(s)”. In the above embodiments, L^{7A}, R^{L7A.1}, R^{L7A.2}, and R^{L7A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{7A}, R^{L7A.1}, R^{L7A.2}, and R^{L7A.3}, respectively.

In embodiments, when L^{7B} is substituted, L^{7B} is substituted with one or more first substituent groups denoted by R^{L7B.1} as explained in the definitions section above in the description of “first substituent group(s)”. In embodiments, when an R^{L7B.1} substituent group is substituted, the R^{L7B.1} substituent group is substituted with one or more second substituent groups denoted by R^{L7B.2} as explained in the definitions section above in the description of “first substituent group(s)”. In embodiments, when an R^{L7B.2} substituent group is substituted, the R^{L7B.2} substituent group is substituted with one or more third substituent groups denoted by R^{L7B.3} as explained in the definitions section above in the description of “first substituent group(s)”. In the above embodiments, L^{7B}, R^{L7B.1}, R^{L7B.2}, and R^{L7B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{7B}, R^{L7B.1}, R^{L7B.2}, and R^{L7B.3}, respectively.

In embodiments, when L^{8A} is substituted, L^{8A} is substituted with one or more first substituent groups denoted by R^{L8A.1} as explained in the definitions section above in the description of “first substituent group(s)”. In embodiments, when an R^{L8A.1} substituent group is substituted, the R^{L8A.1} substituent group is substituted with one or more second substituent groups denoted by R^{L8A.2} as explained in the definitions section above in the description of “first substituent group(s)”. In embodiments, when an R^{L8A.2} substituent group is substituted, the R^{L8A.2} substituent group is substituted with one or more third substituent groups denoted by R^{L8A.3} as explained in the definitions section above in the description of “first substituent group(s)”. In the above embodiments, L^{8A}, R^{LA.1}, R^{LA.2}, and R^{L8A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{8A}, R^{LA.1}, R^{LA.2}, and R^{LA.3}, respectively.

In embodiments, when L^{8B} is substituted, L^{8B} is substituted with one or more first substituent groups denoted by R^{LB.1} as explained in the definitions section above in the description of “first substituent group(s)”. In embodiments, when an R^{L8B.1} substituent group is substituted, the R^{L8B.1} substituent group is substituted with one or more second substituent groups denoted by R^{L8B.2} as explained in the definitions section above in the description of “first substituent group(s)”. In embodiments, when an R^{L8B.2} substituent group is substituted, the R^{L8B.2} substituent group is substituted with one or more third substituent groups denoted by R^{L8B.3} as explained in the definitions section above in the description of “first substituent group(s)”. In the above embodiments, L^{8B}, R^{L8B.1}, R^{L8B.2}, and R^{L8B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{8B}, R^{L8B.1}, R^{L8B.2}, and R^{L8B.3}, respectively.

In embodiments, when L^{9A} is substituted, L^{9A} is substituted with one or more first substituent groups denoted by R^{L9A.1} as explained in the definitions section above in the description of “first substituent group(s)”. In embodiments, when an R^{L9A.1} substituent group is substituted, the R^{L9A.1} substituent group is substituted with one or more second substituent groups denoted by R^{L9A.2} as explained in the definitions section above in the description of “first substituent group(s)”. In embodiments, when an R^{L9A.2} substituent group is substituted, the R^{L9A.2} substituent group is substituted with one or more third substituent groups denoted by R^{L9A.3} as explained in the definitions section above in the description of “first substituent group(s)”. In the above embodiments, L^{9A}, R^{L9A.1}, R^{L9A.2}, and R^{L9A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{9A}, R^{L9A.1}, R^{L9A.2}, and R^{L9A.3}, respectively.

In embodiments, when L^{9B} is substituted, L^{9B} is substituted with one or more first substituent groups denoted by R^{L9B.1} as explained in the definitions section above in the description of “first substituent group(s)”. In embodiments, when an R^{L9B.1} substituent group is substituted, the R^{L9B.1} substituent group is substituted with one or more second substituent groups denoted by R^{L9B.2} as explained in the definitions section above in the description of “first substituent group(s)”. In embodiments, when an R^{L9B.2} substituent group is substituted, the R^{L9B.2} substituent group is substituted with one or more third substituent groups denoted by R^{L9B.3} as explained in the definitions section above in the description of “first substituent group(s)”. In the above embodiments, L^{9B}, R^{L9B.1}, R^{L9B.2}, and R^{L9B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{9B}, R^{L9B.1}, R^{L9B.2}, and R^{L9B.3}, respectively.

In embodiments, when L^{10A} is substituted, L^{10A} is substituted with one or more first substituent groups denoted by R^{L10A.1} as explained in the definitions section above in the description of “first substituent group(s)”. In embodiments, when an R^{L10A.1} substituent group is substituted, the R^{L10A.1} substituent group is substituted with one or more second substituent groups denoted by R^{L10A.2} as explained in the definitions section above in the description of “first substituent group(s)”. In embodiments, when an R^{L10A.2} substituent group is substituted, the R^{L10A.2} substituent group is substituted with one or more third substituent groups denoted by R^{L10A.3} as explained in the definitions section above in the description of “first substituent group(s)”. In the above embodiments, L^{10A}, R^{L10A.1}, R^{L10A.2} and R^{L10A.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{10A}, R^{L10A.1}, R^{L10A.2} and R^{L10A.3}, respectively.

In embodiments, when L^{10B} is substituted, L^{10B} is substituted with one or more first substituent groups denoted by R^{L10B.1} as explained in the definitions section above in the description of “first substituent group(s)”. In embodiments, when an R^{L10B.1} substituent group is substituted, the R^{L10B.1} substituent group is substituted with one or more second substituent groups denoted by R^{L10B.2} as explained in the definitions section above in the description of “first substituent group(s)”. In embodiments, when an R^{L10B.2} substituent group is substituted, the R^{L10B.2} substituent group is substituted with one or more third substituent groups denoted by R^{L10B.3} as explained in the definitions section above in the description of “first substituent group(s)”. In the above embodiments, L^{10B}, R^{L10B.1}, R^{L10B.2} and R^{L10B.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{10B}, R^{L10B.1}, R^{L10B.2} and R^{L10B.3}, respectively.

In embodiments, when L^{11A} is substituted, L^{11A} is substituted with one or more first substituent groups denoted by R^{L11A.1} as explained in the definitions section above in the description of “first substituent group(s)”. In embodiments, when an R^{L11A.1} substituent group is substituted, the R^{L11A.1} substituent group is substituted with one or more second substituent groups denoted by R^{L11A.2} as explained in the definitions section above in the description of “first substituent group(s)”. In embodiments, when an R^{L11A.2} substituent group is substituted, the R^{L11A.2} substituent group is substituted with one or more third substituent groups denoted by R^{L11A.3} as explained in the definitions section above in the description of “first substituent group(s)”. In the above embodiments, L^{11A}, R^{L11A.1}, R^{L11A.2} and R^{L11A.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{11A}, R^{L11A.1}, R^{L11A.2} and R^{L11A.3}, respectively.

In embodiments, when L^{11B} is substituted, L^{11B} is substituted with one or more first substituent groups denoted by R^{L11B.1} as explained in the definitions section above in the description of “first substituent group(s)”. In embodiments, when an R^{L11B.1} substituent group is substituted, the R^{L11B.1} substituent group is substituted with one or more second substituent groups denoted by R^{L11B.2} as explained in the definitions section above in the description of “first substituent group(s)”. In embodiments, when an R^{L11B.2} substituent group is substituted, the R^{L11B.2} substituent group is substituted with one or more third substituent groups denoted by R^{L11B.3} as explained in the definitions section above in the description of “first substituent group(s)”. In the above embodiments, L^{11B}, R^{L11B.1}, R^{L11B.2} and R^{L11B.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{11B}, R^{L11B.1}, R^{L11B.2} and R^{L11B.3}, respectively.

In embodiments, when L^{12A} is substituted, L^{12A} is substituted with one or more first substituent groups denoted by R^{L12A.1} as explained in the definitions section above in the description of “first substituent group(s)”. In embodiments, when an R^{L12A.1} substituent group is substituted, the R^{L12A.1} substituent group is substituted with one or more second substituent groups denoted by R^{L12A.2} as explained in the definitions section above in the description of “first substituent group(s)”. In embodiments, when an R^{L12A.2} substituent group is substituted, the R^{L12A.2} substituent group is substituted with one or more third substituent groups denoted by R^{L12A.3} as explained in the definitions section above in the description of “first substituent group(s)”. In the above embodiments, L^{12A}, R^{L12A.1}, R^{L12A.2} and R^{L12A.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{12A}, R^{L12A.1}, R^{L12A.2} and R^{L12A.3}, respectively.

In embodiments, when L^{12B} is substituted, L^{12B} is substituted with one or more first substituent groups denoted by R^{L12B.1} as explained in the definitions section above in the description of “first substituent group(s)”. In embodiments, when an R^{L12B.1} substituent group is substituted, the R^{L12B.1} substituent group is substituted with one or more second substituent groups denoted by R^{L12B.2} as explained in the definitions section above in the description of “first substituent group(s)”. In embodiments, when an R^{L12B.2} substituent group is substituted, the R^{L12B.2} substituent group is substituted with one or more third substituent groups denoted by R^{L12B.3} as explained in the definitions section above in the description of “first substituent group(s)”. In the above embodiments, L^{12B}, R^{L12B.1}, R^{L12B.2} and R^{L12B.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{12B}, R^{L12B.1}, R^{L12B.2} and R^{L12B.3}, respectively.

In embodiments, when L^{13B} is substituted, L^{13B} is substituted with one or more first substituent groups denoted by R^{L13B.1} as explained in the definitions section above in the description of “first substituent group(s)”. In embodiments, when an R^{L13B.1} substituent group is substituted, the R^{L13B.1} substituent group is substituted with one or more second substituent groups denoted by R^{L13B.2} as explained in the definitions section above in the description of “first substituent group(s)”. In embodiments, when an R^{L13B.2} substituent group is substituted, the R^{L13B.2} substituent group is substituted with one or more third substituent groups denoted by R^{L13B.3} as explained in the definitions section above in the description of “first substituent group(s)”. In the above embodiments, L^{13B}, R^{L13B.1}, R^{L13B.2} and R^{L13B.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{13B}, R^{L13B.1}, R^{L13B.2} and R^{L13B.3}, respectively.

In embodiments, when L¹⁶ is substituted, L¹⁶ is substituted with one or more first substituent groups denoted by R^{L16.1} as explained in the definitions section above in the description of “first substituent group(s)”. In embodiments, when an R^{L16.1} substituent group is substituted, the R^{L16.1} substituent group is substituted with one or more second substituent groups denoted by R^{L16.2} as explained in the definitions section above in the description of “first substituent group(s)”. In embodiments, when an R^{L16.2} substituent group is substituted, the R^{L16.2} substituent group is substituted with one or more third substituent groups denoted by R^{L16.3} as explained in the definitions section above in the description of “first substituent group(s)”. In the above embodiments, L¹⁶, R^{L16.1}, R^{L16.2}, and R^{L16.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁶, R^{L16.1}, R^{L16.2}, and R^{L16.3}, respectively.

In embodiments, when L^{16A} is substituted, L^{16A} is substituted with one or more first substituent groups denoted by R^{L16A.1} as explained in the definitions section above in the description of “first substituent group(s)”. In embodiments, when an R^{L16A.1} substituent group is substituted, the R^{L16A.1} substituent group is substituted with one or more second substituent groups denoted by R^{L16A.2} as explained in the definitions section above in the description of “first substituent group(s)”. In embodiments, when an R^{L16A.2} substituent group is substituted, the R^{L16A.2} substituent group is substituted with one or more third substituent groups denoted by R^{L16A.3} as explained in the definitions section above in the description of “first substituent group(s)”. In the above embodiments, L^{16A}, R^{L16A.1}, R^{L16A.2} and R^{L16A.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{16A}, R^{L16A.1}, R^{L16A.2} and R^{L16A.3}, respectively.

In embodiments, when L^{16B} is substituted, L^{16B} is substituted with one or more first substituent groups denoted by R^{L16B.1} as explained in the definitions section above in the description of “first substituent group(s)”. In embodiments, when an R^{L16B.1} substituent group is substituted, the R^{L16B.1} substituent group is substituted with one or more second substituent groups denoted by R^{L16B.2} as explained in the definitions section above in the description of “first substituent group(s)”. In embodiments, when an R^{L16B.2} substituent group is substituted, the R^{L16B.2} substituent group is substituted with one or more third substituent groups denoted by R^{L16B.3} as explained in the definitions section above in the description of “first substituent group(s)”. In the above embodiments, L^{16B}, R^{L16B.1}, R^{L16B.2} and R^{L16B.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{16B}, R^{L16B.1}, R^{L16B.2} and R^{L16B.3}, respectively.

In embodiments, when L^{16C} is substituted, L^{16C} is substituted with one or more first substituent groups denoted by R^{L16C.1} as explained in the definitions section above in the description of “first substituent group(s)”. In embodiments, when an R^{L16C.1} substituent group is substituted, the R^{L16C.1} substituent group is substituted with one or more second substituent groups denoted by R^{L16C.2} as explained in the definitions section above in the description of “first substituent group(s)”. In embodiments, when an R^{L16C.2} substituent group is substituted, the R^{L16C.2} substituent group is substituted with one or more third substituent groups denoted by R^{L16C.3} as explained in the definitions section above in the description of “first substituent group(s)”. In the above embodiments, L^{16C}, R^{L16C.1}, R^{L16C.2} and R^{L16C.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{16C}, R^{L16C.1}, R^{L16C.2} and R^{L16C.1}, respectively.

In embodiments, when L^{16D} is substituted, L^{16D} is substituted with one or more first substituent groups denoted by R^{L16D.1} as explained in the definitions section above in the description of “first substituent group(s)”. In embodiments, when an R^{L16D.1} substituent group is substituted, the R^{L16D.1} substituent group is substituted with one or more second substituent groups denoted by R^{L16D.2} as explained in the definitions section above in the description of “first substituent group(s)”. In embodiments, when an R^{L16D.2} substituent group is substituted, the R^{L16D.2} substituent group is substituted with one or more third substituent groups denoted by R^{L16D.3} as explained in the definitions section above in the description of “first substituent group(s)”. In the above embodiments, L^{16D}, R^{L16D.1}, R^{L16D.2} and R^{L16D.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{16D}, R^{L16D.1}, R^{L16D.2} and R^{L16D.3}, respectively.

In embodiments, when L^{16E} is substituted, L^{16E} is substituted with one or more first substituent groups denoted by R^{L16E.1} as explained in the definitions section above in the description of “first substituent group(s)”. In embodiments, when an R^{L16E.1} substituent group is substituted, the R^{L16E.1} substituent group is substituted with one or more second substituent groups denoted by R^{L16E.2} as explained in the definitions section above in the description of “first substituent group(s)”. In embodiments, when an R^{L16E.2} substituent group is substituted, the R^{L16E.2} substituent group is substituted with one or more third substituent groups denoted by R^{L16E.3} as explained in the definitions section above in the description of “first substituent group(s)”. In the above embodiments, L^{16E}, R^{L16E.1}, R^{L16E.2} and R^{L16E.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{16E}, R^{L16E.1}, R^{L16E.2} and R^{L16E.3}, respectively.

In embodiments, when L^{16F} is substituted, L^{16F} is substituted with one or more first substituent groups denoted by R^{L16F.1} as explained in the definitions section above in the description of “first substituent group(s)”. In embodiments, when an R^{L16F.1} substituent group is substituted, the R^{L16F.1} substituent group is substituted with one or more second substituent groups denoted by R^{L16F.2} as explained in the definitions section above in the description of “first substituent group(s)”. In embodiments, when an R^{L16F.2} substituent group is substituted, the R^{L16F.2} substituent group is substituted with one or more third substituent groups denoted by R^{L16F.3} as explained in the definitions section above in the description of “first substituent group(s)”. In the above embodiments, L^{16F}, R^{L16F.1}, R^{L16F.2}, and R^{L16F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{16F}, R^{L16F.1}, R^{L16F.2} and R^{L16F.3} respectively.

In embodiments, when L^{17A} is substituted, L^{17A} is substituted with one or more first substituent groups denoted by R^{L17A.1} as explained in the definitions section above in the description of “first substituent group(s)”. In embodiments, when an R^{L17A.1} substituent group is substituted, the R^{L17A.1} substituent group is substituted with one or more second substituent groups denoted by R^{L17A.2} as explained in the definitions section above in the description of “first substituent group(s)”. In embodiments, when an R^{L17A.2} substituent group is substituted, the R^{L17A.2} substituent group is substituted with one or more third substituent groups denoted by R^{L17A.3} as explained in the definitions section above in the description of “first substituent group(s)”. In the above embodiments, L^{17A}, R^{L17A.1}, R^{L17A.2} and R^{L17A.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{17A}, R^{L17A.1}, R^{L17A.2} and R^{L17A.3}, respectively.

In embodiments, when L^{17B}, is substituted, L^{17B}, is substituted with one or more first substituent groups denoted by R^{L17B.1} as explained in the definitions section above in the description of “first substituent group(s)”. In embodiments, when an R^{L17B.1} substituent group is substituted, the R^{L17B.1} substituent group is substituted with one or more second substituent groups denoted by R^{L17B.2} as explained in the definitions section above in the description of “first substituent group(s)”. In embodiments, when an R^{L17B.2} substituent group is substituted, the R^{L17B.2} substituent group is substituted with one or more third substituent groups denoted by R^{L17B.3} as explained in the definitions section above in the description of “first substituent group(s)”. In the above embodiments, L^{17B}, R^{L17B.1}, R^{L17B.2} and R^{L17B.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{17B}, R^{L17B.1}, R^{L17B.2} and R^{L17B.3}, respectively.

In embodiments, when L^{17C} is substituted, L^{17C} is substituted with one or more first substituent groups denoted by R^{L17C.1} as explained in the definitions section above in the description of “first substituent group(s)”. In embodiments, when an R^{L7C.1} substituent group is substituted, the R^{L17C.1} substituent group is substituted with one or more second substituent groups denoted by R^{L17C.2} as explained in the definitions section above in the description of “first substituent group(s)”. In embodiments, when an R^{L17C.2} substituent group is substituted, the R^{L17C.2} substituent group is substituted with one or more third substituent groups denoted by R^{L17C.3} as explained in the definitions section above in the description of “first substituent group(s)”. In the above embodiments, L^{17C}, R^{L17C.1}, R^{L17C.2} and R^{L17C.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{17C}, R^{L17C.1}, R^{L17C.2} and R^{L17C.3}, respectively.

In embodiments, when L^{17D} is substituted, L^{17D} is substituted with one or more first substituent groups denoted by R^{L17D.1} as explained in the definitions section above in the description of “first substituent group(s)”. In embodiments, when an R^{L17D.1} substituent group is substituted, the R^{L17D.1} substituent group is substituted with one or more second substituent groups denoted by R^{L17D.2} as explained in the definitions section above in the description of “first substituent group(s)”. In embodiments, when an R^{L17D.2} substituent group is substituted, the R^{L17D.2} substituent group is substituted with one or more third substituent groups denoted by R^{L17D.3} as explained in the definitions section above in the description of “first substituent group(s)”. In the above embodiments, L^{17D}, R^{L17D.1}, R^{L17D.2} and R^{L17D.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{17D}, R^{L17D.1}, R^{L17D.2} and R^{L17D.3}, respectively.

In embodiments, when L^{17E} is substituted, L^{17E} is substituted with one or more first substituent groups denoted by R^{L17E.1} as explained in the definitions section above in the description of “first substituent group(s)”. In embodiments, when an R^{L17E.1} substituent group is substituted, the R^{L17E.1} substituent group is substituted with one or more second substituent groups denoted by R^{L17E.2} as explained in the definitions section above in the description of “first substituent group(s)”. In embodiments, when an R^{L17E.2} substituent group is substituted, the R^{L17E.2} substituent group is substituted with one or more third substituent groups denoted by R^{L17E.3} as explained in the definitions section above in the description of “first substituent group(s)”. In the above embodiments, L^{17E}, R^{L17E.1}, R^{L17E.2} and R^{L17E.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L^{17E}, R^{L17E.1}, R^{L17E.2} and R^{L17E.3} respectively.

In embodiments, when L^{17F} is substituted, L^{17F} is substituted with one or more first substituent groups denoted by R^{L17F.1} as explained in the definitions section above in the description of “first substituent group(s)”. In embodiments, when an R^{L17F.1} substituent group is substituted, the R^{L17F.1} substituent group is substituted with one or more second substituent groups denoted by R^{L17F.2} as explained in the definitions section above in the description of “first substituent group(s)”. In embodiments, when an R^{L17F.2} substituent group is substituted, the R^{L17F.2} substituent group is substituted with one or more third substituent groups denoted by R^{L17F.3} as explained in the definitions section above in the description of “first substituent group(s)”. In the above embodiments, L^{17F}, R^{L17F.1}, R^{L17F.2}, and R^{L17F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{17F}, R^{L17F.1}, R^{L17F.2} and R^{L17F.3} respectively.

In embodiments, the compound contacts the Switch 2 region of human Gαs protein. In embodiments, the human Gαs protein is a human Gαs wildtype protein, a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein. In embodiments, the human Gαs protein is a human Gαs wildtype protein. In embodiments, the human Gαs protein is a human Gαs R201C protein. In embodiments, the human Gαs protein is a human Gαs R201H protein. In embodiments, the human Gαs protein is a human Gαs R201S protein. In embodiments, the human Gαs protein is a human Gαs R201L protein. In embodiments, the human Gαs protein is a human Gαs Q227R protein. In embodiments, the human Gαs protein is a human Gαs Q227H protein. In embodiments, the human Gαs protein is a human Gαs Q227K protein. In embodiments, the human Gαs protein is a human Gαs Q227E protein. In embodiments, the human Gαs protein is a human Gαs Q227L protein.

In embodiments, the compound binds a human Gαs wildtype protein more strongly than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 2-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 5-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 10-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 20-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 40-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 60-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 80-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 100-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions. In embodiments, the compound binds a human Gαs wildtype protein at least 500-fold stronger than the compound binds a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein under identical conditions.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the pharmaceutical composition includes an effective amount of a second agent, wherein the second agent is an anti-cancer agent. In embodiments, the anti-cancer agent is a MEK inhibitor (e.g., X^{L518}, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, or BAY 869766) or an EGFR inhibitor (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, or BMS—599626).

### Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### IV. Methods of Use

In an aspect is provided a method of treating a cancer in a patient in need of such treatment, the method including administering a therapeutically effective amount of a compound described herein, or a pharmaceutically acceptable salt thereof, to the patient.

In embodiments, the patient is a human. In embodiments, the cancer is selected from human cancers and carcinomas, sarcomas, adenocarcinomas, lymphomas, leukemias, and the like. In embodiments, the cancer is a solid cancer or tumor. In embodiments, the cancer is pancreatic cancer. In embodiments, the cancer is a pituitary tumor. In embodiments, the cancer is a bone tumor. In embodiments, the cancer is pancreatic cancer, pituitary cancer, or bone cancer.

In an aspect is provided a method of treating a bone condition in a patient in need of such treatment, the method including administering a therapeutically effective amount of a compound described herein, or a pharmaceutically acceptable salt thereof, to the patient.

In embodiments, the patient is a human. In embodiments, the bone condition is fibrous dysplasia. In embodiments, the fibrous dysplasia is monostotic fibrous dysplasia or polystotic fibrous dysplasia. In embodiments, the fibrous dysplasia is monostotic fibrous dysplasia. In embodiments, the fibrous dysplasia is polystotic fibrous dysplasia.

In an aspect is provided a method of treating McCune-Albright syndrome in a patient in need of such treatment, the method including administering a therapeutically effective amount of a compound described herein, or a pharmaceutically acceptable salt thereof, to the patient. In embodiments, the patient is a human.

In an aspect is provided a method of treating cholera in a patient in need of such treatment, the method including administering a therapeutically effective amount of a compound described herein, or a pharmaceutically acceptable salt thereof, to the patient. In embodiments, the patient is a human.

In an aspect is provided a method of treating a G protein-associated disease in a patient in need of such treatment, the method including administering a therapeutically effective amount of a compound described herein, or a pharmaceutically acceptable salt thereof, to the patient.

In embodiments, the patient is a human. In embodiments, the G protein-associated disease is fibrous dysplasia. In embodiments, the fibrous dysplasia is monostotic fibrous dysplasia or polystotic fibrous dysplasia. In embodiments, the fibrous dysplasia is monostotic fibrous dysplasia. In embodiments, the fibrous dysplasia is polystotic fibrous dysplasia. In embodiments, the G protein-associated disease is McCune-Albright syndrome.

In an aspect is provided a method of modulating (e.g., reducing) the activity of a human Gαs protein, the method including contacting the human Gαs protein with an effective amount of a compound described herein, or a pharmaceutically acceptable salt thereof. In embodiments, the activity of Gαs is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the activity of Gαs is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the human Gαs protein is a human Gαs wildtype protein, a human Gαs R201C protein, a human Gαs R201H protein, a human Gαs R201S protein, a human Gαs R201L protein, a human Gαs Q227R protein, a human Gαs Q227H protein, a human Gαs Q227K protein, a human Gαs Q227E protein, or a human Gαs Q227L protein. In embodiments, the human Gαs protein is a human Gαs wildtype protein. In embodiments, the human Gαs protein is a human Gαs R201C protein. In embodiments, the human Gαs protein is a human Gαs R201H protein. In embodiments, the human Gαs protein is a human Gαs Q227R protein. In embodiments, the human Gαs protein is a human Gαs Q227H protein. In embodiments, the human Gαs protein is a human Gαs Q227K protein. In embodiments, the human Gαs protein is a human Gαs Q227E protein. In embodiments, the human Gαs protein is a human Gαs Q227L protein.

In embodiments, the human Gαs protein includes an R201 mutation. In embodiments, the human Gαs protein includes a R201C mutation. In embodiments, the human Gαs protein includes a R201H mutation. In embodiments, the human Gαs protein includes a R201S mutation. In embodiments, the human Gαs protein includes a R201L mutation. In embodiments, the human Gαs protein includes a Q227 mutation. In embodiments, the human Gαs protein includes a Q227R mutation. In embodiments, the human Gαs protein includes a Q227H mutation. In embodiments, the human Gαs protein includes a Q227K mutation. In embodiments, the human Gαs protein includes a Q227E mutation. In embodiments, the human Gαs protein includes a Q227L mutation.

In embodiments, the activity of the human Gαs protein is GTPase activity or cellular signaling. In embodiments, the activity of the human Gαs protein is GTPase activity. In embodiments, the activity of the human Gαs protein is cellular signaling.

### V. Embodiments

Embodiment P1. A compound having the formula:

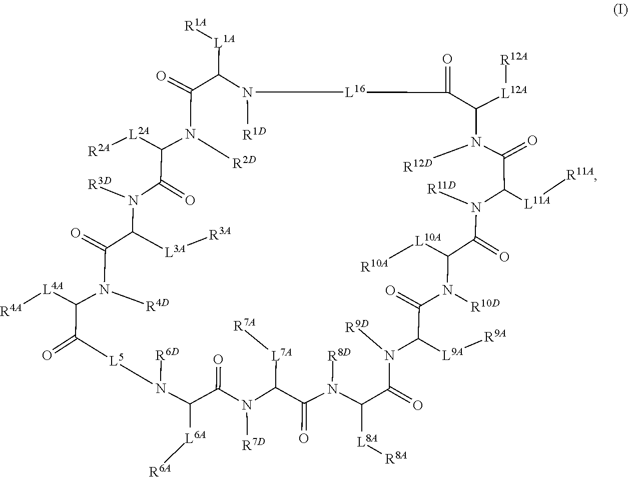

or a pharmaceutically acceptable salt thereof; wherein
L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene, or substituted or unsubstituted heteroalkylene;
L⁵ is

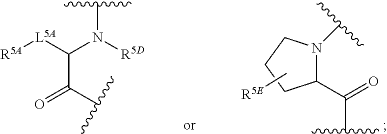

R^{1A} is substituted or unsubstituted aryl;
R^{2A} and R^{5A} are independently hydrogen, —OH, —NH₂, substituted or unsubstituted alkyl, substituted or unsubstituted cycloalkyl, substituted or unsubstituted aryl, or substituted or unsubstituted heteroaryl;
R^{3A}, R^{4A} and R^{11A} are independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R^{6A} is —NH₂, —CONH₂, substituted or unsubstituted alkyl, substituted or unsubstituted heteroalkyl, or substituted or unsubstituted aryl;
R^{7A}, R^{8A}, and R^{12A} are independently hydrogen, substituted or unsubstituted alkyl, substituted or unsubstituted cycloalkyl, substituted or unsubstituted aryl, or substituted or unsubstituted heteroaryl;
R^{9A} is substituted or unsubstituted cycloalkyl, substituted or unsubstituted heterocycloalkyl, substituted or unsubstituted aryl, or substituted or unsubstituted heteroaryl;
R^{10A} is hydrogen or substituted or unsubstituted alkyl;
R^{11D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₅ alkyl;
R^{5E} is hydrogen, —OH, —NH₂, substituted or unsubstituted alkyl, or substituted or unsubstituted heteroalkyl; and
L¹⁶ is a covalent linker.

Embodiment P2. The compound of embodiment P1, having the formula:

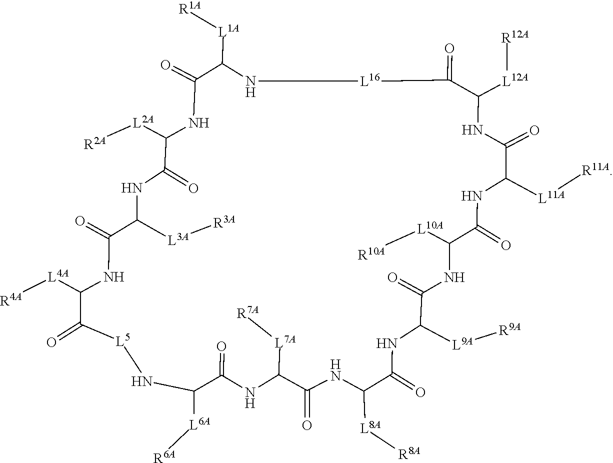

Embodiment P3. The compound of embodiment P2, having the formula:

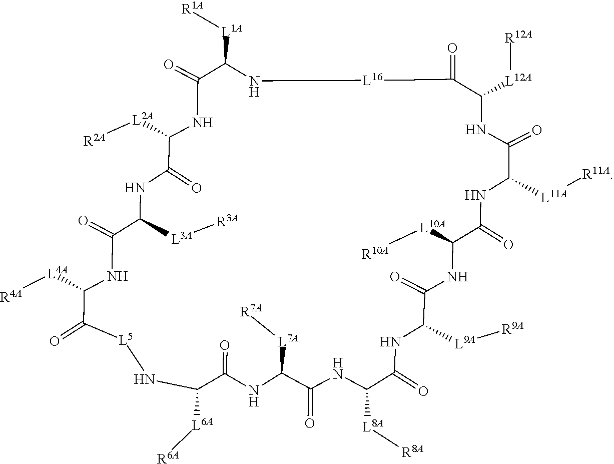

Embodiment P4. The compound of embodiment P1, having the formula:

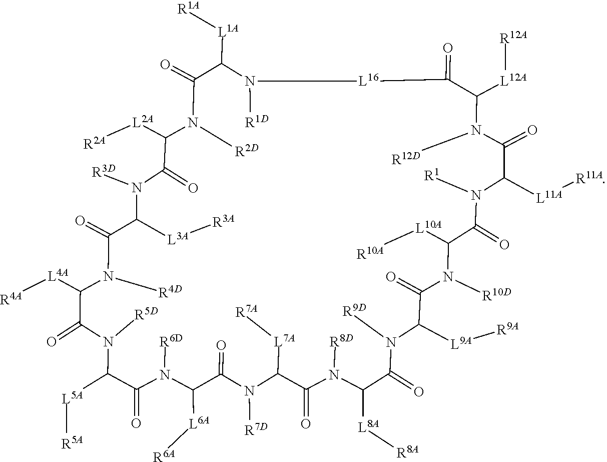

Embodiment P5. The compound of embodiment P4, having the formula:

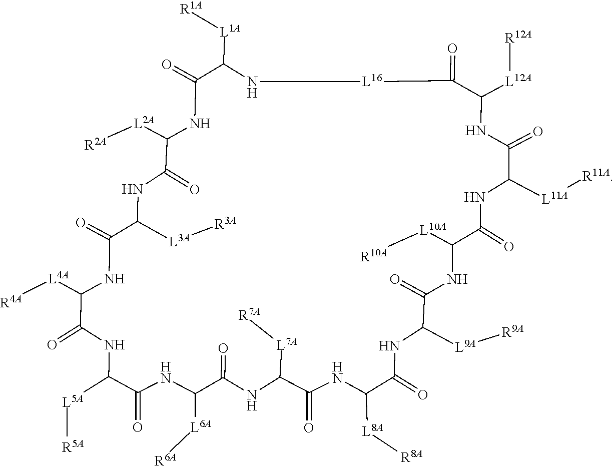

Embodiment P6. The compound of embodiment P5, having the formula:

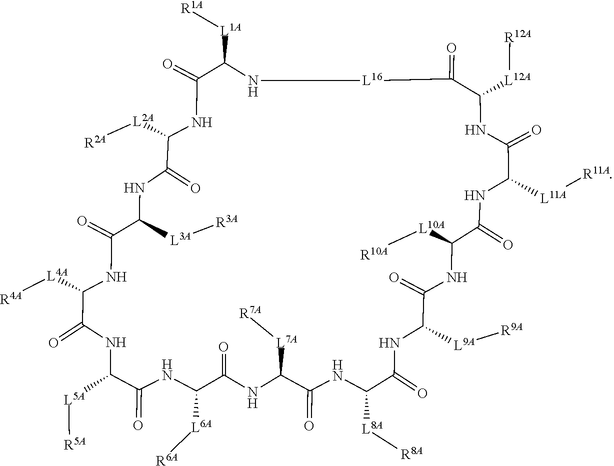

Embodiment P7. The compound of one of embodiments P1 to P6, wherein -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A},-R^{7A}-L^{8A}-R^{8A}, -L^{9A}-R^{9A}-L^{10A}-R^{10A}-L^{11A}-R^{11A}, or -L^{12A}-R^{12A} are independently a natural amino acid side chain or an unnatural amino acid side chain.

Embodiment P8. The compound of embodiment P7, wherein -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A},-R^{7A}-L^{8A}-R^{8A}-L^{9A}-R^{9A}-L^{10A}-R^{10A}-L^{11A}-R^{11A} or -L^{12A}-R^{12A} are independently a natural amino acid side chain.

Embodiment P9. The compound of embodiment P1, having the formula:

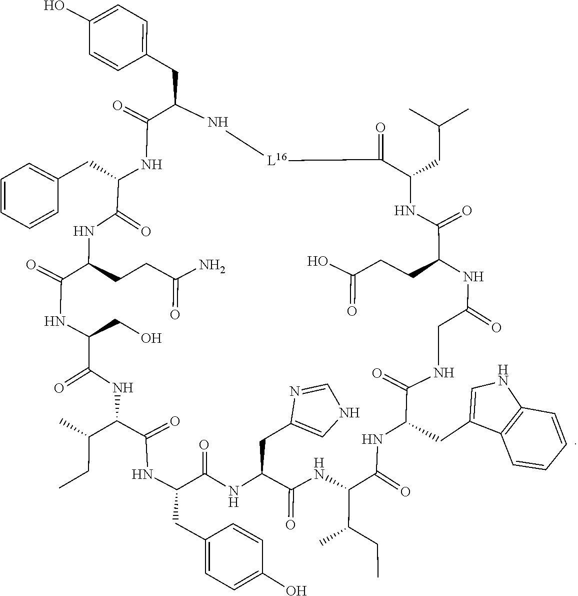

Embodiment P10. The compound of one of embodiments P1 to P9, wherein L¹⁶ is a bioconjugate linker.

Embodiment P11. The compound of one of embodiments P1 to P9, wherein L¹⁶ is a substituted or unsubstituted divalent amino acid.

Embodiment P12. The compound of one of embodiments P1 to P9, wherein L¹⁶ is -L^{16A}-L^{16B}-L^{16C}-L^{16D}-L^{16E}-L^{16F}-; and L^{16A}, L^{16B}, L^{16C}, L^{16D}, L^{16E} and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P13. The compound of embodiment P12, wherein L¹⁶ is —NH-L^{16B}-L^{16C}-L^{16D}-L^{16E}-C(O)—;
L^{16B} is

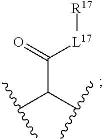

L^{16C}, L^{16D}, and L^{16E} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein a detectable moiety, or a drug moiety.

Embodiment P14. The compound of embodiment P12, wherein L¹⁶ is a bond,

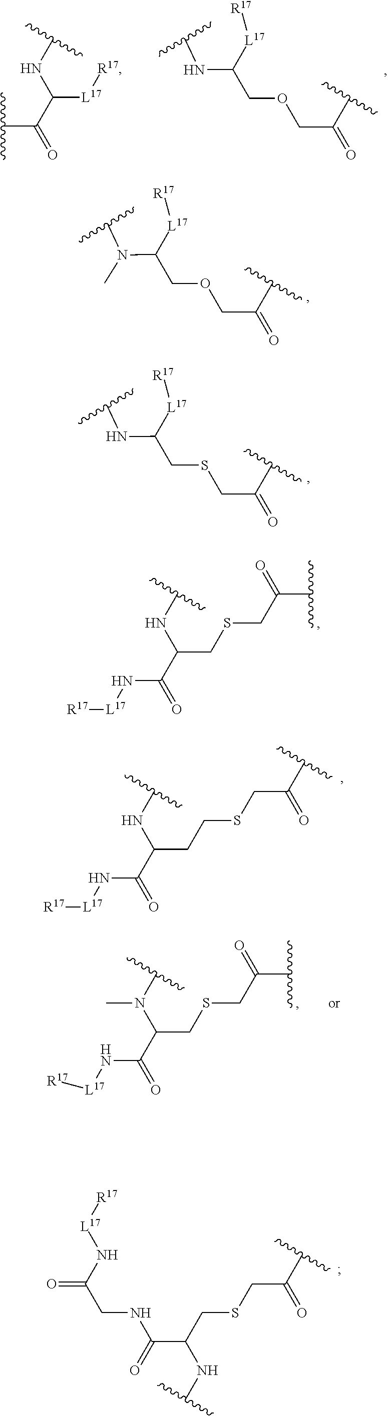

L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

Embodiment P15. The compound of embodiment P12, wherein L¹⁶ is a bond,

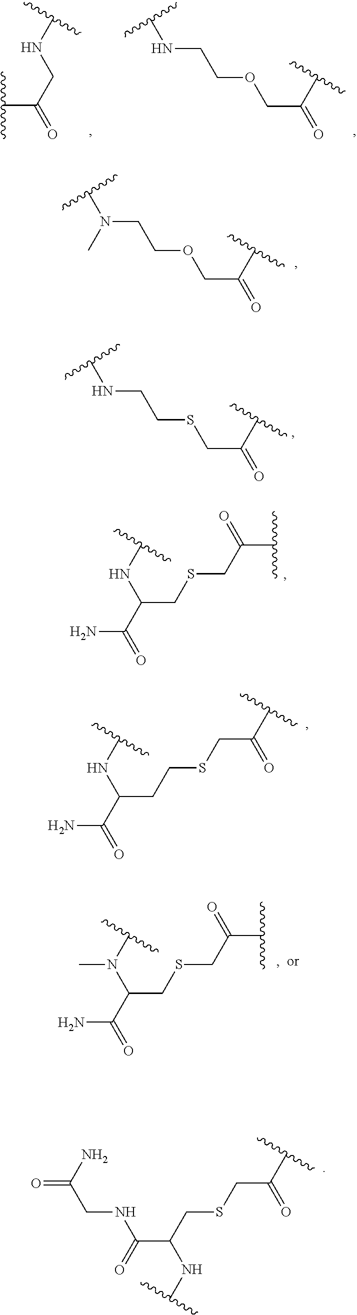

Embodiment P16. The compound of embodiment P1, having the formula:

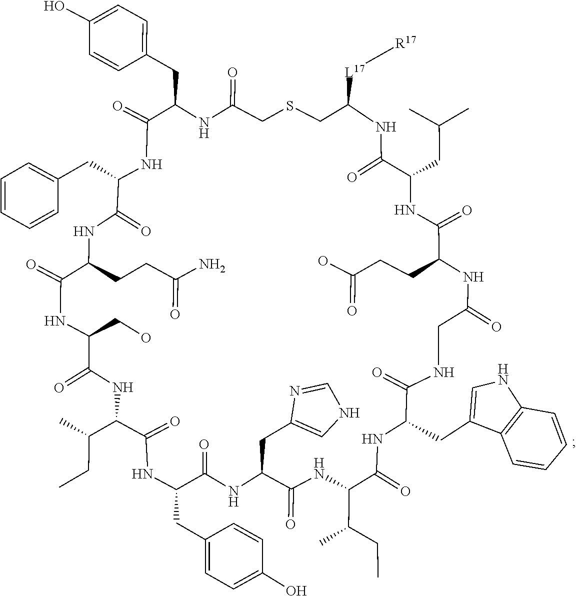

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment P17. The compound of embodiment P1, having the formula:

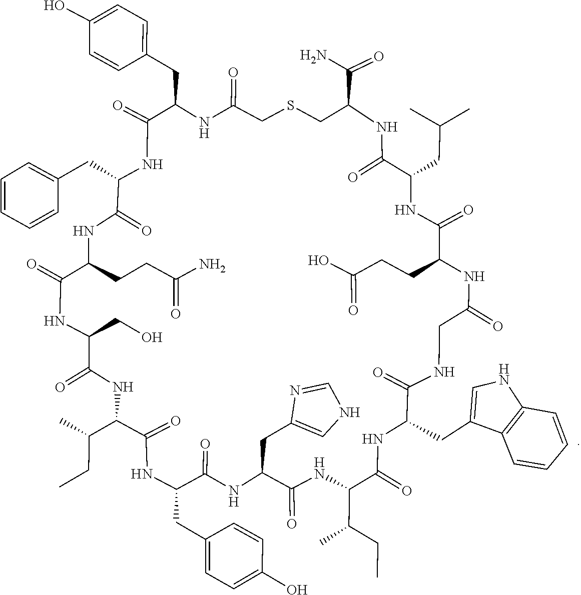 [SMILES: CC[C@H](C)[C@@H]1NC(=O)[C@H](Cc2c[nH]cn2)NC(=O)[C@H](Cc2ccc(O)cc2)NC(=O)[C@H]([C@@H](C)CC)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(N)=O)NC(=O)[C@H](CC(C)C)NC(=O)[C@H](CCC(=O)O)NC(=O)CNC(=O)[C@H](Cc2c[nH]c3ccccc23)NC1=O]

Embodiment P18. The compound of one of embodiments P1 to P17, wherein said compound binds a human Gαs protein-GTP complex more strongly than said compound binds a human Gαs protein-GDP complex under identical conditions.

Embodiment P19. The compound of embodiment P18, wherein said compound binds a human Gαs protein-GTP complex at least 2-fold stronger than said compound binds a human Gαs protein-GDP complex under identical conditions.

Embodiment P20. The compound of embodiment P18, wherein said compound binds a human Gαs protein-GTP complex at least 5-fold stronger than said compound binds a human Gαs protein-GDP complex under identical conditions.

Embodiment P21. The compound of embodiment P18, wherein said compound binds a human Gαs protein-GTP complex at least 40-fold stronger than said compound binds a human Gαs protein-GDP complex under identical conditions.

Embodiment P22. The compound of embodiment P18, wherein said compound binds a human Gαs protein-GTP complex at least 100-fold stronger than said compound binds a human Gαs protein-GDP complex under identical conditions.

Embodiment P23. A compound having the formula:

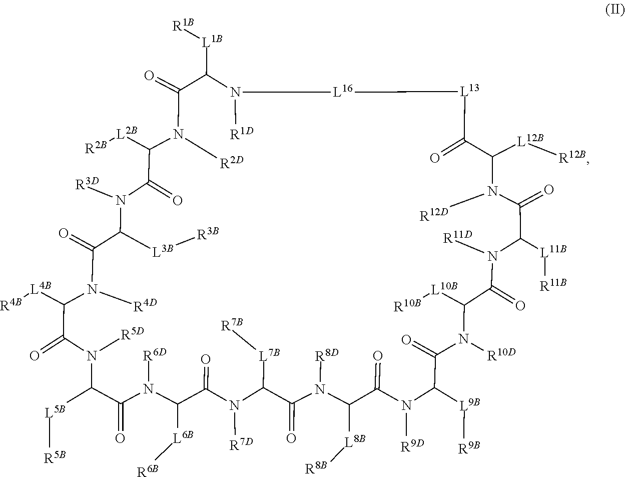

or a pharmaceutically acceptable salt thereof; wherein
L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene;
L¹³ is a bond,

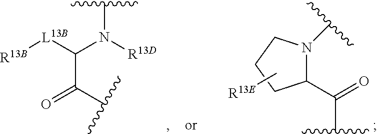

R^{1B} is substituted or unsubstituted aryl;
R^{2B}, R^{4B}, R^{5B}, R^{8B}, R^{9B}, and R^{13B} are independently hydrogen, —OH, —NH₂, —C(O)OH, —C(O)NH₂, —NO₂, —SO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3B} is hydrogen, —OH, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{6B}, R^{7B}, R^{10B}, R^{11B}, and R^{12B} are independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1D}, R^{2D}, R^{3D}, R^{4D}, R^{5D}, R^{6D}, R^{7D}, R^{8D}, R^{9D}, R^{10D}, R^{11D}, R^{12D}, and R^{13D} are independently hydrogen or unsubstituted C₁-C₈ alkyl;
R^{13E} is hydrogen, —OH, —NH₂, substituted or unsubstituted alkyl, or substituted or unsubstituted heteroalkyl; and
L¹⁶ is a covalent linker.

Embodiment P24. The compound of embodiment P23, wherein -L^{1B}-R^{1B}-L^{2B}-R^{2B}-L^{3B}-R^{3B}-L^{4B}-R^{4B}-L^{5B}-R^{5B}-L^{6B}-R^{6B}-L^{7B}-R^{7B}-L^{8B}-R^{8B}-L^{9B}-R^{9B}-L^{10B}-R^{10B}-L^{11B}-R^{11B}-L^{12B}-R^{12B} or -L^{13B}-R^{13B} are independently a natural amino acid side chain or an unnatural amino acid side chain.

Embodiment P25. The compound of embodiment P24, wherein -L^{1B}-R^{1B}-L^{2B}-R^{2B}-L^{3B}-R^{3B}-L^{4B}-R^{4B}-L^{5B}-R^{5B}-L^{6B}-R^{6B}-L^{7B}-R^{7B}, -L^{8B}-R^{8B}-L^{9B}-R^{9B}-L^{10B}-R^{10B}-L^{11B}-R^{11B}-L^{12B}-R^{12B} or -L^{13B}-R^{13B} are independently a natural amino acid side chain.

Embodiment P26. The compound of embodiment P23, having the formula:

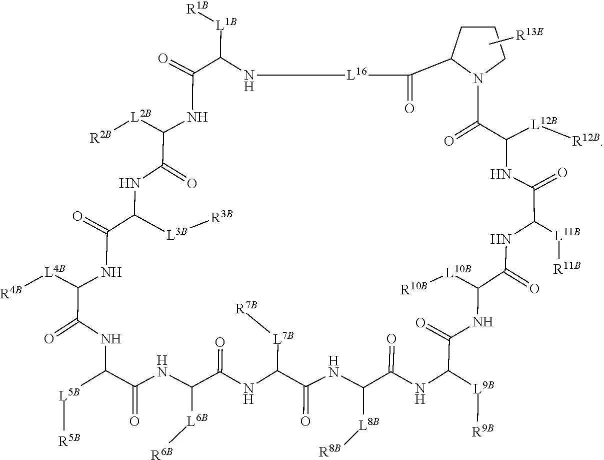

Embodiment P27. The compound of embodiment P26, having the formula.

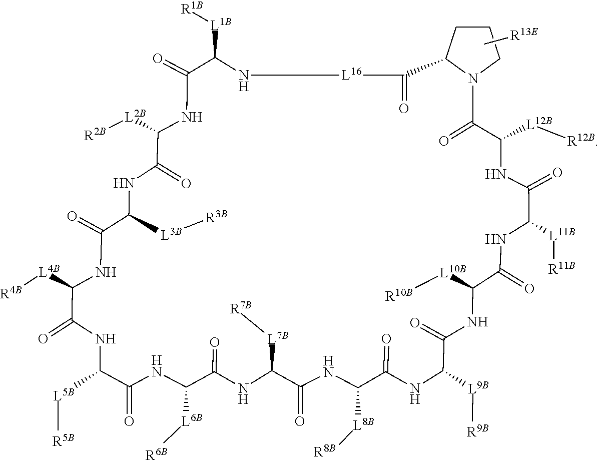

Embodiment P28. The compound of embodiment P27, having the formula:

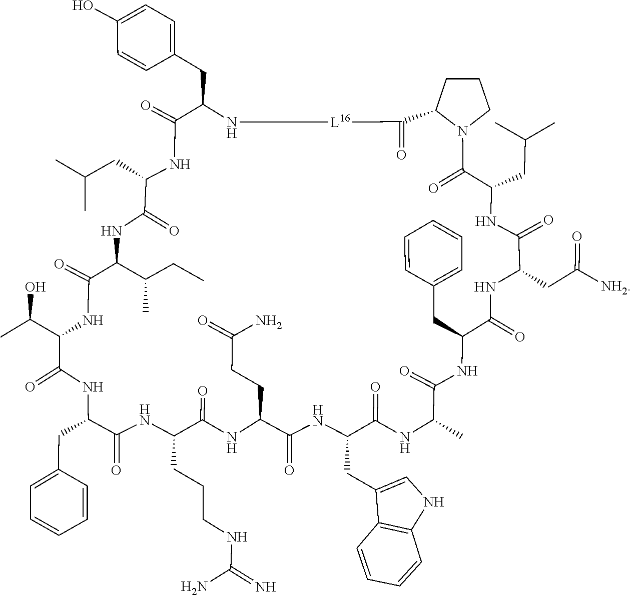

Embodiment P29. The compound of one of embodiments P23 to P28, wherein L¹⁶ is a bioconjugate linker.

Embodiment P30. The compound of one of embodiments P23 to P28, wherein L¹⁶ is a substituted or unsubstituted divalent amino acid.

Embodiment P31. The compound of one of embodiments P23 to P28, wherein L¹⁶ is -L^{6A}-L^{16B}-L^{16C}-L^{16D}-L^{16E}-L^{16F}-; and L^{16A}, L^{16B}, L^{16C}, L^{16D}, L^{16E} and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P32. The compound of embodiment P31, wherein
L¹⁶ is —NH-L^{16B}-L^{16C}-L^{16D}-L^{16E}-C(O)—;
L^{16B} is

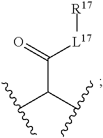

L^{16C}, L^{16D}, and L^{16E} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein a detectable moiety, or a drug moiety.

Embodiment P33. The compound of embodiment P31, wherein L¹⁶ is a bond,

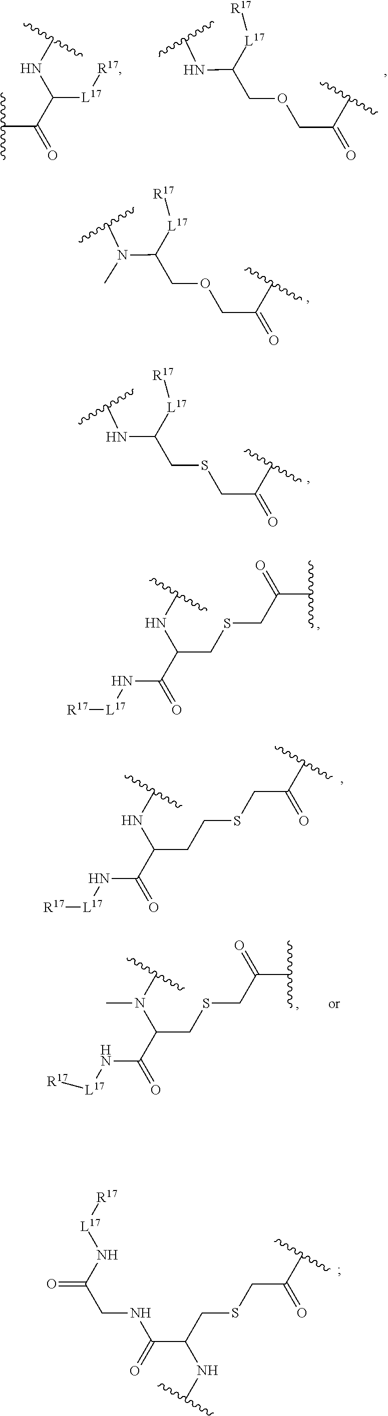

L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

Embodiment P34. The compound of embodiment P31, wherein L¹⁶ is a bond,

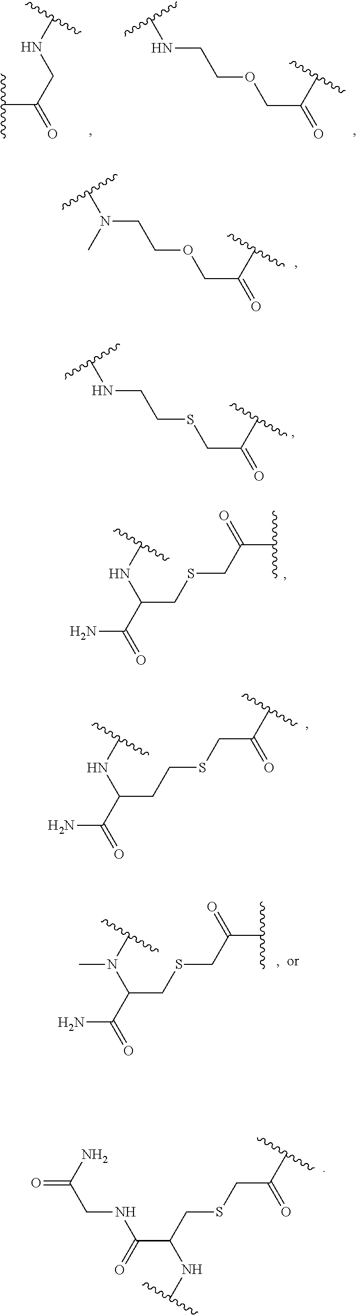

Embodiment P35. The compound of embodiment P23, having the formula:

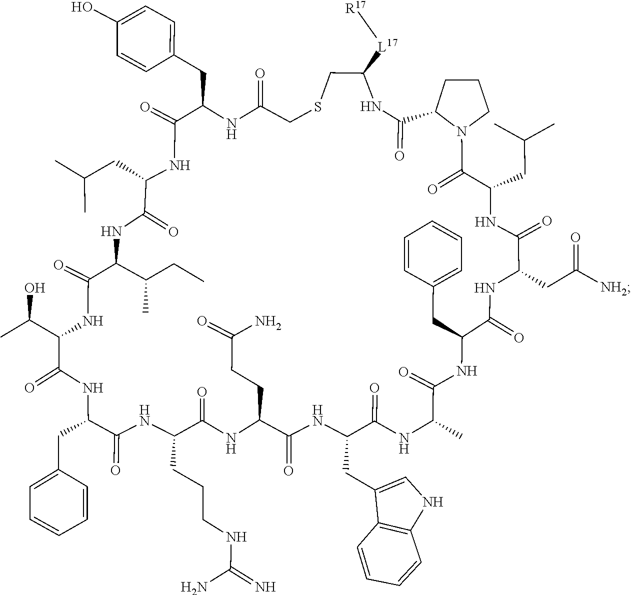

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment P36. The compound of embodiment P23, having the formula:

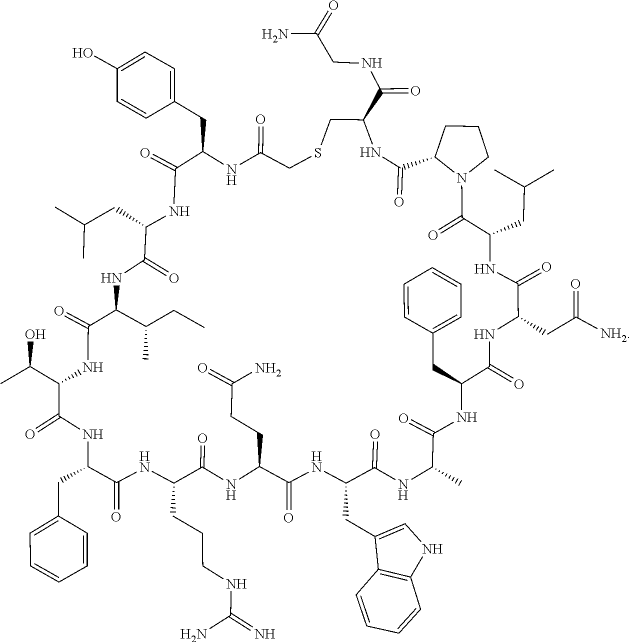 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](Cc2ccccc2)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

Embodiment P37. The compound of one of embodiments P23 to P36, wherein said compound binds a human Gαs protein-GDP complex more strongly than said compound binds a human Gαs protein-GTP complex under identical conditions.

Embodiment P38. The compound of embodiment P37, wherein said compound binds a human Gαs protein-GDP complex at least 2-fold stronger than said compound binds a human Gαs protein-GTP complex under identical conditions.

Embodiment P39. The compound of embodiment P37, wherein said compound binds a human Gαs protein-GDP complex at least 5-fold stronger than said compound binds a human Gαs protein-GTP complex under identical conditions.

Embodiment P40. The compound of embodiment P37, wherein said compound binds a human Gαs protein-GDP complex at least 40-fold stronger than said compound binds a human Gαs protein-GTP complex under identical conditions.

Embodiment P41. The compound of embodiment P37, wherein said compound binds a human Gαs protein-GDP complex at least 100-fold stronger than said compound binds a human Gαs protein-GTP complex under identical conditions.

Embodiment P42. The compound of one of embodiments P1 to P41, wherein said compound contacts the Switch 2 region of human Gαs protein.

Embodiment P43. The compound of embodiment P42, wherein the human Gαs protein is a human Gαs wildtype protein, a human Gαs R201C protein, a human Gαs R201H protein, a human Gαs Q227R protein, a human Gαs Q227H protein, a human Gαs Q227K protein, a human Gαs Q227E protein, or a human Gαs Q227L protein.

Embodiment P44. A pharmaceutical composition comprising the compound of one of embodiments P1 to P43, or a pharmaceutically acceptable salt thereof, and a pharmaceutically acceptable excipient.

Embodiment P45. A method of treating a cancer in a patient in need of such treatment, said method comprising administering a therapeutically effective amount of a compound of one of embodiments P1 to P43 to said patient.

Embodiment P46. The method of embodiment P45, wherein the cancer is pancreatic cancer, pituitary cancer, or bone cancer.

Embodiment P47. A method of treating a bone condition in a patient in need of such treatment, said method comprising administering a therapeutically effective amount of a compound of one of embodiments P1 to P43 to said patient.

Embodiment P48. The method of embodiment P47, wherein the bone condition is fibrous dysplasia.

Embodiment P49. The method of embodiment P48, wherein the fibrous dysplasia is monostotic fibrous dysplasia or polystotic fibrous dysplasia.

Embodiment P50. A method of treating McCune-Albright syndrome in a patient in need of such treatment, said method comprising administering a therapeutically effective amount of a compound of one of embodiments P1 to P43 to said patient.

Embodiment P51. A method of treating cholera in a patient in need of such treatment, said method comprising administering a therapeutically effective amount of a compound of one of embodiments P1 to P43 to said patient.

Embodiment P52. A method of modulating the activity of a human Gαs protein, said method comprising contacting said human Gαs protein with an effective amount of a compound of one of embodiments P1 to P43.

Embodiment P53. The method of embodiment P52, wherein said human Gαs protein is a human Gαs wildtype protein, a human Gαs R201C protein, a human Gαs R201H protein, a human Gαs Q227R protein, a human Gαs Q227H protein, a human Gαs Q227K protein, a human Gαs Q227E protein, or a human Gαs Q227L protein.

### VI. Additional Embodiments

Embodiment 1. A compound having the formula:

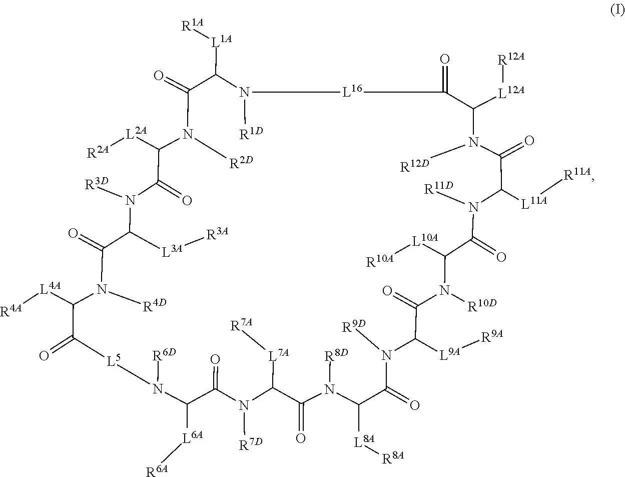

or a pharmaceutically acceptable salt thereof;
wherein
L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene, or substituted or unsubstituted heteroalkylene;
L⁵ is

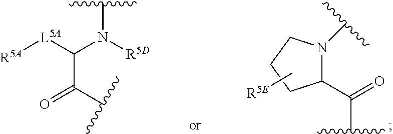

R^{1A} is substituted or unsubstituted aryl;
R^{2A} and R^{5A} are independently hydrogen, —OH, —NH₂, substituted or unsubstituted alkyl, substituted or unsubstituted cycloalkyl, substituted or unsubstituted aryl, or substituted or unsubstituted heteroaryl;
R^{3A}, R^{4A}, and R^{11A} are independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R^{6A} is —NH₂, —CONH₂, substituted or unsubstituted alkyl, substituted or unsubstituted heteroalkyl, or substituted or unsubstituted aryl;
R^{7A}, R^{8A}, and R^{12A} are independently hydrogen, substituted or unsubstituted alkyl, substituted or unsubstituted cycloalkyl, substituted or unsubstituted aryl, or substituted or unsubstituted heteroaryl;
R^{9A} is substituted or unsubstituted cycloalkyl, substituted or unsubstituted heterocycloalkyl, substituted or unsubstituted aryl, or substituted or unsubstituted heteroaryl;
R^{10A} is hydrogen or substituted or unsubstituted alkyl;
R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₈ alkyl;
R^{5E} is hydrogen, —OH, —NH₂, substituted or unsubstituted alkyl, or substituted or unsubstituted heteroalkyl; and
L¹⁶ is a covalent linker.

Embodiment 2. The compound of embodiment 1, having the formula:

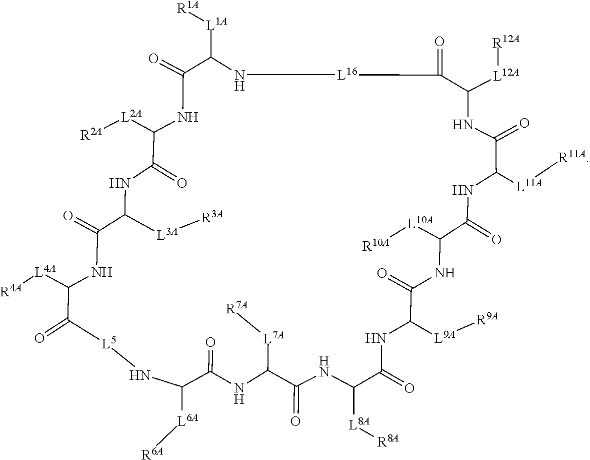

Embodiment 3. The compound of one of embodiments 1 to 2, having the formula:

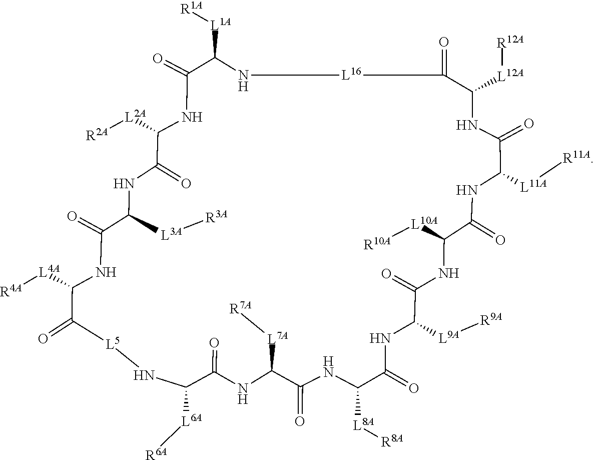

Embodiment 4. The compound of one of embodiments 1 to 3, having the formula:

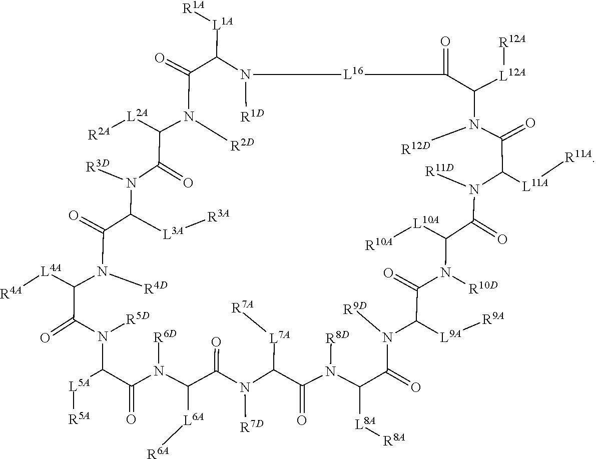

Embodiment 5. The compound of one of embodiments 1 to 4, having the formula:

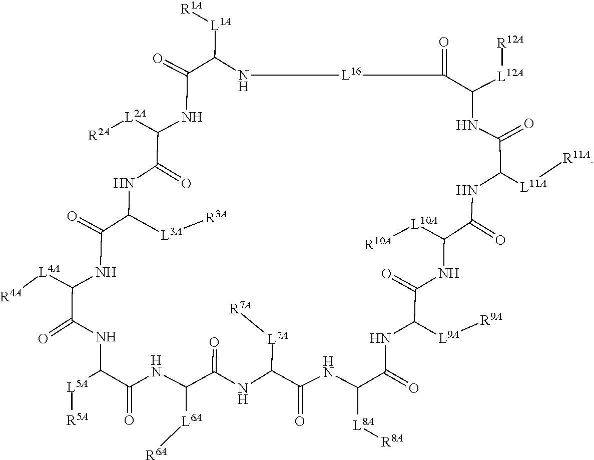

Embodiment 6. The compound of one of embodiments 1 to 5, having the formula:

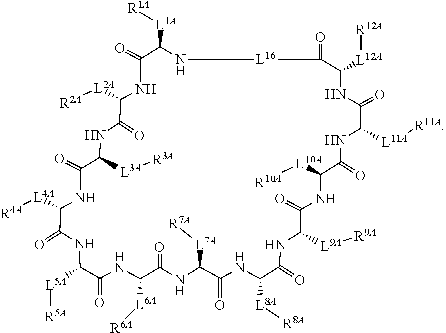

Embodiment 7. The compound of one of embodiments 1 to 6, wherein -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A},-R^{7A}-L^{8A}-R^{8A}, -L^{9A}-R^{9A}-L^{10A}-R^{10A}-L^{11A}-R^{11A}, or -L^{12A}-R^{12A} are independently a natural amino acid side chain or an unnatural amino acid side chain.

Embodiment 8. The compound of one of embodiments 1 to 7, wherein -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A},-R^{7A}-L^{8A}-R^{8A}, -L^{9A}-R^{9A}-L^{10A}-R^{10A}-L^{11A}-R^{11A}, or -L^{12A}-R^{12A} are independently a natural amino acid side chain.

Embodiment 9. The compound of one of embodiments 1 to 8, having the formula:

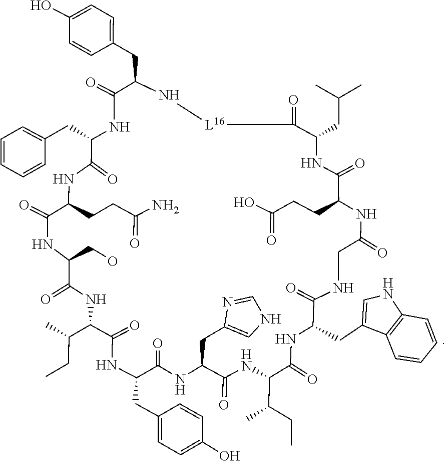

Embodiment 10. The compound of one of embodiments 1 to 9, wherein L¹⁶ is a bioconjugate linker.

Embodiment 11. The compound of one of embodiments 1 to 9, wherein L¹⁶ is a substituted or unsubstituted divalent amino acid.

Embodiment 12. The compound of one of embodiments 1 to 9, wherein
L¹⁶ is -L^{6A}-L^{16B}-L^{16C}-L^{16D}-L^{16E}-L^{16F}-; and
L^{16A}, L^{16B}, L^{16C}, L^{16D}, L^{16E} and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 13. The compound of embodiment 12, wherein
L¹⁶ is —NH-L^{16B}-L^{16C}-L^{16D}-L^{16E}-C(O)—;
L^{16B} is

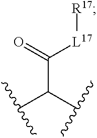

L^{16C}, L^{16D}, and L^{16E} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 14. The compound of one of embodiments 1 to 9, wherein L¹⁶ is a bond

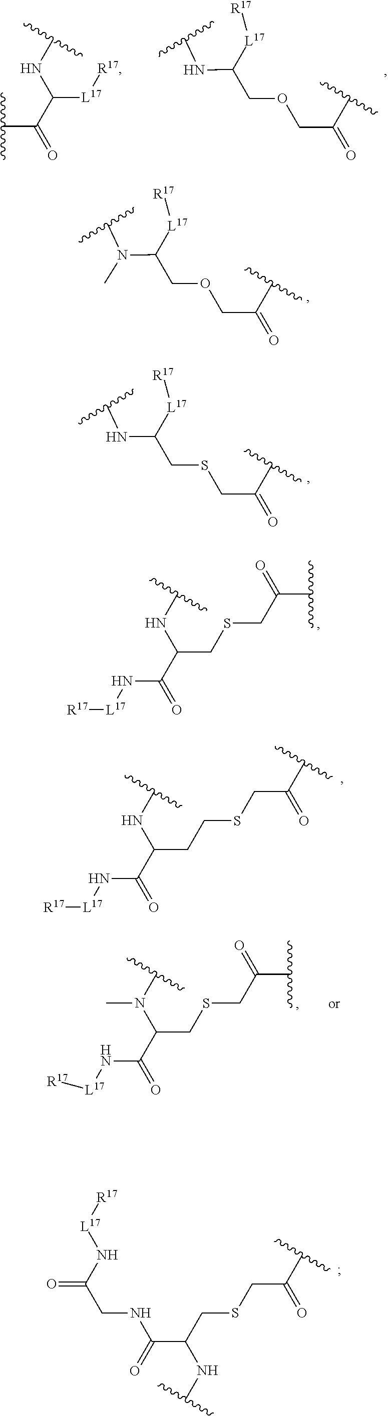

L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

Embodiment 15. The compound of one of embodiments 1 to 9, wherein L¹⁶ is a bond,

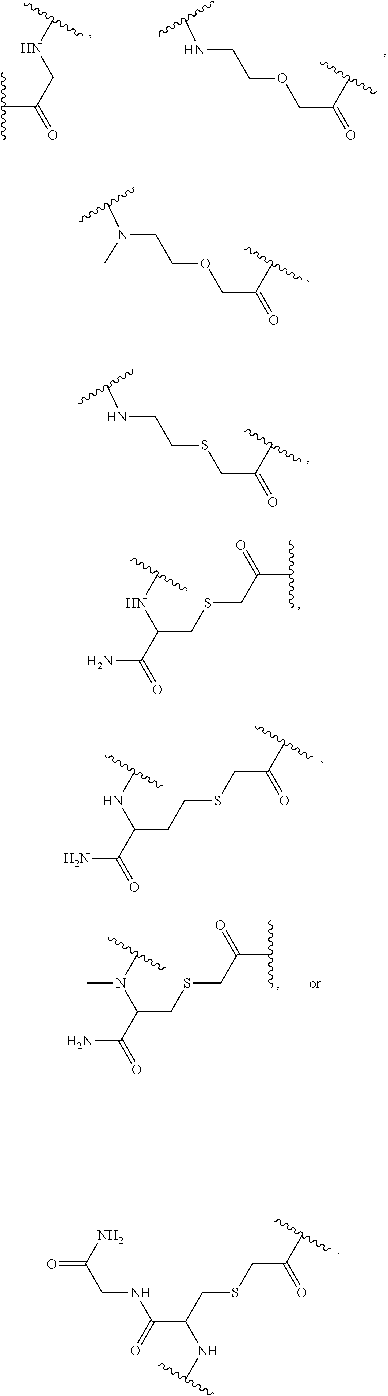

Embodiment 16. The compound of one of embodiments 1 to 9, having the formula:

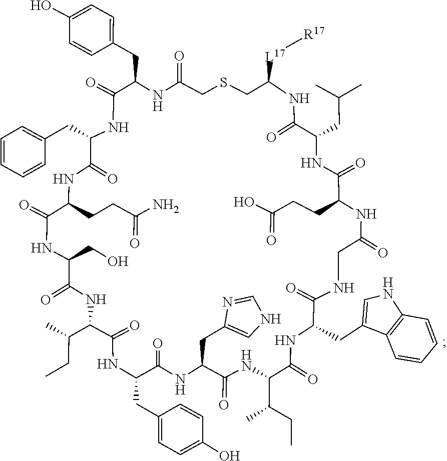

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 17. The compound of one of embodiments 1 to 9, having the formula:

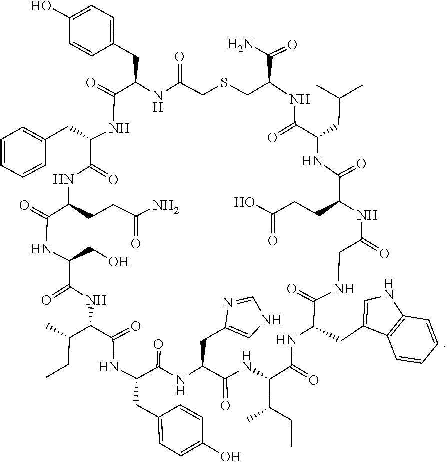 [SMILES: CC[C@H](C)[C@@H]1NC(=O)[C@H](Cc2c[nH]cn2)NC(=O)[C@H](Cc2ccc(O)cc2)NC(=O)[C@H]([C@@H](C)CC)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(N)=O)NC(=O)[C@H](CC(C)C)NC(=O)[C@H](CCC(=O)O)NC(=O)CNC(=O)[C@H](Cc2c[nH]c3ccccc23)NC1=O]

Embodiment 18. The compound of one of embodiments 1 to 17, wherein said compound binds a human Gαs protein-GTP complex more strongly than said compound binds a human Gαs protein-GDP complex under identical conditions.

Embodiment 19. The compound of one of embodiments 1 to 18, wherein said compound binds a human Gαs protein-GTP complex at least 2-fold stronger than said compound binds a human Gαs protein-GDP complex under identical conditions.

Embodiment 20. The compound of one of embodiments 1 to 19, wherein said compound binds a human Gαs protein-GTP complex at least 5-fold stronger than said compound binds a human Gαs protein-GDP complex under identical conditions.

Embodiment 21. The compound of one of embodiments 1 to 20, wherein said compound binds a human Gαs protein-GTP complex at least 40-fold stronger than said compound binds a human Gαs protein-GDP complex under identical conditions.

Embodiment 22. The compound of one of embodiments 1 to 21, wherein said compound binds a human Gαs protein-GTP complex at least 100-fold stronger than said compound binds a human Gαs protein-GDP complex under identical conditions.

Embodiment 23. A compound having the formula:

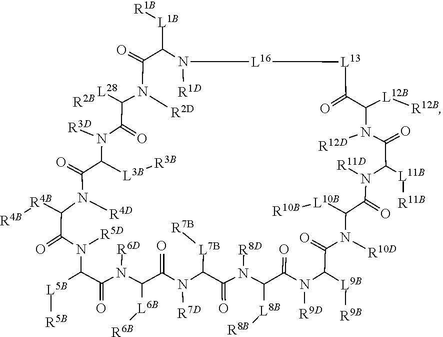

or a pharmaceutically acceptable salt thereof;
wherein
L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene;
L¹³ is a bond,

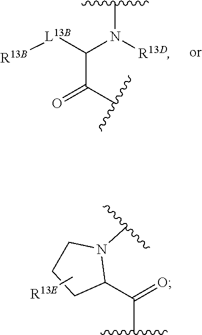

R^{1B} is substituted or unsubstituted aryl;
R^{2B}, R^{4B}, R^{5B}, R^{8B}, R^{9B}, and R^{13B} are independently hydrogen, —OH, —NH₂, —C(O)OH, —C(O)NH₂, —NO₂, —SO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3B} is hydrogen, —OH, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{6B}, R^{7B}, R^{10B}, R^{11B}, and R^{12B} are independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1D}, R^{2D}, R^{3D}, R^{4D}, R^{5D}, R^{6D}, R^{7D}, R^{8D}, R^{9D}, R^{10D}, R^{11D}, R^{12D}, and R^{13D} are independently hydrogen or unsubstituted C₁-C₈ alkyl;
R^{13E} is hydrogen, —OH, —NH₂, substituted or unsubstituted alkyl, or substituted or unsubstituted heteroalkyl; and
L¹⁶ is a covalent linker.

Embodiment 24. The compound of embodiment 23, wherein -L^{1B}-R^{1B}-L^{2B}-R^{2B}-L^{3B}-R^{3B}-L^{4B}-R^{4B}-L^{5B}-R^{5B}-L^{6B}-R^{6B}-L^{7B}-R^{7B}-L^{8B}-R^{8B}-L^{9B}-R^{9B}-L^{10B}-R^{10B}-L^{11B}-R^{11B}-L^{12B}-R^{12B}, or -L^{13B}-R^{13B} are independently a natural amino acid side chain or an unnatural amino acid side chain.

Embodiment 25. The compound of one of embodiments 23 to 24, wherein -L^{1B}-R^{1B}-L^{2B}-R^{2B}-L^{3B}-R^{3B}-L^{4B}-R^{4B}-L^{5B}-R^{5B}-L^{6B}-R^{6B}-L^{7B}-R^{7B}-L^{8B}-R^{8B}-L^{9B}-R^{9B}-L^{10B}-R^{10B}-L^{11B}-R^{11B}-L^{12B}-R^{12B}, or -L^{13B}-R^{13B} are independently a natural amino acid side chain.

Embodiment 26. The compound of one of embodiments 23 to 25, having the formula:

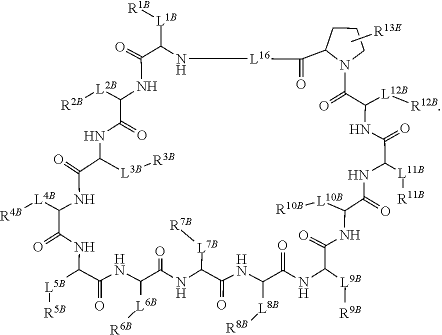

Embodiment 27. The compound of one of embodiments 23 to 26, having the formula:

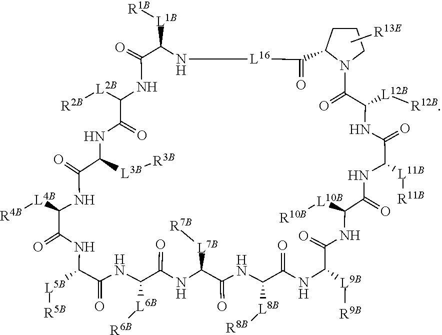

Embodiment 28. The compound of one of embodiments 23 to 27, having the formula:

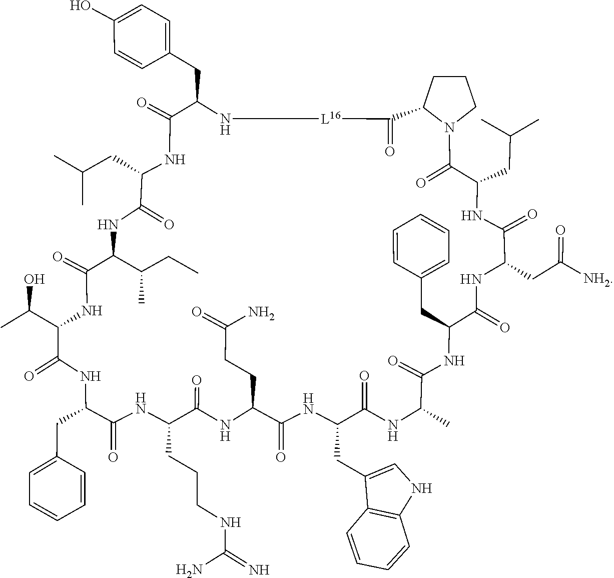

Embodiment 29. The compound of one of embodiments 23 to 27, having the formula:

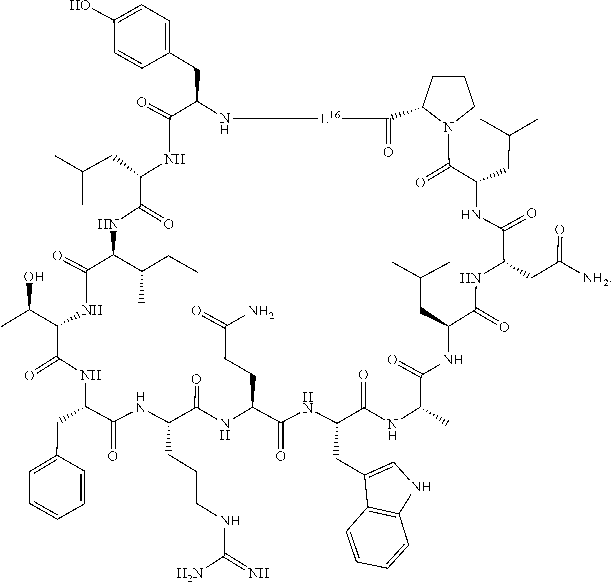

Embodiment 30. The compound of one of embodiments 23 to 29, wherein L¹⁶ is a bioconjugate linker.

Embodiment 31. The compound of one of embodiments 23 to 29, wherein L¹⁶ is a substituted or unsubstituted divalent amino acid.

Embodiment 32. The compound of one of embodiments 23 to 29, wherein
L¹⁶ is -L^{6A}-L^{16B}-L^{16C}-L^{16D}-L^{16E}-L^{16F}-; and
L^{16A}, L^{16B}, L^{16C}, L^{16D}, L^{16E}, and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 33. The compound of embodiment 32, wherein
L¹⁶ is —NH-L^{16B}-L^{16C}-L^{16D}-L^{16E}-C(O)—;
L^{16B} is

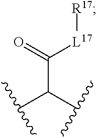

L^{16C}, L^{16D}, and L^{16E} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein a detectable moiety, or a drug moiety.

Embodiment 34. The compound of one of embodiments 23 to 29, wherein L¹⁶ is a bond,

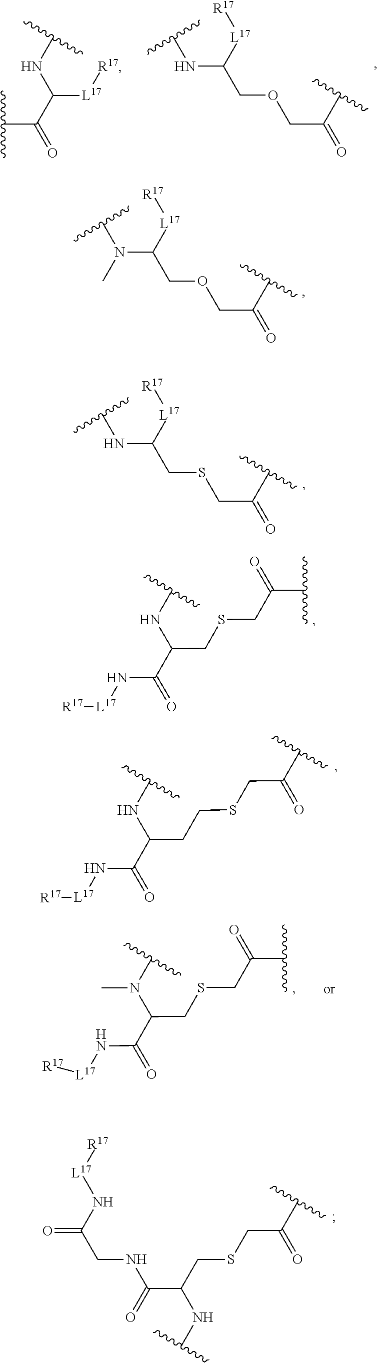

L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

Embodiment 35. The compound of one of embodiments 23 to 29, wherein L¹⁶ is a bond

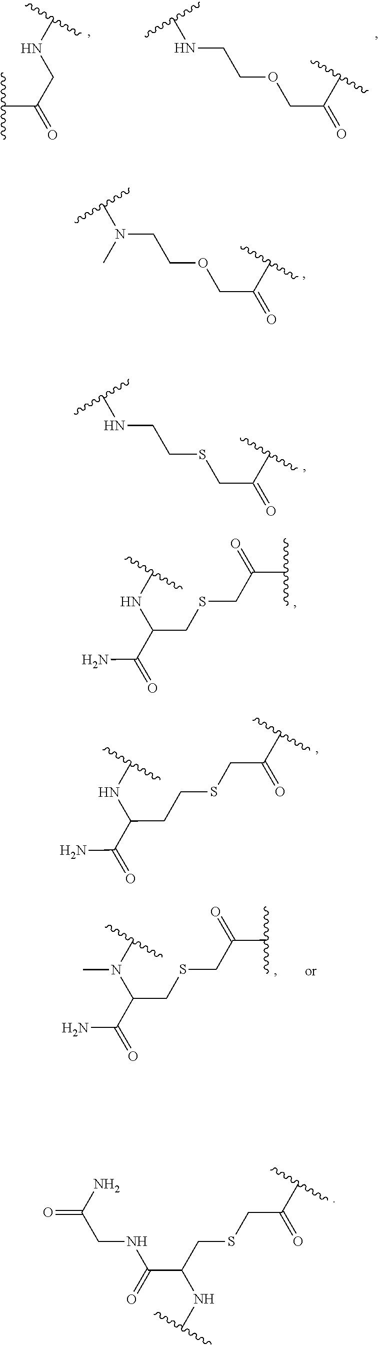

Embodiment 36. The compound of one of embodiments 23 to 27, having the formula:

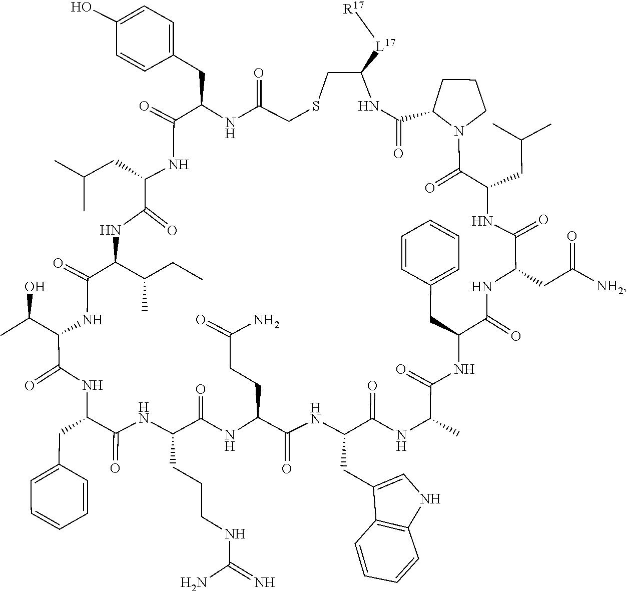

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 37. The compound of one of embodiments 23 to 27, having the formula:

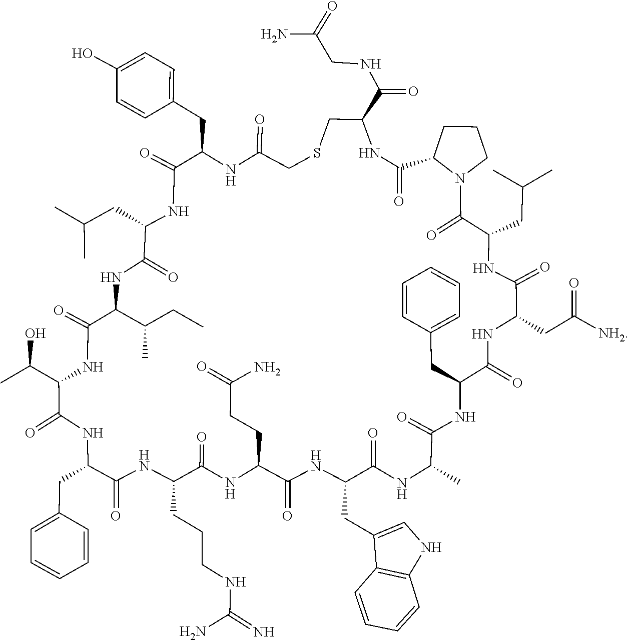 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](Cc2ccccc2)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

Embodiment 38. The compound of one of embodiments 23 to 27, having the formula:

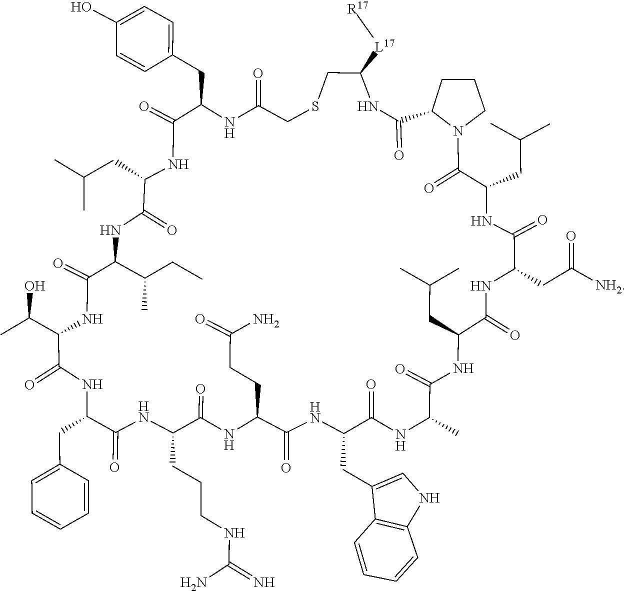

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E} and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 39. The compound of one of embodiments 23 to 27, having the formula:

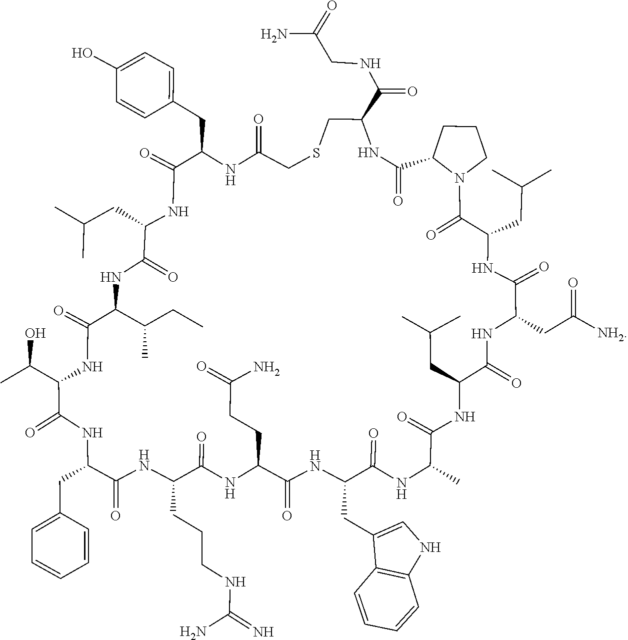 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](CC(C)C)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

Embodiment 40. The compound of one of embodiments 23 to 39, wherein said compound binds a human Gαs protein-GDP complex more strongly than said compound binds a human Gαs protein-GTP complex under identical conditions.

Embodiment 41. The compound of one of embodiments 23 to 40, wherein said compound binds a human Gαs protein-GDP complex at least 2-fold stronger than said compound binds a human Gαs protein-GTP complex under identical conditions.

Embodiment 42. The compound of one of embodiments 23 to 41, wherein said compound binds a human Gαs protein-GDP complex at least 5-fold stronger than said compound binds a human Gαs protein-GTP complex under identical conditions.

Embodiment 43. The compound of one of embodiments 23 to 42, wherein said compound binds a human Gαs protein-GDP complex at least 40-fold stronger than said compound binds a human Gαs protein-GTP complex under identical conditions.

Embodiment 44. The compound of one of embodiments 23 to 43, wherein said compound binds a human Gαs protein-GDP complex at least 100-fold stronger than said compound binds a human Gαs protein-GTP complex under identical conditions.

Embodiment 45. The compound of one of embodiments 1 to 44, wherein said compound contacts the Switch 2 region of human Gαs protein.

Embodiment 46. The compound of embodiment 45, wherein the human Gαs protein is a human Gαs wildtype protein, a human Gαs R201C protein, a human Gαs R201H protein, a human Gαs Q227R protein, a human Gαs Q227H protein, a human Gαs Q227K protein, a human Gαs Q227E protein, or a human Gαs Q227L protein.

Embodiment 47. A pharmaceutical composition comprising the compound of one of embodiments 1 to 46, or a pharmaceutically acceptable salt thereof, and a pharmaceutically acceptable excipient.

Embodiment 48. A method of treating a cancer in a patient in need of such treatment, said method comprising administering a therapeutically effective amount of a compound of one of embodiments 1 to 46 to said patient.

Embodiment 49. The method of embodiment 48, wherein the cancer is pancreatic cancer, pituitary cancer, or bone cancer.

Embodiment 50. A method of treating a bone condition in a patient in need of such treatment, said method comprising administering a therapeutically effective amount of a compound of one of embodiments 1 to 46 to said patient.

Embodiment 51. The method of embodiment 50, wherein the bone condition is fibrous dysplasia.

Embodiment 52. The method of embodiment 51, wherein the fibrous dysplasia is monostotic fibrous dysplasia or polystotic fibrous dysplasia.

Embodiment 53. A method of treating McCune-Albright syndrome in a patient in need of such treatment, said method comprising administering a therapeutically effective amount of a compound of one of embodiments 1 to 46 to said patient.

Embodiment 54. A method of treating cholera in a patient in need of such treatment, said method comprising administering a therapeutically effective amount of a compound of one of embodiments 1 to 46 to said patient.

Embodiment 55. A method of modulating the activity of a human Gαs protein, said method comprising contacting said human Gαs protein with an effective amount of a compound of one of embodiments 1 to 46.

Embodiment 56. The method of embodiment 55, wherein said human Gαs protein is a human Gαs wildtype protein, a human Gαs R201C protein, a human Gαs R201H protein, a human Gαs Q227R protein, a human Gαs Q227H protein, a human Gαs Q227K protein, a human Gαs Q227E protein, or a human Gαs Q227L protein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: State-Selective Cyclic Peptide Ligands of Gαs

The GNAS gene encodes the Gαs stimulatory subunit of heterotrimeric G proteins, which mediate G-protein-coupled receptor (GPCR) signaling, a central mechanism by which cells sense and respond to extracellular stimuli. Multiple human cancer types exhibit recurrent gain-of-function mutations in the pathway, most frequently targeting GNAS. The most lethal tumor type where GNAS is frequently mutated is the intraductal papillary mucinous neoplasm (IPMN), a precursor of invasive pancreatic cancer. We have developed, inter alia, state-selective Gαs binding molecules which block adenylyl cyclase (AC) activation. Using the Random non-standard Peptide Integrated Discovery (RaPID) platform, we have selected active state and inactive state preferring cyclic peptides against Gαs. We have solved high resolution X-ray co-crystal structures of our function blocking cyclic peptides which explains their nucleotide state specificity and inhibitory activity.

### Example 2: Peptide Selection

### RaPID Selection

Selections were performed with thioether-macrocyclic peptide library against GppNHp-bound Gαs using the GDP-bound Gαs as the negative selection. Thioether-macrocyclic peptide libraries were constructed with N-chloroacetyl-D-tyrosine (ClAc^{D}Tyr) as an initiator by using the flexible in vitro translation (FIT) system (32). The mRNA libraries, ClAc^{D}Tyr-tRNA^{fMet}_{CAU} were prepared as reported (I2). The mRNA library corresponding for the thioether-macrocyclic peptide library was designed to have an AUG initiator codon to incorporate N-chloroacetyl-D-tyrosine (ClAc^{D}Tyr), followed by 8-12 NNK random codons (N=G, C, A or U; K=G or U) to code random proteinogenic amino acids, and then a fixed downstream UGC codon to assign Cys. After in vitro translation, a thioether bond formed spontaneously between the N-terminal ClAc group of the initiator ^{D}Tyr residue and the sulfhydryl group of a downstream Cys residue.

In the first round of selection, the initial cyclic peptide library was formed by adding puromycin ligated mRNA library (225 pmol) to a 150 μL scale flexible in vitro translation system, in the presence of 30 μM of ClAc^{D}Tyr-tRNA^{fMet}_{CAU}. The translation was performed 37° C. for 30 min, followed by an extra incubation at 25° C. for 12 min. After an addition of 15 μL of 200 mM EDTA (pH 8.0) solution, the reaction solution was incubated at 37° C. for 30 min to facilitate cyclization. Then the library was reversed transcribed by M-MLV reverse transcriptase (Promega, Cat #3683) at 42° C. for 1 hour and subject to pre-washed Sephadex G-25 (GE Healthcare, Cat #17003201) columns to remove salts. The desalted solution of peptide-mRNA/cDNA was applied to GppNHp-bound Gαs-immobilized Dynabeads M280 streptavidin magnetic beads (Thermo Fisher Scientific, Cat #11206D) and rotated at 4° C. for 1 hour in selection buffer (25 mM HEPES pH 7.5, 150 mM NaCl, 1 mM MgCl₂ and 0.05% Tween 20) containing 0.5 mM GppNHp (Sigma, Cat #G0635) and 0.1% acetylated BSA (Nacalai Tesque, Cat #01278-44). Bead amounts were chosen that the final concentration of GppNHp-bound Gαs was 200 nM. This process is referred to as positive selection. The selected peptide-mRNA/cDNAs were isolated from the beads by incubating in 1×PCR reaction buffer heated at 95° C. for 5 min. The amount of eluted cDNAs was measured by quantitative PCR (Roche LightCycler 96). The remaining cDNAs were amplified by PCR, purified and transcribed into mRNAs as a library for the next round of selection.

In the subsequent rounds of selection, ligated mRNA from previous round (7.5 pmol) was added to a 5 μL scale reprogrammed in vitro translation system. This was incubated at 37° C. for 30 min and at 25° C. for 12 min. Then 1 μL of 100 mM EDTA (pH 8.0) was added and incubated at 37° C. for 30 min. After reverse transcription and subject to pre-washed Sephadex G-25 columns to remove salts, negative selection was performed by adding the desalted solution of peptide-mRNA/cDNA to GDP-bound Gαs-immobilized Dynabeads M280 streptavidin magnetic beads and rotated at 4° C. for 30 min in selection buffer containing 0.1% acetylated BSA. This process was repeated several times by removing the supernatant to fresh beads immobilized with GDP-bound Gαs. The supernatant from the last negative selection was then added to beads immobilized with GppNHp-bound Gαs (final conc. 200 nM) and rotated at 4° C. for 30 min in selection buffer containing 0.5 mM GppNHp and 0.1% acetylated BSA. As described in the first round of selection, the cDNA was quantified with qPCR, amplified with PCR, transcribed and ligated to puromycin. The subsequent selection was repeated for several rounds until a significant enrichment of cDNA was observed for GppNHp-bound Gαs. The recovered cDNA was then identified by next generation sequencing (Miseq, Illumina).

### Comparison Selection.

In comparison selection, ligated mRNA (7.5 pmol) from last round selection was added to a 5 μL scale reprogrammed in vitro translation system. After translation, cyclization, reverse transcription and prewashed with Sephadex G-25 columns, the desalted solution of peptide-mRNA/cDNA library was split equally into three fractions, and perform three paralleled selections with the same amount of blank, GDP-bound Gαs-immobilized or GppNHp-bound Gαs-immobilized Dynabeads M280 streptavidin magnetic beads, individually. For each of the paralleled selections, the beads were rotate at 4° C. for 30 min, washed three times with selection buffer. The remaining cDNAs were then eluted from the beads, quantified by qPCR, followed by Miseq sequencing. Finally, identified sequences from each paralleled selection were compared by normalization of Miseq abundance of the sequence with the qPCR reads of the paralleled selection.

### Example 3: State-Selective Modulation of Heterotrimeric Gαs Signaling with Macrocyclic Peptides

The G protein-coupled receptor (GPCR) cascade leading to production of the second messenger cAMP is replete with pharmacologically targetable receptors and enzymes with the exception of the Gα subunit, Gαs. GTPases remain largely undruggable given the difficulty of displacing high-affinity guanine nucleotides and the lack of other drug binding sites. We explored a chemical library of 10¹² cyclic peptides in order to expand the chemical search for inhibitors of this enzyme class. We identified two macrocyclic peptides, GN13 and GD20, that antagonize the active and inactive states of Gαs, respectively. Both macrocyclic peptides fine-tune Gαs activity with high nucleotide-binding-state selectivity and G protein class-specificity. Co-crystal structures reveal that GN13 and GD20 distinguish the conformational differences within the switch II/α3 pocket and block effector interactions. The Gαs inhibitors showed strong activity in cellular contexts through binding to crystallographically defined pockets. The discovery of cyclic peptide inhibitors targeting Gαs provides a path for further development of state-dependent GTPase inhibitors.

The family of human GTPases represents a vast but largely untapped source of pharmacological targets. They serve as key molecular switches that control cell growth and proliferation through cycling between tightly regulated ON/OFF states. The role of specific GTPase family members across diverse human diseases have been widely established by cancer genome sequencing (e.g., KRAS, GNAS and others) and by familial studies in neurodegenerative disease (e.g., LRRK2, RAB39B) (Prior et al., 2012; O'Hayre et al., 2013; Alessi and Sammler, 2018; Wilson et al., 2014). Despite the widespread recognition of these disease target relationships, only very recently has the first drug targeting a GTPase K-Ras (G12C) achieved clinical proof of principle (Canon et al., 2019; Hallin et al., 2020). The covalent somatic cysteine mutant-specific nature of the K-Ras (G12C) drugs has opened the potential for targeting a GTPase for the first time.

Several peptide-based probes that non-covalently target GTPases have been reported, but they either lack proper drug-like properties or have limited target scope (Takasaki, et al., 2004; Ja and Roberts, 2004; Johnston et al., 2005; Johnston et al., 2005; Johnston et al., 2006; Ja et al., 2006; Austin et al., 2008). Short linear peptides have shown good ability to target the switch II/α3 helix region in the heterotrimeric G protein α-subunit (Ga) with high nucleotide binding state selectivity. However, linear peptides have relatively poor cell-permeability and inherent instability in cells.

Cyclic peptides are promising candidates for GTPase drug development. Like linear peptides, cyclic peptides are also capable of targeting protein-protein interfaces (Sohrabi et al., 2020). Peptide cyclization stabilizes the peptide sequence and constrains the flexible peptide conformations for better cell penetration (Dougherty et al., 2019). Cyclic peptide inhibitors of Gα proteins have been reported: for instance, the macrocyclic depsipeptide natural product YM-254890 targets GDP-bound Gαq with high specificity and potency in cells (Nishimura et al., 2010). Despite the highly conserved structure of G proteins and the recent chemical tractability of fully synthetic YM-254890, efforts to use this natural macrocycle as a scaffold from which to discover inhibitors of other G proteins (Gαs, Gαi) have not been successful (Kaur et al., 2015; Xiong et al., 2016; Zhang et al., 2017), likely because of the limited chemical diversity of available YM-254890 analogs. We therefore reasoned that screening an ultra-large chemical library of cyclic peptides against a given nucleotide binding state of Gαs might allow us to discover Gαs nucleotide-binding-state-selective inhibitors that discriminate between the active and inactive states of Gαs and potentially open the remainder of the GTPase family to pharmacological studies.

The Random nonstandard Peptide Integrated Discovery (RaPID) system (Yamagishi et al., 2011) is an in vitro display system which merges the flexibility of an in vitro translation system (FIT) (Murakami et al., 2006; Murakami et al., 2003., Ramaswamy et al., 2004; Xiao et al., 2008) with mRNA display, enabling the screening of exceptionally large macrocyclic peptide libraries (>10¹² molecules) against challenging targets (Passioura and Suga, 2017). Here we report, inter alia, the discovery by the RaPID system of GN13 and GD20, two macrocyclic peptides that are the first known cell-permeable, nucleotide-state-selective inhibitors of Gαs, with high selectivity over other G protein subfamilies and nucleotide binding states.

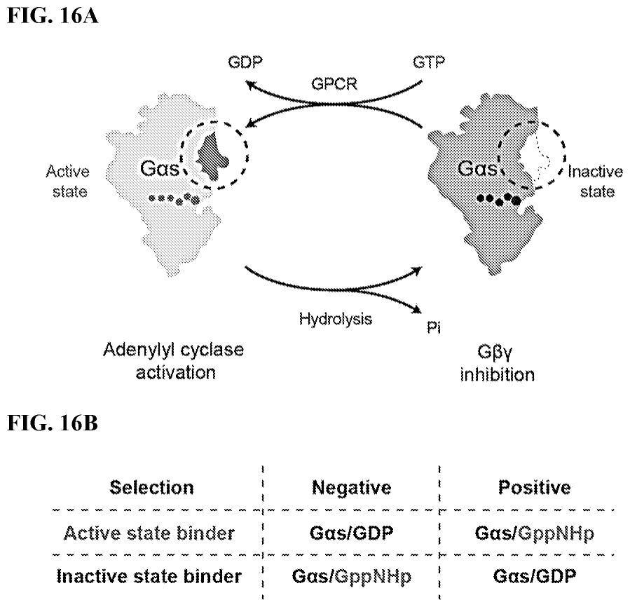

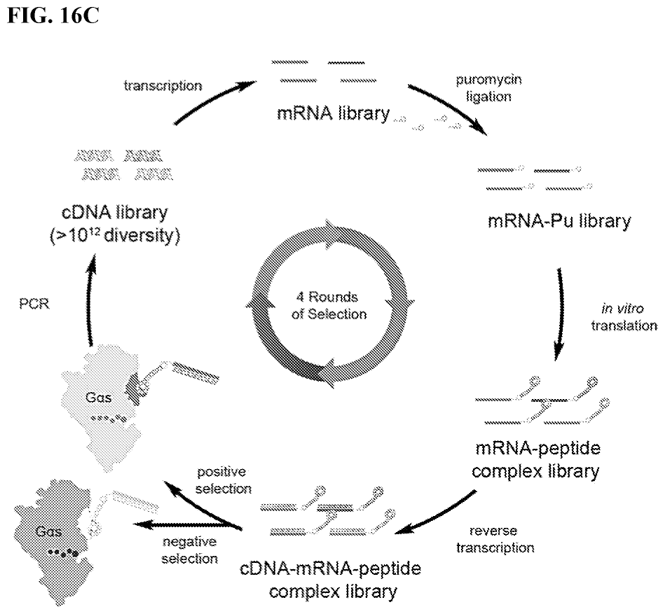

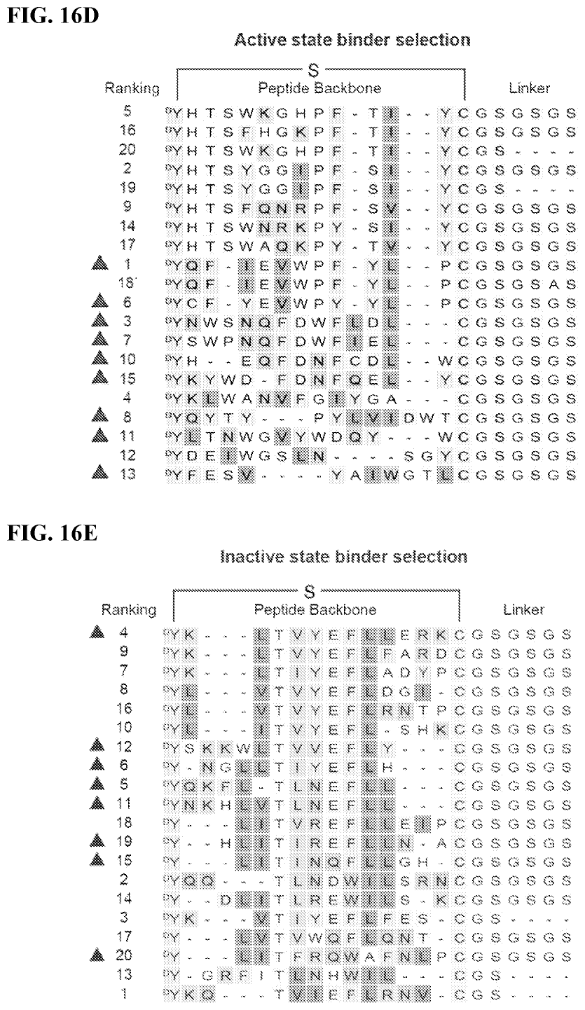

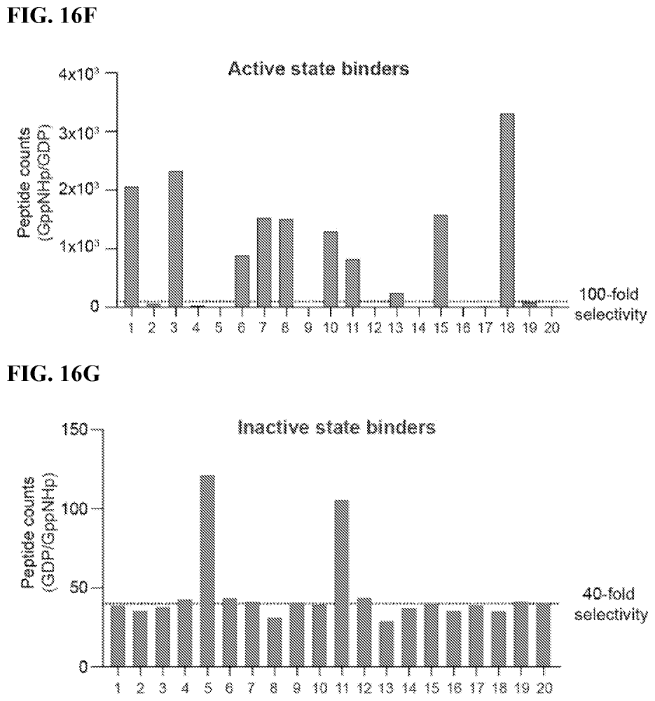

Selection of state-selective cyclic peptides that bind to the active or inactive state of Gαs. Affinity screening hits emerging from the RaPID cyclic peptide selection process against Gαs could theoretically bind anywhere on the surface of the protein and so might or might not perturb its function. To increase the probability of selecting a function-perturbing hit, we took advantage of the fact that when Gαs switches from the GDP-bound inactive state to the GTP-bound active state significant conformational changes occur on one face of Gαs, comprising the so-called switch I, II and III regions (Lambright et al., 1994), which are known to bind inhibitor or effector protein partners such as Gβγ or adenylyl cyclases (Tesmer et al., 1997; Liu et al., 2019) (FIG. 16A). We reasoned that performing both a positive selection against one state of Gαs and a negative selection against the other state would enrich for binders to the switch regions, and that these binders would be likely to state-selectively disrupt Gαs function.

In order to select a Gαs active state binding peptide, we performed the positive selection with wild-type Gαs (WT Gαs) bound to the non-hydrolyzable GTP analogue GppNHp (5′-guanylyl imidodiphosphate) and the negative selection against GDP-bound WT Gαs. A parallel Gαs inactive state binder selection was performed using GDP-bound WT Gαs as the positive selection and GppNHp-bound WT Gαs as the negative selection (FIG. 16B). Starting from a cDNA library, each round of selection included PCR amplification of the cDNA library, in vitro transcription into an mRNA library, ligation with a puromycin linker, and translation to generate a peptide library covalently conjugated with its encoding mRNA library (FIG. 16C). The library encoded peptides contain an N-chloroacetyl-D-tyrosine at the N-terminus, followed by 8-12 random proteinogenic amino acids encoded by NNK codons, a cysteine residue and a GSGSGS linker (FIGS. 16D-16E). Cyclization occurs spontaneously between the chloroacetyl group and the thiol group of the downstream cysteine residue (or possibly those appeared in the random region). The peptide-ligated mRNA library was further reverse-transcribed into a cDNA-mRNA-peptide library, subjected to a negative selection against one state of Gαs, then followed by a positive selection against the other state of Gαs (FIG. 16C).

After four rounds of selection (R¹-R⁴), cyclic peptide binders for the GppNHp-bound or GDP-bound Gαs were enriched and identified by next generation sequencing (NGS). The sequences of the top 20 hits were aligned and shown in FIGS. 16D-16E. Selective cyclic peptides from the R⁴ pool were identified and characterized by comparison selection against the respective positive and negative protein baits (FIGS. 16F-16G). Nine of the top twenty hits from the active state binder selection (with more than 100-fold selectivity for GppNHp-bound over GDP-bound Gαs, indicated by triangles in FIG. 16D) and eight of the top twenty hits from the inactive state binder selection (with more than 40-fold selectivity for GDP-bound over GppNHp-bound Gαs, indicated by triangles in FIG. 16E) were chosen for further analysis. To evaluate the cyclic peptide hits without the appended DNA/mRNA duplex, residues from the N-chloroacetyl-D-tyrosine to the glycine (after the anchor cysteine residue) of the selected peptides were synthesized using solid phase peptide synthesis followed by in situ cyclization.

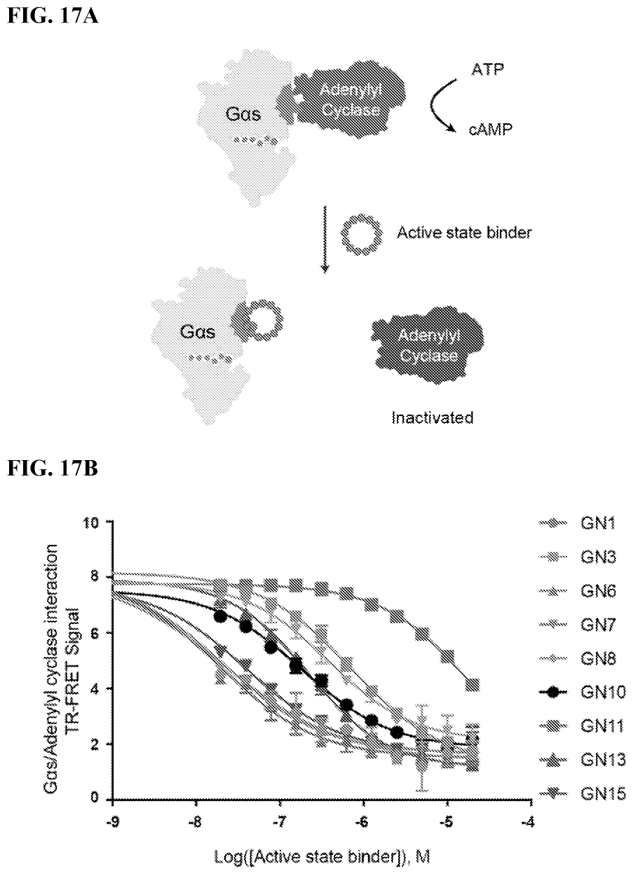

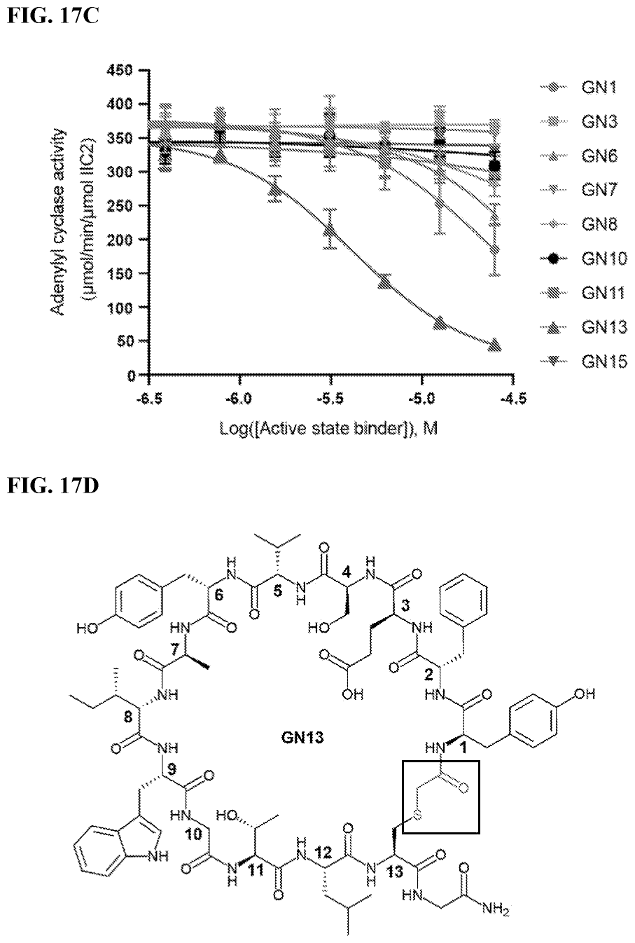

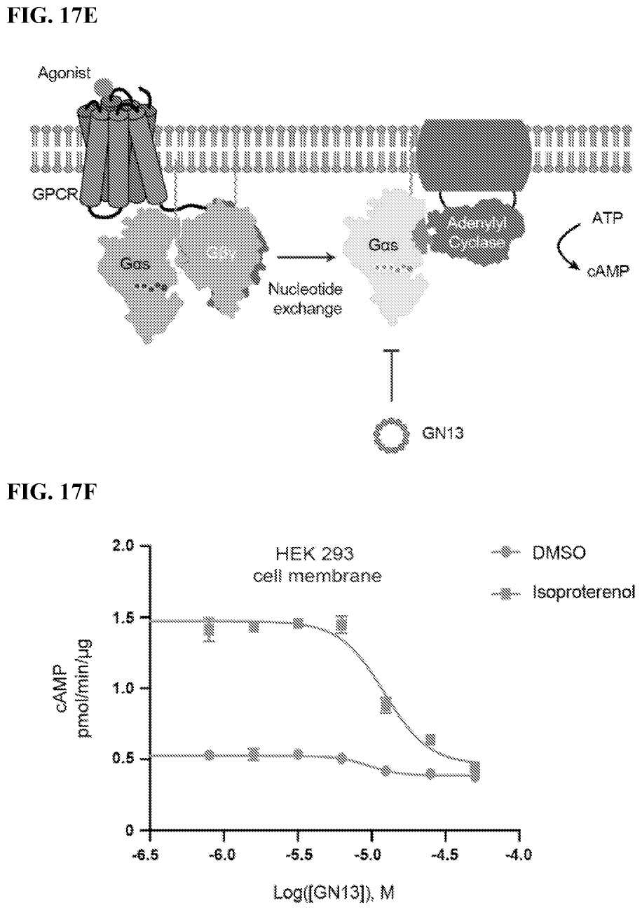

Active state binding cyclic peptide GN13 blocks Gαs-mediated adenylyl cyclase activation. In order to determine whether (Gαs/GppNHp) specific binders inhibit Gαs activity, we assayed the ability of Gαs to activate its downstream effector adenylyl cyclase (AC) (FIG. 17A). We refer to resynthesized active state binding cyclic peptides with a “GN” (Gαs/GppNHp) preceding their ranking number. We first tested the physical interaction between GppNHp-bound Gαs and adenylyl cyclase in the presence of the active state binders using a fluorescence resonance energy transfer (FRET) assay. Eight out of nine GN peptides showed strong, dose-dependent inhibition of the interaction between Gαs and adenylyl cyclase (FIG. 17B). We then evaluated the inhibitory effect of the top hits on Gαs-mediated adenylyl cyclase activation by measuring production of cAMP in a reconstituted Gαs activity assay (FIG. 17A). Among these nine active state binders, GN13 showed the greatest inhibition, with an IC₅₀ of 4.15±1.13 μM (FIGS. 17C-17D). GN13 did not inhibit forskolin-mediated, Gαs-independent adenylyl cyclase activation, suggesting a Gαs-dependent mechanism of inhibition.

The inhibitory Gα protein, Gαi, is a negative regulator in the cAMP pathway and possesses a structure similar to that of Gαs (Gilman., 1987). To assess whether GN13 was capable of discriminating between Gαs and Gαi, we measured the binding kinetics of GN13 to Gαi and Gαs using biolayer interferometry (BLI). We found that GN13 binds to immobilized GppNHp-bound Gαs with a K_{D} value of 0.19±0.02 μM. By contrast, GN13 showed no detectable binding to GDP-bound Gαs, GDP-bound Gαi1 or GppNHp-bound Gαi1 at the highest concentration tested, confirming the state-selectivity and class-specificity of GN13 for the active state of Gαs.

We sought to test the efficacy of GN13 in B2-adrenergic receptor (β2AR) mediated second messenger stimulation in cell membranes. Membrane anchored GDP-bound Gαs is inhibited by Gβγ in the resting state. It can undergo GPCR mediated GDP to GTP exchange upon agonist stimulation (Weis and Kobilka, 2018). The presence of GN13 could potentially capture the newly generated GTP-bound Gαs and inhibit its activation (FIG. 17E). To test this idea, we incubated cell membranes prepared from HEK293 cells with GN13 and examined cAMP accumulation with or without stimulation of β2-adrenergic receptor (β2AR) by isoproterenol (ISO). GN13 inhibited ISO-stimulated Gαs activity to a background level, with an IC₅₀ of 12.21±2.51 μM (FIG. 17F). These results suggested that GN13 can modulate Gαs activation by agonist stimulated β2AR.

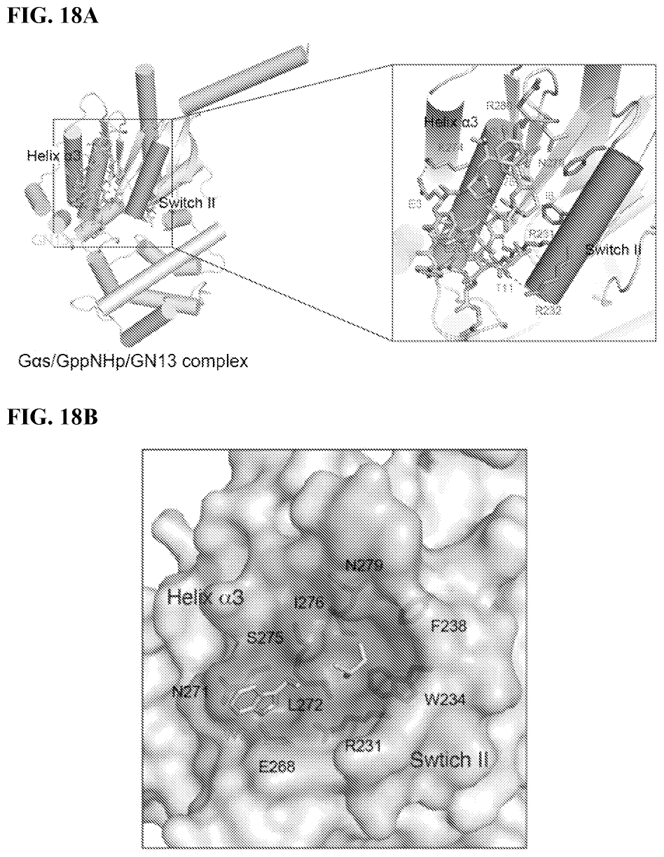

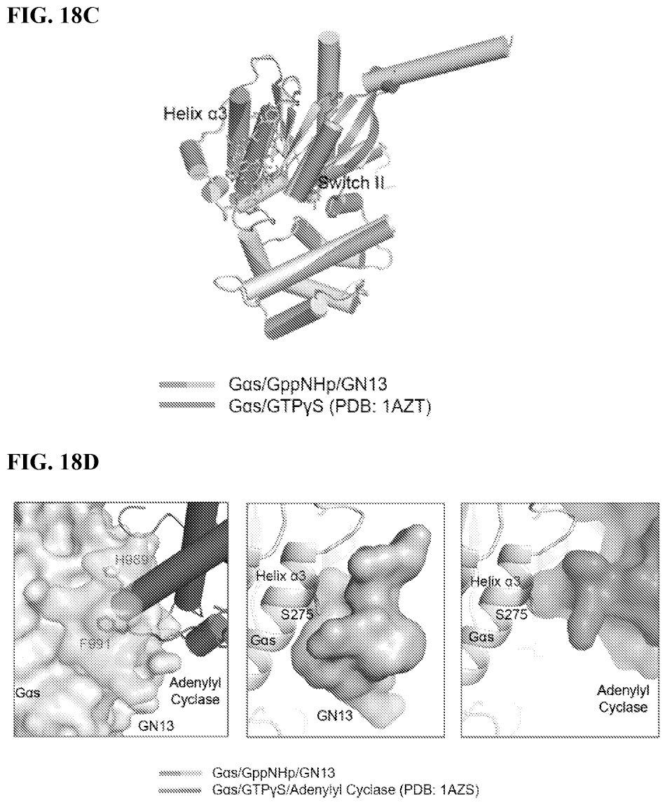

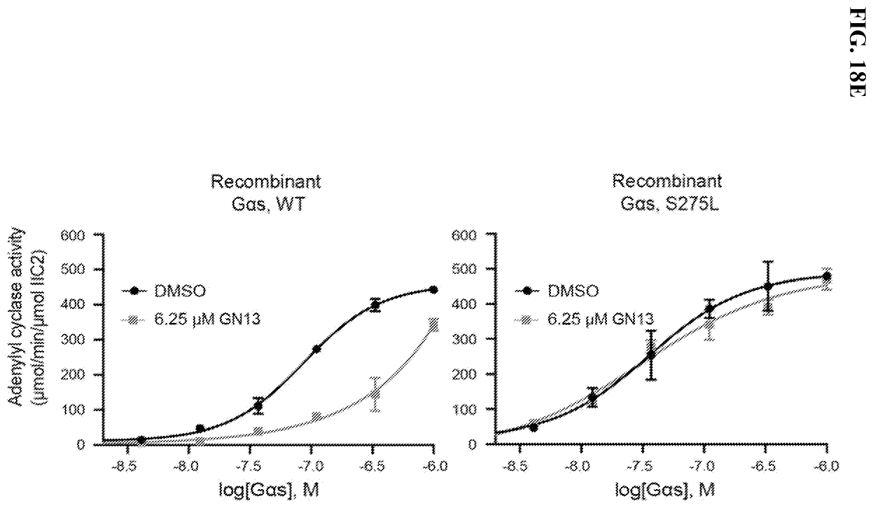

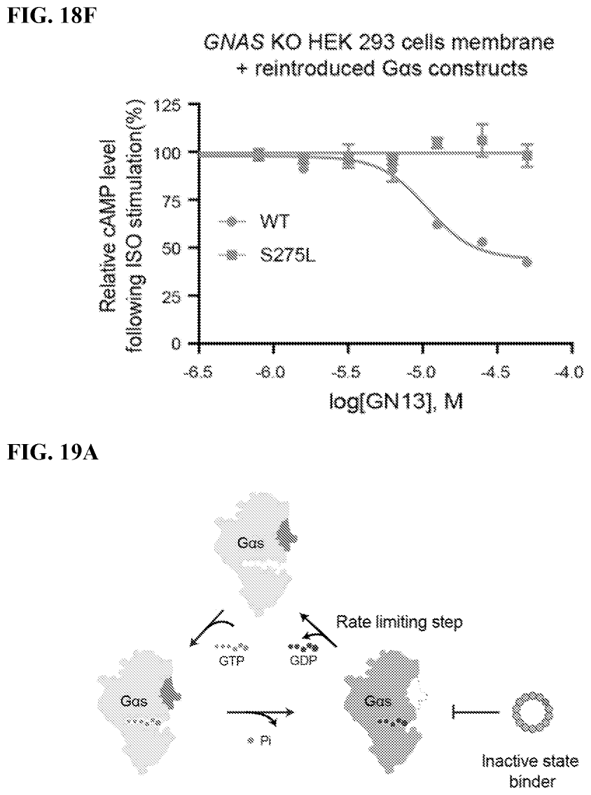

The crystal structure of GppNHp-bound Gαs in complex with GN13. To elucidate how the cyclic peptide GN13 binds to Gαs and inhibits Gαs-mediated adenylyl cyclase activation, we solved a co-crystal structure of the Gαs/GppNHp/GN13 complex. The structure was determined by molecular replacement and refined to 1.57 Å (Table 1). The overall structure is shown in FIG. 18A. GN13 assumes a highly ordered structure through extensive intramolecular and intermolecular hydrogen bonding networks with three well-defined water molecules. One molecule of GN13 binds to a pocket between switch II and the α3 helix of GppNHp-bound Gαs through hydrogen bonding and hydrophobic interactions (FIGS. 18A-18B). Specifically, the side chain of residue E3 in GN13 accepts a hydrogen bond from the ε-amino group of K274 in Gαs; the indole ring of residue W9 in GN13 donates a hydrogen bond to the side chain of E268 in Gαs; the main chain carbonyl oxygens of residues V5, W9 and T11, and the main chain amide of W9 in GN13 form five hydrogen bonds with residues N279, R280, R231, R232, and S275 on switch II and the α3 helix in Gαs (FIG. 18A inset). The side chains of residues 18 and W9 (IW motif) in GN13 dock into two hydrophobic pockets on Gαs (FIG. 18B), giving rise to a high Gαs binding affinity.

Structural analysis reveals that residue W9 in GN13 is centrally located within the interface between GN13 and Gαs, contributing three hydrogen bonds as well as hydrophobic interactions with the switch II/α3 pocket (FIGS. 18A-18B). Analogous to this key tryptophan in GN13, PDEγ (effector of G protein transducin, Gt) residue W70 (Slep et al., 2001) and adenylyl cyclase II (effector of Gαs) residue F991 (Tesmer et al., 1997) are inserted into the same switch II/α3 clefts of Gt and Gαs (FIG. 18D left panel). Comparison between the Gαs/GppNHp/GN13 complex structure and a Gαs/GTPγS/adenylyl cyclase complex structure (PDB: 1AZS) suggested that GN13 directly occludes the hydrophobic interaction between Gαs and adenylyl cyclase (Tesmer et al., 1997), which accounts for the inhibitory effect of GN13 (FIG. 18D left panel).

Structural basis for the nucleotide state-selectivity and the G protein class-specificity of GN13. The Gαs/GppNHp/GN13 complex strongly resembles the Gαs/GTPγS structure (PDB: 1AZT) (Sunahara et al., 1997), suggesting that GN13 recognizes the active state conformation and does not induce significant conformational change upon binding (FIG. 18C). We aligned our structure with the structure of inactive GDP-bound Gαs (chain I in PDB 6EG8) (Liu et al., 2019). In comparison with our structure, the N-terminus of switch II in the GDP-bound Gαs is unstructured and close to the α3 helix, with nearly half of the GN13/Gαs interface disrupted. In particular, R²³² of switch II (shown in space filling) is predicted to create a steric clash with 18 of GN13. Therefore, the GN13-bound complex structure explains the state-selectivity of GN13 for the active state of Gαs.

GN13 showed excellent G protein class-specificity, although we did not include other Gα proteins, such as Gαi, as a part of the negative selection. To identify the G-protein specificity determinants of GN13, we aligned our structure with the structure of active state Gαi in complex with a Gαi-specific linear peptide, KB1753 (PDB: 2G83) (Johnston et al., 2006). The affinity determinant, IW motif, was presented in both GN13 and KB1573, but Gαs-specific binding of GN13 was mainly determined by charge interactions (FIG. 18A inset): (1) D229 in Gαs (homologous residue in Gαi: S206) confines the orientation of switch II R232, which forms a hydrogen bond with a main chain carbonyl oxygen of T11 in GN13. (2) K274 in Gαs (homologous residue in Gαi: D251) interacts with the negatively charged side chain of E3 in GN13. Homologous residues in Gαi either do not engage with or even repulse against GN13, rendering the lack of GN13 binding.

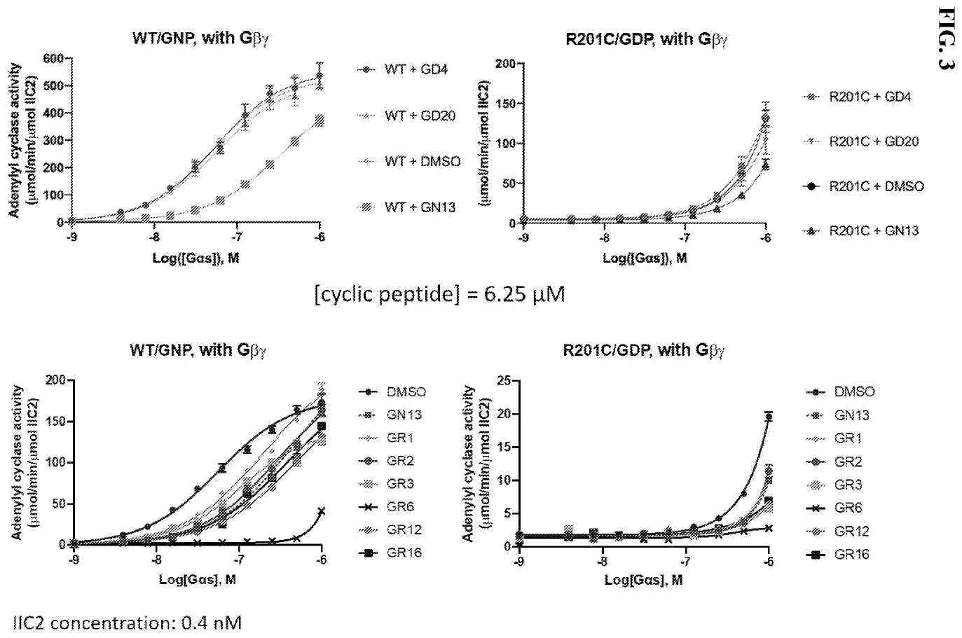

To further assess the cellular specificity of GN13, we designed a GN13-resistant Gαs mutant based on structural analysis. We examined the structures of the Gαs/GN13 complex and Gαs/AC complex and noticed that serine 275 in Gαs makes close contact with GN13, but does not contact adenylyl cyclase (FIG. 18D middle and right panels). We reasoned that mutating this serine residue to a bulky residue should create a drug-resistant Gαs mutant that blocks the GN13-Gαs interaction but would have little effect on adenylyl cyclase activation. Indeed, Gαs S275L mutant maintained a similar level of biochemical activity but was resistant to GN13 inhibition (FIG. 18E). We tested GN13 in the membranes of GNAS-knockout (GNAS-KO) HEK 293 cells that did not express endogenous Gαs protein (Stallaert et al., 2017). GN13 was able to inhibit isoproterenol-mediated cAMP production in cell membranes in a dose-dependent manner when WT Gαs was reintroduced into the GNAS KO cells by transient transfection. By contrast, when the drug resistant mutant Gαs S275L was reintroduced into GNAS KO cells, the inhibitory effect of GN13 was abolished (FIG. 3I), providing further evidence that the observed pharmacological activity is due to GN13 binding to the switch II/α3 pocket in Gαs.

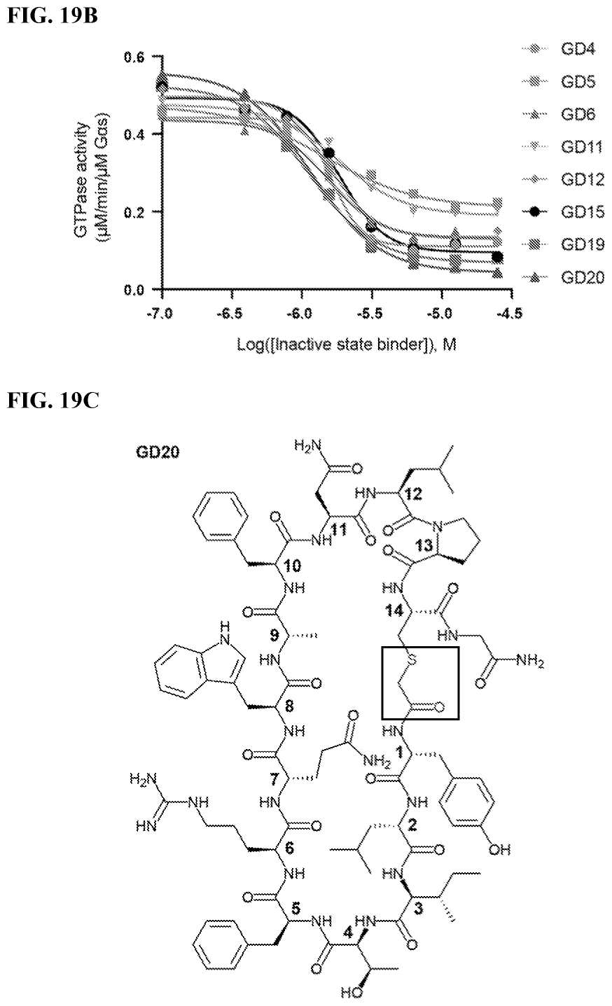

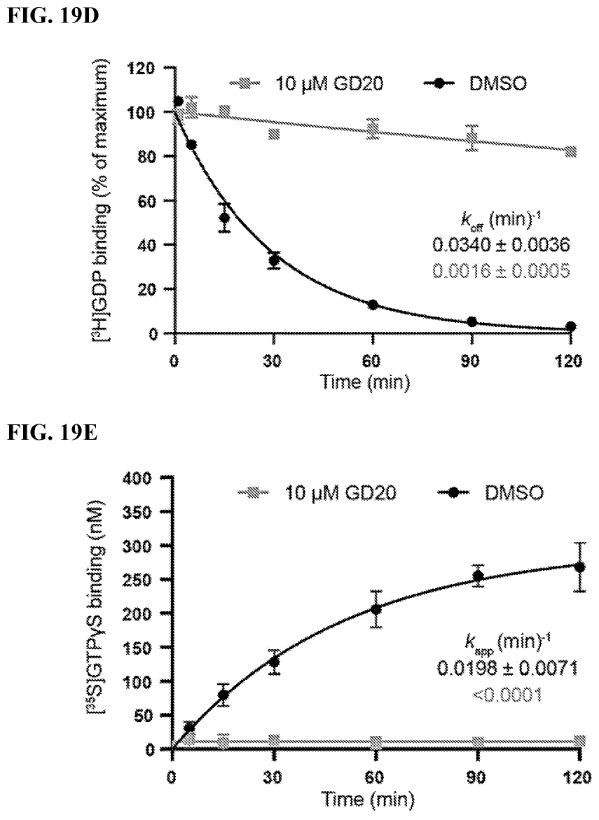

Inactive state binding cyclic peptide GD20 is a Gαs specific guanine nucleotide dissociation inhibitor (GDI). The activation of G protein signaling is often rapid and temporary. Gα GTPase activity promotes GTP hydrolysis to GDP and rearranges the switch regions to adopt a GDP-bound inactive conformation. This precisely orchestrated nucleotide binding conformation prevents GDP release, which makes GDP dissociation the rate-limiting step of G protein activation (Dror et al., 2015). An inactive state binder could hypothetically modulate GDP-bound Gαs either by inhibiting GDP release as a guanine nucleotide dissociation inhibitor (GDI) or by facilitating GDP to GTP switch as a guanine nucleotide exchange factor (GEF) (Ghosh et al., 2017). In order to understand how inactive state binders control GDP-bound Gαs function, we carried out a multiple turnover assay to evaluate Gαs steady-state GTPase activity in the presence of top hits from the inactive state binder selection (FIG. 19A). The inactive state selected cyclic peptides are indicated with a “GD” (Gαs/GDP) preceding their ranking number, and all of the tested GD peptides showed strong, dose-dependent inhibition against Gαs steady-state GTPase activity. GD20 showed the greatest inhibition, with an IC₅₀ of 1.15±0.16 μM (FIGS. 19B-19C).

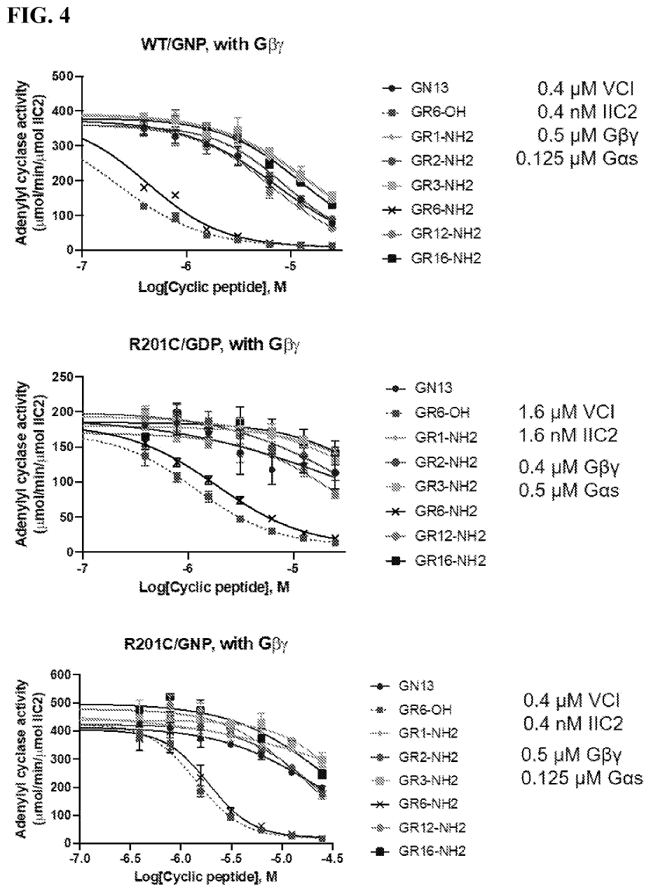

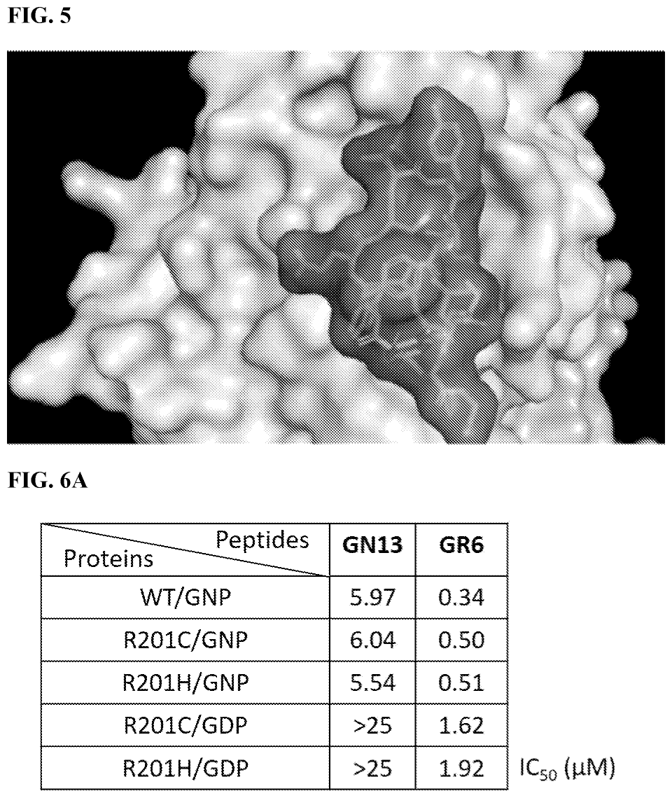

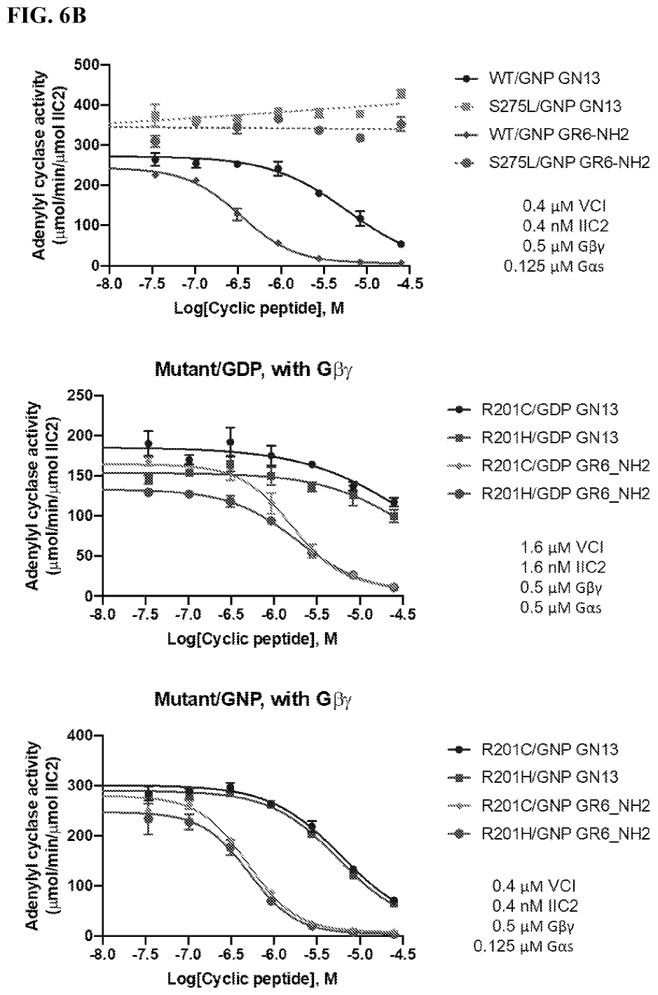

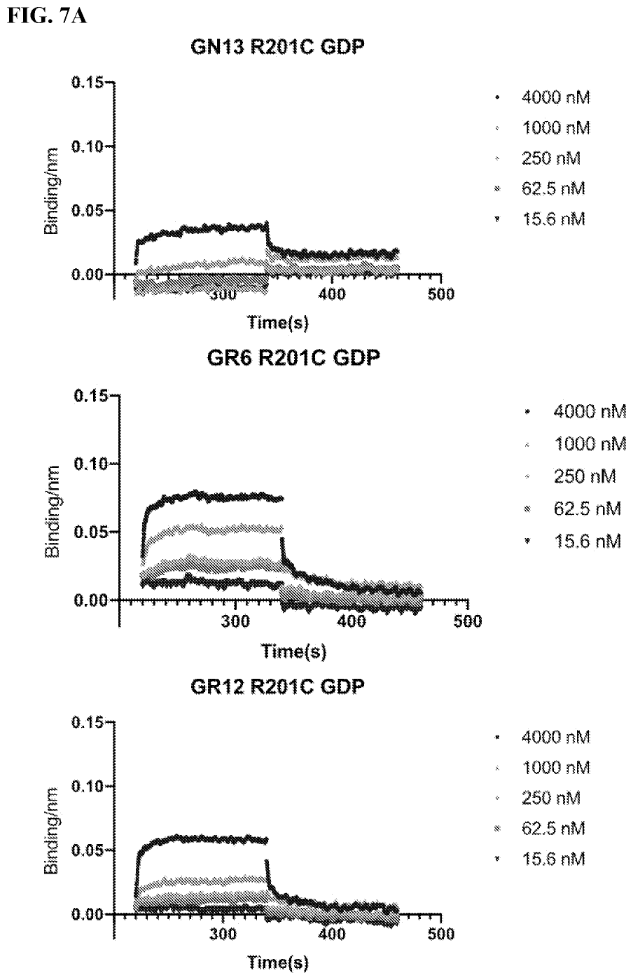

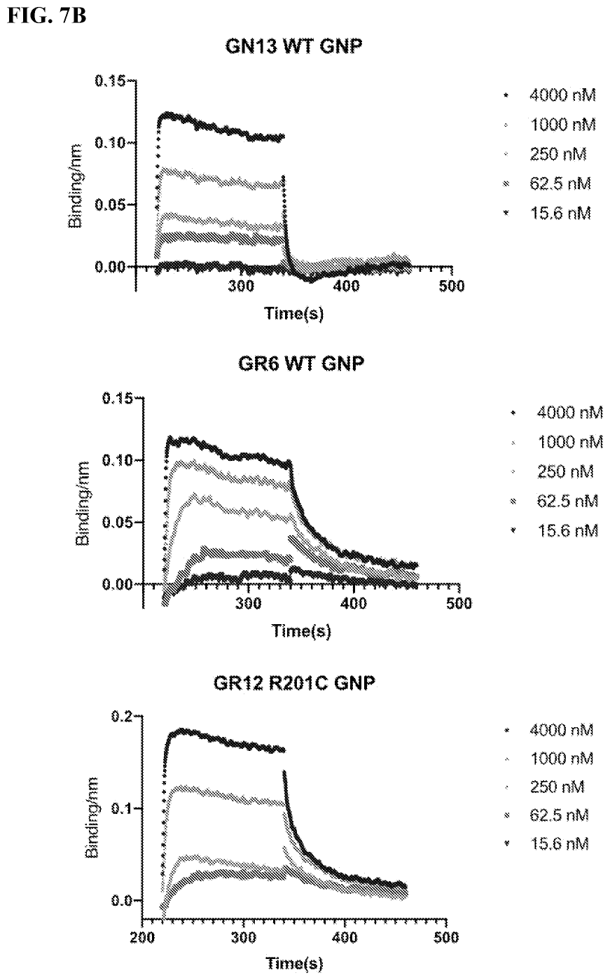

We determined rate constants for two individual steps in the GTPases cycle, GD20 displayed profound GDI activity towards Gαs by drastically reducing Gαs GDP dissociation rates (k_{off}) and the apparent rate of GTPγS binding (kₐₚₚ) to Gαs (FIGS. 4D and 4E). On the contrary, GN13 only slightly influenced Gαs GDP dissociation, however, increased the apparent rate of GTPγS binding (kₐₚₚ) to Gαs. The discrepancy between GD20, a synthetic Gαs GDI, and GN13, a synthetic Gαs GEF, exemplified how state-selective Gαs binders fine-tune Gαs enzymatic activity. The precise regulation of Gαs by cyclic peptides not only appears at the nucleotide binding state level, but also at the G protein family level. The nucleotide exchange step of a homologous G protein Gαi, is much less sensitive towards GD20 and GN13, confirming the class-specificity of both GD20 and GN13 for Gαs.

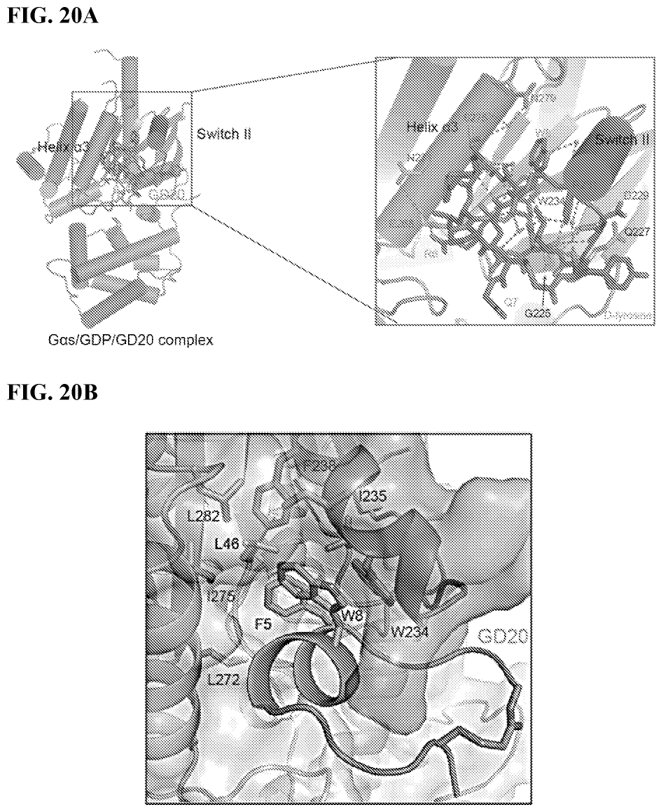

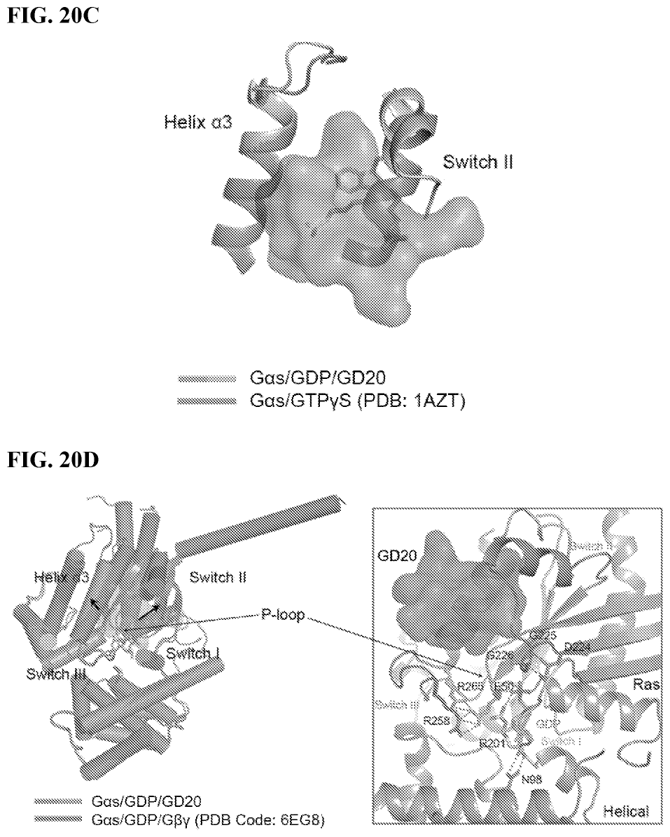

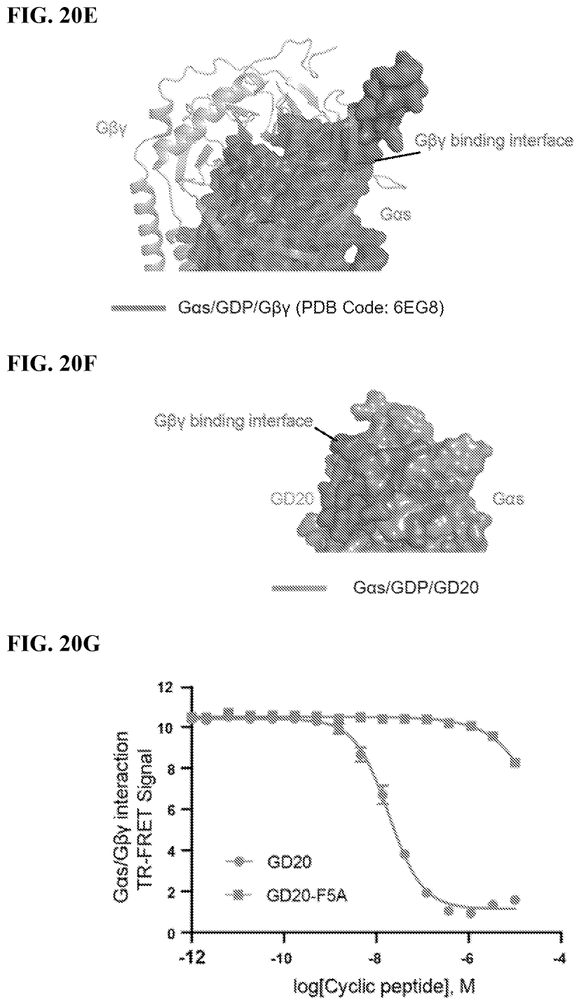

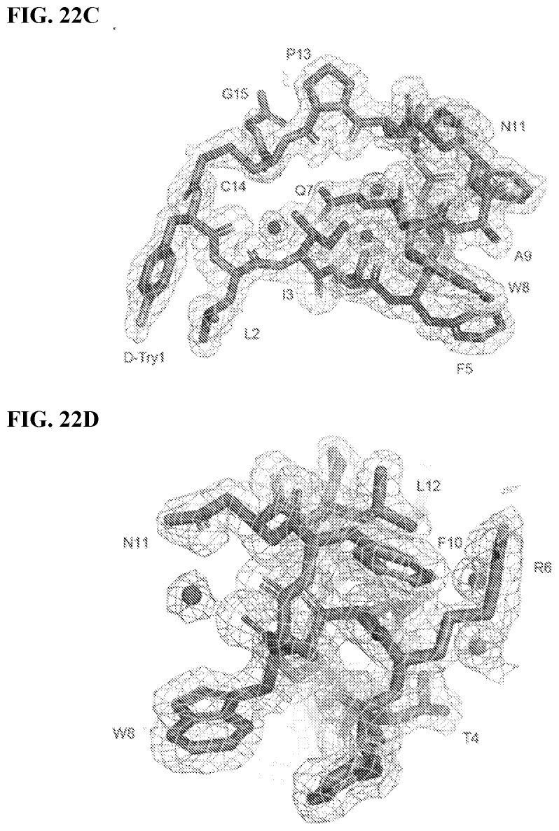

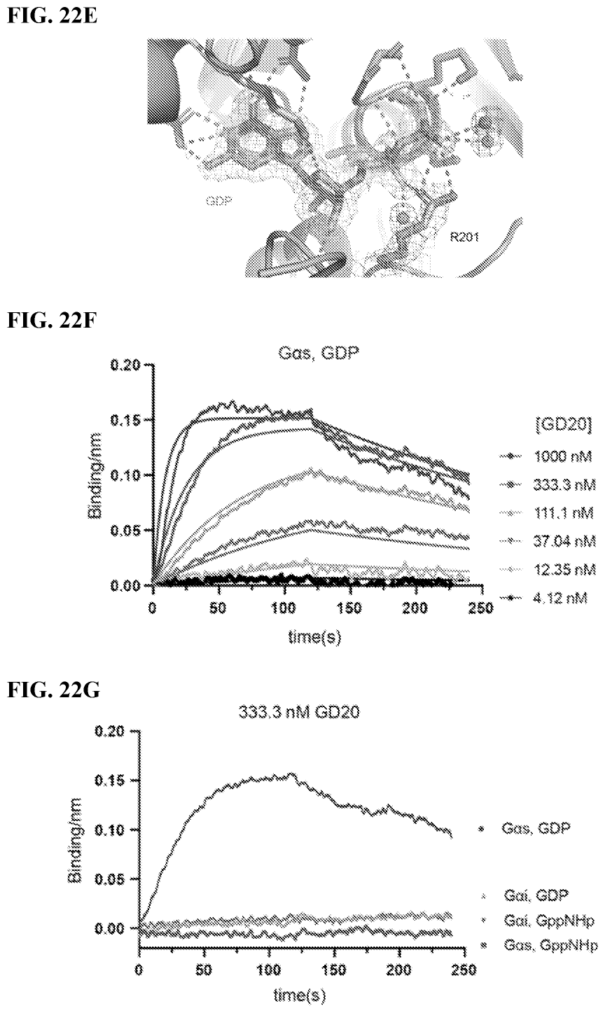

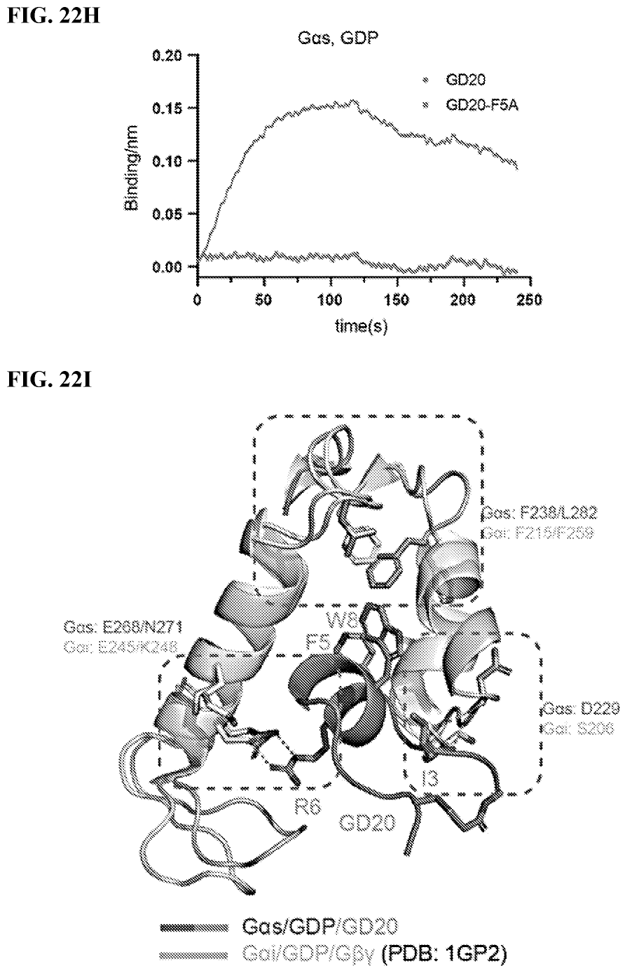

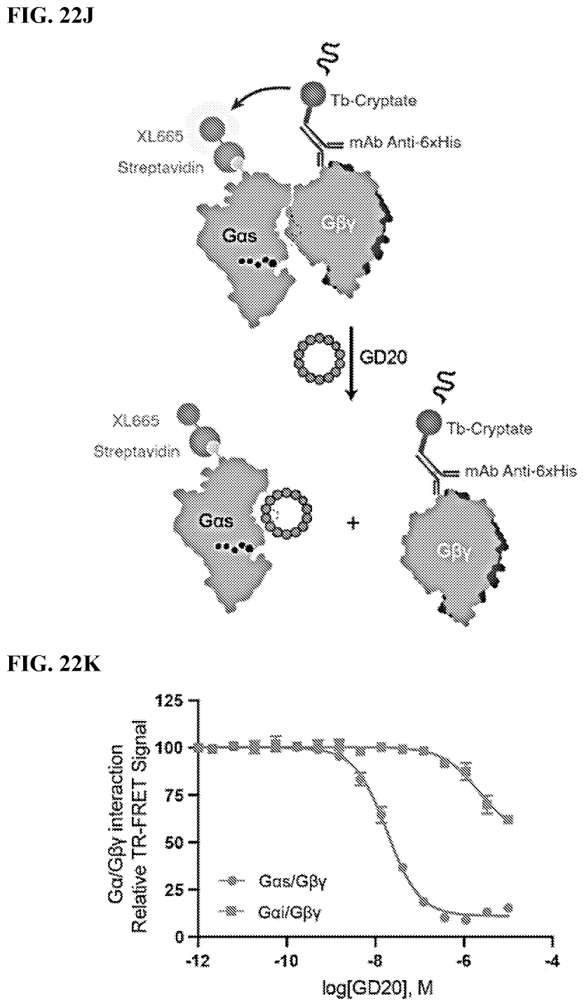

The crystal structure of GDP-bound Gαs in complex with GD20. To understand the underlying molecular determinants of how cyclic peptide GD20 favors GDP-bound Gαs and inhibits GDP dissociation. We solved a co-crystal structure of the Gαs/GDP/GD20 complex. The structure was determined by molecular replacement and refined to 1.95 Å (Table 2). The overall structure is shown in FIG. 20A. Four well-defined water molecules and a number of intramolecular hydrogen bonds constructed a unique helical secondary structure in GD20 (FIGS. 22A-22E). One molecule of GD20 binds to the cleft between switch II and the α3 helix of GDP-bound Gαs through electrostatic interactions, hydrogen bonding, and hydrophobic interactions (FIGS. 20A-20B). Specifically, the side chain of residue R6 in GD20 forms a salt bridge with Gαs E268, and this ion pair is further stabilized by Gαs N271; the main chain carbonyl oxygen of F10 in GD20 forms a hydrogen bond network with S275 and N279 from the α3 helix in Gαs; R231 and W234 on Gαs switch II coordinate a complex hydrogen bond network with W8, N11, L12, C13 and D-tyrosine in GN13; deep inside of the GD20 binding pocket, the main chains of I3 and F5 form multiple hydrogen bonds with G225, Q227, and D229 in Gαs (FIG. 20A inset). These charge and hydrogen bonding interactions rearrange the flexible Gαs switch II motif and bury residues F5 and W8 of GD20 inside of a hydrophobic pocket (FIG. 20B). These solidified hydrophobic interactions between GD20 and Gαs likely contribute to the high Gαs binding affinity.

We measured the binding kinetics of GD20 to Gαs using BLI to quantify the binding event. We found that GD20 bound to immobilized GDP-bound Gαs with a K_{D} value of 31.4±0.7 nM (FIG. 22F). By contrast, GD20 had no detectable binding to GppNHp-bound Gαs, GDP-bound Gαi1 or GppNHp-bound Gαi1 at the highest concentration tested (FIG. 22G), confirming the state-selectivity and class-specificity of GD20 for the inactive state of Gαs. A single point mutation F5A nearly completely abolished Gαs binding affinity of GD20, confirming the importance of hydrophobic interactions mediated by F5 (FIG. 22H).

Structural basis for the binding selectivity and biochemical activity of GD20. The high resolution GD20-bound Gαs complex structure elucidated the molecular mechanism by which GD20 distinguishes GDP-bound Gαs over other protein or nucleotide binding states. First, we aligned our GD20-bound Gαs structure with the structure of active GTPγS-bound Gαs (PDB: 1AZT) (Sunahara et al., 1997). The presence of a rigidified switch II motif in the GTPγS-bound Gαs clashes with GD20 (FIG. 20C). In particular, R231, R232 and W234 of switch II (shown in space filling) are predicted to create a steric clash with GD20. Second, we compared our GD20-bound Gαs structure with the inactive GDP-bound Gαs structure in complex with G07 (chain I in PDB 6EG8) (FIG. 20D) (Liu et al., 2019). GD20 binding expands the switch II/α3 pocket by repositioning both motifs. However, structural motifs (such as switch I, III, and the P loop) that are critical for GDP binding remain unchanged with no discernible difference, indicating the GDP-state selective of GD20. Finally, we compared our GD20-bound Gαs structure with the GDP-bound Gαi structure in complex with Gβγ (PDB: 1Gβ2) (FIG. 22I) (Wall et al., 1995). The specificity of GD20 is determined by three elements which involves electrostatic interactions, Van der Waals interactions, and hydrogen bonding. (1) E268 in Gαs (homologous residue in Gαi: E245) interacts with the positively charged side chain of R6 in GD20. At the same location in Gαi, the presence of a nearby K248 neutralizes the surface charge of Gαi and disfavors the salt bridge formation between Gαi and GD20. (2) F5 and W8 in GD20 dock into a hydrophobic pocket made of two non-polar residues F238 and L282 from Gαs. A L282 to F259 substitution in Gαi structure sterically reshapes the positioning between F238 and L282 in the hydrophobic pocket, dislocates the correct orientation for GD20 Van der Waals interactions. The subtle difference in this pocket between Gαs and Gαi also controls the specificity of other Gα effectors (Chen et al., 2005; Tesmer et al., 2005). (3) The reshaped hydrophobic pockets also influence the residues on the switch II motif. The main chain amide of D229 in Gαs, and the homologous residue S206 in Gαi form different hydrogen bonding patterns. Aspartate 229 in Gαs, but not S206 in Gαi, forms two hydrogen bonds with the main chain amide of I3 in GD20. Homologous residues in Gαi either do not enhance or may even diminish the binding with GD20, resulting in a strong selectivity for Gαs over Gαi.

The GD20-bound Gαs complex structure also demonstrates the molecular basis of GD20 GDI activity (FIG. 20D inset). GDP dissociation from Gαs nucleotide binding site requires both conformational changes that weaken the guanine nucleotide affinity and Ras/Helical domain separation that allows GDP release (Dror et al., 2015). GD20 does not engage the GDP exit tunnel, therefore is not directly occluding GDP release. Instead, GD20 phenocopies the effect of Gβγ GDI activity, rigidifying the juxtapositions of switch I, III, and the P loop. Such a conformational lock not only orients Gαs R201 and E50 to directly capture the β-phosphate of GDP, but also inhibits the spontaneously Ras/Helical domain separation by stabilizing two hydrogen bonds between Gαs R201 and N98. As a result, GD20 strongly antagonizes GDP dissociation from Gαs.

When we compared our GD20-bound Gαs structure with another GDP-bound Gαs structure in complex with Gβγ (FIG. 20D) (Liu et al., 2019), we noticed that GD20 induces a significant conformational shift of the Gβγ binding surface, and GD20 occupies the Gβγ binding surface in a competitive manner (FIGS. 20E-20F). We measured the interaction of GDP-bound Gαs and Gβγ in the presence of GD20 or the Gαs binding deficient analog GD20-F5A using a fluorescence resonance energy transfer (FRET) assay (FIG. 22J). GD20, but not GD20-F5A, showed a potent, dose-dependent inhibition of the interaction between Gαs and Gβγ, with an IC₅₀ of 18.4±2.0 nM (FIG. 20G). This inhibitory effect was also Gαs specific, given that GD20 was at least 100-fold more selective for Gαs than Gαi in the FRET assay (FIG. 22K). These data suggested that GD20 selectively captures the GDP-bound Gαs, contributing to the Gαs GDI activity and the inhibitory effect against Gαs/Gβγ complex.

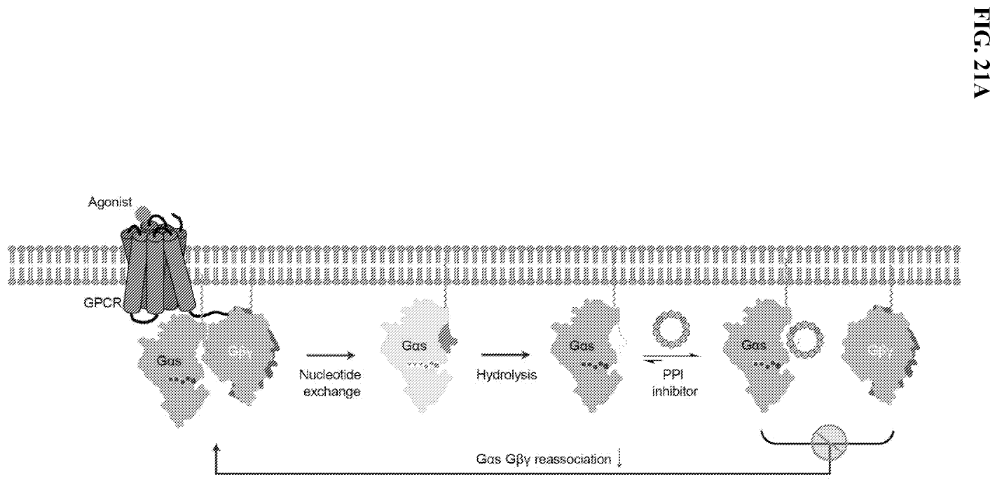

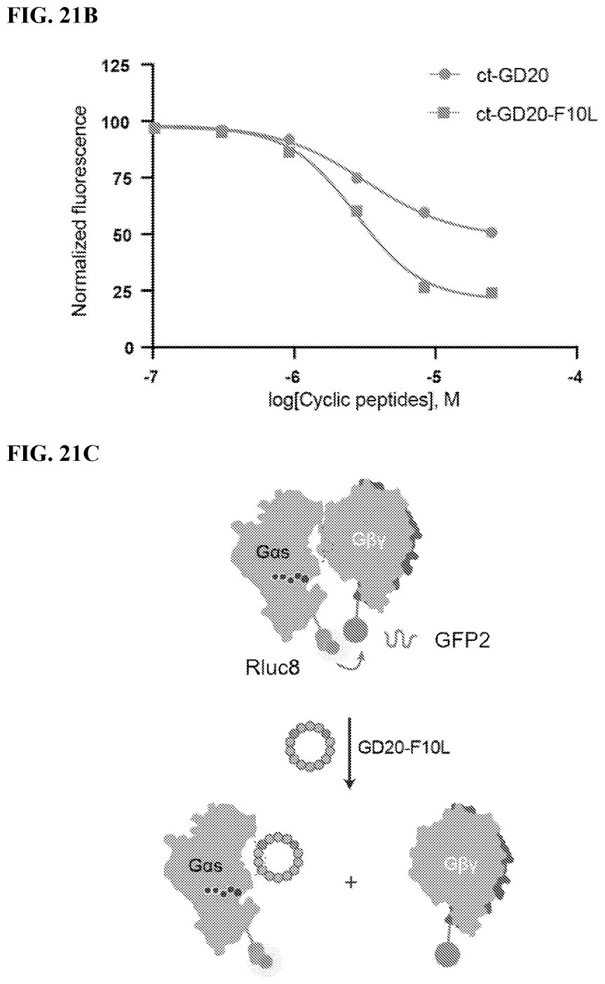

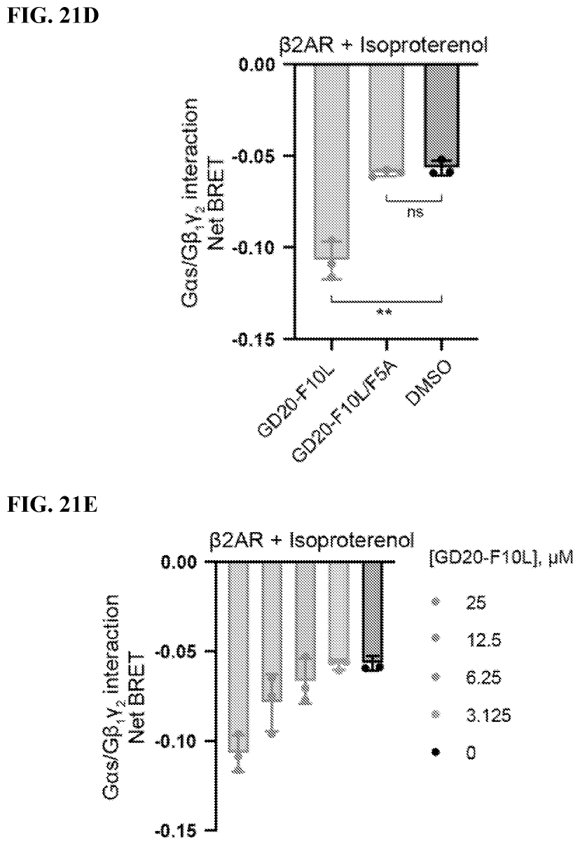

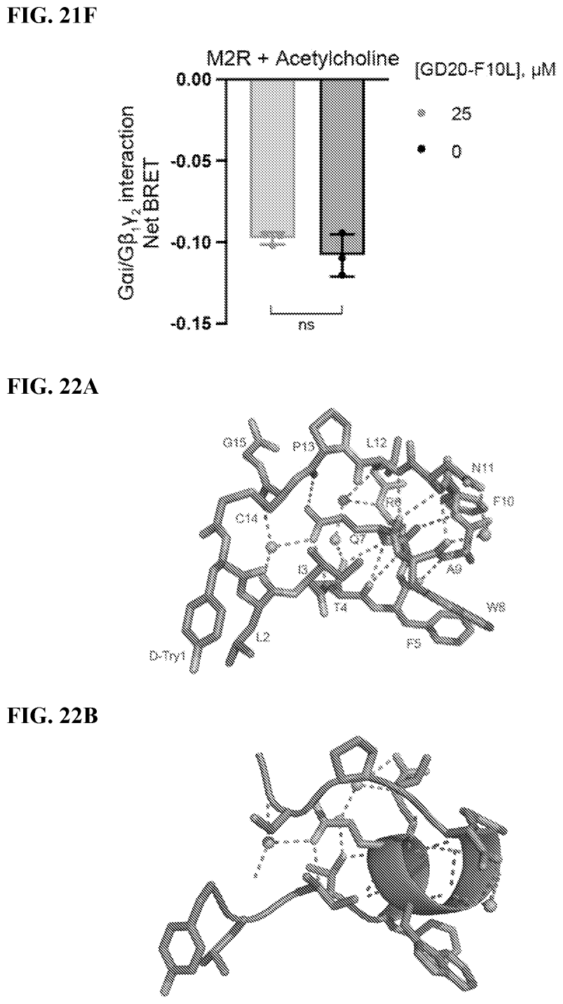

A cell permeable GD20 analog, GD20-F10L, inhibits Gαs/Gβγ interaction in HEK293 cells. Receptor coupled G protein signaling releases GTP-bound Gα and free Gβγ to engage their own effectors to transduce downstream signaling. GDP-bound Gα is a functional “OFF” switch by tightly reassociating with obligate Gβγ dimers and masking the effector binding surfaces on both Gαs and Gβγ (Gulati et al., 2018). A potent Gαs Gβγ protein-protein interaction (PPI) inhibitor should potentially block Gαs Gβγ reassociation and further extend the lifetime of free Gβγ (FIG. 21A). With a potent Gαs Gβγ PPI inhibitor, GD20, functioning in vitro, we next asked whether GD20 could reduce association of Gαs and Gβγ following receptor stimulation in the cells.

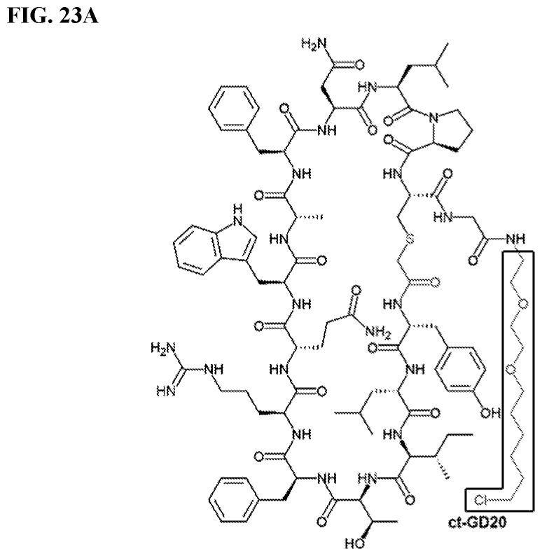

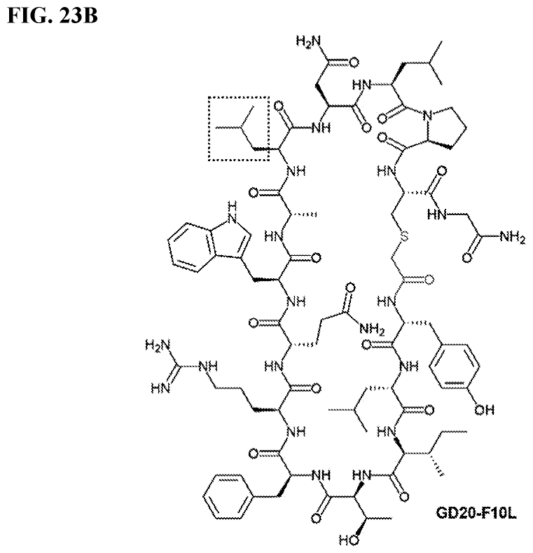

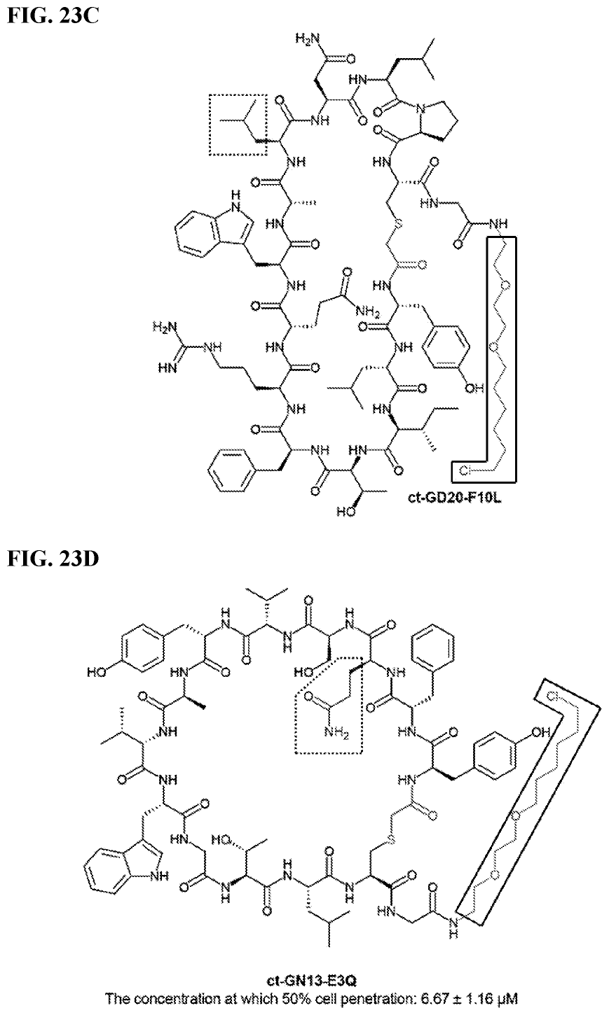

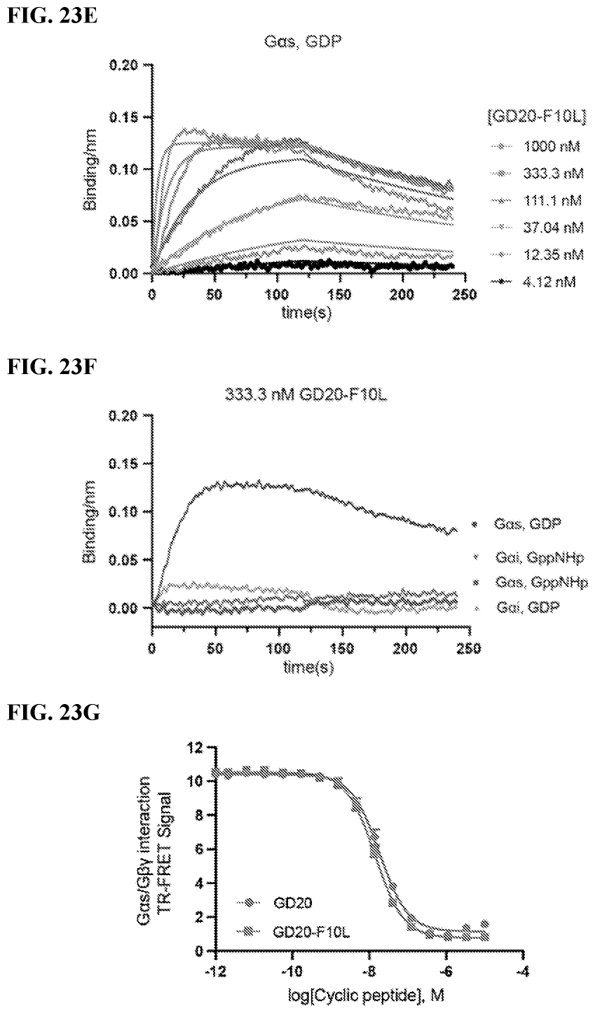

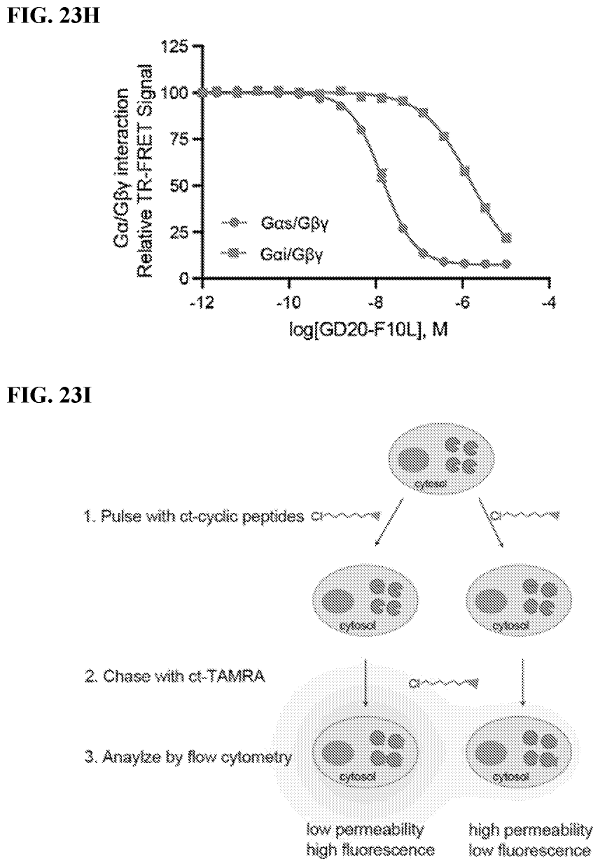

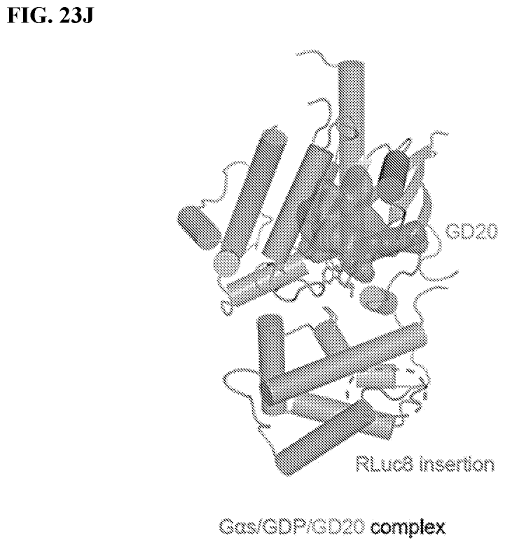

We first tested the cell permeability of GD20, as peptide-based chemical probes often suffer from poor cell permeability. Several G protein-specific linear peptides exhibit in vitro activities but have no reported cellular efficacy, likely due to their low cell permeability (Ja and Roberts, 2004; Johnston et al., 2005; Johnston et al., 2005; Johnston et al., 2006; Austin et al., 2008). In order to quantitatively evaluate the cell permeability of GD20, we used a recently developed HaloTag-based assay known as a chloroalkane penetration assay (CAPA) (Figure S6I) (Peraro et al., 2018). HeLa cells stably expressing HaloTag localized to the mitochondrial outer membrane were pulsed with chloroalkane-tagged molecules (ct-molecule), washed, chased with chloroalkane-tagged TAMRA fluorophore (ct-TAMRA), and finally analyzed by flow cytometry. A lower ct-TAMRA fluorescent signal indicates competition from a higher cytosolic concentration of ct-molecule. The carboxyl terminus of GD20 (G15) was conjugated with a chloroalkane tag to make ct-GD20 (FIG. 23A). While ct-GD20 is cell permeable, a single substitution F10L significantly improved cell penetration (FIG. 21B, see also FIGS. 23B-23C). Cyclic peptide GD20-F10L retains a similar level of binding affinity for GDP-bound Gαs with a K_{D} value of 14.5±0.4 nM (FIG. 23E), and comparable biochemical activity and class-specificity against Gαs Gβγ PPI, with an IC₅₀ of 14.0±0.6 nM for Gαs and a 100-fold selectivity over Gαi (FIGS. 23F-23H).

We next tested GD20-F10L in HEK 293 cells overexpressing both β2AR and Gαs/Gβγ. Rluc8 was inserted within a flexible loop region between the αB-αC helices of Gαs (FIG. 23J) and GFP2 was inserted at the N-terminus of Gγ2 to capture Gαβγ heterotrimer interaction. A decrease in the bioluminescence resonance energy transfer (BRET2) signal between labeled G protein subunits can detect Gαβγ dissociation (FIG. 21C) in live cells (Olsen et al., 2020). We examined Gαβγ trimer dissociation elicited by the GPCR agonist at various concentrations of cyclic peptides by monitoring the net BRET2 signal. In cells that were transiently transfected with a Gαs-coupled β2-adrenergic receptor (β2AR), GαsShort_Rluc8, Go 1, and Gγ-GFP2, ISO application stimulated a basal net BRET response. Pretreatment with GD20-F10L induced a greater net BRET2 signal between Gαs and Gγ. This effect was GD20-F10L dose dependent (FIGS. 21D-21E). In comparison, the Gαs binding deficient mutant GD20-F10L/F5A failed to induce a larger BRET2 response (FIG. 21D). To assess the specificity of GD20-F10L at the G protein level, we tested it against the Gαi/Gβγ interaction. HEK 293 cells transiently expressing Gαi-coupled muscarinic acetylcholine receptor M2 (M2R), Gαi1_Rluc8, Gβ1, and Gγ-GFP2 were challenged with M2R agonist, acetylcholine. Pretreatment with GD20-F10L did not induce a net BRET2 signal change between Gαi and Gβγ (FIG. 21F). These data suggested that GD20-F10L can specifically capture the monomeric state of Gαs after G protein activation and interfere with Gαs/Gβγ reassociation

GPCRs and G proteins comprise the largest family of signal transducing proteins in the human genome. Although approximately 35% of approved drugs target GPCRs, directly targeting the downstream integrator G proteins has the potential for broader efficacy via blocking converged pathways shared by multiple GPCRs (Bonacci et al., 2006; Gulati et al., 2018). However, there is a striking absence of drug-like chemical matter that specifically targets the Gα proteins in cells. Cyclic peptides bridge the chemical space between small molecules and biologics, and are therefore capable of recognizing shallow effector binding pockets at PPI interfaces while maintaining optimal pharmacological properties. This is demonstrated here by the development of Gαs selective cyclic peptide inhibitors GN13 and GD20, which specifically recognize the Gαs switch II/α3 pocket, the site where downstream effectors bind. Cyclization of the peptide sequence and introduction of a non-canonical amino acid (D-tyrosine) provide these Gαs inhibitors better cell permeability and chemical stability (FIG. 21B, see also FIG. 23D, Table 4, and Table 5), making them comparable to small molecule drugs. Moreover, in contrast with the complex cyclic peptide natural product YM-254890, our Gαs-binding cyclic peptides can be easily derivatized through side-chain substitutions. The high-resolution co-crystal structures that we obtained of Gαs with our cyclic peptides enable us to program the protein-inhibitor interaction for desired biological effects. This tunability is exemplified by two GD20 analogs, GD20-F10L and GD20-F5A, in which a single point substitution drastically changed the biochemical and pharmacological properties of a given cyclic peptide, providing opportunities for further optimization.

Both GN13 and GD20 bind at the switch II/α3 pocket in Gαs. This pocket is evolutionally conserved and is commonly used for effector binding, with subtle differences conferred by sequence variability between homologous Gα proteins and by binding of different nucleotides (Wall et al., 1995; Tesmer et al., 1997; Slep et al., 2001; Tesmer et al., 2005; Chen et al., 2005; Liu et al., 2019). Our extremely diverse chemical library along with both positive and negative selection enabled us to survey the sequence space of cyclic peptides and discover selective binders that capture specific, subtly different conformations of the switch II/α3 pocket. The resulting Gαs-cyclic peptide recognitions are highly class-specific and state-selective, allowing for a precise modulation of Gαs signaling.

Gαs is one of the most frequently mutated G proteins in human cancer. Hotspot mutations in Gαs (Q227) disrupt its GTPase activity, thereby locking Gαs in the GTP-bound active conformation (Zachary et al., 1990). We found that the cyclic peptide GN13 recognized this particular Gαs conformation and inhibited GTP- and GppNHp-bound Gαs Q227L mutant in the adenylyl cyclase activation assay. To our knowledge, this is the first demonstration of the ligandability of oncogenic Gαs.

The inactive state inhibitors GD20 and GD20-F10L similarly provide lead molecules to probe GDP-bound Gαs and exemplify a new mode of pharmacological intervention in GPCR signaling. The cell-penetrating cyclic peptide GD20-F10L captures a flexible switch-II conformation that is only available when Gαs is in the GDP bound state. This molecular recognition could be useful for developing biosensors directly detecting Gαs/GDP in cells. For example, a fluorescently tagged GD20-F10L could potentially be used for tracking real-time translocation of endogenous Gαs following receptor activation and internalization, which bypasses the need of G protein overexpression or genetic modification (Maziarz et al., 2020; Olsen et al., 2020).

GD20-F10L also offers a new angle to study the role of Gβγ signaling during GPCR stimulation. Gαs-GD20-F10L interaction sterically occludes Gβγ binding to Gαs. After acute stimulation of a Gαs-coupled receptor (β2AR), GD20-F10L functions as a dual-effect G protein PPI inhibitor by sequestering monomeric Gαs and releasing Gβγ from the natural inhibition of Gαs/GDP. As a result, GD20-F10L co-treatment with the β2AR agonist, isoproterenol generates a higher Gβγ concentration, which is comparable to the Gβγ concentration following M2R (a Gαi-coupled receptor) activation (FIGS. 21D-21E). It was hypothesized that the level of free Gβγ upon receptor activation confers receptor signaling specificity (Touhara and MacKinnon, 2018). Therefore, GD20-F10L could potentially provide a novel approach to elucidate or even rewire the downstream signaling of Gαs-coupled receptors via activating Gβγ dependent pathways. Last, rapid Gα Gβγ reassociation terminates canonical GPCR-dependent G protein signaling within seconds (Ghosh et al., 2017). However, the slow dissociating Gαs-GD20-F10L interaction (FIG. 23E and Table 3) offers the opportunity to trap the inactive state Gαs for a longer time and consequently prolong one branch of GPCR signaling the Gβγ heterodimer.

Our demonstration of the use of the RaPID cyclic peptide platform through both positive and negative selection steps provides proof of principle for a path to discovering other cell-permeable, class-specific and state-selective inhibitors of the remainder of the GTPase family. The state-selective Gαs inhibitors GN13 and GD20 also provide novel pharmacological strategies for understanding and modulating GPCR signaling.

GN13 and GD20-F10L are strong binders to Gαs, with K_{D} values in the nanomolar range. However, there is difficulty in measuring potency due to the competing tight protein-protein interactions in cell membranes and the relatively lower cell penetration of cyclic peptides. Optimizing cyclic peptides with non-canonical residues could potentially improve the potency and cell permeability of GN13 and GD20-F10L to overcome this limitation.

### Example 4: Experimental Procedures

### Cell Lines

HeLa cells stably expressing the Halo-Tag-GFP-Mito construct were provided by the Kritzer lab (Peraro et al., 2018). Wild-type HEK293, GNAS KO HEK293 were provided by the Inoue lab. These cells are female in origin. Wild-type HEK293, GNAS KO HEK293 and HeLa cells were cultured at 37° C., 5% CO₂ in DMEM (Thermo Fisher Scientific, Cat #11995073) supplemented with 10% heat-inactivated FBS (AxeniaBiologix).

WT Gαs, all the mutants of Gαs, the C₁ domain (residues 442-658, VC1) of human ADCY5 (adenylyl cyclase V) and the C₂ domain (residues 871-1082, IIC2) of human ADCY2 (adenylyl cyclase II) were overexpressed in Escherichia coli BL21(DE3) cultured in Terrific Broth (TB) Medium. Human GNB1 (Go 1) and GNG2 (Gγ2) were co-expressed in Sf9 insect cells cultured in Sf-900 III SFM medium at 28° C.

### Protein Expression and Purification

Proteins used in the adenylyl cyclase assay, the radioactivity assay, and the steady state GTPase asssy. The wild-type and S275L mutant of Gαs, C2 domain of human ADCY2, C1 domain of human ADCY5, and human Gβ1/Gγ2(C68S) complex used in the adenylyl cyclase activity assay were cloned, expressed and purified as described (Hu and Shokat, 2018).

### Gαs Used in the RaPID Selection

The gene encoding residues 7-380 of the short isoform of human Gαs (GNAS, accession number in PubMed: NP_536351) with an Avi tag and a TEV cleavage site at its N-terminus was cloned into the multiple cloning site 1 of the pETDuet vector. The resulting protein sequence is as follows:

(SEQ ID NO: 5)
MGSSHHHHHHSGMSGLNDIFEAQKIEWHESSGENLYFQGMSKTEDQRNEE
KAQREANKKIEKQLQKDKQVYRATHRLLLLGAGESGKSTIVKQMRILHVN
GFNGDSEKATKVQDIKNNLKEAIETIVAAMSNLVPPVELANPENQFRVDY
ILSVMNVPDFDFPPEFYEHAKALWEDEGVRACYERSNEYQLIDCAQYFLD
KIDVIKQADYVPSDQDLLRCRVLTSGIFETKFQVDKVNFHMFDVGGQRDE
RRKWIQCFNDVTAIIFVVASSSYNMVIREDNQTNRLQEALNLFKSIWNNR
WLRTISVILFLNKQDLLAEKVLAGKSKIEDYFPEFARYTTPEDATPEPGE
DPRVTRAKYFIRDEFLRISTASGDGRHYCYPHFTCAVDTENIRRVFNDCR
DIIQRMHLRQYELL.

In the same pETDuet plasmid, the gene encoding BirA (accession number in PubMed: NP_418404.1) was inserted between NdeI and XhoI sites of the multiple cloning site 2. This plasmid was transformed into Escherichia coli BL21(DE3). The transformed cells were grown in TB medium supplemented with 50 μg/mL carbenicillin at 37° C. until OD600 reached 0.5, and then cooled to 22° C. followed by addition of 40 μM β-D-thiogalactopyranoside. After overnight incubation, 50 μM biotin was added into the culture for 2 hours. The cells were harvested by centrifugation, resuspended in lysis buffer (150 mM NaCl, 25 mM Tris 8.0, 1 mM MgCl₂, 250 μM biotin, protease inhibitor, and then lysed by a microfluidizer. The cell lysate was centrifuged at 14000 g for 1 hour at 4° C. The supernatant was incubated with TALON Resin at 4° C. for 2 hours, then the resin was washed by 500 mM NaCl, 25 mM Tris 8.0, 1 mM MgCl₂ and 5 mM imidazole 8.0. Gαs was eluted by 25 mM Tris 8.0, 1 mM MgCl₂, 250 mM imidazole 8.0, 10% glycerol and 0.1 mM GDP. After adding 5 mM Dithiothreitol (DTT), the eluate was loaded onto a Source-15Q column. Gαs was eluted by a linear gradient from 100% IEC buffer A (25 mM Tris 8.0, 1 mM MgCl₂) to 40% IEC Buffer B (25 mM Tris 8.0, 1 M NaCl, 1 mM MgCl₂). The peak fractions were pooled and supplemented with 5 mM DTT. One half of peak fractions was mixed with equal volume of GppNHp exchange buffer (150 mM NaCl, 25 mM HEPES 8.0, 2 mM EDTA, 2 mM GppNHp, 5 mM DTT) for 2 hours, followed by addition of 5 mM MgCl₂. GppNHp-bound Gαs and GDP-bound Gαs were concentrated and purified by gel filtration (Superdex 200 increase, 10/30) with SEC buffer (150 mM NaCl, 20 mM HEPES 8.0, 5 mM MgCl₂ and 1 mM EDTA-Na 8.0). The peak fractions were pooled and concentrated for biochemical assay.

WT Gαs, Gαs S275L mutant and full-length Gαi used in the TR-FRET assay and the bio-layer interferometry assay. The gene of residues 7-380 of the short isoform of human Gαs (GNAS, accession number in PubMed: NP_536351) with a stop codon at its end was cloned into the NdeI/XhoI site of a modified pET15b vector, in which a Drice cleavage site (AspGluValAsp↓Ala) and an Avi tag were inserted at the N-terminus. The resulting protein sequence after Drice protease cleavage is as follows:

(SEQ ID NO: 6)
AHMGLNDIFEAQKIEWHESKTEDQRNEEKAQREANKKIEKQLQKDKQVYR
ATHRLLLLGAGESGKSTIVKQMRILHVNGFNGDSEKATKVQDIKNNLKEA
IETIVAAMSNLVPPVELANPENQFRVDYILSVMNVPDFDFPPEFYEHAKA
LWEDEGVRACYERSNEYQLIDCAQYFLDKIDVIKQADYVPSDQDLLRCRV
LTSGIFETKFQVDKVNFHMFDVGGQRDERRKWIQCFNDVTAIIFVVASSS
YNMVIREDNQTNRLQEALNLFKSIWNNRWLRTISVILFLNKQDLLAEKVL
AGKSKIEDYFPEFARYTTPEDATPEPGEDPRVTRAKYFIRDEFLRISTAS
GDGRHYCYPHFTCAVDTENIRRVENDCRDIIQRMHLRQYELL.

The AviTagged Gαs S275L mutant plasmid was constructed using quick-change mutagenesis from the AviTagged WT Gαs plasmid. The resulting protein sequence after Drice protease cleavage is as follows:

(SEQ ID NO: 7)
AHMGLNDIFEAQKIEWHESKTEDQRNEEKAQREANKKIEKQLQKDKQVYR
ATHRLLLLGAGESGKSTIVKQMRILHVNGFNGDSEKATKVQDIKNNLKEA
IETIVAAMSNLVPPVELANPENQFRVDYILSVMNVPDFDFPPEFYEHAKA
LWEDEGVRACYERSNEYQLIDCAQYFLDKIDVIKQADYVPSDQDLLRCRV
LTSGIFETKFQVDKVNFHMFDVGGQRDERRKWIQCFNDVTAIIFVVASSS
YNMVIREDNQTNRLQEALNLFKLIWNNRWLRTISVILFLNKQDLLAEKVL
AGKSKIEDYFPEFARYTTPEDATPEPGEDPRVTRAKYFIRDEFLRISTAS
GDGRHYCYPHFTCAVDTENIRRVFNDCRDIIQRMHLRQYELL.

The gene of residues 2-354 of the short isoform of human Gαi1 (GNAI1, accession number in PubMed: NP_002060.4) with a stop codon at its end was cloned into the NdeI/XhoI site of a modified pET15b vector, in which a Drice cleavage site (AspGluValAsp↓Ala) and an Avi tag were inserted at the N-terminus. The resulting protein sequence after Drice protease cleavage is as follows:

(SEQ ID NO: 8)
AHMGLNDIFEAQKIEWHEGCTLSAEDKAAVERSKMIDRNLREDGEKAARE
VKLLLLGAGESGKSTIVKQMKIIHEAGYSEEECKQYKAVVYSNTIQSIIA
IIRAMGRLKIDFGDSARADDARQLFVLAGAAEEGFMTAELAGVIKRLWKD
SGVQACFNRSREYQLNDSAAYYLNDLDRIAQPNYIPTQQDVLRTRVKTTG
IVETHFTFKDLHFKMFDVGGQRSERKKWIHCFEGVTAIIFCVALSDYDLV
LAEDEEMNRMHESMKLFDSICNNKWFTDTSIILFLNKKDLFEEKIKKSPL
TICYPEYAGSNTYEEAAAYIQCQFEDLNKRKDTKEIYTHFTCATDTKNVQ
FVFDAVTDVIIKNNLKDCGLF.

The above-mentioned plasmids were transformed into Escherichia coli BL21(DE3), respectively. The transformed cells were grown in TB medium supplemented with 50 μg/mL carbenicillin at 37° C. until OD600 reached 0.4, and then cooled to 22° C. followed by addition of 100 μM IPTG. After overnight incubation, the cells were harvested by centrifugation, resuspended in lysis buffer (150 mM NaCl, 25 mM Tris 8.0, 1 mM MgCl₂, protease inhibitor cocktail), and then lysed by a microfluidizer. The cell lysate was centrifuged at 14000 g for 1 hour at 4° C. The supernatant was incubated with TALON resin at 4° C. for 1 hour, then the resin was washed by 500 mM NaCl, 25 mM Tris 8.0, 1 mM MgCl₂ and 5 mM imidazole 8.0. G protein was eluted by 25 mM Tris 8.0, 1 mM MgCl₂, 250 mM imidazole 8.0, 10% glycerol and 0.1 mM GDP. After adding 5 mM Dithiothreitol (DTT), the eluate was incubated with Drice protease at 4° C. overnight to remove the hexahistidine tag. Purified BirA (A gift from the Wells lab) and biotin were added at 4° C. until LC-MS showed complete biotinylation. G protein was loaded onto a Source-15Q column and eluted by a linear gradient from 100% IEC buffer A (25 mM Tris 8.0, 1 mM MgCl₂) to 40% IEC Buffer B (25 mM Tris 8.0, 1 M NaCl, 1 mM MgCl₂). The peak fractions were pooled, nucleotide exchanged, and supplemented with 5 mM DTT and 0.1 mM nucleotide, and then concentrated and purified by gel filtration (Superdex 200 increase, 10/30) with SEC buffer (150 mM NaCl, 20 mM HEPES 8.0, 5 mM MgCl₂ and 1 mM EDTA-Na 8.0). The peak fractions were pooled and concentrated for biochemical assay.

### RaPID Selection

Selections were performed with thioether-macrocyclic peptide library against biotinylated Gαs. Thioether-macrocyclic peptide libraries were constructed with N-chloroacetyl-D-tyrosine (ClAc^{D}Tyr) as an initiator by using the flexible in vitro translation (FIT) system (Goto et al., 2011). The mRNA libraries, ClAc^{D}Tyr-tRNA^{fMet}_{CAU} were prepared as reported (Yamagishi et al., 2011). The mRNA library corresponding for the thioether-macrocyclic peptide library was designed to have an AUG initiator codon to incorporate N-chloroacetyl-D-tyrosine (ClAc^{D}Tyr), followed by 8-12 NNK random codons (N=G, C, A or U; K=G or U) to code random proteinogenic amino acids, and then a fixed downstream UGC codon to assign Cys. After in vitro translation, a thioether bond formed spontaneously between the N-terminal ClAc group of the initiator ^{D}Tyr residue and the sulfhydryl group of a downstream Cys residue.

In the first round of selection, the initial cyclic peptide library was formed by adding puromycin ligated mRNA library (225 pmol) to a 150 μL scale flexible in vitro translation system, in the presence of 30 μM of ClAc^{D}Tyr-tRNA^{fMet}_{CAU}. The translation was performed 37° C. for 30 min, followed by an extra incubation at 25° C. for 12 min. After an addition of L of 200 mM EDTA (pH 8.0) solution, the reaction solution was incubated at 37° C. for 30 min to facilitate cyclization. Then the library was reversed transcribed by M-MLV reverse transcriptase at 42° C. for 1 hour and subject to pre-washed Sephadex G-25 columns to remove salts. The desalted solution of peptide-mRNA/cDNA was applied to Gαs (positive selection state)-immobilized Dynabeads M280 streptavidin magnetic beads and rotated at 4° C. for 1 hour in selection buffer (25 mM HEPES pH 7.5, 150 mM NaCl, 1 mM MgCl₂ and 0.05% Tween 20) containing 0.5 mM corresponding nucleotide and 0.1% acetylated BSA. Bead amounts were chosen that the final concentration of Gαs protein was 200 nM. This process is referred to as positive selection. The selected peptide-mRNA/cDNAs were isolated from the beads by incubating in 1×PCR reaction buffer heated at 95° C. for 5 min. The amount of eluted cDNAs was measured by quantitative PCR. The remaining cDNAs were amplified by PCR, purified and transcribed into mRNAs as a library for the next round of selection.

In the subsequent rounds of selection, ligated mRNA from previous round (7.5 pmol) was added to a 5 μL scale reprogrammed in vitro translation system. This was incubated at 37° C. for 30 min and at 25° C. for 12 min. Then 1 μL of 100 mM EDTA (pH 8.0) was added and incubated at 37° C. for 30 min. After reverse transcription and subject to pre-washed Sephadex G-25 columns to remove salts, negative selection was performed by adding the desalted solution of peptide-mRNA/cDNA to Gαs (negative selection state)-immobilized Dynabeads M280 streptavidin magnetic beads and rotated at 4° C. for 30 min in selection buffer containing 0.1% acetylated BSA. This process was repeated several times by removing the supernatant to fresh beads immobilized with Gαs (negative selection state). The supernatant from the last negative selection was then added to beads immobilized with the positive selection state of Gαs (final conc. 200 nM) and rotated at 4° C. for 30 min in selection buffer containing 0.5 mM corresponding nucleotide and 0.1% acetylated BSA. As described in the first round of selection, the cDNA was quantified with qPCR, amplified with PCR, transcribed and ligated to puromycin. The subsequent selection was repeated for several rounds until a significant enrichment of cDNA was observed for positive selection state. The recovered cDNA was then identified by next generation sequencing (Miseq, Illumina).

### Comparison Selection

In comparison selection, ligated mRNA (7.5 pmol) from last round selection was added to a 5 μL scale reprogrammed in vitro translation system. After translation, cyclization, reverse transcription and prewashed with Sephadex G-25 columns, the desalted solution of peptide-mRNA/cDNA library was split equally into three fractions, and perform three paralleled selections with the same amount of blank, GDP-bound Gαs-immobilized or GppNHp-bound Gαs-immobilized Dynabeads M280 streptavidin magnetic beads, individually. For each of the paralleled selections, the beads were rotate at 4° C. for 30 min, washed three times with selection buffer. The remaining cDNAs were then eluted from the beads, quantified by qPCR, followed by Miseq sequencing. Finally, identified sequences from each paralleled selection were compared by normalization of Miseq abundance of the sequence with the qPCR reads of the paralleled selection.

### Bio-Layer Interferometry (BLI)

BLI experiments were performed using an OctetRED384 instrument from ForteBio. All experiments were performed at 25° C. using BLI buffer (10 mM HEPES pH 7.4, 150 mM NaCl, 1 mM MgCl₂, 0.05% Tween-20, 0.1% DMSO, 0.2 mM GppNHp or GDP). Cyclic peptides were diluted to a series of concentrations in BLI buffer plus 10 μM Biotin. Assays were conducted in Greiner 384well, black, flat bottom polypropylene plates containing the protein solutions, BLI buffer plus 10 μM Biotin for dissociation, and serial dilutions of cyclic peptides to be tested.

Biotinylated proteins were immobilized on Streptavidin biosensors by dipping sensors into plate wells containing protein solutions at a concentration of 100-150 nM. Protein loading is around 2-3 nm. Sensors loaded with proteins were moved and dipped into wells with BLI buffer plus 10 μM Biotin to block unlabeled Streptavidin. Association-dissociation cycles of compounds were started by moving and dipping sensors to cyclic peptides dilutions and BLI buffer plus 10 μM Biotin wells alternatively. Association and dissociation times were carefully determined to ensure full association and dissociation.

Raw kinetic data collected were processed with the Data Analysis software provided by the manufacturer using single reference subtraction in which buffer-only reference was subtracted (For GN13 analysis). Because GD20 analogs have a low level of background binding, we used a double reference subtraction (buffer-only reference and non-protein-loading reference) method to calculate their kinetics values. The resulting data were analyzed based on a 1:1 binding model from which kₒₙ and k_{off} values were obtained and then K_{d} values were calculated.

### Adenylyl Cyclase Activity Assay

Cyclic peptides dose dependent inhibition (FIG. 17C).

Cyclic peptides GN1, GN3, GN6, GN7, GN8, GN10, GN11, GN13, GN15 (4 mM stock in DMSO) were diluted to 4× stocks with a series of concentrations (0, 1.56, 3.12, 6.25, 12.5, 25, 50, 100 μM) in reaction buffer (1× PBS 7.4, 0.1% BSA). WT Gαs at a concentration of 8.5 mg/mL (about 190 μM) in 20 mM HEPES 8.0, 150 mM NaCl, 5 mM MgCl₂, 1 mM EDTA-Na 8.0 was diluted to 0.5 μM in dilution buffer (1× PBS 7.4, 0.1% BSA, 1 mM EDTA-Na 8.0, 2 mM DTT, 0.1 mM MgCl₂) plus 1 mM GppNHp. After incubation at room temperature for 1 hour to allow nucleotide exchange, 2.5 μL of Gαs dilution was mixed with 1 μL MgCl₂ stock (20 mM MgCl₂, 1× PBS 7.4, 0.1% BSA) in an OptiPlate-384, White Opaque 384-well Microplate to lock Gαs in GppNHp-bound state. 2 μL of adenylyl cyclase stock (2 μM VC1, 2 nM IIC2, 150 μM FSK, 1× PBS 7.4, 0.1% BSA) was added, followed by addition of 2.5 μL 4× cyclic peptides stock. Reaction mixture was further incubated at room temperature for 2 hours and placed on ice for 5 minutes. cAMP production was initiated by addition of 2 L of ATP stock (1 mM ATP, 1× PBS 7.4, 0.1% BSA). The reaction was carried out at 30° C. for 10 minutes in a PCR machine and stopped by heating at 95° C. for 3 minutes. The cAMP concentrations were measured by the LANCE Ultra cAMP kit.

GN13 inhibition of Gαs proteins at various concentrations (FIG. 18E).

WT Gαs and S275L mutant at a concentration of 8.5 mg/mL (about 190 μM) in 20 mM HEPES 8.0, 150 mM NaCl, 5 mM MgCl₂, 1 mM EDTA-Na 8.0 were diluted to a series of concentrations (4 μM, 1.33 μM, 0.44 μM, 0.15 μM, 49.4 nM, 16.5 nM, 5.5 nM, 0 nM) in dilution buffer (1×PBS 7.4, 0.1% BSA, 1 mM EDTA-Na 8.0, 2 mM DTT, 0.1 mM MgCl₂) plus 1 mM GppNHp. After incubation at room temperature for 1 hour to allow nucleotide exchange, 2.5 μL of each sample was then mixed with 1 μL of MgCl₂ stock (20 mM MgCl₂, 1× PBS 7.4, 0.1% BSA) in an OptiPlate-384, White Opaque 384-well Microplate. 2 μL of adenylyl cyclase/Gβγ stock (2 μM VC1, 2 nM IIC2, 150 μM FSK, 1× PBS 7.4, 0.1% BSA, 10 μM Gβ1/γ2(C68S)) was added, followed by addition of 2.5 μL 25 μM GN13 stock in 1× PBS 7.4, 0.1% BSA. Reaction mixture was further incubated at room temperature for 2 hours and placed on ice for 5 minutes. cAMP production was initiated by addition of 2 μL of ATP stock (1 mM ATP, 1× PBS 7.4, 0.1% BSA). The reaction was carried out at 30° C. for 10 minutes in a PCR machine and stopped by heating at 95° C. for 3 minutes. The cAMP concentrations were measured by the LANCE Ultra cAMP kit.

GN13 inhibition of Gαs proteins in HEK293 cell membranes (FIG. 17F and FIG. 18F).

a. Cell membrane preparation: HEK293cells, GNAS KO HEK293 cells were plated two day before transfection at a density of 1M cells per 10 cm plate. One plate of GNAS KO HEK293 cells was transfected with 4 μg of GNAS WT or GNAS S275L plasmids. After overnight transfection, cells were lifted with TypLE, washed, resuspended in stimulation buffer (1× PBS, protease inhibitor cocktail, 5 mM MgCl₂). Cell membranes were disrupted by using the Dounce homogenizer for 25 strokes. Nuclei and unbroken cells were removed by centrifugation for 5 min at 500 g. The supernatant suspension was carefully removed and centrifuged for 30 min at 45K g. Cell membranes were suspended in stimulation buffer. The protein concentrations were measured using BCA, and were normalized to 750 μg/mL. A final concentration of 0.1% BSA was added into the cell membrane suspension. b: Adenylyl cyclase activity assay in cell membranes: 600 μL of cell membrane suspension was mixed with 60 μL of GTP/GDP stock (stock concentration: 10 mM/1 mM). 5.5 μL of the mixture from last step was mixed with 5.5 μL of GN13 and incubated at room temperature. After 2 hours, membrane/cyclic peptide mixture was transferred on ice for 5 minutes, followed by the addition of 2 μL of IBMX/ISO/ATP or IBMX/DMSO/ATP stock (5 mM IBMX, 0.2 mM ISO or DMSO, 2.5 mM ATP in stimulation buffer with 0.1% BSA). The reaction was carried out at 30° C. for 30 minutes in a PCR machine and stopped by heating at 95° C. for 3 minutes. The cAMP concentrations were measured by the LANCE Ultra cAMP kit.

cAMP concentrations measurement by the LANCE Ultra cAMP kit.

A cAMP standard curve was generated in the same plate using the 50 μM cAMP standard in the kit. Before the measurement, the samples were diluted by stimulation buffer (1× PBS 7.4, 0.1% BSA) to 1/60, 1/120, 1/240 or 1/480 to make sure the cAMP concentrations were in the dynamic range of the cAMP standard curve. 10 μL of each diluted sample was mixed with 5 μL of 4× Eu-cAMP tracer and 5 μL of 4× ULight-anti-cAMP in a white, opaque Optiplate-384 microplate, incubated for 1 hour at room temperature, and the time-resolved fluorescence resonance energy transfer (TR-FRET) signals were read on a Spark 20M plate reader. The cAMP standard curve was fitted by the software GraphPad Prism using the following equation in which “Y” is the TR-FRET signal and “X” is the log of cAMP standard concentration (M):

$$Y = Bottom + ( Top - Bottom ) / ( 1 + 10 \hat{} ( {( Log IC 50 - X )}^{*} HillSlope ) )$$

After obtained the values of the four parameters “Bottom”, “Top”, “LogIC50” and “HillSlope”, we used this equation to convert the TR-FRET signals of the samples into cAMP production values. The cyclic peptides dose dependent inhibition curves were fitted by the following equation to calculate the IC50 of each cyclic peptide:

$$Y = Bottom + ( Top - Bottom ) / ( 1 + 10 \hat{} ( {( Log IC 50 - X )}^{*} HillSlope ) ) ,$$

in which “Y” is the cAMP production value, “X” is the log of cyclic peptide concentration (M).

### FRET Based Gαs/Adenylyl Cyclase Interaction Assay

Cyclic peptides GN13 (4 mM stock in DMSO) were diluted to 5× stocks with a series of concentrations (0, 0.0034, 0.0102, 0.0305, 0.0914, 0.274, 0.823, 2.47, 7.41, 22.2, 66.7, 200 μM) in 1×PBS 7.4, 0.1% BSA, 2 mM DTT, 2 mM MgCl₂. WT Gαs and Gαs S275L mutant at a concentration of 4.6 mg/mL (about 100 μM) in 20 mM HEPES 8.0, 150 mM NaCl, 5 mM MgCl₂ were diluted to 4 μM in EDTA GppNHp buffer (1× PBS 7.4, 0.1% BSA, 2 mM EDTA-Na 8.0, 2 mM DTT, 0.1 mM MgCl₂, 1 mM GppNHp). After incubation at room temperature for 1 hour to allow nucleotide exchange, Gαs dilutions were mixed with equal volume of MgCl₂ stock (3.8 mM MgCl₂, 1× PBS 7.4, 0.1% BSA, 2 mM DTT) to lock Gαs in GppNHp-bound state. GppNHp-bound Gαs proteins were then diluted to 500 nM (5× stocks) in 1×PBS 7.4, 0.1% BSA, 2 mM DTT, 2 mM MgCl₂ plus 0.5 mM GppNHp. In an OptiPlate-384 White Opaque 384-well Microplate, 5× Gαs proteins were mixed with 5× GN13 serial dilution stocks, 5× streptavidin XL665 stock (125 nM), 5× adenylyl cyclase stock (VC1:100 nM, IIC2:200 nM, FSK 0.5 mM) and 5× anti-6His-Tb cryptate stock (0.26 g/mL) in 1×PBS 7.4, 0.1% BSA, 2 mM DTT, 2 mM MgCl₂ for 1 hour at room temperature. The plate was read on a TECAN Spark 20 M plate reader using the TR-FRET mode with the following parameters: Lag time: 70 s, Integration time: 500 s, Read A: Ex 320(25) nm (filter), Em 610(20) nm (filter), Gain 130, Read B: Ex 320(25) nm (filter), Em 665(8) nm (filter), Gain 165. FRET Signal was calculated as the ratio of [Read B]/[Read A].

### Steady-State GTPase Assay

WT Gαs was diluted to 6 μM (4×) in GTPase assay buffer (20 mM HEPES 7.5, 150 mM NaCl, 1 mM MgCl₂). The protein was 1:1 (v:v) diluted with 4× cyclic peptide stock in GTPase assay buffer, and incubated at 37° C. for an hour. The samples were then 1:1 (v:v) diluted with reaction buffer (20 mM HEPES 7.5, 150 mM NaCl, 1 mM MgCl₂, and 1 mM GTP) and incubated at 37° C. After 30, 50, 70, 90 minutes, 50 μL of the sample was removed to measure the inorganic phosphate (Pi) concentration by PiColorLock™ Phosphate Detection kit. A standard curve was made using the 0.1 mM Pi stock in the kit.

### GDP Dissociation Assay

Gα proteins were diluted to 400 nM in the EDTA buffer (20 mM HEPES 7.5, 150 mM NaCl, 1 mM EDTA-Na 8.0, 2 mM DTT). [³H]GDP (1 mCi/mL, 25.2 μM) was added to a final concentration of 1.2 μM, followed by cyclic peptides addition. After incubation at 20° C. for 30 minutes, the same volume of assay buffer (20 μM HEPES-Na 7.5, 150 mM NaCl, 2 mM MgCl₂, and 1 mM GDP) was added to initiate [³H]GDP dissociation. At various points, L of the sample was removed and mixed with 390 μL of ice-cold wash buffer (20 mM HEPES 7.5, 150 mM NaCl, 20 mM MgCl₂). The mixture was immediately filtered through a mixed cellulose membrane (25 mm, 0.22 μm) held by a microanalysis filter holder (EMD Millipore). The membrane was washed by ice-cold wash buffer (500 μL×3), put in a 6-mL plastic vial and air-dried (room temperature 1.5 h). 5 mL of CytoScint-ES Liquid Scintillation Cocktail was added to each vial. After incubation overnight at room temperature, the vial was used for liquid scintillation counting with a LS 6500 Multi-Purpose Scintillation Counter.

The GDP dissociation curves were fitted by the software GraphPad Prism using the following equation to calculate the dissociation rates (k_{off}):

$$Y = Y 0 * exp ( - {k}_{off} * X )$$

in which “Y” is the radioactivity (Counts per minute) of the sample at time “X” (minutes), and Y0 is the calculated radioactivity of the sample at the time point 0.

### GTPγS Binding Assay

Gα proteins were diluted to 10 μM with dilution buffer (20 mM HEPES 7.5, 150 mM NaCl, 1 mM MgCl₂, 2 mM DTT, and 20 μM GDP) and incubated with 5× stocks of cyclic peptides at room temperature for 2 hours. GTPγS binding was initiated by mixing with the reaction buffer at room temperature (50 nM [35S]GTPγS and 100 μM GTPγS in dilution buffer) at room temperature. At various time points, 10 μL of the sample was removed and mixed with 390 μL of ice-cold wash buffer (20 mM HEPES 7.5, 150 mM NaCl, 20 mM MgCl₂). The mixture was filtered through a mixed cellulose membrane (25 mm, 0.22 μm). The membrane was washed by ice-cold wash buffer (500 μL×3), put in a 6-mL plastic vial and air-dried (room temperature 1.5 h). 5 mL of CytoScint-ES Liquid Scintillation Cocktail (MP Biomedicals) was added to each vial. After incubation overnight at room temperature, the vial was used for liquid scintillation counting with a LS 6500 Multi-Purpose Scintillation Counter. A standard curve was generated using [35S]GTPγS. The radioactive activity (Counts per minute) of the samples were converted to the GTPγS concentration. The GTPγS binding curves were fitted by the software GraphPad Prism using the following equation to calculate the apparent GTPγS binding rates (kₐₚₚ):

$$Y = Plateau * ( 1 - exp ( - {k}_{app} * X ) )$$

in which “Y” is the concentration of GTPγS that bound to Gα protein at time “X” (minutes).

### FRET Based Gα/Gβ7 Interaction Assay

Biotinylated avi-Gαs (6-end, WT) and avi-Gαi (FL, WT) were diluted to 32 nM (8×) using assay buffer (1× PBS 7.4, 2 mM DTT, 0.1% BSA, 2 mM MgCl₂, 0.05% Tween plus 0.5 mM GDP), followed by mixing with a same volume of 8× streptavidin XL665 stock (32 nM in the assay buffer). 8× His-Gβ/γ (C68S) stock (16 nM) and 8× anti-6His-Tb cryptate stock (0.4 μg/mL) were added into the Gα/XL665 mixtures. Finally, 2× stocks of cyclic peptides were added with the protein mixtures. After incubation at room temperature for 2 hour at room temperature. The plate was read on a TECAN Spark 20 M plate reader using the TR-FRET mode with the following parameters: Lag time: 70 s, Integration time: 500 s, Read A: Ex 320(25) nm (filter), Em 610(20) nm (filter), Gain 130, Read B: Ex 320(25) nm (filter), Em 665(8) nm (filter), Gain 165. FRET Signal was calculated as the ratio of [Read B]/[Read A].

### Crystallization

GN13/GppNHp/Gαs complex: Wild type Gαs (residues 7-380) that was preloaded with GppNHp and purified by gel filtration was concentrated to 10 mg/mL. The protein was then mixed with 1 mM of GppNHp (50 mM stock in H₂O) and 0.42 mM of the cyclic peptide GN13 (14 mM stock in DMSO). For crystallization, 0.2 μL of the protein sample was mixed with 0.2 μL of the well buffer containing 0.1 M HEPES 7.2, 20% PEG4000, 10% v/v 2-propanol. Crystals were grown at 20° C. in a 96-well plate using the hanging-drop vapour-diffusion method, transferred to a cryoprotectant solution (0.1 M HEPES 7.2, 20% PEG4000, 10% v/v 2-propanol, 150 mM NaCl, 20 mM HEPES 8.0, 5 mM MgCl₂, 1 mM GppNHp, 25% v/v glycerol), and flash-frozen in liquid nitrogen.

GD20/GDP/Gαs complex: Wild type Gαs (NCBI Reference Sequence: NP_536351.1, residues 35-380) was preloaded with GDP, purified by gel filtration and then concentrated to 11.6 mg/mL. Before crystallization, the protein was mixed with 5 mM of Dithiothreitol (0.5 M stock in H₂O), 1 mM of GDP (50 mM stock in H₂O) and 0.76 mM of the cyclic peptide GD20 (42.6 mM stock in DMSO). For crystallization, 1.5 μL of the protein sample was mixed with 1.5 μL of the well buffer containing 0.1 M Tris 8.2, 26% PEG4000, 0.8 M LiCl. Crystals were grown at 20° C. in a 15-well plate using the hanging-drop vapour-diffusion method, and flash-frozen in liquid nitrogen.

### Data Collection and Structure Determination

The data set was collected at the Advanced Light Source beamline 8.2.1 with X-ray at a wavelength of 0.999965 Å. Then the data set was integrated using the HKL2000 package (Otwinowski and Minor, 1997), scaled with Scala (Evans., 2006) and solved by molecular replacement using Phaser (McCoy et al., 2007) in CCP4 software suite (Winn et al., 2011). The crystal structure of GDP-bound human Gαs R201C/C237 mutant (PDB code: 6AU6) was used as the initial model. The structure was manually refined with Coot (Emsley et al., 2010) and PHENIX (Adams et al., 2010). Data collection and refinement statistics are shown in Table 1 and Table 2.

### Chloroalkane Penetration Assay (CAPA)

The cell lines used for CAPA were HeLa cell lines, generated by Chenoweth and co-workers, that stably express HaloTag exclusively in the cytosol (Peraro et al., 2018). Cells were seeded in a 96-well plate the day before the experiment at a density of 4×10⁴ cells per well. The day of the experiment the media was aspirated, and 100 μL of cyclic peptide dilutions in DMEM were added to the cells. Plate was incubated for 19.5 h at 37° C. with 5% CO₂. The contents of the wells were aspirated off, and wells were washed using fresh Opti-MEM for 15 min. The wash was aspirated off, and the cells were chased using 5 μM ct-TAMRA for 15 min, except for the No-ct-TAMRA control wells, which were incubated with Opti-MEM alone. The contents of the wells were aspirated and washed with fresh Opti-MEM for 30 min. After aspiration, cells were rinsed once with phosphate-buffered saline (PBS). The cells were then trypsinized, quenched with DMEM, resuspended in PBS, and analyzed using a benchtop flow cytometer (CytoFLEX, Beckman).

### BRET2 Based Gα Gβγ Interaction Assay

The plasmids encoding M2R was a gift from Dr. Roderick Mackinnon. The plasmids encoding Gα-RLuc8, Gβ1, and Gγ1-GFP2 were gifts from Dr. Bryan Roth. The plasmid encoding Gγ2-GFP2 was generated by replacing the Gγ1 sequence of pcDNA3.1-GGammal-GFP2 by digestion with BamHI/XbaI and subsequent insertion of the Gγ2 sequence (MASNNTASIAQARKLVEQLKMEANIDRIKVSKAAADLMAYCEAHAKEDPLLTPVP ASENPFREKKFFCAIL (SEQ ID NO: 9)). All plasmids were sequenced to ensure their identities.

The BRET2 assay was conducted as reported (Olsen et al., 2020). Cells were plated in 10 cm dishes at 3 million cells per dish the night before transfection. Cells were transfected using a 6:6:3:1 DNA ratio of receptor:Gα-RLuc8:Gβ:Gγ-GFP2 (750:750:375:125 ng for 10 cm dishes). Transit 2020 was used to complex the DNA at a ratio of 3 μL Transit per μg DNA, in OptiMEM (Gibco-ThermoFisher) at a concentration of 10 ng DNA per μl OptiMEM. 16 hours after transfection, cells were harvested from the plate using TrypLE and plated in poly-D-lysine-coated white, clear-bottom 96-well assay plates (Greiner Bio-One) at a density of 30,000 cells per well.

8 hours after plating in 96-well assay plates, media was replaced with 100 μL of cyclic peptide dilutions in DMEM with 1% dialyzed FBS. 16 hours after drug treatment at 37° C. with 5% CO₂, white backings (PerkinElmer) were applied to the plate bottoms, and growth medium was carefully aspirated and replaced immediately with 60 μL of drug dilutions in assay buffer (1×Hank's balanced salt solution (HBSS)+20 mM HEPES, pH 7.4), followed by a 10 μl addition of freshly prepared 50 μM coelenterazine 400a. After a 5 min equilibration period, cells were treated with 30 μL of GPCR agonist or DMSO dilutions in assay buffer for an additional 5 min. Plates were then read in a TECAN Spark 20 M plate reader with 395 nm (RLuc8-coelenterazine 400a) and 510 nm (GFP2) emission filters, at integration times of 1 s per well. Plates were read serially six times, and measurements from the fourth read were used in all analyses. BRET2 ratios were computed as the ratio of the GFP2 emission to RLuc8 emission.

Chemical Stability Assay in DMEM with 10% FBS or Human Plasma

These assays were conducted by Pharmaron Beijing CO., Ltd. Cyclic peptides working solutions were prepared at 10 μM in DMEM with 10% FBS (Avantor, Cat #76294-180) or human plasma (Pooled, Male & Female, BioIVT, Cat #HMN666664). The assays were performed in duplicate. Vials were incubated at 37° C. at 60 rpm in a water bath and taken at designated time points including 0, 480, 1080 and 1440 min. For each time point, the initiation of the reaction was staggered so all the time points were terminated with cold acetonitrile containing internal standards (IS, 100 nM alprazolam, 200 nM labetalol, 200 nM Imipramine and 2 μM ketoplofen) at the same time. Samples were vortexed then centrifuged at 4° C. to remove proteins. The supernatants from centrifugation were diluted by ultra-pure H₂O and used for LC-MS/MS analysis. All calculations were carried out using GraphPad Prism. Remaining percentages of parent compounds at each time point were estimated by determining the peak area ratios from extractedion chromatograms.

### Chemical Synthesis

### Solid Phase Synthesis of Cyclic Peptides

Macrocyclic peptides (25 μmol scale) were synthesized by a standard Fmoc solid phase peptide synthesis method using a Syro Wave automated peptide synthesizer (Biotage) (Morimoto et al., 2012). After addition of a chloroacetyl group onto the N-terminal amide group (for the formation of cyclic peptide), peptides were cleaved from the NovaPEG Rink Amide resin (Novabiochem) by a solution of 92.5% trifluoroacetic acid (TFA), 2.5% 3,6-Dioxa-1,8-octanedithiol ethanedithiol (DODT), 2.5% triisopropylsilane (TIPS) and 2.5% water and precipitated by diethyl ether. To conduct the macrocyclization reaction, the peptide pellet was dissolved in 10 ml DMSO containing 10 mM tris(2-carboxyethyl)phosphine hydrochloride (TCEP), adjusted to pH>8 by addition of triethylamine (TEA) and incubated at 25° C. for 1 hour. This cyclization reaction was quenched by acidification of the solution with TFA. The crude products were purified by reverse-phase HPLC (RP-HPLC) (Shimadzu) with a Chromolith RP-18 100-25 prep column. Molecular masses were verified by a time-of-flight mass spectrometer (Waters Xevo G2-XS), and the purity was verified by analytical HPLC on a Waters Acquity UPLC BEH C18 1.7 μm column.

### General Synthesis Route of Chloroalkane Tagged Cyclic Peptides

In this work, we prepared a chloroalkane tag (ct) that has been previously used with the HaloTag system (Neklesa et al., 2011). Instead of using the Rink amide resin, peptides were synthesized using the Fmoc-Wang resin (Anaspec, AS-20058) to generate a carboxylate at the C-terminus. To cap the C-terminus with the chloroalkane tag (ct), 10 equiv of chloroalkane tag (ct), 5 equiv of HATU, and 20 equiv of DIPEA were dissolved in DMF and stirred for 1 hour at room temperature. Crude peptides were purified by reverse-phase HPLC (Waters XBridge C18 column 5 m particle size 30×250 mm, 5-95% acetonitrile-water+0.1% formic acid, 40 min, 20 mL/min) to afford the chloroalkane tagged peptides.

### Characterization Data for Cyclic Peptides

### Mass Spectrometry

GN13: HRMS (ESI): Calcd for (C₇₉H₁₀₆N₁₆O₂₁S+2H)²⁺:824.3798, Found: 824.3973.

GD20: HRMS (ESI): Calcd for (C90H126N22O₂₀S+2H)²⁺:934.4698, Found: 934.4844.

GD20-F10L: HRMS (ESI): Calcd for (C₈₇H₁₂₈N₂₂O₂₀S+2H)²⁺:917.4776, Found: 917.4901.

GD20-F5A: HRMS (ESI): Calcd for (C₈₄H₁₂₈N₂₂O₂₀S+2H)²⁺:896.4542, Found: 896.4604.

GD20-F10L/F5A: HRMS (ESI): Calcd for (C₈₁H₁₂₄N₂₂O2₀S+2H)²⁺:879.4620, Found: 879.4648.

ct-GN13-E3Q: HRMS (ESI): Calcd for (C₈₉H₁₂₆ClN₁₇O₂₂S+2H)²⁺:926.9416, Found: 926.9422.

ct-GD20: HRMS (ESI): Calcd for (C₁₀₀H₁₄₅ClN₂₂O₂₂S+2H)²⁺:1037.5235, Found: 1037.5303.

ct-GD20-F10L: HRMS (ESI): Calcd for (C₉₇H₁₄₇ClN₂₂O₂₂S+2H)²⁺:1020.5313, Found: 1020.5193.

Absorbance was recorded at 280 nm.

### Quantification and Statistical Analysis

All of the curves in Figures except those from the BLI experiments were fitted by GraphPad Prism. Raw kinetic data collected from the BLI experiments were processed with the Data Analysis software provided by the manufacturer. All the details can be found in the figure legends and in the Method Details. The data collection and refinement statistics of the crystal structures can be found in Table 1 and Table 2 (related to FIGS. 18A-18F, FIGS. 20A-20G, and FIGS. 22A-22K).

TABLE 1
Data collection and refinement statistics for the
Gαs/GppNHp/GN13 complex (related to FIGS. 18A-18F).

| | Gαs/GppNHp/GN13 complex |
|---|---|
| Data collection | |
| Space group | P 21 21 21 |
| Cell dimensions | |
| a, b, c (Å) | 68.905, 78.332, 80.043 |
| α, β, γ (°) | 90, 90, 90 |
| Resolution (Å) | 50.00-1.57 (1.60-1.57)^{a} |
| R_{merge}, Rₘₑₐₛ, and Rₚᵢₘ | 0.074 (1.040), 0.086 (0.972), 0.023 (0.405) |
| I/σ(I) | 27.3 (1.32) |
| CC_{1/2} | 0.997 (0.783) |
| Completeness (%) | 96.9 (71.2) |
| Total reflections | 58703 |
| Unique reflections | 57627 |
| Redundancy | 12.4 (4.0) |
| Refinement | |
| Resolution (Å) | 43.45-1.574 |
| No. reflections | 51161 |
| R_{work} | 0.1970 |
| R_{free} | 0.2291 |
| No. atoms | |
| Protein | 3079 |
| Ligand/ion (specify/describe) | 44 |
| Water | 132 |
| B factors | |
| Protein | 21.98 |
| Ligand/ion | 13.41 |
| Water | 22.43 |
| R.m.s. deviations | |
| Bond lengths (Å) | 0.014 |
| Bond angles (°) | 1.39 |
| Ramachandran analysis | |
| Favored (%) | 98.91 |
| Allowed (%) | 0.82 |
| Outliers (%) | 0.27 |
| Rotamer outliers (%) | 0.60 |
| Clashscore | 3.57 |

^{a}Values in parentheses are for highest-resolution shell.

TABLE 2
Data collection and refinement statistics for the
Gαs/GDP/GD20 complex (related to FIGS. 20A-20G).

| | Gαs/GDP/GD20 complex |
|---|---|
| Data collection | |
| Space group | P1 |
| Cell dimensions | |
| a, b, c (Å) | 58.105, 81.771, 76.912 |
| α, β, γ (°) | 81.266, 83.844, 90.698 |
| Resolution (Å) | 50.00-1.95 (1.98-1.95)^{a} |
| R_{merge}, Rₘₑₐₛ, and Rₚᵢₘ | 0.081 (1.202), 0.113 (1.260), 0.059 (0.762) |
| I/σ(I) | 11.40 (0.815) |
| CC_{1/2} | 0.991 (0.422) |
| Completeness (%) | 97.0 (87.7) |
| Total reflections | 99153 |
| Unique reflections | 95974 |
| Redundancy | 3.4 (2.0) |
| Refinement | |
| Resolution (Å) | 48.54-1.95 |
| No. reflections | 82224 |
| R_{work} | 0.2178 |
| R_{free} | 0.2580 |
| No. atoms | |
| Protein | 11279 |
| Ligand/ion | 132 |
| Water | 329 |
| B factors | |
| Protein | 29.36 |
| Ligand/ion | 18.22 |
| Water | 21.27 |
| R.m.s. deviations | |
| Bond lengths (Å) | 0.004 |
| Bond angles (°) | 0.70 |
| Ramachandran analysis | |
| Favored (%) | 97.45 |
| Allowed (%) | 2.25 |
| Outliers (%) | 0.30 |
| Rotamer outliers (%) | 0.98 |
| Clashscore | 3.86 |

^{a}Values in parentheses are for highest-resolution shell.

TABLE 3
Kinetics analysis of cyclic peptides-Gαs interaction by BLI
(related to FIG. 17C, FIG. 19B, FIGS. 22A-22K, and FIGS. 23A-23J).

| | K_{D} (nM) | Kₒₙ (M⁻¹s⁻¹) | K_{off} (s⁻¹) |
|---|---|---|---|
| GN13/GppNHp/Gαs | 190 ± 16 | 2.71 × 10⁵ ± 2.13 × 10⁴ | 5.13 × 10⁻² ± 1.31 × 10⁻³ |
| GD20/GDP/Gαs | 31.4 ± 0.7 | 1.09 × 10⁵ ± 1.28 × 10³ | 3.43 × 10⁻³ ± 6.97 × 10⁻⁵ |
| GD20-F10L/GDP/Gαs | 14.5 ± 0.4 | 2.47 × 10⁵ ± 3.71 × 10³ | 3.58 × 10⁻³ ± 8.50 × 10⁻⁵ |

TABLE 4
Chemical stability of Gαs binding cyclic peptides
in DMEM with 10% FBS (related to STAR Methods).

| | Half-life (hour) ^{a} |
|---|---|
| GN13 | >76 |
| GD20 | >142 |
| GD20-F10L | 37.37-50.32 |

^{a} Values represent 95% confidence intervals. The data were analyzed from two independent replicates.

TABLE 5
Plasma stability of Gαs binding cyclic
peptides (related to STAR Methods).

| | Half-life (hour) ^{a} |
|---|---|
| GN13 | >82 |
| GD20 | 16.37-27.13 |
| GD20-F10L | 7.16-12.74 |

^{a} Values represent 95% confidence intervals. The data were analyzed from two independent replicates.

### REFERENCES

1. Adams, P. D., Afonine, P. v., Bunkóczi, G., Chen, V. B., Davis, I. W., Echols, N., Headd, J. J., Hung, L. W., Kapral, G. J., Grosse-Kunstleve, R. W., et al. (2010). Acta Crystallogr. D Biol. Crystallogr. 66, 213-221. 2. Alessi, D. R., and Sammler, E. (2018). Science 360, 36-37. 3. Austin, R. J., Ja, W. W., and Roberts, R. W. (2008). J. Mol. Biol. 377, 1406-1418. 4. Bonacci, T. M., Mathews, J. L., Yuan, C., Lehmann, D. M., Malik, S., Wu, D., Font, J. L., Bidlack, J. M. and Smrcka, A. V., 2006. Science 312, 443-446. 5. Canon, J., Rex, K., Saiki, A. Y., Mohr, C., Cooke, K., Bagal, D., Gaida, K., Holt, T., Knutson, C. G., Koppada, N., et al. (2019). Nature 575, 217-223. 6. Chen, Z., Singer, W. D., Sternweis, P. C., and Sprang, S. R. (2005). Nat. Struct. Mol. Biol. 12, 191-197. 7. Dougherty, P. G., Sahni, A., and Pei, D. (2019). Chem. Rev. 119, 10241-10287. 8. Dror, R. O., Mildorf, T. J., Hilger, D., Manglik, A., Borhani, D. W., Arlow, D. H., Philippsen, A., Villanueva, N., Yang, Z., Lerch, M. T., et al. (2015). Science 348, 1361-1365. 9. Emsley, P., Lohkamp, B., Scott, W. G., and Cowtan, K. (2010). Acta Crystallogr. D Biol. Crystallogr. 66, 486-501. 10. Evans, P. (2006). Scaling and assessment of data quality. In Acta Crystallogr. D Biol. Crystallogr., pp. 72-82. 11. Ghosh, P., Rangamani, P., and Kufareva, I. (2017). Cell Cycle 16, 607-612. 12. Gilman, A. G. (1987). Annu Rev Biochem. 56, 615-649. 13. Goto, Y., Katoh, T., and Suga, H. (2011). Nat Protoc. 6, 779-790. 14. Gulati, S., Jin, H., Masuho, I., Orban, T., Cai, Y., Pardon, E., Martemyanov, K. A., Kiser, P. D., Stewart, P. L., Ford, C. P. and Steyaert, J., (2018). Nat. Commun. 9, 1-15. 15. Hallin, J., Engstrom, L. D., Hargi, L., Calinisan, A., Aranda, R., Briere, D. M., Sudhakar, N., Bowcut, V., Baer, B R., Ballard, J. A., et al. (2020). Cancer Discov. 10, 54-71. 16. Hu, Q., and Shokat, K. M. (2018). Cell 173, 1254-1264.el 1. 17. Ja, W. W., and Roberts, R. W. (2004). Biochemistry 43, 9265-9275. 18. Ja, W. W., Wiser, O., Austin, R. J., Jan, L. Y., and Roberts, R. W. (2006). ACS Chem. Biol. 1, 570-574. 19. Johnston, C. A., Willard, F. S., Jezyk, M. R., Fredericks, Z., Bodor, E. T., Jones, M. B., Blaesius, R., Watts, V. J., Harden, T. K., Sondek, J., et al. (2005). Structure 13, 1069-1080. 20. Johnston, C. A., Ramer, J. K., Blaesius, R., Fredericks, Z., Watts, V. J., and Siderovski, D. P. (2005). FEBS Letters. 579, 5746-5750. 21. Johnston, C. A., Lobanova, E. S., Shavkunov, A. S., Low, J., Ramer, J. K., Blaesius, R., Fredericks, Z., Willard, F. S., Kuhlman, B., Arshavsky, V. Y. and Siderovski, D. P., (2006). Biochemistry 45, 11390-11400. 22. Kaur, H., Harris, P. W. R., Little, P. J., and Brimble, M. A. (2015). Org. Lett. 17, 492-495. 23. Lambright, D. G., Noel, J. P., Hammt Be, H. E., and Sigler, P. B. (1994). Nature 369, 621-628. 24. Liu, X., Xu, X., Hilger, D., Aschauer, P., Tiemann, J. K., Du, Y., Liu, H., Hirata, K., Sun, X., Guixà-González, R. and Mathiesen, J. M., (2019). Cell 177, 1243-1251. 25. Maziarz, M., Park, J. C., Leyme, A., Marivin, A., Garcia-Lopez, A., Patel, P. P., and Garcia-Marcos, M. (2020). Cell 182, 770-785.e16. 26. McCoy, A. J., Grosse-Kunstleve, R. W., Adams, P. D., Winn, M. D., Storoni, L. C., and Read, R. J. (2007). J Appl Crystallogr. 40, 658-674. 27. Morimoto, J., Hayashi, Y., and Suga, H. (2012). Angew Chem Int Ed Engl. 51, 3423-3427. 28. Murakami, H., Saito, H., and Suga, H. (2003). Chem Biol. 10, 655-662. 29. Murakami, H., Ohta, A., Ashigai, H., and Suga, H. (2006). Nat Methods. 3, 357-359. 30. Neklesa, T. K., Tae, H. S., Schneekloth, A. R., Stulberg, M. J., Corson, T. W., Sundberg, T. B., Raina, K., Holley, S. A., and Crews, C. M. (2011). Nat. Chem. Biol. 7, 538-543. 31. Nishimura, A., Kitano, K., Takasaki, J., Taniguchi, M., Mizuno, N., Tago, K., Hakoshima, T., Itoh, H., and Gilman, A. G. (2010). Proc. Natl. Acad. Sci. U.S.A. 107, 13666-13671. 32. O'Hayre, M., Vázquez-Prado, J., Kufareva, I., Stawiski, E. W., Handel, T. M., Seshagiri, S., and Gutkind, J. S. (2013). Nat. Rev. Cancer. 13, 412-424. 33. Olsen, R. H. J., DiBerto, J. F., English, J. G., Glaudin, A. M., Krumm, B. E., Slocum, S. T., Che, T., Gavin, A. C., McCorvy, J. D., Roth, B. L., et al. (2020). Nat. Chem. Biol. 16, 841-849. 34. Otwinowski, Z., and Minor, W. (1997). [20] Processing of X-ray diffraction data collected in oscillation mode. In Methods in enzymology (Vol. 276, pp. 307-326). Academic press. 35. Passioura, T., and Suga, H. (2017). Chem. Commun. (Camb.). 53, 1931-1940. 36. Peraro, L., Deprey, K. L., Moser, M. K., Zou, Z., Ball, H. L., Levine, B., and Kritzer, J. A. (2018). J. Am. Chem. Soc. 140, 11360-11369. 37. Prior, I. A., Lewis, P. D., and Mattos, C. (2012). Cancer Res. 72, 2457-2467. 38. Ramaswamy, K., Saito, H., Murakami, H., Shiba, K., and Suga, H. (2004). J Am Chem Soc. 126, 11454-11455. 39. Slep, K. C., Kercher, M. A., Hek, W., Cowan, C. W., Wenselk, T. G., and Sigler, P. B. (2001). Nature 409, 1071-1077. 40. Sohrabi, C., Foster, A., and Tavassoli, A. (2020). Nat. Rev. Chem. 4, 90-101. 41. Stallaert, W., van der Westhuizen, E. T., Schönegge, A. M., Plouffe, B., Hogue, M., Lukashova, V., Inoue, A., Ishida, S., Aoki, J., le Gouill, C., et al. (2017). Mol. Pharmacol. 91, 533-544. 42. Sunahara, R. K., Tesmer, J. J., Gilman, A. G. and Sprang, S. R., (1997). Science 278, 1943-1947. 43. Takasaki, J., Saito, T., Taniguchi, M., Kawasaki, T., Moritani, Y., Hayashi, K., and Kobori, M. (2004). J. Biol. Chem. 279, 47438-47445. 44. Tesmer, J. J. G., Sunahara, R. K., Gilman, A. G., and Sprang, S. R. (1997). Science 278, 1907-1916. 45. Tesmer, V. M., Kawano, T., Shankaranarayanan, A., Kozasa, T. and Tesmer, J. J., (2005). Science 310, 1686-1690. 46. Touhara, K. K. and MacKinnon, R., (2018). Elife 7, e42908. 47. Wall, M. A., Coleman, D. E., Lee, E., IAiguez-Lluhi, J. A., Posner, B. A., Gilman, A. G., and Ft Sprang, S. (1995). Cell 83, 1047-1058. 48. Weis, W. I., and Kobilka, B. K. (2018). Annu. Rev. Biochem. 87, 897-919. 49. Wilson, G. R., Sim, J. C. H., McLean, C., Giannandrea, M., Galea, C. A., Riseley, J. R., Stephenson, S. E. M., Fitzpatrick, E., Haas, S. A., Pope, K., et al. (2014). Am. J. Hum. Genet. 95, 729-735. 50. Winn, M. D., Ballard, C. C., Cowtan, K. D., Dodson, E. J., Emsley, P., Evans, P. R., Keegan, R. M., Krissinel, E. B., Leslie, A. G. W., McCoy, A., et al. (2011). Acta Crystallogr. D Biol. Crystallogr. 67, 235-242. 51. Xiao, H., Murakami, H., Suga, H., and Ferré-D'Amaré, A. R. (2008). Nature 454, 358-361. 52. Xiong, X. F., Zhang, H., Underwood, C. R., Harpsøe, K., Gardella, T. J., Wöldike, M. F., Mannstadt, M., Gloriam, D. E., Bräuner-Osborne, H., and Stromgaard, K. (2016). Nat Chem. 8, 1035-1041. 53. Yamagishi, Y., Shoji, I., Miyagawa, S., Kawakami, T., Katoh, T., Goto, Y., and Suga, H. (2011). Chem Biol. 18, 1562-1570. 54. Zachary, I., Masters, S. B. and Bourne, H. R., (1990). Biochem. Biophys. Res. Commun. 168, 1184-1193. 55. Zhang, H., Xiong, X. F., Boesgaard, M. W., Underwood, C. R., Bräuner-Osborne, H., and Strømgaard, K. (2017). ChemMedChem 12, 830-834.

1. A compound having the formula: 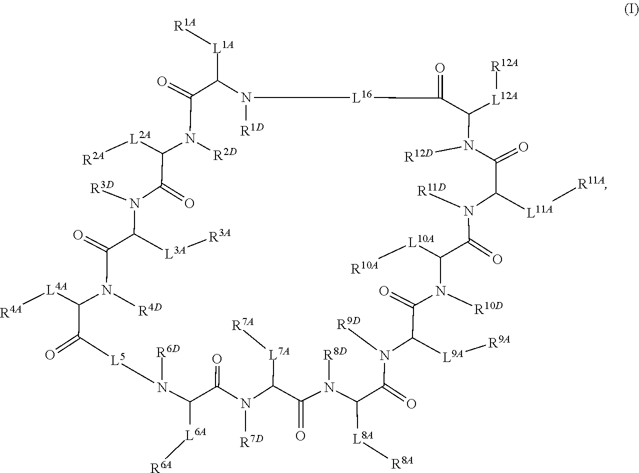
or a pharmaceutically acceptable salt thereof,
wherein
L^{1A}, L^{2A}, L^{3A}, L^{4A}, L^{5A}, L^{6A}, L^{7A}, L^{8A}, L^{9A}, L^{10A}, L^{11A} and L^{12A} are independently a bond, substituted or unsubstituted alkylene, or substituted or unsubstituted heteroalkylene;
L⁵ is 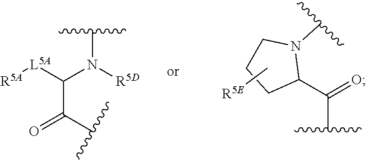
R^{11A} is substituted or unsubstituted aryl;
R^{2A} and R^{5A} are independently hydrogen, —OH, —NH₂, substituted or unsubstituted alkyl, substituted or unsubstituted cycloalkyl, substituted or unsubstituted aryl, or substituted or unsubstituted heteroaryl;
R^{3A}, R^{4A}, and R^{11A} are independently hydrogen, —OH, —NH₂, —COOH, —CONH₂, —NO₂, —SH, —SO₃H, —OSO₃H, —SO₂NH₂, —NINI₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R^{6A} is —NH₂, —CONH₂, substituted or unsubstituted alkyl, substituted or unsubstituted heteroalkyl, or substituted or unsubstituted aryl;
R^{7A}, R^{8A}, and R^{12A} are independently hydrogen, substituted or unsubstituted alkyl, substituted or unsubstituted cycloalkyl, substituted or unsubstituted aryl, or substituted or unsubstituted heteroaryl;
R^{9A} is substituted or unsubstituted cycloalkyl, substituted or unsubstituted heterocycloalkyl, substituted or unsubstituted aryl, or substituted or unsubstituted heteroaryl;
R^{10A} is hydrogen or substituted or unsubstituted alkyl;
R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₈ alkyl;
R^{5E} is hydrogen, —OH, —NH₂, substituted or unsubstituted alkyl, or substituted or unsubstituted heteroalkyl; and
L¹⁶ is a covalent linker.

2-6. (canceled)

7. The compound of claim 1, wherein -L^{1A}-R^{1A}-L^{2A}-R^{2A}-L^{3A}-R^{3A}-L^{4A}-R^{4A}-L^{5A}-R^{5A}-L^{6A}-R^{6A}-L^{7A},-R^{7A}, L^{8A}-R^{8A}-L^{9A}-R^{9A}-L^{10A}-R^{10A}-L^{11A}-R^{11A}, or -L^{12A}-R^{12A} are independently a natural amino acid side chain or an unnatural amino acid side chain.

8. (canceled)

9. The compound of claim 1, having the formula:

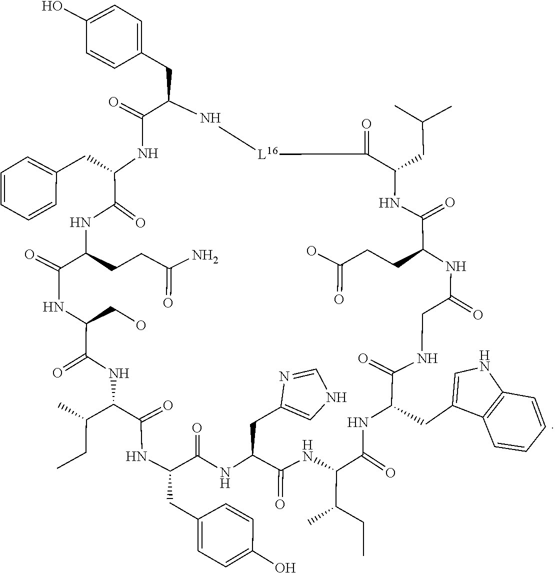

10. (canceled)

11. (canceled)

12. The compound of claim 1, wherein
L¹⁶ is -L^{16A}-L^{16B}-L^{16C}-L^{16D}-L^{16E}-L^{16F}-; and
L^{16A}, L^{16B}, L^{16C}, L^{16D}, L^{16E}, and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

13. (canceled)

14. The compound of claim 1, wherein L¹⁶ is a bond, 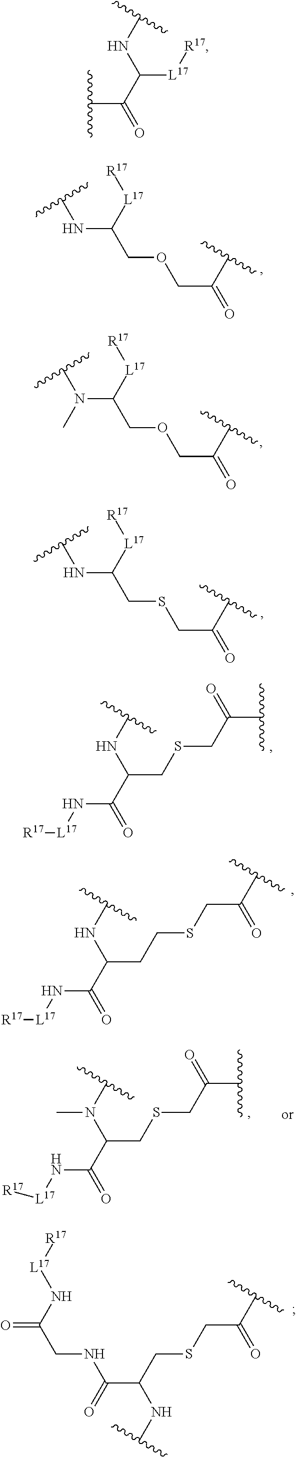
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

15. (canceled)

16. The compound of claim 1, having the formula: 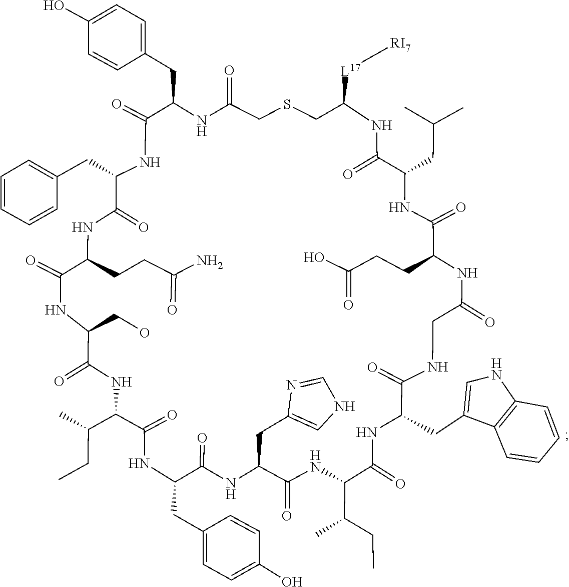
wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

17. The compound of claim 1, having the formula:

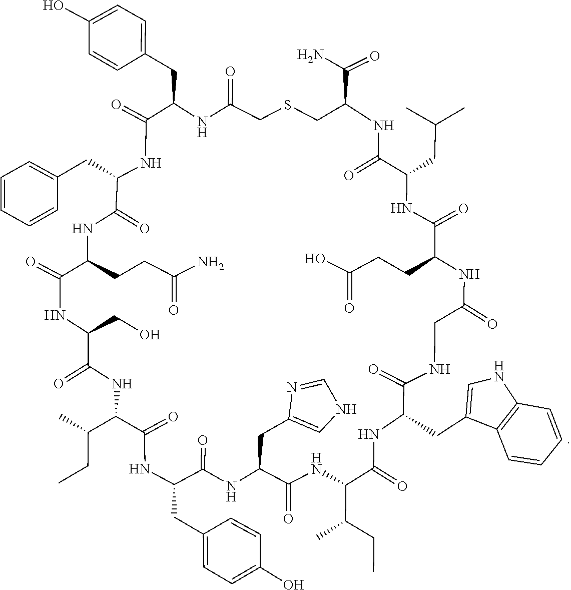 [SMILES: CC[C@H](C)[C@@H]1NC(=O)[C@H](Cc2c[nH]cn2)NC(=O)[C@H](Cc2ccc(O)cc2)NC(=O)[C@H]([C@@H](C)CC)NC(=O)[C@H](CO)NC(=O)[C@H](CCC(N)=O)NC(=O)[C@H](Cc2ccccc2)NC(=O)[C@@H](Cc2ccc(O)cc2)NC(=O)CSC[C@@H](C(N)=O)NC(=O)[C@H](CC(C)C)NC(=O)[C@H](CCC(=O)O)NC(=O)CNC(=O)[C@H](Cc2c[nH]c3ccccc23)NC1=O]

18.-24. (canceled)

25. A compound having the formula: 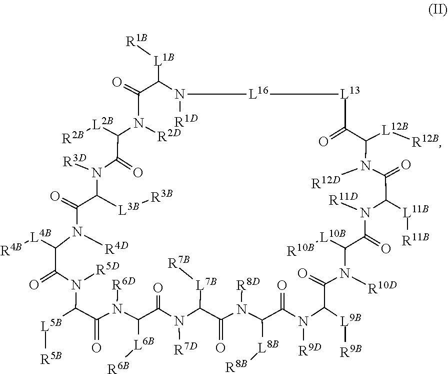
or a pharmaceutically acceptable salt thereof,
wherein
L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene;
L¹³ is a bond, 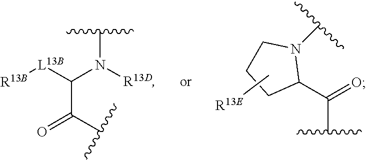
R^{1B} is substituted or unsubstituted aryl;
R^{2B}, R^{4B}, R^{5B}, R^{8B}, R^{9B}, and R^{13B} are independently hydrogen, —OH, —NH₂, —C(O)OH, —C(O)NH₂, —NO₂, —SO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3B} is hydrogen, —OH, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{6B}, R^{7B}, R^{10B}, R^{11B}, and R^{12B} are independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1D}, R^{2D}, R^{3D}, R^{4D}, R^{5D}, R^{6D}, R^{7D}, R^{8D}, R^{9D}, R^{10D}, R^{11D}, R^{12D}, and R^{13D} are independently hydrogen or unsubstituted C₁-C₈ alkyl;
R^{13E} is hydrogen, —OH, —NH₂, substituted or unsubstituted alkyl, or substituted or unsubstituted heteroalkyl; and
L¹⁶ is a covalent linker.

26.-29. (canceled)

30. The compound of claim 25, having the formula:

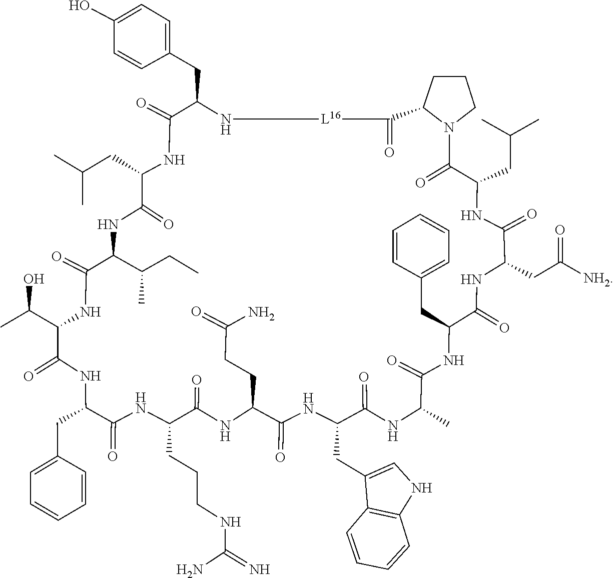

31. The compound of claim 25, having the formula:

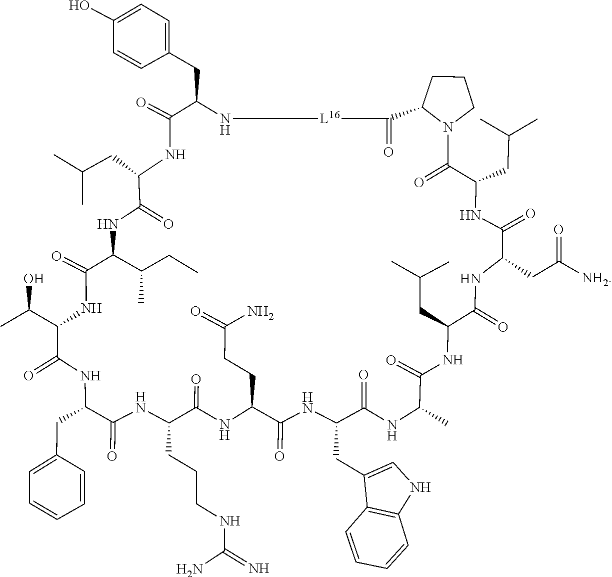

32.-38. (canceled)

39. The compound of claim 25, having the formula:

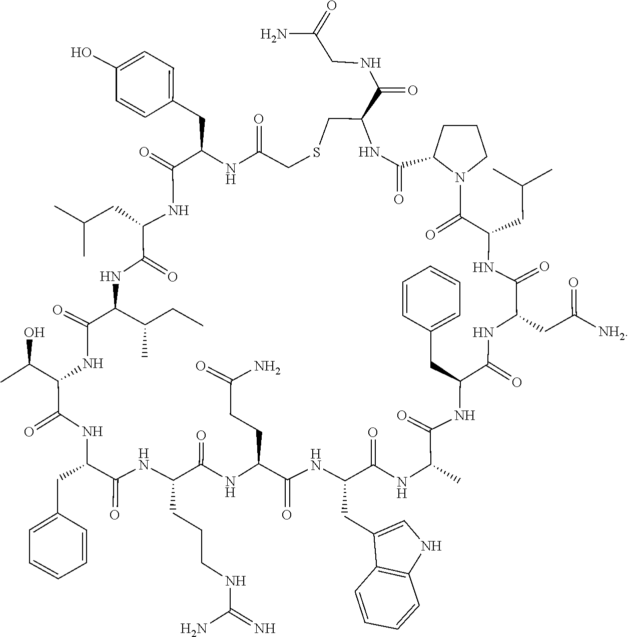 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](Cc2ccccc2)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

40. (canceled)

41. The compound of claim 25, having the formula:

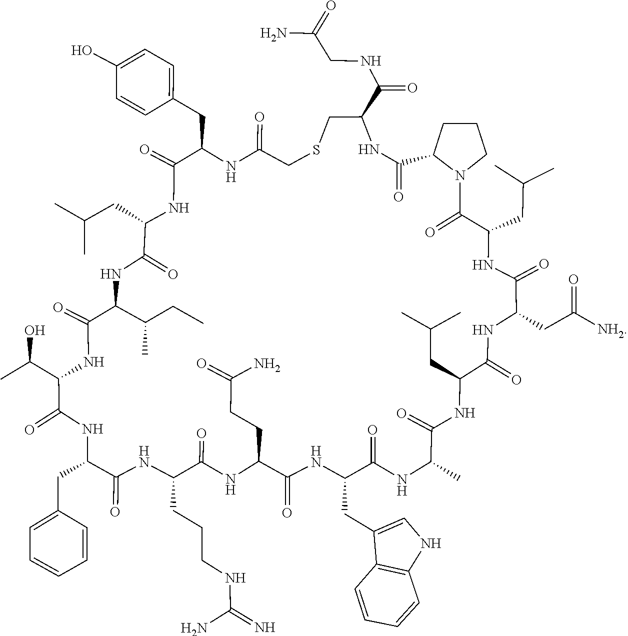 [SMILES: CC[C@H](C)[C@@H]1NC(=O)[C@H](CC(C)C)NC(=O)[C@@H](Cc2ccc(O)cc2)NC(=O)CSC[C@@H](C(=O)NCC(N)=O)NC(=O)[C@@H]2CCCN2C(=O)[C@H](CC(C)C)NC(=O)[C@H](CC(N)=O)NC(=O)[C@H](CC(C)C)NC(=O)[C@H](C)NC(=O)[C@H](Cc2c[nH]c3ccccc23)NC(=O)[C@H](CCC(N)=O)NC(=O)[C@H](CCCNC(=N)N)NC(=O)[C@H](Cc2ccccc2)NC(=O)[C@H]([C@@H](C)O)NC1=O]

42.-48. (canceled)

49. A pharmaceutical composition comprising the compound of claim 1, or a pharmaceutically acceptable salt thereof, and a pharmaceutically acceptable excipient.

50. A method of treating a cancer in a patient in need of such treatment, said method comprising administering a therapeutically effective amount of a compound of claim 1 to said patient.

51. The method of claim 50, wherein the cancer is pancreatic cancer, pituitary cancer, or bone cancer.

52. A method of treating a bone condition in a patient in need of such treatment, said method comprising administering a therapeutically effective amount of a compound of claim 1 to said patient.

53. The method of claim 52, wherein the bone condition is fibrous dysplasia.

54. (canceled)

55. A method of treating McCune-Albright syndrome in a patient in need of such treatment, said method comprising administering a therapeutically effective amount of a compound of claim 1 to said patient.

56. A method of treating cholera in a patient in need of such treatment, said method comprising administering a therapeutically effective amount of a compound of claim 1 to said patient.

57. A method of modulating the activity of a human Gαs protein, said method comprising contacting said human Gαs protein with an effective amount of a compound of claim 1.

58. (canceled)
